SEQUENCE LISTING (4 sequences)

SEQ ID NO: 1 - DNA, 23 bp, Artificial Sequence
  Description of Artificial Sequence Synthetic oligonucleotide
caguugcgcagccugaaugttcu

SEQ ID NO: 2 - DNA, 21 bp, Artificial Sequence
  Description of Artificial Sequence Synthetic oligonucleotide
caguugcgcagccugaaugtt

SEQ ID NO: 3 - DNA, 21 bp, Artificial Sequence
  Description of Artificial Sequence Synthetic oligonucleotide
cauucaggcugcgcaacugtt

SEQ ID NO: 4 - protein, 4 aa, Artificial Sequence
  Description of Artificial Sequence Synthetic peptide
DRDC

(19) United States
(12) Patent Application Publication
(10) Pub. No.: US 2012/0022245 A1
(43) Pub. Date: Jan. 26, 2012

(54) FOLATE TARGETING OF NUCLEOTIDES

(75) Inventors: Philip Stewart Low, West Lafayette, IN (US); Mini Thomas, West Lafayette, IN (US); Christopher Paul Leamon, West Lafayette, IN (US); Iontcho Radoslavov Vlahov, West Lafayette, IN (US); Paul Joseph Kleindl, Lebanon, IN (US); Longwu Qi, West Lafayette, IN (US)

(73) Assignees: PURDUE RESEARCH FOUNDATION, West Lafayette, IN (US); ENDOCYTE, INC., West Lafayette, IN (US)

(21) Appl. No.: 13/124,408

(22) PCT Filed: Oct. 16, 2009
(86) PCT No.: PCT/US09/61049

§ 371 Date: Apr. 15, 2011

Related U.S. Application Data
(60) Provisional application No. 61/106,452, filed on Oct. 17, 2008.
(60) Provisional application No. 61/187,416, filed on Jun. 16, 2009.
(60) Provisional application No. 61/196,489, filed on Oct. 17, 2008.
(60) Provisional application No. 61/196,408, filed on Oct. 17, 2008.

(51) Int. Cl. C07H 21/02; C07K 9/00; C07H 21/00

(52) U.S. Cl. 536/24.5; 536/23.1; 530/322

(57) ABSTRACT

The present invention relates to compounds, compositions, kits, and methods of use in targeting nucleotides, such as siRNA's, to cancer cells or to immune system cells involved in inflammation. More particularly, the invention is directed to receptor binding ligand-nucleotide delivery conjugates for use in specifically targeting the conjugates to cancer cells or to immune system cells, methods of treatment with these conjugates, methods of preparation of these conjugates, and methods of reducing the expression of a gene in vitro or in vivo with the conjugates described herein.

### CROSS REFERENCE TO RELATED APPLICATIONS

This application claims priority under 35 USC §119(e) to U.S. Provisional Application Ser. No. 61/106,452, filed on Oct. 17, 2008, U.S. Provisional Application Ser. No. 61/196,408 filed on Oct. 17, 2008, U.S. Provisional Application Ser. No. 61/196,489, filed on Oct. 17, 2008, and U.S. Provisional Application Ser. No. 61/187,416, filed on Jun. 16, 2009, the entire disclosure of each of which is incorporated herein by reference.

### TECHNICAL FIELD

The present invention relates to compounds, compositions, and methods for use in targeting nucleotides to cancer cells or to immune system cells. More particularly, the invention is directed to receptor binding ligand-nucleotide delivery conjugates for use in specifically targeting the conjugates to cancer cells or to immune system cells.

### BACKGROUND AND SUMMARY OF THE INVENTION

The mammalian immune system provides a means for the recognition and elimination of tumor cells, other pathogenic cells, and invading foreign pathogens. While the immune system normally provides a strong line of defense, there are many instances where cancer cells or other pathogenic cells evade a host immune response and proliferate or persist with concomitant host pathogenicity. Chemotherapeutic agents and radiation therapies have been developed to eliminate, for example, replicating neoplasms. However, many of the currently available chemotherapeutic agents and radiation therapy regimens have adverse side effects because they work not only to destroy pathogenic cells, but they also affect normal host cells, such as cells of the hematopoietic system.

Researchers have developed therapeutic protocols for destroying pathogenic cells by targeting cytotoxic compounds to such cells. Many of these protocols utilize toxins conjugated to antibodies that bind to antigens unique to or overexpressed by the pathogenic cells in an attempt to minimize delivery of the toxin to normal cells. Using this approach, certain immunotoxins have been developed consisting of antibodies directed to specific antigens on pathogenic cells, the antibodies being linked to toxins such as ricin, Pseudomonas exotoxin, Diptheria toxin, and tumor necrosis factor. These immunotoxins target pathogenic cells, such as tumor cells, bearing the specific antigens recognized by the antibody (Olsnes, S., Immunol. Today, 10, pp. 291-295, 1989; Melby, E. L., Cancer Res., 53(8), pp. 1755-1760, 1993; Better, M. D., PCT Publication Number WO 91/07418, published May 30, 1991). However, antibody conjugates are expensive to produce, and their large size and affinity for serum proteins may result in reduced delivery to the tumor. The side effects of chemotherapeutic agents and radiation, and the disadvantages of antibody conjugates highlight the need for the development of new conjugates selective for pathogenic cell populations and with reduced host toxicity.

The mammalian immune system provides a means for the recognition and elimination of foreign pathogens. Macrophages and monocytes are generally the first cells to encounter foreign pathogens, and accordingly, they play an important role in the immune response. However, activated macrophages or monocytes can contribute to the pathophysiology of disease in some instances. Activated macrophages nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response.

While the immune system normally provides a line of defense against foreign pathogens, there are many instances where the immune response itself is involved in the progression of disease. Exemplary of diseases caused or worsened by the host's own immune response are autoimmune diseases such as multiple sclerosis, lupus erythematosus, psoriasis, pulmonary fibrosis, and rheumatoid arthritis and diseases or injuries in which the immune response contributes to pathogenesis such as atherosclerosis, osteoarthritis, osteoporosis, fibromyalgia, osteomyelitis, ulcerative colitis, Sjögren's syndrome, glomerulonephritis, Crohn's disease, sarcoidosis, systemic sclerosis, head/spinal cord injuries, fatty liver disease, reperfusion injury, scleroderma, proliferative retinopathy, prosthesis osteolysis, vasculitis, obesity, gout, restenosis, graft versus host disease often resulting in organ transplant rejection, and other inflammatory diseases. Thus, there is a need for the development of new therapies that are specifically directed at immune cells, such as activated monocytes and activated macrophages, involved in the progression of inflammatory diseases.

The folate receptor (FR) is a 38 KDa GPI-anchored protein that binds the vitamin folic acid with high affinity (<1 nM). Following receptor binding, rapid endocytosis delivers the vitamin into the cell, where it is unloaded in an endosomal compartment at low pH. Importantly, covalent conjugation of small molecules to folic acid does not prevent the vitamin from binding to the folate receptor, and therefore, folate conjugates can enter cells by receptor-mediated endocytosis.

Because most cells use an unrelated reduced folate carrier (RFC) to acquire the necessary folic acid, expression of the folate receptor is restricted to a few cell types. With the exception of kidney and placenta, normal tissues express low or nondetectable levels of FR. It has recently been reported that FR-β, the nonepithelial isoform of the folate receptor, is expressed on activated (but not resting) synovial macrophages. Thus, Applicants have attempted to utilize folate-linked nucleotides, such as siRNA, to develop a method for specifically targeting these nucleotides to tumors or cells of the immune system overexpressing folate receptors and causing cancer, or inflammatory diseases, respectively.

Small interfering RNA (siRNA) is a class of short (e.g., 20 to 30 nucleotides), double stranded RNA molecules that play a variety of roles in the regulation of genes and corresponding proteins. siRNAs are well-defined double stranded RNA structures with 2-nucleotide 3′ overhangs on either end. Each siRNA strand has a 5′ phosphate group and a 3′ hydroxyl group. This structure is the result of processing by dicer, an enzyme that converts either long dsRNAs or small hairpin RNAs into siRNAs. siRNAs can also be exogenously introduced into cells by various methods to bring about the specific knockdown of a gene of interest. For example, any gene of which the sequence in known can be targeted based on sequence complementarity with an appropriately tailored siRNA molecule.

Generally, siRNA is involved in the RNA interference (RNAi) pathway where it interferes with the expression of a specific gene. These siRNAs can bind to specific RNA molecules, resulting in an increase or decrease in the expression of a specific gene. Therefore, siRNAs can be effective therapeutic agents for the treatment of multiple disease states, for example, Parkinson's disease, Lou Gehrig's disease, viral infection, including HIV infection, type 2 diabetes, obesity, hypercholesterolemia, rheumatoid arthritis, and various types of cancer.

Importantly, Applicants have shown that expression of the high affinity FR-α on tumor cells or FR-β on immune system cells can be exploited in vivo or in vitro to specifically target nucleotides, such as siRNAs, to tumors or cells of the immune system at sites of inflammation.

In one embodiment of the invention, a compound of the formula

B-L-N

is described wherein B is a vitamin receptor binding ligand that binds to a vitamin receptor, where the vitamin receptor is overexpressed or selectively expressed on a pathogenic cell, L is a linker that comprises one or more hydrophilic spacer linkers, and N is a nucleotide.

In another embodiment, a compound comprising a vitamin receptor binding ligand; a linker; and a nucleotide; wherein the vitamin receptor binding ligand is covalently attached to the linker; the nucleotide is attached to the linker; the linker comprises at least one releasable linker; and wherein the vitamin receptor is overexpressed or selectively expressed on pathogenic cells is described.

In another embodiment, a method of specifically targeting a nucleotide to pathogenic cells, the method comprising the step of administering a compound of any one of the compound embodiments described herein to an animal where the pathogenic cells overexpress or selectively expresses a vitamin receptor is described.

In another embodiment, a method is described of reducing the expression of a gene in a cell using a receptor binding ligand-nucleotide conjugate, the method comprising the step of providing the compound of any one of the compound embodiments described herein to the cell; wherein the compound binds to and is internalized into the cell; and wherein expression of the gene is reduced.

In another embodiment, a method of treating a patient harboring a population of pathogenic cells is described, the method comprising the step of administering to the patient a composition comprising a therapeutically effective amount of any one of the compounds or compositions described herein.

In another embodiment, a process for preparing the compounds described herein, the process comprising the step of forming an activated-thiol intermediate of the formula B-(L′)a-S-Lg or an activated-thiol intermediate of the formula N-(L″)a′-S-Lg;

and reacting the activated-thiol intermediate with a compound of the formula B-(L′)a-SH or N-(L″)a′-SH wherein L′ and L″ are, independently, divalent linkers through which the sulfur is linked to B and N, respectively; at least one of L′ or L″ comprises a hydrophilic linker; Lg is a leaving group; and a is 0 or 1; a′ is 0; and a+a′ is 1 or 2 is described.

In another embodiment, a process for preparing compound embodiments described herein, the method comprising the step of forming an activated-thiol intermediate of the formula vitamin receptor binding ligand-(L′″)b-S-Lg or an activated-thiol intermediate of the formula nucleotide-(L″″)b′-S-Lg;

and reacting the activated-thiol intermediate with a compound of the formula vitamin receptor binding ligand-(L′″)b-SH or nucleotide-(L″″)b′-SH wherein L′″ and L″″ are, independently, divalent linkers through which the sulfur is linked to the vitamin receptor binding ligand and the nucleotide, respectively; at least one of L′″ or L″″ comprises a releasable linker; Lg is a leaving group; and b is 0 or 1; b′ is 0; and b+b′ is 1 or 2 is described.

### BRIEF DESCRIPTION OF THE DRAWINGS

FIG. 1. Overlay of white-light and florescence images of mice obtained 24 h after retro-orbital injection of DY647-Folate β-Gal siRNA. Left mouse was injected with 7.5 nmoles of folate conjugate while the animal on the right was injected with 15 nmoles. The images indicate that the targeted conjugate exhibits tissue selectivity for the tumor (receptor-based) and for the kidney. The images indicate a positive dose response relationship between dose and signal.

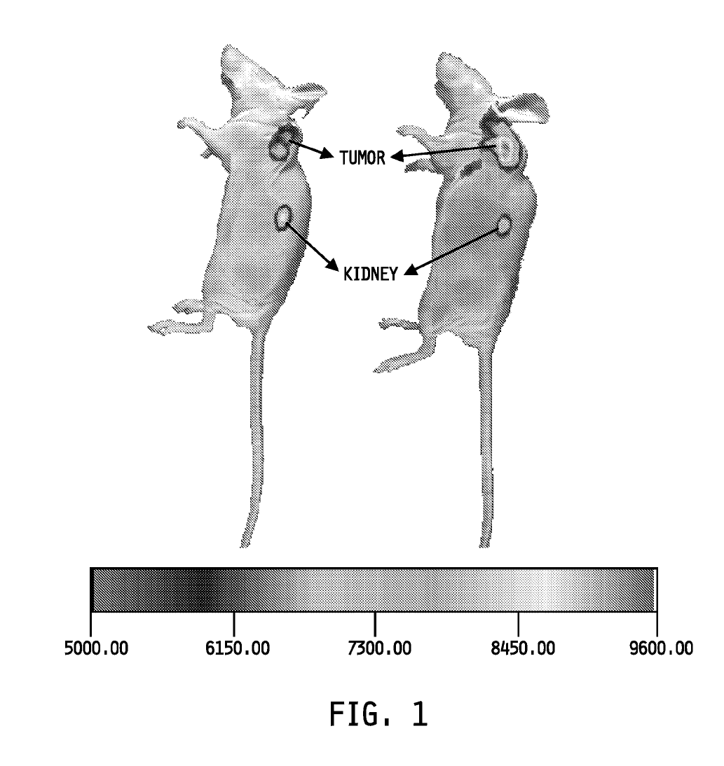

FIG. 2. Overlay of white-light and florescence images of mice obtained 24 h after retro-orbital injection of siRNA. Left mouse was injected with 15 nmoles of folate-targeted siRNA while the animal on the right was injected with 15 nmoles of non-targeted siRNA. The images indicate that the targeted conjugate exhibits a more intense signal in the tumor compared to the un-targeted conjugate.

FIG. 3. Overlay of white-light and florescence images of major organs of mice that were collected 24 h after retro-orbital injection of DY647-Folate β-Gal siRNA showing bio-distribution of folate-targeted siRNA (top, 15 nmols; bottom, 7.5 nmols). Average intensity for kidney (top panel, 15 nmols) 32486, kidney (bottom panel, 7.5 nmols) 34527, tumor (top panel, 15 nmols) 12175, tumor (bottom panel, 7.5 nmols) 9480. Tumor ratio (top/bottom) 1.3. The images indicate that the targeted conjugate exhibits tissue selectivity for the tumor (receptor-based) and for the kidney (non-specific). No appreciable signal is observed for other major organs (liver, spleen, intestine, muscle, lung, heart, blood). The images indicate a positive dose response relationship between dose and signal.

FIG. 4. Comparison of uptake of siRNA by tumor with that by major organs excluding kidney (overlay of white-light and florescence images). Average intensity for 34527, tumor (top panel, 15 nmols) 12175, tumor (bottom panel, 7.5 nmols) 9480. Tumor ratio (top/bottom) 1.3. The images indicate that the targeted conjugate exhibits tissue selectivity for the tumor (receptor-based). No appreciable signal is observed for other major organs (liver, spleen, intestine, muscle, lung, heart, blood). The images indicate a positive dose response relationship between dose and signal.

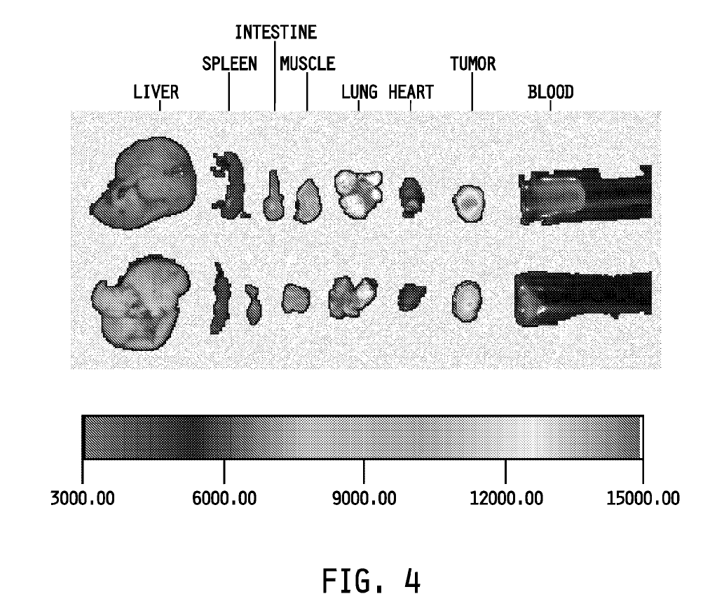

FIG. 5. Comparison of bio-distribution of targeted (top) vs non-targeted (bottom) siRNA (overlay of white-light and florescence images). Average intensity for kidney (top panel, targeted) 32486, kidney (bottom panel, un-targeted) 13902, tumor (top panel, targeted) 12175, tumor (bottom panel, un-targeted) 3787. Tumor ratio (targeted/un-targeted) 3.2. The images indicate that the targeted conjugate exhibits tissue selectivity for the tumor (receptor-based) and for the kidney (non-specific) with a more intense signal than the un-targeted conjugate. No appreciable signal is observed for other major organs (liver, spleen, intestine, muscle, lung, heart, blood).

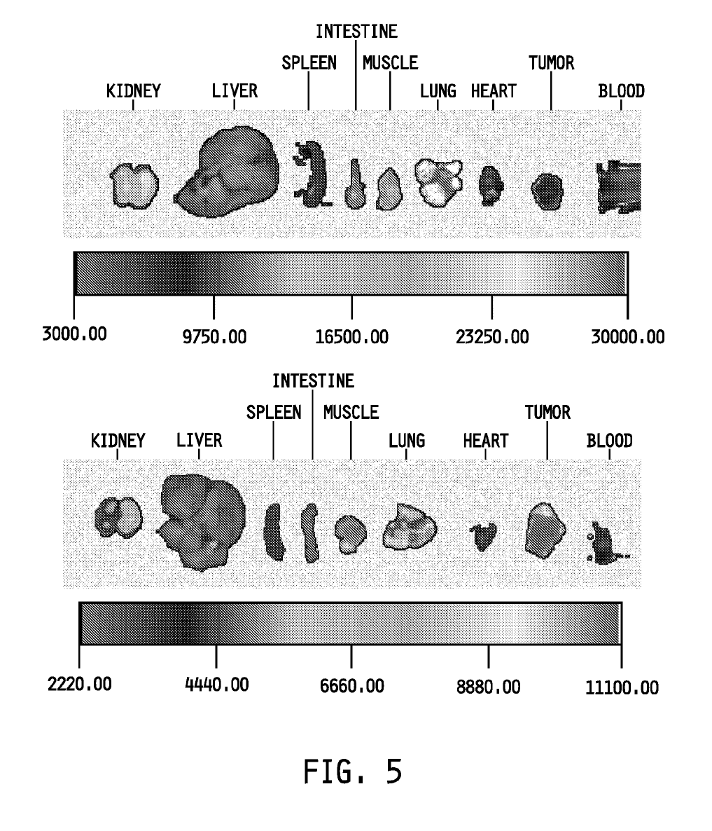

FIG. 6. Targeted vs non-targeted siRNA: Comparison of uptake by tumor with that by major organs excluding kidney (overlay of white-light and florescence images). Average intensity for tumor (top panel, targeted) 12175, tumor (bottom panel, un-targeted) 3787. Tumor ratio (targeted/un-targeted) 3.2. The images indicate that the targeted conjugate exhibits tissue selectivity for the tumor (receptor-based) with a more intense signal than the un-targeted conjugate. No appreciable signal is observed for other major organs (liver, spleen, intestine, muscle, lung, heart, blood).

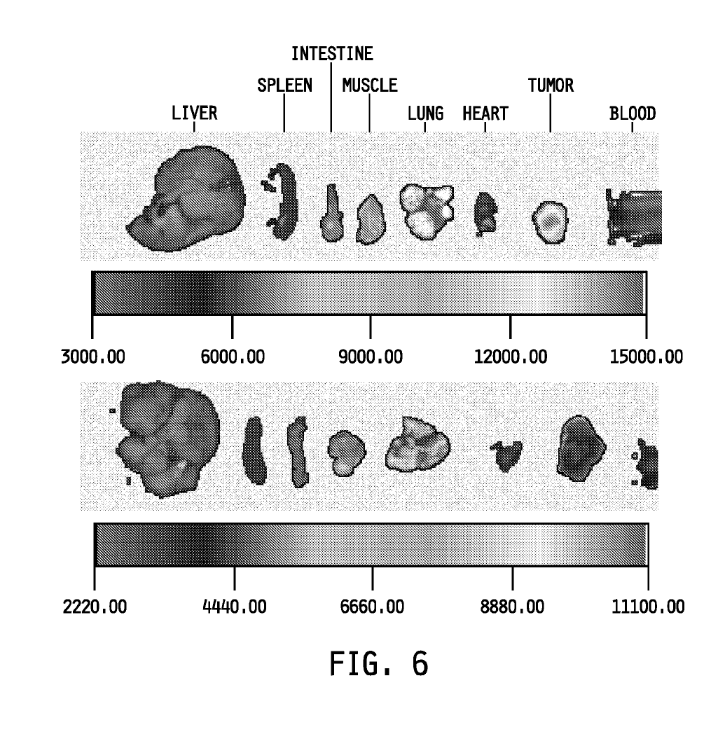

FIG. 7. Uptake of folate-siRNA-Cy3-cholesterol 4 hours later: 60 nmoles of folate-siRNA-Cy3-Cholesterol were injected into the tail vein of an athymic nu/nu mice containing a KB cell tumor on the left shoulder. The mouse was imaged four hours later on a Kodak Imaging Station.

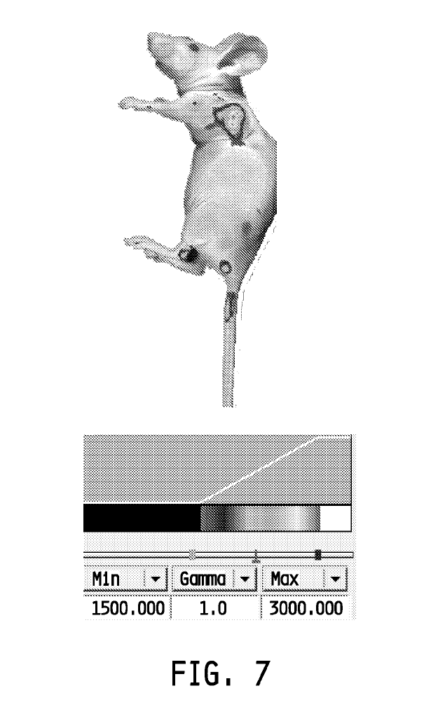

FIG. 8. Uptake of folate-siRNA-Cy3-cholesterol four days later: 120 nmoles of folate-siRNA-Cy3-Cholesterol were injected into the tail vein of an athymic nu/nu mice containing a KB cell tumor on the left shoulder. The mouse was imaged four days later on a Kodak Imaging Station.

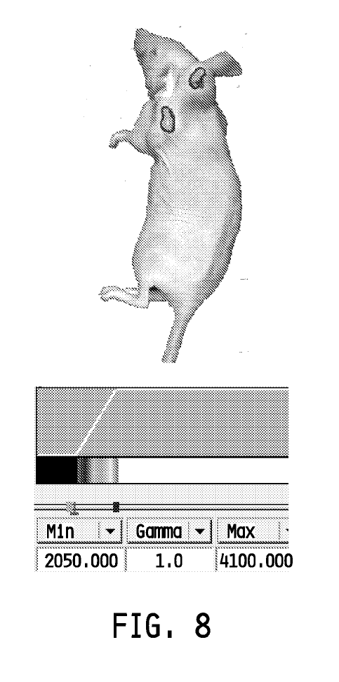

FIG. 9. Folate-targeted delivery of siRNAs to cancer cells in vitro. FR-expressing RAW264.7 cells were incubated with 400 nM DY647-labeled folate-targeted siRNA (A), Cy5-labeled folate-targeted 21-mer oligonucleotide duplex (B), or the control non-targeted Cy5-labeled oligonucleotide duplex (C) for 1 h (A) or 2 h (B & C) at 37° C., then washed 3×, and imaged using a confocal scanning fluorescence microscope. The bottom panel in each case (D-F) corresponds to the transmission image of the same cells.

FIG. 10. Accumulation of folate-targeted siRNAs in endosomes of GFP-tubulin transfected HeLa cells. FR expressing GFP-tubulin HeLa cells were treated with fluorescently labeled folate targeted siRNA, washed, and then imaged alive using a confocal scanning fluorescence microscope. Cells were monitored for far-red fluorescence from siRNA (A) and green fluorescence from GFP (B). Overlay of A and B is presented in panel C. In color images the cytoskeleton appears green and endosomes containing siRNA appear red.

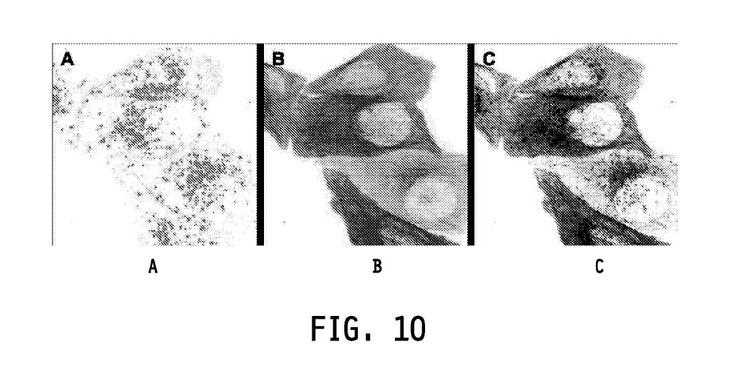

FIG. 11. β-Gal siRNA-Folate conjugate used for in vivo targeting and fluorescent imaging.

FIG. 12. siRNA targeting in mouse to atherosclerotic plaque in model of atherosclerosis (ApoE−/−). Left: Mice were injected retroorbitally with 15 nmoles of Fol-b-Gal-Dy647 siRNA and imaged 4 h later. Right: The aortic arch was removed from the same animal and imaged.

FIG. 13. siRNA uptake in mouse skeletal muscle injury model. Tibialis anterior muscles of C57/BL6 mice were injected with cardiotoxin from Naja naja mossambica, and 48 h later, 15 nmols of Fol-b-Gal-siRNA-Dy647 was injected retro-orbitally. Fluorescence images were acquired 4 h post injection of siRNA.

FIG. 14. Imaging of Folate-DY647-b-Gal siRNA in guinea pig osteoarthritis model. Two year old male guinea pig was injected i.p with 15 nmoles of siRNA. The images were taken 4 h post injection.

FIG. 15. Images of excised stifle joints.

### DETAILED DESCRIPTION OF ILLUSTRATIVE EMBODIMENTS

The present invention relates to compounds, compositions, and methods for use in targeting nucleotides to pathogenic cells (e.g., cancer cells or immune system cells involved in inflammation). Methods of treating cancer or inflammation with the compounds and compositions described herein are also provided. Also provided are methods of preparing the compounds and compositions described herein. More particularly, the invention is directed to receptor binding ligand-nucleotide delivery conjugates for use in specifically targeting the conjugates to pathogenic cells, such as cancer cells or immune system cells involved in inflammation.

In one embodiment, the pathogenic cells that are specifically targeted using the receptor binding ligand-nucleotide conjugates of the invention are cancer cells. In various embodiments, the population of pathogenic cells may be a cancer cell population that is tumorigenic, including benign tumors and malignant tumors, or it can be non-tumorigenic. The cancer cell population may arise spontaneously or by such processes as mutations present in the germline of the host animal or somatic mutations, or it may be chemically-, virally-, or radiation-induced. In illustrative embodiments, cancers that can be targeted are carcinomas, sarcomas, lymphomas, Hodgekin's disease, melanomas, mesotheliomas, Burkitt's lymphoma, nasopharyngeal carcinomas, leukemias, or myelomas. Illustratively, the cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, and lung cancers.

In another embodiment, the pathogenic cells that are specifically targeted are immune system cells involved in inflammation (e.g., activated monocytes and/or activated macrophages). In these embodiments, the immune response itself may be involved in the progression of the inflammation. Exemplary of inflammatory diseases and injuries in which the immune response contributes to pathogenesis and which can be treated in accordance with the invention are multiple sclerosis, lupus erythematosus, psoriasis, pulmonary fibrosis, rheumatoid arthritis, atherosclerosis, osteoarthritis, osteoporosis, fibromyalgia, osteomyelitis, ulcerative colitis, Sjögren's syndrome, glomerulonephritis, Crohn's disease, sarcoidosis, systemic sclerosis, head/spinal cord injuries, fatty liver disease, reperfusion injury, scleroderma, proliferative retinopathy, prosthesis osteolysis, vasculitis, obesity, gout, restenosis, graft versus host disease often resulting in organ transplant rejection, and other inflammatory diseases.

In accordance with the invention, the phrases “specifically targeting”, “specific targeting”, and “specifically targeted” mean that the receptor binding ligand-nucleotide delivery conjugates described herein are preferentially targeted to cell types (e.g., tumor cells and activated immune cells involved in inflammatory disease or injuries) that overexpress a ligand receptor, such as the folate receptor, as evidenced by the ability to detect accumulation of the receptor binding ligand-nucleotide delivery conjugates in the specifically targeted cell type (e.g., tumor cells and activated immune cells involved in inflammatory diseases) over accumulation in normal tissues that do not overexpress the receptor for the ligand (e.g., the folate receptor). The phrases “specifically targeting”, “specific targeting”, and “specifically targeted” do not preclude the detectable targeting of the receptor binding ligand-nucleotide delivery conjugates described herein to normal tissues, such as the kidney and placenta, that overexpress a ligand receptor, such as the folate receptor.

As used herein, the term “nucleotide” (N) includes an oligonucleotide, an iRNA, an siRNA, a microRNA, a ribozyme, an antisense molecule, or analogs or derivatives thereof. The nucleotide N can be RNA or DNA, or combinations thereof, and can be single or double-stranded. If the nucleotide N is double-stranded, the nucleotide N contains a sense strand and an antisense strand. If the nucleotide N is single-stranded, the strand is preferably an antisense strand. Typically, the nucleotide strands, if the nucleotide is double-stranded, are two separate molecules rather than two separate sequences on the same nucleotide strand. The receptor binding ligand can be coupled to the sense strand or the antisense strand, or both.

In one embodiment, each strand of the nucleotide N includes about 15 to about 49 bases. In another embodiment, each strand of the nucleotide N includes about 19 to about 25 bases. In another embodiment, each strand of the nucleotide N includes about 15 to about 23 bases. In another embodiment, each strand of the nucleotide N includes about 21 to about 23 bases. In another embodiment, each strand of the nucleotide N includes about 21 to about 23 bases, with a duplex region of about 15 to about 23 base pairs. In another embodiment, the nucleotide N includes a single-stranded overhang at the 5′ and/or the 3′ end including about 2 to about 3 bases. Preferably, the single-stranded overhang is a 3′ overhang including about 2 to about 3 bases. In another embodiment, the nucleotide N is blunt-ended at least one end of the nucleotide. In another embodiment, the nucleotide N is a small interfering RNA, also referred to as siRNA.

In each of the forgoing, it is to be understood that nucleotide N may include not only natural bases, such as A, C, T, U, and G, but also may contain non-natural analogs and derivatives of such bases. For example, bases or analogs and derivatives of bases that may further stabilize the nucleotide against degradation (e.g., make the nucleotide nuclease resistant) or metabolism can be used. In another embodiment, other derivatives of the nucleotide N may be used, including 2′-F or 2′-OMe sugar modifications, 5-alkylamino or 5-allylamino base modifications, or other derivatives of naturally occurring bases, or phosphorothioate, P-alkyl, phosphonate, phosphoroselenate, or phosphoroamidate modifications of the nucleotide backbone or modifications of the backbone or a terminal phosphate with these or other phosphate analogs, or combinations thereof. The modifications can be made at any position in the nucleotide N, and can be any of the modifications described, for example, in WO 2009/082606, incorporated herein by reference. Methods of modifying nucleotides to stabilize nucleotides are well-known in the art. The nucleotide N described herein can be synthesized by methods well-known in the art such as those described in Trufert et al., Tetrahedron, 52:3005 (1996), Martin, Helv. Chim. Acta, 78, 486-504 (1995), or WO 2009/082606, each incorporated herein by reference.

In various illustrative embodiments, any nucleotide N (e.g., siRNA) that is complementary to the specific target gene of interest can be attached to a binding ligand as herein described. Exemplary of nucleotides N that can be used to target specific genes of interest to alter gene expression are described in International Publication No. WO 2009/082606, U.S. Pat. Nos. 7,517,846 and 7,022,828, and U.S. Publication Nos. 20090253774, 20090253773, 20090253772, 20090247613, 20090247606, 20090233983, 20090192105, 20090192104, 20090156533, 20090143325, 20090143324, 20090137513, 20090137512, 2009137511, 20090137510, 20090137509, 20090137508, 20090137507, 20090105178, 20090099119, 20090099117, 20090099116, 20090099115, 20090093437, 20090093436, 20090093435, and 20090023676, each incorporated herein by reference.

The receptor binding ligand nucleotide delivery conjugates described herein can be formed from, for example, a wide variety of vitamins or receptor-binding vitamin analogs/derivatives, linkers, and nucleotides N. The binding ligand nucleotide delivery conjugates described herein are capable of specifically targeting a population of pathogenic cells in the host animal due to preferential expression of a receptor for the binding ligand, such as a vitamin, accessible for ligand binding, on the pathogenic cells. Illustrative vitamin moieties that can be used as the receptor binding ligand (B) include carnitine, inositol, lipoic acid, pyridoxal, ascorbic acid, niacin, pantothenic acid, folic acid, riboflavin, thiamine, biotin, vitamin B₁₂, and the lipid soluble vitamins A, D, E and K. These vitamins, and their receptor-binding analogs and derivatives, constitute an illustrative targeting entity that can be coupled with the nucleotide by a bivalent linker (L) to form a binding ligand (B) nucleotide delivery conjugate as described herein. The term vitamin is understood to include vitamin analogs and/or derivatives, unless otherwise indicated. Illustratively, pteroic acid which is a derivative of folate, biotin analogs such as biocytin, biotin sulfoxide, oxybiotin and other biotin receptor-binding compounds, and the like, are considered to be vitamins, vitamin analogs, and vitamin derivatives. In one embodiment, vitamins that can be used as the binding ligand (B) in the binding ligand nucleotide delivery conjugates described herein include those that bind to vitamin receptors expressed specifically on activated macrophages or activated monocytes or on cancer cells, such as the folate receptor, which binds folate, or an analog or derivative thereof as described herein.

In addition to the vitamins described herein, it is appreciated that other binding ligands may be coupled with the nucleotides and linkers described and contemplated herein to form binding ligand nucleotide delivery conjugates capable of facilitating delivery of the nucleotide to a desired target. These other binding ligands, in addition to the vitamins and their analogs and derivatives described, may be used to form binding ligand nucleotide delivery conjugates capable of binding to target cells. In general, any binding ligand (B) of a overexpressed or preferentially expressed cell surface receptor may be advantageously used as a targeting ligand to which a linker nucleotide conjugate can be attached.

The binding ligand (B) nucleotide delivery conjugates can be used to target a pathogenic cell population in the host animal wherein the members of the pathogenic cell population have an accessible binding site for the binding ligand (B), or analog or derivative thereof, wherein the binding site is uniquely expressed, overexpressed, or preferentially expressed by the pathogenic cells (e.g., cancer cells or cells of the immune system involved in inflammation). The specific targeting of the pathogenic cells is mediated by the binding of the ligand moiety of the binding ligand (B) nucleotide delivery conjugate to a ligand receptor, transporter, or other surface-presented protein that specifically binds the binding ligand (B), or analog or derivative thereof, and which is uniquely expressed, overexpressed, or preferentially expressed by the pathogenic cells (e.g., cancer cells or immune cells involved in inflammation).

A surface-presented protein uniquely expressed, overexpressed, or preferentially expressed by the pathogenic cells is a receptor not present or present at lower concentrations on non-pathogenic cells providing a means for specific targeting of the pathogenic cells. For example, surface-expressed vitamin receptors, such as the high-affinity folate receptor, are overexpressed on cancer cells. Epithelial cancers of the ovary, mammary gland, colon, lung, nose, throat, and brain have all been reported to express elevated levels of the folate receptor. In fact, greater than 90% of all human ovarian tumors are known to express large amounts of this receptor. Accordingly, the binding ligand nucleotide delivery conjugates described herein can be used to target a variety of tumor cell types, as described herein, as well as other types of pathogenic cells, that preferentially express vitamin receptors, and, thus, have surface accessible binding sites for ligands, such as vitamins or vitamin analogs or derivatives.

The binding ligand (B) nucleotide delivery conjugates described herein can be used for both human (e.g., a human patient) and veterinary applications. Thus, the host animal harboring the population of pathogenic cells and targeted with the binding ligand nucleotide delivery conjugates, such as a vitamin nucleotide delivery conjugate, can be human or, in the case of veterinary applications, can be a laboratory, agricultural, domestic, or wild animal. The compositions, compounds, and methods described herein can be applied to host animals including, but not limited to, humans, laboratory animals such as rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

In one embodiment, the binding ligand nucleotide delivery conjugates can be internalized into the targeted pathogenic cells upon binding of the binding ligand moiety to a receptor, transporter, or other surface-presented protein that specifically binds the ligand and which is preferentially expressed on the pathogenic cells. Such internalization can occur, for example, through receptor-mediated endocytosis. If the binding ligand (B) nucleotide delivery conjugate contains a releasable linker, the binding ligand moiety and the nucleotide can dissociate intracellularly and the nucleotide can act on its intracellular target.

In one embodiment, the nucleotide could be released by a protein disulfide isomerase inside the cell where the releasable linker is a disulfide group. The nucleotide may also be released by a hydrolytic mechanism, such as acid-catalyzed hydrolysis, as described herein for certain beta elimination mechanisms, or by an anchimerically assisted cleavage through an oxonium ion or lactonium ion producing mechanism. The selection of the releasable linker or linkers will dictate the mechanism by which the nucleotide is released from the conjugate. It is appreciated that such a selection can be pre-defined by the conditions wherein the nucleotide conjugate will be used. Alternatively, the nucleotide delivery conjugates can be internalized into the targeted cells upon binding, and the binding ligand and the nucleotide can remain associated intracellularly with the nucleotide exhibiting its effects without dissociation from the ligand, such as a vitamin moiety.

In one embodiment, the nucleotides for use in the methods described herein remain stable in serum for at least 4 hours. In another embodiment the nucleotides have an IC₅₀ in the nanomolar range, and, in another embodiment, the nucleotides are water soluble. If the nucleotide is not water soluble, the bivalent linkers (L) described herein can be derivatized to enhance water solubility. Nucleotide analogs or derivatives can also be used, such as methylated bases to enhance stability of the nucleotide.

Additionally, more than one type of binding ligand nucleotide delivery conjugate can be used. Illustratively, for example, cells of the host animal can be targeted with conjugates with different vitamins, but the same nucleotide. In other embodiments, the host animal cells can be targeted with conjugates comprising the same binding ligand linked to different nucleotides, or various binding ligands linked to various nucleotides. In another illustrative embodiment, binding ligand nucleotide delivery conjugates with the same or different vitamins, and the same or different nucleotides comprising multiple vitamins and multiple nucleotides as part of the same nucleotide delivery conjugate can be used.

In one embodiment, a method of treating a patient harboring a population of pathogenic cells is provided. The method comprises the step of administering to the patient a composition comprising a therapeutically effective amount of any of the binding ligand nucleotide delivery conjugates described herein. In another illustrative embodiment, a method of specifically targeting a nucleotide to pathogenic cells in a host animal is provided. The method comprises the step of administering any of the binding ligand nucleotide delivery conjugates described herein to the animal where the pathogenic cells overexpress or selectively expresses a a receptor for the ligand.

In another illustrative embodiment, a method is provided of reducing the expression of a gene in a cell using a receptor binding ligand nucleotide delivery conjugate. The method comprises the step of providing the receptor binding ligand nucleotide delivery conjugate of the invention to the cell wherein the conjugate binds to and is internalized into the cell, and wherein expression of the gene is reduced. In one embodiment, the reduction in expression of the gene is complete and in another embodiment, the reduction in expression of the gene is partial. In this embodiment of the invention, gene expression can be reduced in vitro, such as in a cell type (e.g., primary cells) or a cell line (e.g., a transformed cell line) or in vivo, such as in an animal or a human or in a tissue. In one illustrative embodiment, the reduction of expression occurs in vitro and the reduction in expression occurs in a cell that has been genetically modified using molecular biology techniques. Such techniques are described in Sambrook et al., “Molecular Cloning: A Laboratory Manual”, 3rd Edition, Cold Spring Harbor Laboratory Press, (2001), incorporated herein by reference. In one embodiment, the reduction in gene expression that occurs in vitro or in vivo can be reduction in expression of a reporter gene, such as β-galactosidase, green fluorescent protein, or luciferase.

In still another embodiment, a process for preparing the compounds described herein is provided. The process comprises the step of forming an activated-thiol intermediate of the formula B-(L′)a-S-Lg or an activated-thiol intermediate of the formula N-(L″)a′-S-Lg and reacting the activated-thiol intermediate with a compound of the formula B-(L′)a-SH or N-(L″)a′-SH wherein L′ and L″ are, independently, divalent linkers through which the sulfur is linked to B and N, respectively, at least one of L′ or L″ comprises a hydrophilic linker, Lg is a leaving group, and a is 0 or 1, a′ is 0, and a+a′ is 1 or 2.

In another embodiment, a process for preparing the compounds described herein is provided. The method comprises the step of forming an activated-thiol intermediate of the formula vitamin receptor binding ligand-(L′″)b-S-Lg or an activated-thiol intermediate of the formula nucleotide-(L″″)b′-S-Lg, and reacting the activated-thiol intermediate with a compound of the formula vitamin receptor binding ligand-(L′″)b-SH or nucleotide-(L″″)b′-SH wherein L′″ and L″″ are, independently, divalent linkers through which the sulfur is linked to the vitamin receptor binding ligand and the nucleotide, respectively, Lg is a leaving group, and b is 0 or 1, b′ is 0, and b+b′ is 1 or 2.

In yet another embodiment, a kit is provided. The kit can comprise a container, a composition comprising any of the binding ligand nucleotide delivery conjugates described herein, a sterile package containing the composition, and instructions for use.

### Conjugates Including Releasable Linkers and Spacer Linkers

Receptor binding ligand-nucleotide delivery conjugates are described herein consisting of a binding ligand (B), a bivalent linker (L), and a nucleotide (N), such as an oligonucleotide, an siRNA, an antisense molecule, a microRNA, or a ribozyme, an iRNA, or an analog or derivative thereof. The nucleotide can be an RNA or a DNA molecule. As used herein, the term “nucleotide” means an oligonucleotide, an siRNA, an antisense molecule, an iRNA, a microRNA, or a ribozyme, or an analog or derivative thereof. As used herein, the term “siRNA” means an siRNA or an analog or derivative thereof.

The receptor binding ligand (B) is covalently attached to the bivalent linker (L), and the nucleotide (N), is also covalently attached to the bivalent linker (L). The bivalent linker (L) comprises one or more spacer linkers and/or releasable linkers, and combinations thereof, in any order. In one variation, releasable linkers, and optional spacer linkers are covalently bonded to each other to form the linker. In another variation, a releasable linker is directly attached to the nucleotide, or analog or derivative thereof. In another variation, a releasable linker is directly attached to the receptor binding ligand. In another variation, either or both the receptor binding ligand and the nucleotide, or analog or derivative thereof, is attached to a releasable linker through one or more spacer linkers. In another variation, each of the receptor binding ligand and the nucleotide, or analog or derivative thereof, is attached to a releasable linker, each of which may be directly attached to each other, or covalently attached through one or more spacer linkers. From the foregoing, it should be appreciated that the arrangement of the receptor binding ligand, and the nucleotide, or analog or derivative thereof, and the various releasable and optional spacer linkers may be varied widely. In one aspect, the receptor binding ligand, and the nucleotide, or analog or derivative thereof, and the various releasable and optional spacer linkers are attached to each other through heteroatoms, such as nitrogen, oxygen, sulfur, phosphorus, silicon, and the like. In variations, the heteroatoms, excluding oxygen, may be in various states of oxidation, such as N(OH), S(O), S(O)₂, P(O), P(O)₂, P(O)₃, and the like. In another variation, the heteroatoms may be grouped to form divalent radicals, such as for example hydroxylamines, hydrazines, hydrazones, sulfonates, phosphinates, phosphonates, and the like.

In one aspect, the receptor binding ligand (B) is a vitamin, or analog or derivative thereof, or another vitamin receptor binding compound.

In another embodiment, the bivalent linker (L) is a chain of atoms selected from C, N, O, S, Si, and P that covalently connects the binding ligand (B) to the nucleotide (N). The linker may have a wide variety of lengths, such as in the range from about 2 to about 100 atoms. The atoms used in forming the linker may be combined in all chemically relevant ways, such as chains of carbon atoms forming alkylene, alkenylene, and alkynylene groups, and the like; chains of carbon and oxygen atoms forming ethers, polyoxyalkylene groups, or when combined with carbonyl groups forming esters and carbonates, and the like; chains of carbon and nitrogen atoms forming amines, imines, polyamines, hydrazines, hydrazones, or when combined with carbonyl groups forming amides, ureas, semicarbazides, carbazides, and the like; chains of carbon, nitrogen, and oxygen atoms forming alkoxyamines, alkoxylamines, or when combined with carbonyl groups forming urethanes, amino acids, acyloxylamines, hydroxamic acids, and the like; and others. In addition, it is to be understood that the atoms forming the chain in each of the foregoing illustrative embodiments may be either saturated or unsaturated, such that for example, alkanes, alkenes, alkynes, imines, and the like may be radicals that are included in the linker. In addition, it is to be understood that the atoms forming the linker may also be cyclized upon each other to form divalent cyclic structures that form the linker, including cyclo alkanes, cyclic ethers, cyclic amines, arylenes, heteroarylenes, and the like in the linker.

In another embodiment, the linker includes radicals that form at least one releasable linker, and optionally one or more spacer linkers. As used herein, the term releasable linker refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis reaction, for example, at physiological pH, or as a result of compartmentalization into a cellular organelle such as an endosome having a lower pH than cytosolic pH.

It is understood that a cleavable bond can connect two adjacent atoms within the releasable linker and/or connect other linkers or B and/or N, as described herein, at either or both ends of the releasable linker. In the case where a cleavable bond connects two adjacent atoms within the releasable linker, following breakage of the bond, the releasable linker is broken into two or more fragments. Alternatively, in the case where a cleavable bond is between the releasable linker and another moiety, such as an additional heteroatom, a spacer linker, another releasable linker, the nucleotide, or analog or derivative thereof, or the binding ligand, following breakage of the bond, the releasable linker is separated from the other moiety. Accordingly, it is also understood that each of the spacer and releasable linkers are polyvalent, such as bivalent.

Illustrative releasable linkers include methylene, 1-alkoxyalkylene, 1-alkoxycycloalkylene, 1-alkoxyalkylenecarbonyl, 1-alkoxycycloalkylenecarbonyl, carbonylarylcarbonyl, carbonyl(carboxyaryl)carbonyl, carbonyl(biscarboxyaryl)carbonyl, haloalkylenecarbonyl, alkylene(dialkylsilyl), alkylene(alkylarylsilyl), alkylene(diarylsilyl), (dialkylsilyl)aryl, (alkylarylsilyl)aryl, (diarylsilyl)aryl, oxycarbonyloxy, oxycarbonyloxyalkyl, sulfonyloxy, oxysulfonylalkyl, iminoalkylidenyl, carbonylalkylideniminyl, iminocycloalkylidenyl, carbonylcycloalkylideniminyl, alkylenethio, alkylenearylthio, and carbonylalkylthio, wherein each of the releasable linkers is optionally substituted with a substituent X², as defined below.

In the preceding embodiment, the releasable linker may include oxygen, and the releasable linkers can be methylene, 1-alkoxyalkylene, 1-alkoxycycloalkylene, 1-alkoxyalkylenecarbonyl, and 1-alkoxycycloalkylenecarbonyl, wherein each of the releasable linkers is optionally substituted with a substituent X², as defined below, and the releasable linker is bonded to the oxygen to form an acetal or ketal. Alternatively, the releasable linker may include oxygen, and the releasable linker can be methylene, wherein the methylene is substituted with an optionally-substituted aryl, and the releasable linker is bonded to the oxygen to form an acetal or ketal. Further, the releasable linker may include oxygen, and the releasable linker can be sulfonylalkyl, and the releasable linker is bonded to the oxygen to form an alkylsulfonate.

In another embodiment of the above releasable linker embodiment, the releasable linker may include nitrogen, and the releasable linkers can be iminoalkylidenyl, carbonylalkylideniminyl, iminocycloalkylidenyl, and carbonylcycloalkylideniminyl, wherein each of the releasable linkers is optionally substituted with a substituent X², as defined below, and the releasable linker is bonded to the nitrogen to form an hydrazone. In an alternate configuration, the hydrazone may be acylated with a carboxylic acid derivative, an orthoformate derivative, or a carbamoyl derivative to form various acylhydrazone releasable linkers.

Alternatively, the releasable linker may include oxygen, and the releasable linkers can be alkylene(dialkylsilyl), alkylene(alkylarylsilyl), alkylene(diarylsilyl), (dialkylsilyl)aryl, (alkylarylsilyl)aryl, and (diarylsilyl)aryl, wherein each of the releasable linkers is optionally substituted with a substituent X², as defined below, and the releasable linker is bonded to the oxygen to form a silanol. In another variation, the nucleotide can include an oxygen atom, and the releasable linker can be haloalkylenecarbonyl, optionally substituted with a substituent X², and the releasable linker is bonded to the nucleotide oxygen to form an ester.

In the above releasable linker embodiment, the nucleotide can include a nitrogen atom, the releasable linker may include nitrogen, and the releasable linkers can be carbonylarylcarbonyl, carbonyl(carboxyaryl)carbonyl, carbonyl(biscarboxyaryl)carbonyl, and the releasable linker can be bonded to the heteroatom nitrogen to form an amide, and also bonded to the nucleotide nitrogen to form an amide. In one variation, the nucleotide can include a nitrogen atom, and the releasable linker can be haloalkylenecarbonyl, optionally substituted with a substituent X², and the releasable linker is bonded to the nucleotide nitrogen to form an amide. In another variation, the nucleotide can include a double-bonded nitrogen atom, and in this embodiment, the releasable linkers can be alkylenecarbonylamino and 1-(alkylenecarbonylamino)succinimid-3-yl, and the releasable linker can be bonded to the nucleotide nitrogen to form an hydrazone.

In another variation, the nucleotide can include a sulfur atom, and in this embodiment, the releasable linkers can be alkylenethio and carbonylalkylthio, and the releasable linker can be bonded to the nucleotide sulfur to form a disulfide. Alternatively, the nucleotide can include an oxygen atom, the releasable linker may include nitrogen, and the releasable linkers can be carbonylarylcarbonyl, carbonyl(carboxyaryl)carbonyl, carbonyl(biscarboxyaryl)carbonyl, and the releasable linker can form an amide, and also bonded to the nucleotide oxygen to form an ester.

The substituents X² can be alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides. In this embodiment the releasable linker can include nitrogen, and the substituent X² and the releasable linker can form an heterocycle.

The heterocycles can be pyrrolidines, piperidines, oxazolidines, isoxazolidines, thiazolidines, isothiazolidines, pyrrolidinones, piperidinones, oxazolidinones, isoxazolidinones, thiazolidinones, isothiazolidinones, and succinimides.

In another embodiment, the bivalent linker (L) includes a disulfide releasable linker. In another embodiment, the bivalent linker (L) includes at least one releasable linker that is not a disulfide releasable linker.

In one aspect, the releasable and spacer linkers may be arranged in such a way that subsequent to the cleavage of a bond in the bivalent linker, released functional groups chemically assist the breakage or cleavage of additional bonds, also termed anchimeric assisted cleavage or breakage. An illustrative embodiment of such a bivalent linker or portion thereof includes compounds having the formulae:

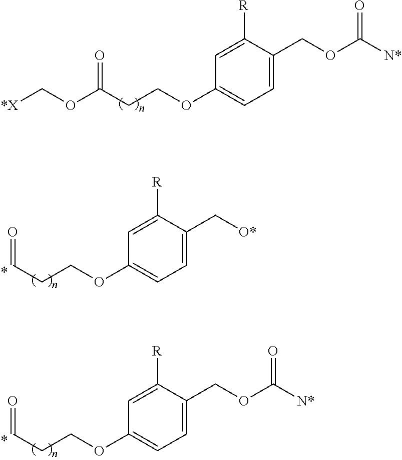

where X is an heteroatom, such as nitrogen, oxygen, or sulfur, or a carbonyl group; n is an integer selected from 0 to 4; illustratively 2; R is hydrogen, or a substituent, including a substituent capable of stabilizing a positive charge inductively or by resonance on the aryl ring, such as alkoxy and the like, including methoxy; and the symbol (*) indicates points of attachment for additional spacer, heteroatom, or releasable linkers forming the bivalent linker, or alternatively for attachment of the nucleotide, or analog or derivative thereof, or the vitamin, or analog or derivative thereof. In one embodiment, n is 2 and R is methoxy. It is appreciated that other substituents may be present on the aryl ring, the benzyl carbon, the alkanoic acid, or the methylene bridge, including but not limited to hydroxy, alkyl, alkoxy, alkylthio, halo, and the like. Assisted cleavage may include mechanisms involving benzylium intermediates, benzyne intermediates, lactone cyclization, oxonium intermediates, beta-elimination, and the like. It is further appreciated that, in addition to fragmentation subsequent to cleavage of the releasable linker, the initial cleavage of the releasable linker may be facilitated by an anchimeric ally assisted mechanism.

Illustrative examples of intermediates useful in forming such linkers include:

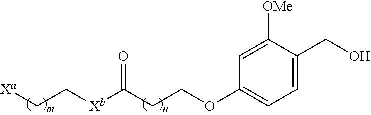

where X^{a} is an electrophilic group such as maleimide, vinyl sulfone, activated carboxylic acid derivatives, and the like, X^{b} is NH, O, or S; and m and n are each independently selected integers from 0-4. In one variation, m and n are each independently selected integers from 0-2. Such intermediates may be coupled to nucleotides, receptor binding ligands, or other linkers via nucleophilic attack onto electrophilic group X^{a}, and/or by forming ethers or carboxylic acid derivatives of the benzylic hydroxyl group. In one embodiment, the benzylic hydroxyl group is converted into the corresponding activated benzyloxycarbonyl compound with phosgene or a phosgene equivalent. This embodiment may be coupled to nucleotides, receptor binding ligands, or other linkers via nucleophilic attack onto the activated carbonyl group.

The releasable linker includes at least one bond that can be broken or cleaved under physiological conditions (e.g., a pH-labile, acid-labile, oxidatively-labile, or enzyme-labile bond). The cleavable bond or bonds may be present in the interior of a cleavable linker and/or at one or both ends of a cleavable linker. It is appreciated that the lability of the cleavable bond may be adjusted by including functional groups or fragments within the bivalent linker L that are able to assist or facilitate such bond breakage, also termed anchimeric assistance. In addition, it is appreciated that additional functional groups or fragments may be included within the bivalent linker L that are able to assist or facilitate additional fragmentation of the receptor binding nucleotide conjugates after bond breaking of the releasable linker.

The lability of the cleavable bond can be adjusted by, for example, substitutional changes at or near the cleavable bond, such as including alpha branching adjacent to a cleavable disulfide bond, increasing the hydrophobicity of substituents on silicon in a moiety having a silicon-oxygen bond that may be hydrolyzed, homologating alkoxy groups that form part of a ketal or acetal that may be hydrolyzed, and the like.

Illustrative mechanisms for cleavage of the bivalant linkers described herein include the following 1,4 and 1,6 fragmentation mechanisms

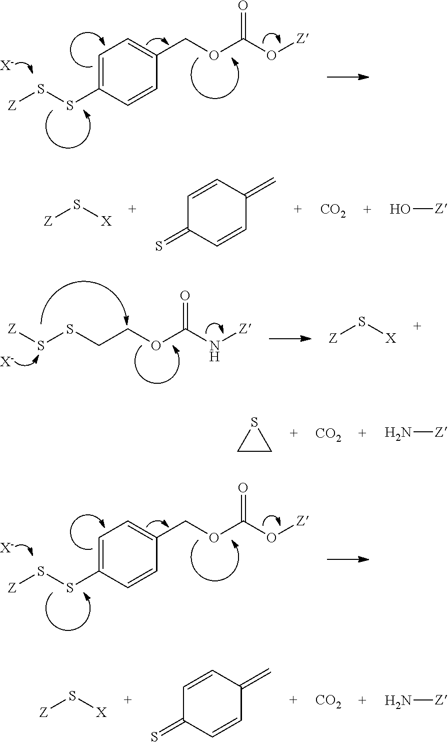

where X is an exogenous or endogenous nucleophile, glutathione, or bioreducing agent, and the like, and either of Z or Z′ is the vitamin, or analog or derivative thereof, or the nucleotide, or analog or derivative thereof, or a vitamin or nucleotide moiety in conjunction with other portions of the polyvalent linker. It is to be understood that although the above fragmentation mechanisms are depicted as concerted mechanisms, any number of discrete steps may take place to effect the ultimate fragmentation of the polyvalent linker to the final products shown. For example, it is appreciated that the bond cleavage may also occur by acid-catalyzed elimination of the carbamate moiety, which may be anchimerically assisted by the stabilization provided by either the aryl group of the beta sulfur or disulfide illustrated in the above examples. In those variations of this embodiment, the releasable linker is the carbamate moiety. Alternatively, the fragmentation may be initiated by a nucleophilic attack on the disulfide group, causing cleavage to form a thiolate. The thiolate may intermolecularly displace a carbonic acid or carbamic acid moiety and form the corresponding thiacyclopropane. In the case of the benzyl-containing polyvalent linkers, following an illustrative breaking of the disulfide bond, the resulting phenyl thiolate may further fragment to release a carbonic acid or carbamic acid moiety by forming a resonance stabilized intermediate. In any of these cases, the releasable nature of the illustrative polyvalent linkers described herein may be realized by whatever mechanism may be relevant to the chemical, metabolic, physiological, or biological conditions present.

Other illustrative mechanisms for bond cleavage of the releasable linker include oxonium-assisted cleavage as follows:

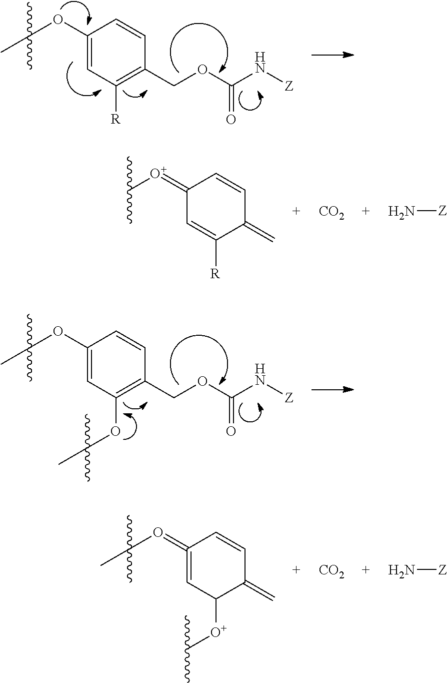

where Z is the vitamin, or analog or derivative thereof, or the nucleotide, or analog or derivative thereof, or each is a vitamin or nucleotide moiety in conjunction with other portions of the polyvalent linker, such as a nucleotide or vitamin moiety including one or more spacer linkers and/or other releasable linkers. Without being bound by theory, in this embodiment, acid catalysis, such as in an endosome, may initiate the cleavage via protonation of the urethane group. In addition, acid-catalyzed elimination of the carbamate leads to the release of CO₂ and the nitrogen-containing moiety attached to Z, and the formation of a benzyl cation, which may be trapped by water, or any other Lewis base.

Other illustrative linkers include compounds of the formulae:

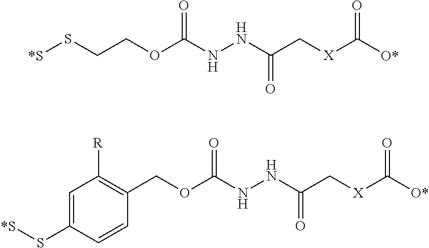

where X is NH, CH₂, or O; R is hydrogen, or a substituent, including a substituent capable of stabilizing a positive charge inductively or by resonance on the aryl ring, such as alkoxy and the like, including methoxy; and the symbol (*) indicates points of attachment for additional spacer, heteroatom, or releasable linkers forming the bivalent linker, or alternatively for attachment of the nucleotide, or analog or derivative thereof, or the vitamin, or analog or derivative thereof.

Illustrative mechanisms for cleavage of such bivalent linkers described herein include the following 1,4 and 1,6 fragmentation mechanisms followed by anchimerically assisted cleavage of the acylated Z′ via cyclization by the hydrazide group:

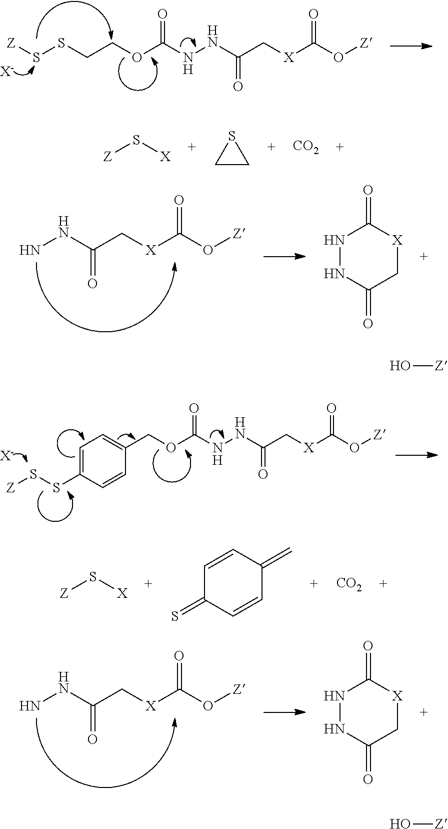

where X is an exogenous or endogenous nucleophile, glutathione, or bioreducing agent, and the like, and either of Z or Z′ is the vitamin, or analog or derivative thereof, or the nucleotide, or analog or derivative thereof, or a vitamin or nucleotide moiety in conjunction with other portions of the polyvalent linker. It is to be understood that although the above fragmentation mechanisms are depicted as concerted mechanisms, any number of discrete steps may take place to effect the ultimate fragmentation of the polyvalent linker to the final products shown. For example, it is appreciated that the bond cleavage may also occur by acid-catalyzed elimination of the carbamate moiety, which may be anchimerically assisted by the stabilization provided by either the aryl group of the beta sulfur or disulfide illustrated in the above examples. In those variations of this embodiment, the releasable linker is the carbamate moiety. Alternatively, the fragmentation may be initiated by a nucleophilic attack on the disulfide group, causing cleavage to form a thiolate. The thiolate may intermolecularly displace a carbonic acid or carbamic acid moiety and form the corresponding thiacyclopropane. In the case of the benzyl-containing polyvalent linkers, following an illustrative breaking of the disulfide bond, the resulting phenyl thiolate may further fragment to release a carbonic acid or carbamic acid moiety by forming a resonance stabilized intermediate. In any of these cases, the releasable nature of the illustrative polyvalent linkers described herein may be realized by whatever mechanism may be relevant to the chemical, metabolic, physiological, or biological conditions present. Without being bound by theory, in this embodiment, acid catalysis, such as in an endosome, may also initiate the cleavage via protonation of the urethane group. In addition, acid-catalyzed elimination of the carbamate leads to the release of CO₂ and the nitrogen-containing moiety attached to Z, and the formation of a benzyl cation, which may be trapped by water, or any other Lewis base, as is similarly described herein.

In one embodiment, the polyvalent linkers described herein are compounds of the following formulae

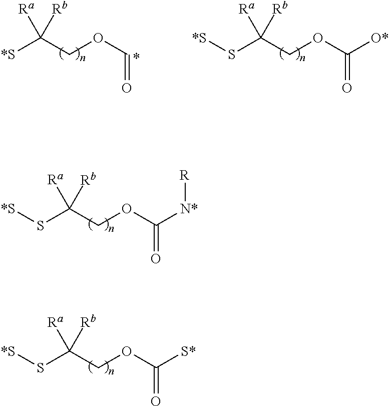

where n is an integer selected from 1 to about 4; R^{a} and R^{b} are each independently selected from the group consisting of hydrogen and alkyl, including lower alkyl such as C₁-C₄ alkyl that are optionally branched; or R^{a} and R^{b} are taken together with the attached carbon atom to form a carbocyclic ring; R is an optionally substituted alkyl group, an optionally substituted acyl group, or a suitably selected nitrogen protecting group; and (*) indicates points of attachment for the nucleotide, vitamin, other polyvalent linkers, or other parts of the conjugate.

In another embodiment, the polyvalent linkers described herein include compounds of the following formulae

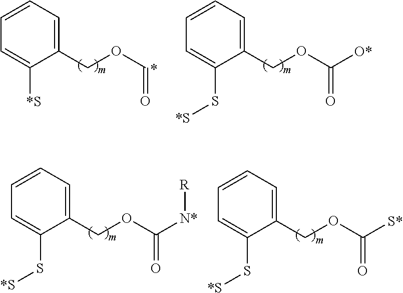

where m is an integer selected from 1 to about 4; R is an optionally substituted alkyl group, an optionally substituted acyl group, or a suitably selected nitrogen protecting group; and (*) indicates points of attachment for the nucleotide, vitamin, other polyvalent linkers, or other parts of the conjugate.

In another embodiment, the polyvalent linkers described herein include compounds of the following formulae

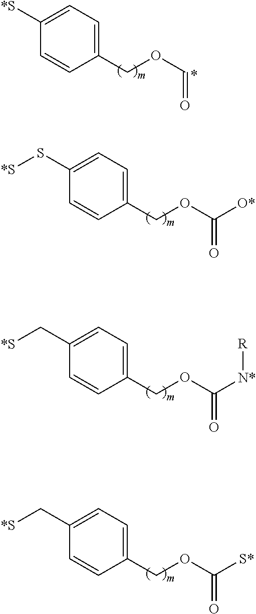

where m is an integer selected from 1 to about 4; R is an optionally substituted alkyl group, an optionally substituted acyl group, or a suitably selected nitrogen protecting group; and (*) indicates points of attachment for the nucleotide, vitamin, other polyvalent linkers, or other parts of the conjugate.

Another illustrative mechanism involves an arrangement of the releasable and spacer linkers in such a way that subsequent to the cleavage of a bond in the bivalent linker, released functional groups chemically assist the breakage or cleavage of additional bonds, also termed anchimeric assisted cleavage or breakage. An illustrative embodiment of such a bivalent linker or portion thereof includes compounds having the formula:

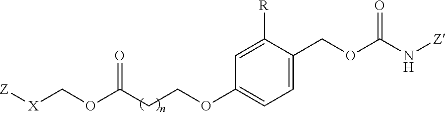

where X is an heteroatom, such as nitrogen, oxygen, or sulfur, n is an integer selected from 0, 1, 2, and 3, R is hydrogen, or a substituent, including a substituent capable of stabilizing a positive charge inductively or by resonance on the aryl ring, such as alkoxy, and the like, and either of Z or Z′ is the vitamin, or analog or derivative thereof, or the nucleotide, or analog or derivative thereof, or a vitamin or nucleotide moiety in conjunction with other portions of the bivalent linker. It is appreciated that other substituents may be present on the aryl ring, the benzyl carbon, the carbamate nitrogen, the alkanoic acid, or the methylene bridge, including but not limited to hydroxy, alkyl, alkoxy, alkylthio, halo, and the like. Assisted cleavage may include mechanisms involving benzylium intermediates, benzyne intermediates, lactone cyclization, oxonium intermediates, beta-elimination, and the like. It is further appreciated that, in addition to fragmentation subsequent to cleavage of the releasable linker, the initial cleavage of the releasable linker may be facilitated by an anchimerically assisted mechanism.

In this embodiment, the hydroxyalkanoic acid, which may cyclize, facilitates cleavage of the methylene bridge, by for example an oxonium ion, and facilitates bond cleavage or subsequent fragmentation after bond cleavage of the releasable linker. Alternatively, acid catalyzed oxonium ion-assisted cleavage of the methylene bridge may begin a cascade of fragmentation of this illustrative bivalent linker, or fragment thereof. Alternatively, acid-catalyzed hydrolysis of the carbamate may facilitate the beta elimination of the hydroxyalkanoic acid, which may cyclize, and facilitate cleavage of methylene bridge, by for example an oxonium ion. It is appreciated that other chemical mechanisms of bond breakage or cleavage under the metabolic, physiological, or cellular conditions described herein may initiate such a cascade of fragmentation. It is appreciated that other chemical mechanisms of bond breakage or cleavage under the metabolic, physiological, or cellular conditions described herein may initiate such a cascade of fragmentation.

In another embodiment, the releasable and spacer linkers may be arranged in such a way that subsequent to the cleavage of a bond in the polyvalent linker, released functional groups chemically assist the breakage or cleavage of additional bonds, also termed anchimeric assisted cleavage or breakage. An illustrative embodiment of such a polyvalent linker or portion thereof includes compounds having the formula:

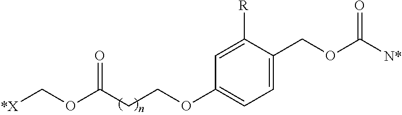

where X is an heteroatom, such as nitrogen, oxygen, or sulfur, n is an integer selected from 0, 1, 2, and 3, R is hydrogen, or a substituent, including a substituent capable of stabilizing a positive charge inductively or by resonance on the aryl ring, such as alkoxy, and the like, and the symbol (*) indicates points of attachment for additional spacer, heteroatom, or releasable linkers forming the polyvalent linker, or alternatively for attachment of the nucleotide, or analog or derivative thereof, or the vitamin, or analog or derivative thereof. It is appreciated that other substituents may be present on the aryl ring, the benzyl carbon, the alkanoic acid, or the methylene bridge, including but not limited to hydroxy, alkyl, alkoxy, alkylthio, halo, and the like. Assisted cleavage may include mechanisms involving benzylium intermediates, benzyne intermediates, lactone cyclization, oxonium intermediates, beta-elimination, and the like. It is further appreciated that, in addition to fragmentation subsequent to cleavage of the releasable linker, the initial cleavage of the releasable linker may be facilitated by an anchimerically assisted mechanism.

Another illustrative embodiment of the linkers described herein, include releasable linkers that cleave under the conditions described herein by a chemical mechanism involving beta elimination. In one aspect, such releasable linkers include beta-thio, beta-hydroxy, and beta-amino substituted carboxylic acids and derivatives thereof, such as esters, amides, carbonates, carbamates, and ureas. In another aspect, such releasable linkers include 2- and 4-thioarylesters, carbamates, and carbonates.

In another illustrative embodiment, the linker includes one or more amino acids. In one variation, the linker includes a single amino acid. In another variation, the linker includes a peptide having from 2 to about 50, 2 to about 30, or 2 to about 20 amino acids. In another variation, the linker includes a peptide having from about 4 to about 8 amino acids. Such amino acids are illustratively selected from the naturally occurring amino acids, or stereoisomers thereof. The amino acid may also be any other amino acid, such as any amino acid having the general formula:

—N(R)—(CR′R″)_{q}—C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornithine, threonine, and the like. In one variation, the releasable linker includes at least 2 amino acids selected from asparagine, aspartic acid, cysteine, glutamic acid, lysine, glutamine, arginine, serine, ornithine, and threonine. In another variation, the releasable linker includes between 2 and about 5 amino acids selected from asparagine, aspartic acid, cysteine, glutamic acid, lysine, glutamine, arginine, serine, ornithine, and threonine. In another variation, the releasable linker includes a tripeptide, tetrapeptide, pentapeptide, or hexapeptide consisting of amino acids selected from aspartic acid, cysteine, glutamic acid, lysine, arginine, and ornithine, and combinations thereof.

In another illustrative aspect of the receptor binding nucleotide delivery conjugate intermediate described herein, the nucleotide, or an analog or a derivative thereof, includes an alkylthiol nucleophile.

In another embodiment, the spacer linker can be 1-alkylenesuccinimid-3-yl, optionally substituted with a substituent X¹, as defined below, and the releasable linkers can be methylene, 1-alkoxyalkylene, 1-alkoxycycloalkylene, 1-alkoxyalkylenecarbonyl, 1-alkoxycycloalkylenecarbonyl, wherein each of the releasable linkers is optionally substituted with a substituent X², as defined below, and wherein the spacer linker and the releasable linker are each bonded to the spacer linker to form a succinimid-1-ylalkyl acetal or ketal.

The spacer linkers can be carbonyl, thionocarbonyl, alkylene, cycloalkylene, alkylenecycloalkyl, alkylenecarbonyl, cycloalkylenecarbonyl, carbonylalkylcarbonyl, 1-alkylenesuccinimid-3-yl, 1-(carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl, and 1-(carbonyltetrahydrofuranyl)succinimid-3-yl, wherein each of the spacer linkers is optionally substituted with a substituent X¹, as defined below. In this embodiment, the spacer linker may include an additional nitrogen, and the spacer linkers can be alkylenecarbonyl, cycloalkylenecarbonyl, carbonylalkylcarbonyl, 1-(carbonylalkyl)succinimid-3-yl, wherein each of the spacer linkers is optionally substituted with a substituent X¹, as defined below, and the spacer linker is bonded to the nitrogen to form an amide. Alternatively, the spacer linker may include an additional sulfur, and the spacer linkers can be alkylene and cycloalkylene, wherein each of the spacer linkers is optionally substituted with carboxy, and the spacer linker is bonded to the sulfur to form a thiol. In another embodiment, the spacer linker can include sulfur, and the spacer linkers can be 1-alkylenesuccinimid-3-yl and 1-(carbonylalkyl)succinimid-3-yl, and the spacer linker is bonded to the sulfur to form a succinimid-3-ylthiol.

In an alternative to the above-described embodiments, the spacer linker can include nitrogen, and the releasable linker can be a divalent radical comprising alkyleneaziridin-1-yl, carbonylalkylaziridin-1-yl, sulfoxylalkylaziridin-1-yl, or sulfonylalkylaziridin-1-yl, wherein each of the releasable linkers is optionally substituted with a substituent X², as defined below. In this alternative embodiment, the spacer linkers can be carbonyl, thionocarbonyl, alkylenecarbonyl, cycloalkylenecarbonyl, carbonylalkylcarbonyl, 1-(carbonylalkyl)succinimid-3-yl, wherein each of the spacer linkers is optionally substituted with a substituent X¹, as defined below, and wherein the spacer linker is bonded to the releasable linker to form an aziridine amide.

The substituents X¹ can be alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides. In this embodiment the spacer linker can include nitrogen, and the substituent X¹ and the spacer linker to which they are bound to form an heterocycle.

In one aspect of the various vitamin receptor binding nucleotide delivery conjugates described herein, the bivalent linker comprises a spacer linker and a releasable linker taken together to form 3-thiosuccinimid-1-ylalkyloxymethyloxy, where the methyl is optionally substituted with alkyl or substituted aryl.

In another aspect, the bivalent linker comprises a spacer linker and a releasable linker taken together to form 3-thiosuccinimid-1-ylalkylcarbonyl, where the carbonyl forms an acylaziridine with the nucleotide, or analog or derivative thereof.

In another aspect, the bivalent linker comprises an a spacer linker and a releasable linker taken together to form 1-alkoxycycloalkylenoxy.

In another aspect, the bivalent linker comprises a spacer linker and a releasable linker taken together to form alkyleneaminocarbonyl(dicarboxylarylene)carboxylate.

In another aspect, the bivalent linker comprises a releasable linker, a spacer linker, and a releasable linker taken together to form 2- or 3-dithioalkylcarbonylhydrazide, where the hydrazide forms an hydrazone with the nucleotide, or analog or derivative thereof.

In another aspect, the bivalent linker comprises a spacer linker and a releasable linker taken together to form 3-thiosuccinimid-1-ylalkylcarbonylhydrazide, where the hydrazide forms an hydrazone with the nucleotide, or analog or derivative thereof.

In another aspect, the bivalent linker comprises a spacer linker and a releasable linker taken together to form 2- or 3-thioalkylsulfonylalkyl(disubstituted silyl)oxy, where the disubstituted silyl is substituted with alkyl or optionally substituted aryl.

In another aspect, the bivalent linker comprises a plurality of spacer linkers selected from the group consisting of the naturally occurring amino acids and stereoisomers thereof.

In another aspect, the bivalent linker comprises a releasable linker, a spacer linker, and a releasable linker taken together to form 3-dithioalkyloxycarbonyl, where the carbonyl forms a carbonate with the nucleotide, or analog or derivative thereof.

In another aspect, the bivalent linker comprises a releasable linker, a spacer linker, and a releasable linker taken together to form 3-dithioalkyloxycarbonyl, where the carbonyl forms a carbonate with the nucleotide, or analog or derivative thereof.

In another aspect, the bivalent linker comprises a releasable linker, a spacer linker, and a releasable linker taken together to form 2-dithioarylalkyloxycarbonyl, where the carbonyl forms a carbonate with the nucleotide, or analog or derivative thereof, and the aryl is optionally substituted.

In another aspect, the bivalent linker comprises a spacer linker and a releasable linker taken together to form 3-thiosuccinimid-1-ylalkyloxyalkyloxyalkylidene, where the alkylidene forms an hydrazone with the nucleotide, or analog or derivative thereof, each alkyl is independently selected, and the oxyalkyloxy is optionally substituted with alkyl or optionally substituted aryl.

In another aspect, the bivalent linker comprises a releasable linker, a spacer linker, and a releasable linker taken together to form 2- or 3-dithioalkyloxycarbonylhydrazide.

In another aspect, the bivalent linker comprises a releasable linker, a spacer linker, and a releasable linker taken together to form 2- or 3-dithioalkylamino, where the amino forms a vinylogous amide with the nucleotide, or analog or derivative thereof.

In another aspect, the bivalent linker comprises a releasable linker, a spacer linker, and a releasable linker taken together to form 2- or 3-dithioalkylamino, where the amino forms a vinylogous amide with the nucleotide, or analog or derivative thereof, and the alkyl is ethyl.

In another aspect, the bivalent linker comprises a releasable linker, a spacer linker, and a releasable linker taken together to form 2- or 3-dithioalkylaminocarbonyl, where the carbonyl forms a carbamate with the nucleotide, or analog or derivative thereof.

In another aspect, the bivalent linker comprises a releasable linker, a spacer linker, and a releasable linker taken together to form 2- or 3-dithioalkylaminocarbonyl, where the carbonyl forms a carbamate with the nucleotide, or analog or derivative thereof, and the alkyl is ethyl.

In another aspect, the bivalent linker comprises a releasable linker, a spacer linker, and a releasable linker taken together to form 2- or 3-dithioarylalkyloxycarbonyl, where the carbonyl forms a carbamate or a carbamoylaziridine with the nucleotide, or analog or derivative thereof.

In another embodiment, the polyvalent linker includes spacer linkers and releasable linkers connected to form a polyvalent 3-thiosuccinimid-1-ylalkyloxymethyloxy group, illustrated by the following formula

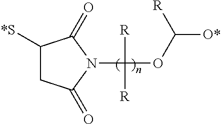

where n is an integer from 1 to 6, the alkyl group is optionally substituted, and the methyl is optionally substituted with an additional alkyl or optionally substituted aryl group, each of which is represented by an independently selected group R. The (*) symbols indicate points of attachment of the polyvalent linker fragment to other parts of the conjugates described herein.

In another embodiment, the polyvalent linker includes spacer linkers and releasable linkers connected to form a polyvalent 3-thiosuccinimid-1-ylalkylcarbonyl group, illustrated by the following formula

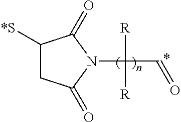

where n is an integer from 1 to 6, and the alkyl group is optionally substituted. The (*) symbols indicate points of attachment of the polyvalent linker fragment to other parts of the conjugates described herein. In another embodiment, the polyvalent linker includes spacer linkers and releasable linkers connected to form a polyvalent 3-thioalkylsulfonylalkyl(disubstituted silyl)oxy group, where the disubstituted silyl is substituted with alkyl and/or optionally substituted aryl groups.

In another embodiment, the polyvalent linker includes spacer linkers and releasable linkers connected to form a polyvalent dithioalkylcarbonylhydrazide group, or a polyvalent 3-thiosuccinimid-1-ylalkylcarbonylhydrazide, illustrated by the following formulae

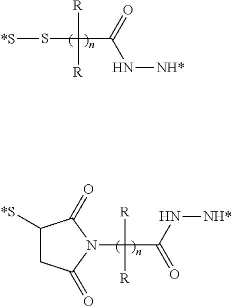

where n is an integer from 1 to 6, the alkyl group is optionally substituted, and the hydrazide forms an hydrazone with (B), (N), or another part of the polyvalent linker (L). The (*) symbols indicate points of attachment of the polyvalent linker fragment to other parts of the conjugates described herein.

In another embodiment, the polyvalent linker includes spacer linkers and releasable linkers connected to form a polyvalent 3-thiosuccinimid-1-ylalkyloxyalkyloxyalkylidene group, illustrated by the following formula

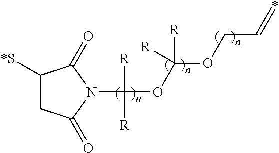

where each n is an independently selected integer from 1 to 6, each alkyl group independently selected and is optionally substituted, such as with alkyl or optionally substituted aryl, and where the alkylidene forms an hydrazone with (B), (N), or another part of the polyvalent linker (L). The (*) symbols indicate points of attachment of the polyvalent linker fragment to other parts of the conjugates described herein.

Additional illustrative spacer linkers include alkylene-amino-alkylenecarbonyl, alkylene-thio-carbonylalkylsuccinimid-3-yl, and the like, as further illustrated by the following formulae:

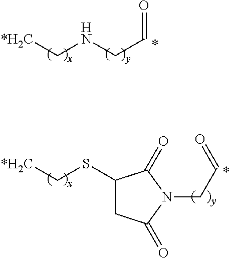

where the integers x and y are 1, 2, 3, 4, or 5:

The term cycloalkylene as used herein refers to a bivalent chain of carbon atoms, a portion of which forms a ring, such as cycloprop-1,1-diyl, cycloprop-1,2-diyl, cyclohex-1,4-diyl, 3-ethylcyclopent-1,2-diyl, 1-methylenecyclohex-4-yl, and the like.

The term heterocycle as used herein refers to a monovalent chain of carbon and heteroatoms, wherein the heteroatoms are selected from nitrogen, oxygen, and sulfur, a portion of which, including at least one heteroatom, form a ring, such as aziridine, pyrrolidine, oxazolidine, 3-methoxypyrrolidine, 3-methylpiperazine, and the like.

The term aryl as used herein refers to an aromatic mono or polycyclic ring of carbon atoms, such as phenyl, naphthyl, and the like. In addition, aryl may also include heteroaryl.

The term heteroaryl as used herein refers to an aromatic mono or polycyclic ring of carbon atoms and at least one heteroatom selected from nitrogen, oxygen, and sulfur, such as pyridinyl, pyrimidinyl, indolyl, benzoxazolyl, and the like.

The term optionally substituted as used herein refers to the replacement of one or more hydrogen atoms, generally on carbon, with a corresponding number of substituents, such as halo, hydroxy, amino, alkyl or dialkylamino, alkoxy, alkylsulfonyl, cyano, nitro, and the like. In addition, two hydrogens on the same carbon, on adjacent carbons, or nearby carbons may be replaced with a bivalent substituent to form the corresponding cyclic structure.

The term iminoalkylidenyl as used herein refers to a divalent radical containing alkylene as defined herein and a nitrogen atom, where the terminal carbon of the alkylene is double-bonded to the nitrogen atom, such as the formulae —(CH)═N—, —(CH₂)₂(CH)═N—, —CH₂C(Me)═N—, and the like.

The term amino acid as used herein refers generally to aminoalkylcarboxylate, where the alkyl radical is optionally substituted, such as with alkyl, hydroxy alkyl, sulfhydrylalkyl, aminoalkyl, carboxyalkyl, and the like, including groups corresponding to the naturally occurring amino acids, such as serine, cysteine, methionine, aspartic acid, glutamic acid, and the like. It is to be understood that such amino acids may be of a single stereochemistry or a particular mixture of stereochemisties, including racemic mixtures. In addition, amino acid refers to beta, gamma, and longer amino acids, such as amino acids of the formula:

—N(R)—(CR′R″)_{q}—C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornithine, threonine, and the like. In another illustrative aspect of the vitamin receptor binding nucleotide delivery conjugate intermediate described herein, the nucleotide, or an analog or a derivative thereof, includes an alkylthiol nucleophile.

It is to be understood that the above-described terms can be combined to generate chemically-relevant groups, such as alkoxyalkyl referring to methyloxymethyl, ethyloxyethyl, and the like, haloalkoxyalkyl referring to trifluoromethyloxyethyl, 1,2-difluoro-2-chloroeth-1-yloxypropyl, and the like, arylalkyl referring to benzyl, phenethyl, a-methylbenzyl, and the like, and others.

The term amino acid derivative as used herein refers generally to an optionally substituted aminoalkylcarboxylate, where the amino group and/or the carboxylate group are each optionally substituted, such as with alkyl, carboxylalkyl, alkylamino, and the like, or optionally protected. In addition, the optionally substituted intervening divalent alkyl fragment may include additional groups, such as protecting groups, and the like.

The term peptide as used herein refers generally to a series of amino acids and/or amino acid analogs and derivatives covalently linked one to the other by amide bonds.

Additional linkers are described in U.S. patent application publication 2005/0002942, the disclosure of which is incorporated herein by reference, and in Tables 1 and 2 below, where the (*) atom is the point of attachment of additional spacer or releasable linkers, the nucleotide, and/or the binding ligand.

TABLE 1
Illustrative spacer linkers.

| 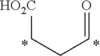 | 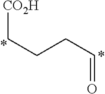 |
|---|---|
| 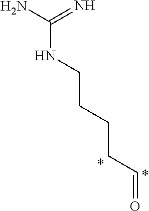 | 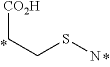 |
| 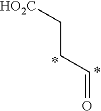 | 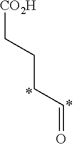 |
| 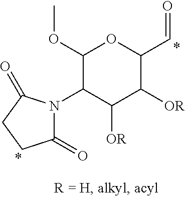 | |
| 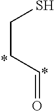 | 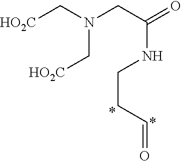 |
| 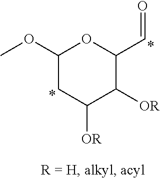 | |
| 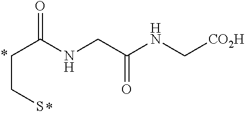 | |
| 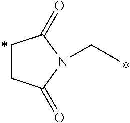 | 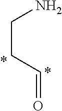 |
| 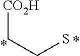 | 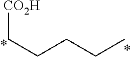 |
| 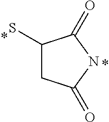 | |
| 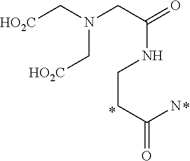 | |
| 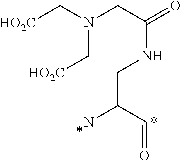 | |
| 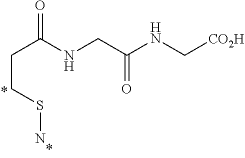 | |
| 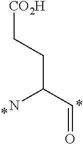 | 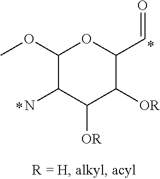 |
| 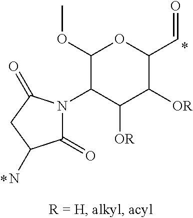 | |
| 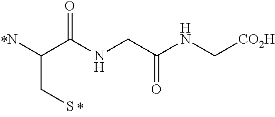 | |
| 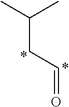 | 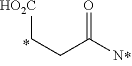 |
| 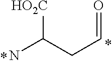 | 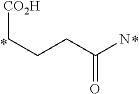 |
| 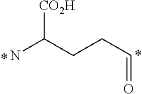 | 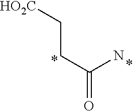 |
| 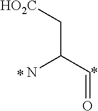 | |
| 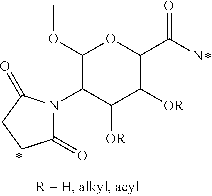 | |
| 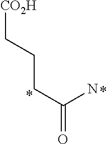 | 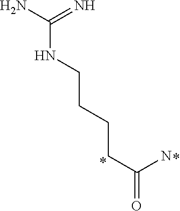 |
| 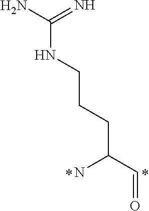 | |
| 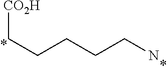 | 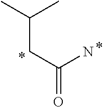 |
| 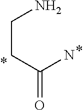 | 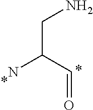 |
| 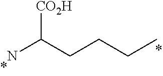 | 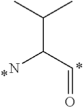 |
| 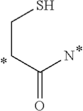 | 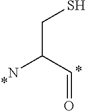 |
| 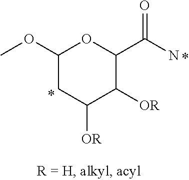 | |
| 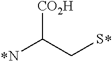 | 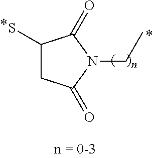 |
| 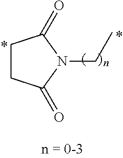 | 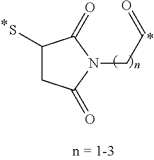 |

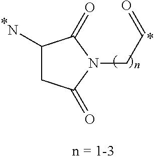

TABLE 2
Illustrative releasable linkers.

| 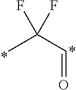 | 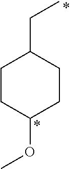 |
|---|---|
| 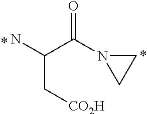 | 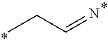 |
| 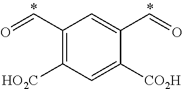 | 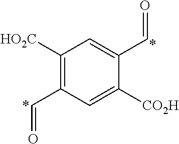 |
| 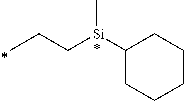 | 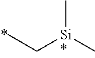 |
| 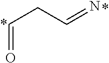 | 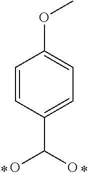 |
| 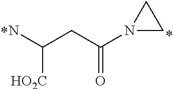 | 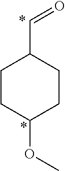 |
| 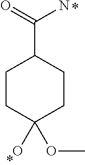 | 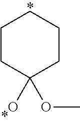 |
| 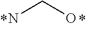 | 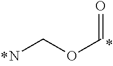 |
| 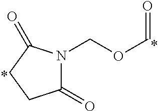 | |
| 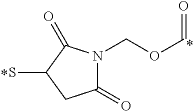 | |
| 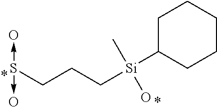 | |
| 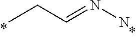 | 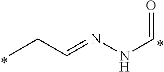 |
| 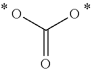 | 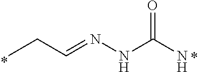 |
| 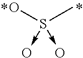 | 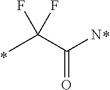 |
| 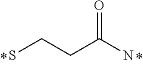 | 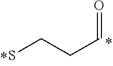 |
| 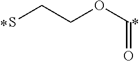 | 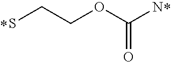 |
| 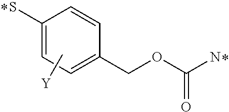 | |
| 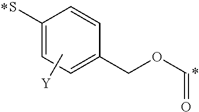 | |
| 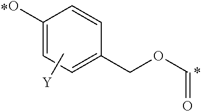 | |
| 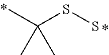 | 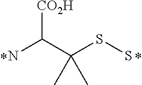 |

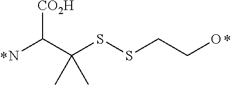

In another embodiment, multi-nucleotide conjugates are described herein. Several illustrative configurations of such multi-nucleotide conjugates are contemplated herein, and include the compounds and compositions described in PCT international publication No. WO 2007/022494, the disclosure of which is incorporated herein by reference. Illustratively, the polyvalent linkers may connect the receptor binding ligand B to the two or more agents A, providing that one agent is a nucleotide, such as a nucleic acid. Such polyvalent conjugates may be in a variety of structural configurations, including but not limited to the following illustrative general formulae:

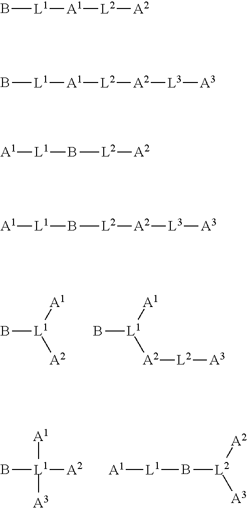

where B is the receptor binding ligand, each of (L¹), (L²), and (L³) is a polyvalent linker, and each of (A¹), (A²), and (A³) is an agent A, or an analog or derivative thereof. In one aspect, the polyvalent linkers include one or more releasable linkers and/or additional spacer linkers. In another aspect, the agents A include at least one nucleotide N. In one variation, the agents A include other compounds, attached to the conjugates by one or more releasable linkers and/or additional spacer linkers. Other variations, including additional agents A, or analogs or derivatives thereof, additional linkers, and additional configurations of the arrangement of each of (B), (L), and (A), are also contemplated herein.

In one variation, more than one receptor binding ligand B is included in the delivery conjugates described herein, including but not limited to the following illustrative general formulae:

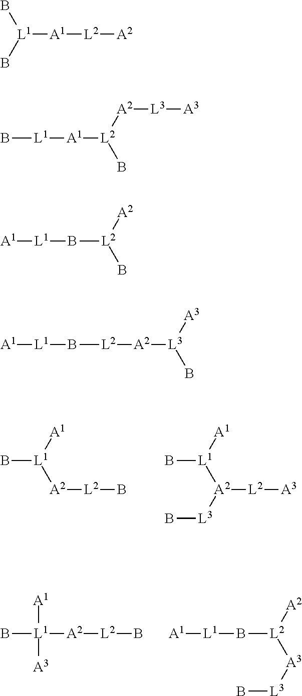

where each B is a receptor binding ligand, each of (L¹), (L²), and (L³) is a polyvalent linker, and each of (A¹), (A²), and (A³) is an agent A, or an analog or derivative thereof. In one aspect, the polyvalent linkers include one or more releasable linkers and/or additional spacer linkers. In another aspect, the agents A include at least one nucleotide N. In one variation, the agents A include other compounds, attached to the conjugates by one or more releasable linkers and/or additional spacer linkers, which may be used in targeting pathogenic cell populations, such as cancers. Other variations, including additional agents A, or analogs or derivatives thereof, additional linkers, and additional configurations of the arrangement of each of (B), (L), and (A), are also contemplated herein. In one variation, the receptor binding ligands B are ligands for the same receptor, and in another variation, the receptor binding ligands B are ligands for different receptors.

The binding site for the receptor binding ligand (B), such as a vitamin, can include receptors for any binding ligand (B), or a derivative or analog thereof, capable of specifically binding to a receptor wherein the receptor or other protein is uniquely expressed, overexpressed, or preferentially expressed by a population of pathogenic cells. A surface-presented protein uniquely expressed, overexpressed, or preferentially expressed by the pathogenic cells (e.g., cancer cells or cells of the immune system involved in inflammation) is typically a receptor that is either not present or present at lower concentrations on non-pathogenic cells providing a means for specific elimination of the pathogenic cells. The receptor binding ligand nucleotide delivery conjugates may be capable of high affinity binding to receptors on pathogenic cells (e.g. cancer cells or cells of the immune system involved in inflammation) that overexpress a receptor such as a vitamin receptor. The high affinity binding can be inherent to the receptor binding ligand or the binding affinity can be enhanced by the use of a chemically modified ligand, such as an analog or a derivative of a vitamin.

The receptor binding ligand nucleotide delivery conjugates described herein can be formed from, for example, a wide variety of vitamins or receptor-binding vitamin analogs/derivatives, linkers, and nucleotides. The binding ligand nucleotide delivery conjugates described herein are capable of selectively targeting a population of pathogenic cells in the host animal due to preferential expression of a receptor for the binding ligand, such as a vitamin, accessible for ligand binding, on the pathogenic cells. Illustrative vitamin moieties that can be used as the receptor binding ligand (B) include carnitine, inositol, lipoic acid, pyridoxal, ascorbic acid, niacin, pantothenic acid, folic acid, riboflavin, thiamine, biotin, vitamin B₁₂, and the lipid soluble vitamins A, D, E and K. These vitamins, and their receptor-binding analogs and derivatives, constitute an illustrative targeting entity that can be coupled with the nucleotide by a bivalent linker (L) to form a binding ligand (B) nucleotide delivery conjugate as described herein. The term vitamin is understood to include vitamin analogs and/or derivatives, unless otherwise indicated. Illustratively, pteroic acid which is a derivative of folate, biotin analogs such as biocytin, biotin sulfoxide, oxybiotin and other biotin receptor-binding compounds, and the like, are considered to be vitamins, vitamin analogs, and vitamin derivatives. It should be appreciated that vitamin analogs or derivatives as described herein refer to vitamins that incorporates an heteroatom through which the vitamin analog or derivative is covalently bound to the bivalent linker (L).

Illustrative vitamin moieties include folic acid, biotin, riboflavin, thiamine, vitamin B₁₂, and receptor-binding analogs and derivatives of these vitamin molecules, and other related vitamin receptor binding molecules.

In one embodiment, the targeting ligand B is a folate, an analog of folate, or a derivative of folate. It is to be understood as used herein, that the term folate is used both individually and collectively to refer to folic acid itself, and/or to such analogs and derivatives of folic acid that are capable of binding to folate receptors.

Illustrative embodiments of folate analogs and/or derivatives include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate. The dideaza analogs include, for example, 1,5-dideaza, 5,10-dideaza, 8,10-dideaza, and 5,8-dideaza analogs of folate. Other folates useful as complex forming ligands include the folate receptor-binding analogs aminopterin, amethopterin (methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). The foregoing folic acid analogs and/or derivatives are conventionally termed folates, reflecting their ability to bind with folate-receptors, and such ligands when conjugated with exogenous molecules are effective to enhance transmembrane transport, such as via folate-mediated endocytosis as described herein.

Additional analogs of folic acid that bind to folic acid receptors are described in U.S. Patent Application Publication Serial Nos. 2005/0227985 and 2004/0242582, the disclosures of which are incorporated herein by reference. Illustratively, such folate analogs have the general formula:

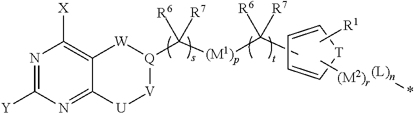

wherein X and Y are each-independently selected from the group consisting of halo, R², OR², SR³, and NR⁴R⁵;

U, V, and W represent divalent moieties each independently selected from the group consisting of —(R^{6a})C═, —(R^{6a})C(R^{7a})—, and —N(R^{4a})—; Q is selected from the group consisting of C and CH; T is selected from the group consisting of S, O, N, and —C═C—;

M¹ and M² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b} and R^{1b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

L is a divalent linker as described herein; and

n, p, r, s and t are each independently either 0 or 1.

As used herein, it is to be understood that the term folate refers both individually to folic acid used in forming a conjugate, or alternatively to a folate analog or derivative thereof that is capable of binding to folate or folic acid receptors.

The vitamin can be folate which includes a nitrogen, and in this embodiment, the spacer linkers can be alkylenecarbonyl, cycloalkylenecarbonyl, carbonylalkylcarbonyl, 1-alkylenesuccinimid-3-yl, 1-(carbonylalkyl)succinimid-3-yl, wherein each of the spacer linkers is optionally substituted with a substituent X¹, and the spacer linker is bonded to the folate nitrogen to form an imide or an alkylamide. In this embodiment, the substituents X¹ can be alkyl, hydroxyalkyl, amino, aminoalkyl, alkylaminoalkyl, dialkylaminoalkyl, sulfhydrylalkyl, alkylthioalkyl, aryl, substituted aryl, arylalkyl, substituted arylalkyl, carboxy, carboxyalkyl, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides.

Illustrative embodiments of vitamin analogs and/or derivatives also include analogs and derivatives of biotin such as biocytin, biotin sulfoxide, oxybiotin and other biotin receptor-binding compounds, and the like. It is appreciated that analogs and derivatives of the other vitamins described herein are also contemplated herein. In one embodiment, vitamins that can be used as the receptor binding ligand (B) in the nucleotide delivery conjugates described herein include those that bind to vitamin receptors expressed specifically on activated macrophages or cancer cells, such as the folate receptor, which binds folate, or an analog or derivative thereof as described herein.

In addition to the vitamins described herein, it is appreciated that other binding ligands may be coupled with the nucleotides and linkers described and contemplated herein to form receptor binding ligand-linker-nucleotide conjugates capable of facilitating delivery of the nucleotide to a desired target. These other binding ligands, in addition to the vitamins and their analogs and derivatives described, may be used to form nucleotide delivery conjugates capable of binding to target cells. In general, any binding ligand (B) of a cell surface receptor may be advantageously used as a targeting ligand to which a linker-nucleotide conjugate can be attached. As used herein, the phrases “receptor binding ligand” and “binding ligand” are interchangeable. The terms “nucleotide N” and “nucleotide” are also interchangeable.

The binding ligand nucleotide delivery conjugates can comprise a binding ligand (B), a bivalent linker (L), a nucleotide, and, optionally, heteroatom linkers to link the binding ligand (B) receptor binding moiety and the nucleotide to the bivalent linker (L). In one illustrative embodiment, it should be appreciated that a vitamin analog or derivative can mean a vitamin that incorporates an heteroatom through which the vitamin analog or derivative is covalently bound to the bivalent linker (L). Thus, in this illustrative embodiment, the vitamin can be covalently bound to the bivalent linker (L) through an heteroatom linker, or a vitamin analog or derivative (i.e., incorporating an heteroatom) can be directly bound to the bivalent linker (L). In similar illustrative embodiments, a nucleotide analog or derivative is a nucleotide, and a nucleotide analog or derivative can mean a nucleotide that incorporates an heteroatom through which the nucleotide analog or derivative is covalently bound to the bivalent linker (L). Thus, in these illustrative aspects, the nucleotide can be covalently bound to the bivalent linker (L) through an heteroatom linker, or a nucleotide analog or derivative (i.e., incorporating an heteroatom) can be directly bound to the bivalent linker (L). The bivalent linker (L) can comprise a spacer linker, a releasable (i.e., cleavable) linker, and an heteroatom linker to link the spacer linker to the releasable linker in conjugates containing both of these types of linkers.

Generally, any manner of forming a conjugate between the bivalent linker (L) and the binding ligand (B), or analog or derivative thereof, between the bivalent linker (L) and the nucleotide, or analog or derivative thereof, including any intervening heteroatom linkers, can be utilized Also, any art-recognized method of forming a conjugate between the spacer linker, the releasable linker, and the heteroatom linker to form the bivalent linker (L) can be used. The conjugate can be formed by direct conjugation of any of these molecules, for example, through complexation, or through hydrogen, ionic, or covalent bonds. Covalent bonding can occur, for example, through the formation of amide, ester, disulfide, or imino bonds between acid, aldehyde, hydroxy, amino, sulfhydryl, or hydrazo groups. The linker (L) can be linked to one nucleic acid strand and the other strand can then be hybridized to form the conjugate containing a double-stranded nucleic acid. The nucleotide can also be targeted to the pathogenic cell population (e.g., cancer cells or cells of the immune system involved in inflammation) using liposomes, dendrimers, carbohydrate modifications of the conjugate, nanoparticles or scaffolds, biodegradable polymers, micelles, and the like.

The nucleotide delivery conjugates described herein can be prepared by art-recognized synthetic methods. The synthetic methods are chosen depending upon the selection of the optionally added heteroatoms or the heteroatoms that are already present on the spacer linkers, releasable linkers, the nucleotide, and/or or the binding ligand. In general, the relevant bond forming reactions are described in Richard C. Larock, “Comprehensive Organic Transformations, a guide to functional group preparations,” VCH Publishers, Inc. New York (1989), and in Theodora E. Greene & Peter G. M. Wuts, “Protective Groups ion Organic Synthesis,” 2d edition, John Wiley & Sons, Inc. New York (1991), the disclosures of which are incorporated herein by reference.

Conjugates with Releasable Linkers and Hydrophilic Spacer Linkers

In one embodiment, compounds of the following formula are described herein:

B-L-N

wherein B is a receptor binding ligand that binds to a target cell receptor, L is a linker that comprises one or more hydrophilic spacer linkers, and N is a nucleotide that is delivered to the cell.

In another embodiment, the receptor binding ligand is a folate, or an analog or derivative thereof. In another embodiment, linker L also includes at least one releasable linker. In one variation of this embodiment, at least one releasable linker is attached to nucleotide N. In another variation, at least one releasable linker is located between the hydrophilic spacer linker and nucleotide N. In another variation, receptor binding ligand B, such as a folate receptor binding ligand, is attached to a hydrophilic spacer linker. In another variation, both nucleotide N and receptor binding ligand B are each attached to a hydrophilic spacer linker, where the spacer linkers are attached to each other through a releasable linker. In another variation, both nucleotide N and receptor binding ligand B are each be attached to a releasable linker, where the releasable linkers are each attached to a hydrophilic spacer linker. Each of these radicals may be connected through existing or additional heteroatoms on binding ligand B, nucleotide N, or any of the releasable, hydrophilic spacer, or additional spacer linkers. Illustrative heteroatoms include nitrogen, oxygen, sulfur, and the formulae —(NHR¹NHR²)—, —SO—, —(SO₂)—, and —N(R³)O—, wherein R¹, R², and R³ are each independently selected from hydrogen, alkyl, aryl, arylalkyl, substituted aryl, substituted arylalkyl, heteroaryl, substituted heteroaryl, and alkoxyalkyl.

The binding ligand nucleotide delivery conjugates described herein can be formed from, for example, a wide variety of folates or folate receptor-binding compounds, linkers, and nucleotides. In another embodiment, the binding ligand nucleotide delivery conjugates of the present invention are capable of specifically targeting a population of pathogenic cells in the host animal due to preferential expression of the receptor for the binding ligand on the pathogenic cells.

Receptor binding ligand B includes a wide variety of ligands for cell surface folate receptors. In one embodiment, B is folic acid, or an analog or derivative of folic acid that binds to folic acid receptors. It is to be understood that analogs and derivatives include folates that incorporate an heteroatom through which the analog or derivative is covalently bound to bivalent linker (L).

Illustrative analogs and derivatives of folic acid that bind to folic acid receptors includes, but is not limited to, folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to analogs having a carbon atom substituted for one or two nitrogen atoms in folic acid structure, including the naturally occurring folic acid structure, or analogs or derivatives thereof. For example, the deaza analogs include the 1-deaza, 3-deaza, 5-deaza, 8-deaza, and 10-deaza analogs of folate. The dideaza analogs include, for example, 1,5-dideaza, 5,10-dideaza, 8,10-dideaza, and 5,8-dideaza analogs of folate. Other folates useful as complex forming ligands for this invention are the folate receptor-binding analogs aminopterin, amethopterin (methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate).

Additional illustrative analogs of folic acid that bind to folic acid receptors are described in US Patent Application Publication Serial Nos. 2005/0227985 and 2004/0242582, the disclosures of which are incorporated herein by reference. Illustratively, such folate analogs have the general formula, where the (*) represents the point of attachment of additional bivalent linker radicals or nucleotide N:

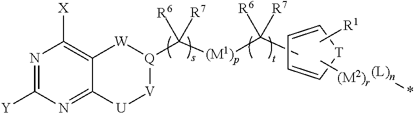

wherein X and Y are each-independently selected from the group consisting of halo, R², OR², SR³, and NR⁴R⁵;

U, V, and W represent divalent moieties each independently selected from the group consisting of —(R^{6a})C═, —N═, and —N(R^{4a})—; Q is selected from the group consisting of C and CH; T is selected from the group consisting of S, O, N, and —C═C—;

M¹ and M² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{1b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

L is a bivalent linker as described herein; and

n, p, r, s and t are each independently either 0 or 1.

In one aspect of such folate analogs, when s is 1, t is 0, and when s is 0, t is 1. In another aspect of such folate analogs, both n and r are 1, and linker L^{a} is a naturally occurring amino acid covalently linked to M² at its alpha-amino group through an amide bond. Illustrative amino acids include aspartic acid, glutamic acid, and the like.

The foregoing folic acid analogs and/or derivatives are conventionally termed “folates,” reflecting their ability to bind with folate-receptors, and such ligands when conjugated with exogenous molecules are effective to enhance transmembrane transport, such as via folate-mediated endocytosis as described herein. Accordingly, as used herein, it is to be understood that the term “folate” refers both individually to folic acid used in forming a conjugate, or alternatively to a folate analog or derivative thereof that is capable of binding to folate or folic acid receptors.

The binding site for the binding ligand (B) is capable of selectively or specifically binding to a receptor wherein the receptor or other protein is uniquely expressed, overexpressed, or preferentially expressed by a population of pathogenic cells (e.g. cancer cells or inflammatory cells). A surface-presented protein uniquely expressed, overexpressed, or preferentially expressed by the pathogenic cells is typically a receptor that is either not present or present at lower concentrations on non-pathogenic cells providing a means for specific elimination of the pathogenic cells. The binding ligand nucleotide delivery conjugates may be capable of high affinity binding to receptors on cancer cells or other types of pathogenic cells (e.g. inflammatory cells). The high affinity binding can be inherent to the binding ligand or the binding affinity can be enhanced by the use of a chemically modified ligand, such as for example by including an analog or a derivative of a folate.

As described herein, nucleotide N includes both RNAs and DNAs of varying lengths. In addition, nucleotide N includes both single stranded or double stranded molecules. Nucleotide N also includes blunt-ended nucleotides and nucleotides that have overhangs of varying lengths, at one or both ends of a double-stranded nucleotide. In one variation, the overhang is at one or both of the 3′ ends of the nucleotide. In another variation, in each of the single and double stranded embodiments, and in each of the blunt-ended and overhang embodiment, one or both of the 3′ ends of the nucleotide terminate in an hydroxyl group. In another variation, in each of the single and double stranded embodiments, and in each of the blunt-ended and overhang embodiment, one or both of the 5′ ends of the nucleotides terminate in a phosphate group.

As described herein, in one embodiment, nucleotide N includes about 15 to about 49 bases. In another embodiment, nucleotide N includes about 19 to about 25 bases. In another embodiment, nucleotide N includes about 15 to about 23 bases. In another embodiment, nucleotide N includes about 21 to about 23 bases. In another embodiment, nucleotide N includes an overhang at each end of a double-stranded nucleotide of about 2 to about 3 bases. In another embodiment, nucleotide N includes small interfering RNA, also referred to as siRNA.

In each of the forgoing, it is to be understood that nucleotide N includes not only natural bases, such as A, C, T, G, and U, but also non-natural analogs and derivatives of such bases. For example, bases or analogs and derivatives of bases that may further stabilize the nucleotide against degradation or metabolism, or other derivatives may be included for nucleotide N, including 2′-F, 2′-OMe, and other derivatives of naturally occurring bases.

The linker L includes one or more hydrophilic spacer linkers. In addition, other optional spacer linkers and/or releasable linkers may be included in L. It is appreciated that additional spacer linkers may be included when predetermined lengths are selected for separating binding ligand B from nucleotide N. It is also appreciated that in certain configurations, releasable linkers may be included. For example, as described herein in one embodiment, the binding ligand conjugates may be used to deliver nucleotides N for treating cancer or other diseases involving pathogenic cells, such as inflammation. In such embodiments, it is appreciated that once delivered, nucleotide N is desirably released from the conjugate. For example, in the configuration where the binding ligand is a folate, the conjugate may bind to a folate receptor. Once bound, the conjugate often undergoes the process of endocytosis, and the conjugate is delivered to the interior of the cell. Cellular mechanisms may biologically degrade the conjugate to release the nucleotide “payload” as well as release the folate compound.

In another alternative configuration, a releasable linker may or may not be included. For example, conjugates that include imaging agents may be delivered to a target cell using the appropriate receptor binding ligand, such as a folate or other folate receptor binding ligand in the absence of a releasable linker In one configuration, the conjugate may undergo endocytosis into the interior of the cell.

Accordingly, in other aspects, the conjugates B-L-N described herein also include the following general formulae:

B-L_{S}-L_{H}-N

B-L_{H}-L_{S}-N

B-L_{S}-L_{H}-L_{S}-N

B-L_{R}-L_{H}-N

B-L_{H}-L_{R}-N

B-L_{R}-L_{H}-L_{R}-N

B-L_{S}-L_{R}-L_{H}-N

B-L_{R}-L_{H}-L_{S}-N

B-L_{R}-L_{S}-L_{H}-L_{R}-N

B-L_{H}-L_{S}-L_{H}-L_{R}-N

where B, L, and N are as described herein, and L_{R} is a releasable linker section, Lₛ is a spacer linker section, and L_{H} is a hydrophilic linker section of bivalent linker L. It is to be understood that the foregoing formulae are merely illustrative, and that other arrangements of the hydrophilic spacer linker sections, releasable linker sections, and spacer linker sections are contemplated. In addition, it is to be understood that additional conjugates are contemplated that include a plurality hydrophilic spacer linkers, and/or a plurality of releasable linkers, and/or a plurality of spacer linkers.

It is appreciated that the arrangement and/or orientation of the various hydrophilic linkers may be in a linear or branched fashion, or both. For example, the hydrophilic linkers may form the backbone of the linker forming the conjugate between the folate and the nucleotide. Alternatively, the hydrophilic portion of the linker may be pendant to or attached to the backbone of the chain of atoms connecting the binding ligand B to the nucleotide N. In this latter arrangement, the hydrophilic portion may be proximal or distal to the backbone chain of atoms.

In another embodiment, the linker is more or less linear, and the hydrophilic groups are arranged largely in a series to form a chain-like linker in the conjugate. Said another way, the hydrophilic groups form some or all of the backbone of the linker in this linear embodiment.

In another embodiment, the linker is branched with hydrophilic groups. In this branched embodiment, the hydrophilic groups may be proximal to the backbone or distal to the backbone. In each of these arrangements, the linker is more spherical or cylindrical in shape. In one variation, the linker is shaped like a bottle-brush. In one aspect, the backbone of the linker is formed by a linear series of amides, and the hydrophilic portion of the linker is formed by a parallel arrangement of branching side chains, such as by connecting monosaccharides, sulfonates, and the like, and derivatives and analogs thereof.

It is understood that the linker may be neutral or ionizable under certain conditions, such as physiological conditions encountered in vivo. For ionizable linkers, under the selected conditions, the linker may deprotonate to form a negative ion, or alternatively become protonated to form a positive ion. It is appreciated that more than one deprotonation or protonation event may occur. In addition, it is understood that the same linker may deprotonate and protonate to form inner salts or zwitterionic compounds.

In another embodiment, the hydrophilic spacer linkers are neutral, i.e. under physiological conditions, the linkers do not significantly protonate nor deprotonate. In another embodiment, the hydrophilic spacer linkers may be protonated to carry one or more positive charges. It is understood that the protonation capability is condition dependent. In one aspect, the conditions are physiological conditions, and the linker is protonated in vivo. In another embodiment, the spacers include both regions that are neutral and regions that may be protonated to carry one or more positive charges. In another embodiment, the spacers include both regions that may be deprotonated to carry one or more negative charges and regions that may be protonated to carry one or more positive charges. It is understood that in this latter embodiment that zwitterions or inner salts may be formed.

In one aspect, the regions of the linkers that may be deprotonated to carry a negative charge include carboxylic acids, such as aspartic acid, glutamic acid, and longer chain carboxylic acid groups, and sulfuric acid esters, such as alkyl esters of sulfuric acid. In another aspect, the regions of the linkers that may be protonated to carry a positive charge include amino groups, such as polyaminoalkylenes including ethylene diamines, propylene diamines, butylene diamines and the like, and/or heterocycles including pyrollidines, piperidines, piperazines, and other amino groups, each of which is optionally substituted. In another embodiment, the regions of the linkers that are neutral include poly hydroxyl groups, such as sugars, carbohydrates, saccharides, inositols, and the like, and/or polyether groups, such as polyoxyalkylene groups including polyoxyethylene, polyoxypropylene, and the like.

In one embodiment, the hydrophilic spacer linkers described herein include are formed primarily from carbon, hydrogen, and oxygen, and have a carbon/oxygen ratio of about 3:1 or less, or of about 2:1 or less. In one aspect, the hydrophilic linkers described herein include a plurality of ether functional groups. In another aspect, the hydrophilic linkers described herein include a plurality of hydroxyl functional groups. Illustrative fragments that may be used to form such linkers include polyhydroxyl compounds such as carbohydrates, polyether compounds such as polyethylene glycol units, and acid groups such as carboxyl and alkyl sulfuric acids. In one variation, oligoamide spacers, and the like may also be included in the linker.

Illustrative carbohydrate spacers include saccharopeptides as described herein that include both a peptide feature and sugar feature; glucuronides, which may be incorporated via click chemistry; 3-alkyl glycosides, such as of 2-deoxyhexapyranoses (2-deoxyglucose, 2-deoxyglucuronide, and the like), and 13-alkyl mannopyranosides. Illustrative PEG groups include those of a specific length range from about 4 to about 20 PEG groups. Illustrative alkyl sulfuric acid esters may also be introduced with click chemistry directly into the backbone. Illustrative oligoamide spacers include EDTA and DTPA spacers, 3-amino acids, and the like.

In another embodiment, the hydrophilic spacer linkers described herein include a polyether, such as the linkers of the following formulae:

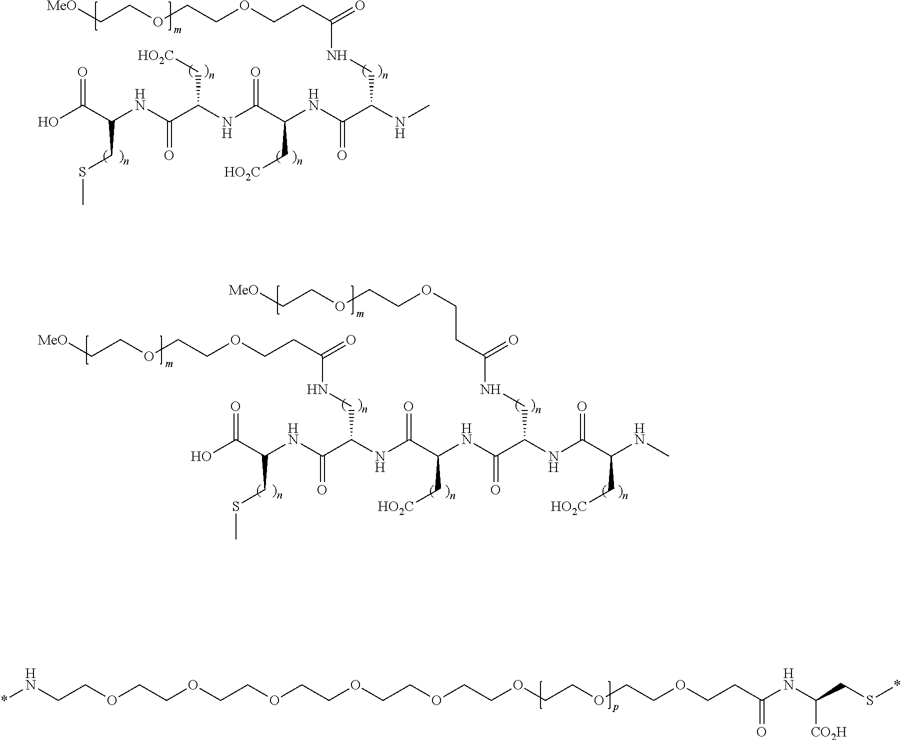

where m is an integer independently selected in each instance from 1 to about 8; p is an integer selected 1 to about 10; and n is an integer independently selected in each instance from 1 to about 3. In one aspect, m is independently in each instance 1 to about 3. In another aspect, n is 1 in each instance. In another aspect, p is independently in each instance about 4 to about 6. Illustratively, the corresponding polypropylene polyethers corresponding to the foregoing are contemplated herein and may be included in the conjugates as hydrophilic spacer linkers. In addition, it is appreciated that mixed polyethylene and polypropylene polyethers may be included in the conjugates as hydrophilic spacer linkers. Further, cyclic variations of the foregoing polyether compounds, such as those that include tetrahydrofuranyl, 1,3-dioxanes, 1,4-dioxanes, and the like are contemplated herein.

In another illustrative embodiment, the hydrophilic spacer linkers described herein include a plurality of hydroxyl functional groups, such as linkers that incorporate monosaccharides, oligosaccharides, polysaccharides, and the like. It is to be understood that the polyhydroxyl containing spacer linkers comprises a plurality of —(CROH)— groups, where R is hydrogen or alkyl.

In another embodiment, the spacer linkers include one or more of the following fragments:

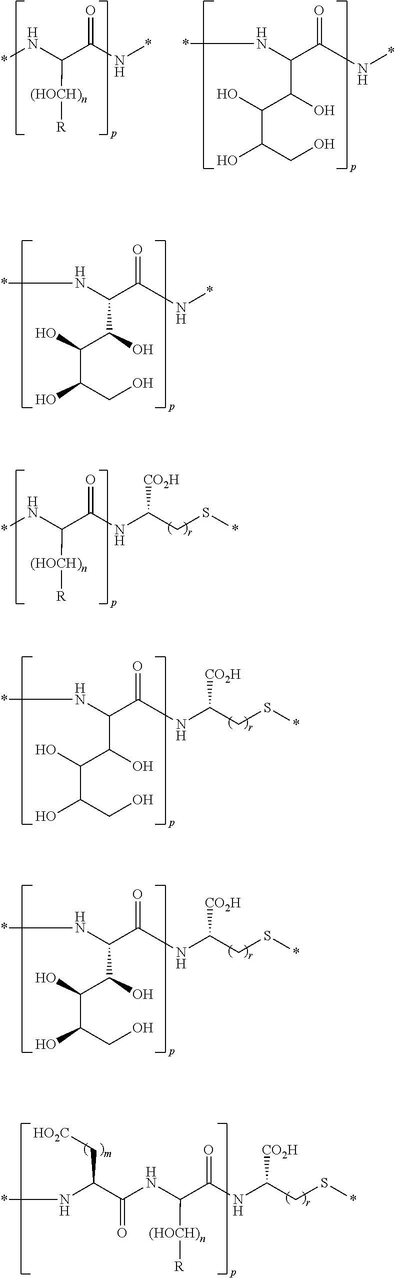

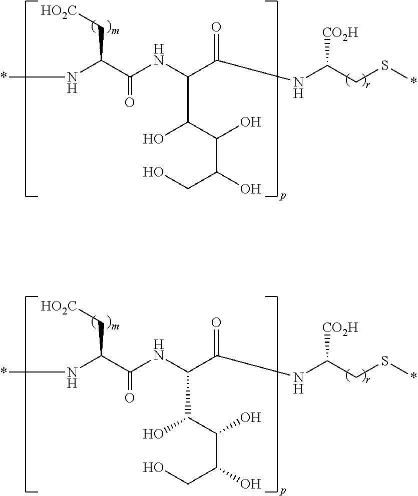

wherein R is H, alkyl, cycloalkyl, or arylalkyl; m is an integer from 1 to about 3; n is an integer from 1 to about 5, p is an integer from 1 to about 5, and r is an integer selected from 1 to about 3. In one aspect, the integer n is 3 or 4. In another aspect, the integer p is 3 or 4. In another aspect, the integer r is 1.

In another embodiment, the spacer linker includes one or more of the following cyclic polyhydroxyl groups:

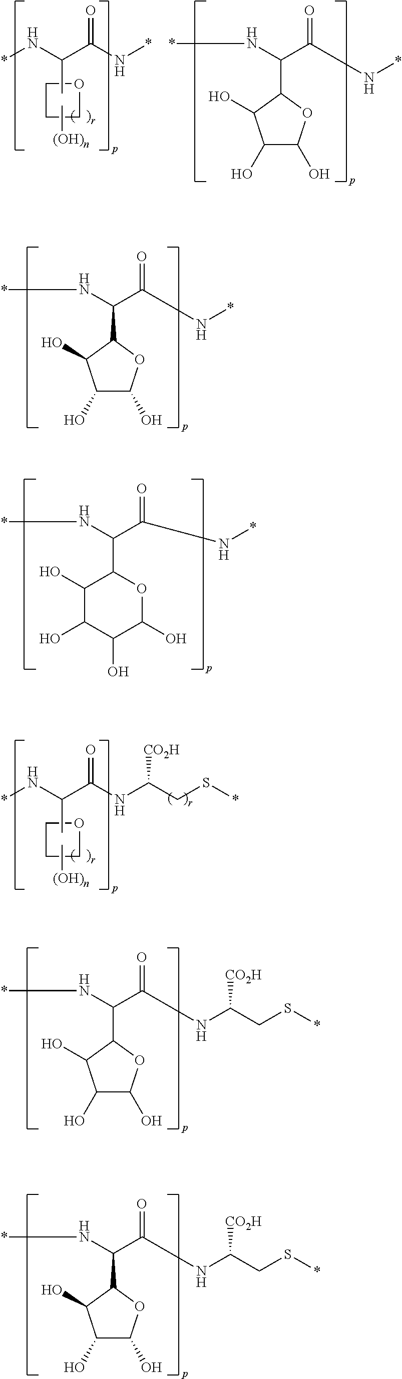

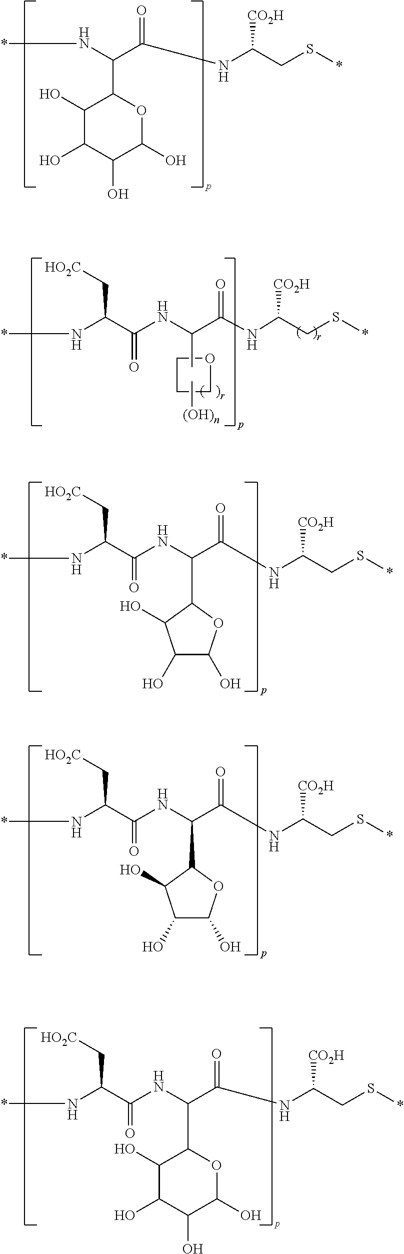

wherein n is an integer from 2 to about 5, p is an integer from 1 to about 5, and r is an integer from 1 to about 4. In one aspect, the integer n is 3 or 4. In another aspect, the integer p is 3 or 4. In another aspect, the integer r is 2 or 3. It is understood that all stereochemical forms of such sections of the linkers are contemplated herein. For example, in the above formula, the section may be derived from ribose, xylose, glucose, mannose, galactose, or other sugar and retain the stereochemical arrangements of pendant hydroxyl and alkyl groups present on those molecules. In addition, it is to be understood that in the foregoing formulae, various deoxy compounds are also contemplated. Illustratively, compounds of the following formulae are contemplated:

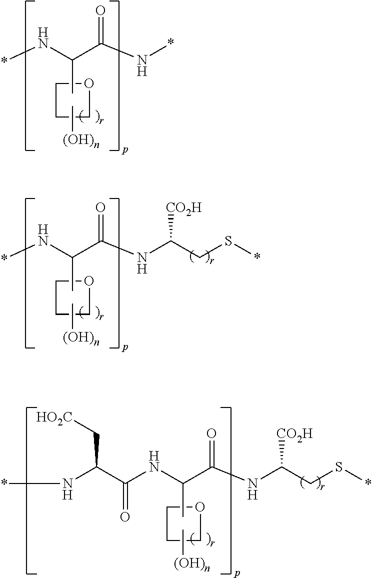

wherein n is equal to or less than r, such as when r is 2 or 3, n is 1 or 2, or 1, 2, or 3, respectively.

In another embodiment, the spacer linker includes a polyhydroxyl compound of the following formula

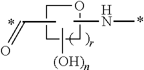

wherein n and r are each an integer selected from 1 to about 3. In one aspect, the spacer linker includes one or more polyhydroxyl compounds of the following formulae:

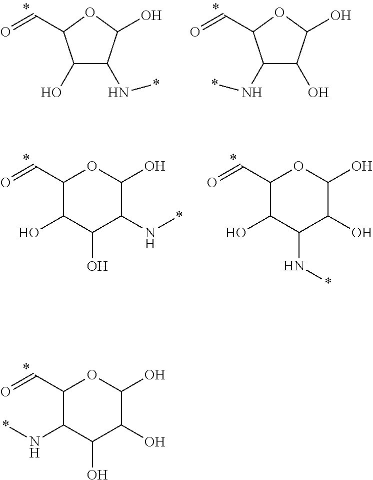

It is understood that all stereochemical forms of such sections of the linkers are contemplated herein. For example, in the above formula, the section may be derived from ribose, xylose, glucose, mannose, galactose, or other sugar and retain the stereochemical arrangements of pendant hydroxyl and alkyl groups present on those molecules.

In another configuration, the hydrophilic linkers L described herein include polyhydroxyl groups that are spaced away from the backbone of the linker Illustratively, such linkers include fragments of the following formulae:

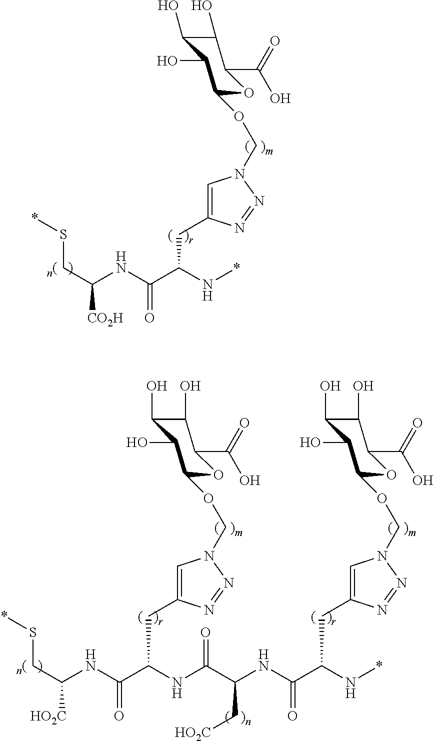

wherein n, m, and r are integers and are each independently selected in each instance from 1 to about 5. In one illustrative aspect, m is independently 2 or 3 in each instance. In another aspect, r is 1 in each instance. In another aspect, n is 1 in each instance. In one variation, the group connecting the polyhydroxyl group to the backbone of the linker is a different heteroaryl group, including but not limited to, pyrrole, pyrazole, 1,2,4-triazole, furan, oxazole, isoxazole, thienyl, thiazole, isothiazole, oxadiazole, and the like. Similarly, divalent 6-membered ring heteroaryl groups are contemplated. Other variations of the foregoing illustrative hydrophilic spacer linkers include oxyalkylene groups, such as the following formulae:

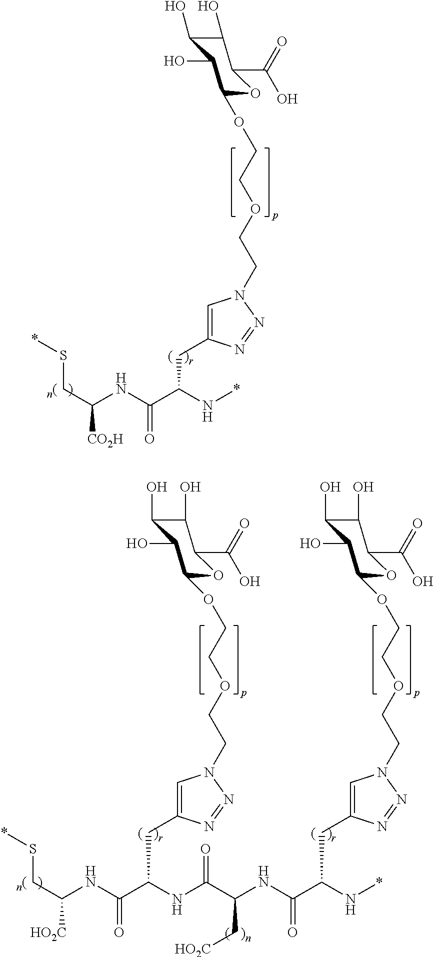

wherein n and r are integers and are each independently selected in each instance from 1 to about 5; and p is an integer selected from 1 to about 4.

In another embodiment, the hydrophilic linkers L described herein include polyhydroxyl groups that are spaced away from the backbone of the linker Illustratively, such linkers include fragments of the following formulae:

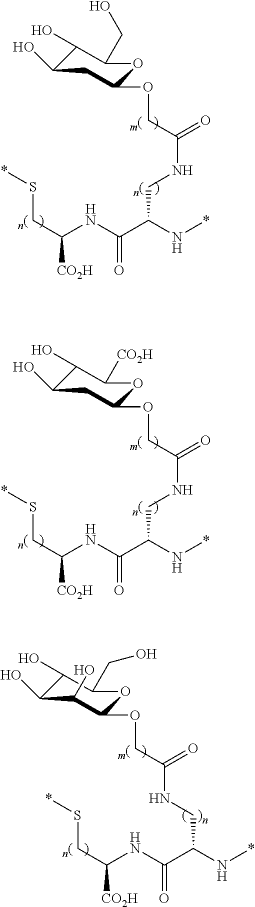

wherein n is an integer selected from 1 to about 3, and m is an integer selected from 1 to about 22. In one illustrative aspect, n is 1 or 2. In another illustrative aspect, m is selected from about 6 to about 10, illustratively 8. In one variation, the group connecting the polyhydroxyl group to the backbone of the linker is a different functional group, including but not limited to, esters, ureas, carbamates, acylhydrazones, and the like. Similarly, cyclic variations are contemplated. Other variations of the foregoing illustrative hydrophilic spacer linkers include oxyalkylene groups, such as the following formulae:

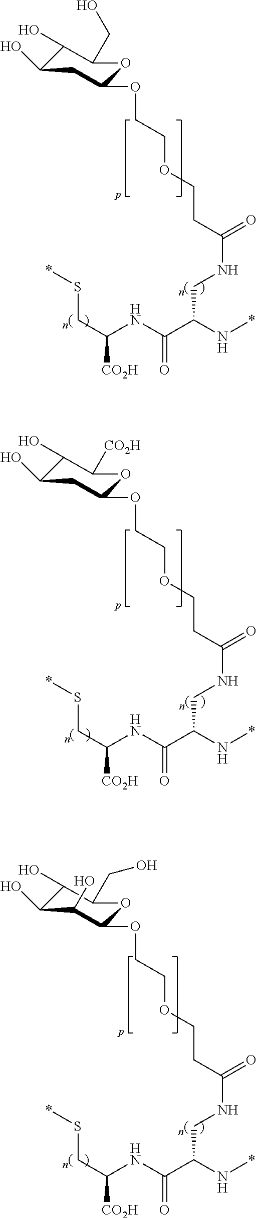

wherein n and r are integers and are each independently selected in each instance from 1 to about 5; and p is an integer selected from 1 to about 4.

In another embodiment, the hydrophilic spacer linker is a combination of backbone and branching side motifs such as is illustrated by the following formulae

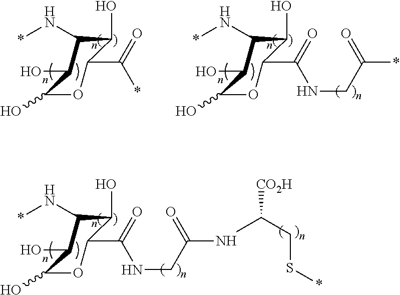

wherein n is an integer independently selected in each instance from 0 to about 3. The above formula are intended to represent 4, 5, 6, and even larger membered cyclic sugars. In addition, it is to be understood that the above formula may be modified to represent deoxy sugars, where one or more of the hydroxy groups present on the formulae are replaced by hydrogen, alkyl, or amino. In addition, it is to be understood that the corresponding carbonyl compounds are contemplated by the above formulae, where one or more of the hydroxyl groups is oxidized to the corresponding carbonyl. In addition, in this illustrative embodiment, the pyranose includes both carboxyl and amino functional groups and (a) can be inserted into the backbone and (b) can provide synthetic handles for branching side chains in variations of this embodiment. Any of the pendant hydroxyl groups may be used to attach other chemical fragments, including additional sugars to prepare the corresponding oligosaccharides. Other variations of this embodiment are also contemplated, including inserting the pyranose or other sugar into the backbone at a single carbon, i.e. a spiro arrangement, at a geminal pair of carbons, and like arrangements. For example, one or two ends of the linker, or the nucleotide N, or the binding ligand B may be connected to the sugar to be inserted into the backbone in a 1,1; 1,2; 1,3; 1,4; 2,3, or other arrangement.

In another embodiment, the hydrophilic spacer linkers described herein include are formed primarily from carbon, hydrogen, and nitrogen, and have a carbon/nitrogen ratio of about 3:1 or less, or of about 2:1 or less. In one aspect, the hydrophilic linkers described herein include a plurality of amino functional groups.

In another embodiment, the spacer linkers include one or more amino groups of the following formulae:

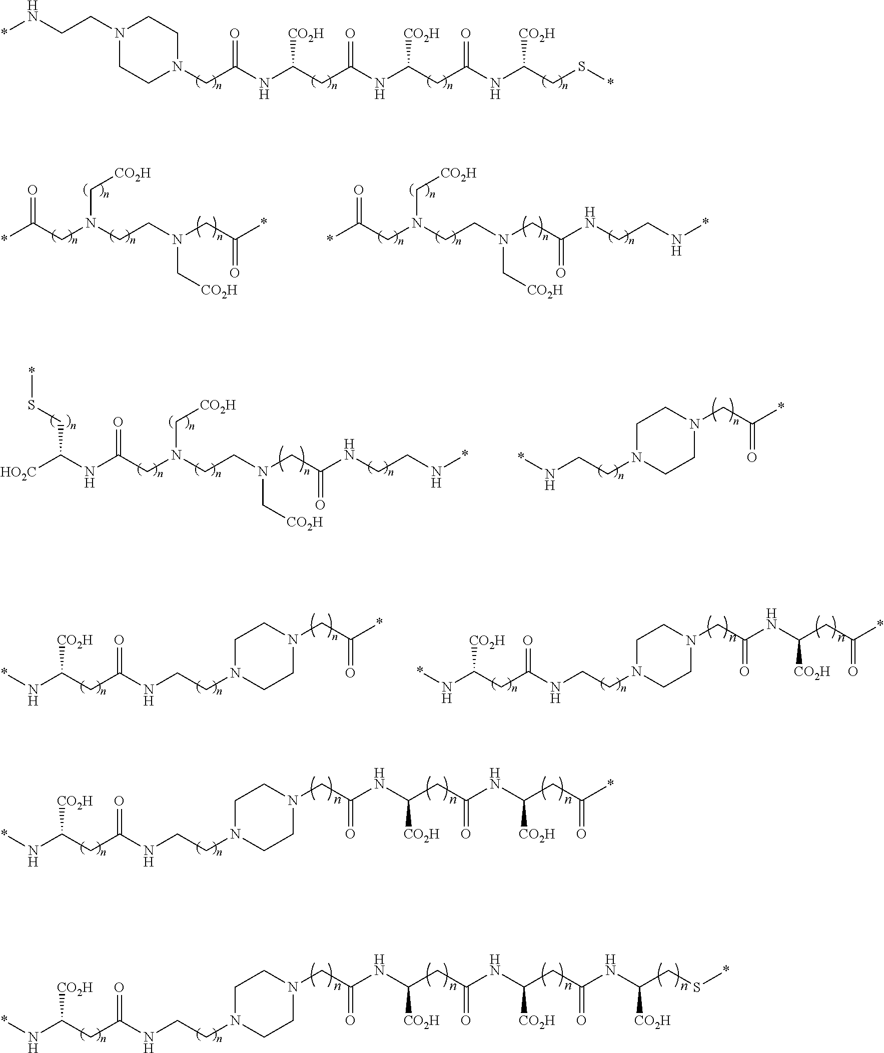

where n is an integer independently selected in each instance from 1 to about 3. In one aspect, the integer n is independently 1 or 2 in each instance. In another aspect, the integer n is 1 in each instance.

In another embodiment, the hydrophilic spacer linker is a sulfuric acid ester, such as an alkyl ester of sulfuric acid. Illustratively, the spacer linker is of the following formula:

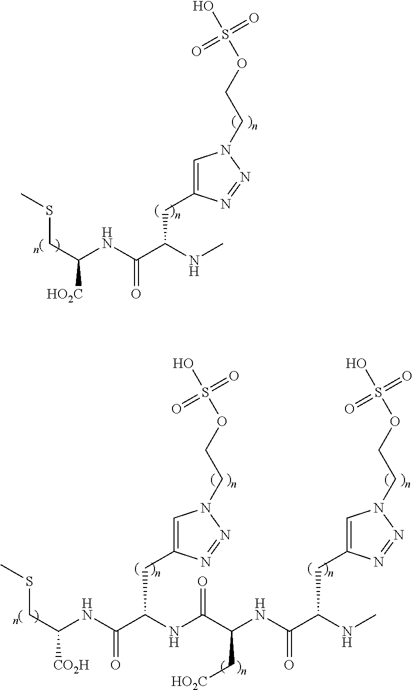

where n is an integer independently selected in each instance from 1 to about 3. Illustratively, n is independently 1 or 2 in each instance.

It is understood, that in such polyhydroxyl, polyamino, carboxylic acid, sulfuric acid, and like linkers that include free hydrogens bound to heteroatoms, one or more of those free hydrogen atoms may be protected with the appropriate hydroxyl, amino, or acid protecting group, respectively, or alternatively may be blocked as the corresponding pro-drugs, the latter of which are selected for the particular use, such as pro-drugs that release the parent drug (i.e., the nucleotide) under general or specific physiological conditions.

In each of the foregoing illustrative examples of linkers L, there are also included in some cases additional spacer linkers L_{S}, and/or additional releasable linkers L_{R}. Those spacer linker and releasable linkers also may include asymmetric carbon atoms. It is to be further understood that the stereochemical configurations shown herein are merely illustrative, and other stereochemical configurations are contemplated. For example in one variation, the corresponding unnatural amino acid configurations may be included in the conjugated described herein as follows:

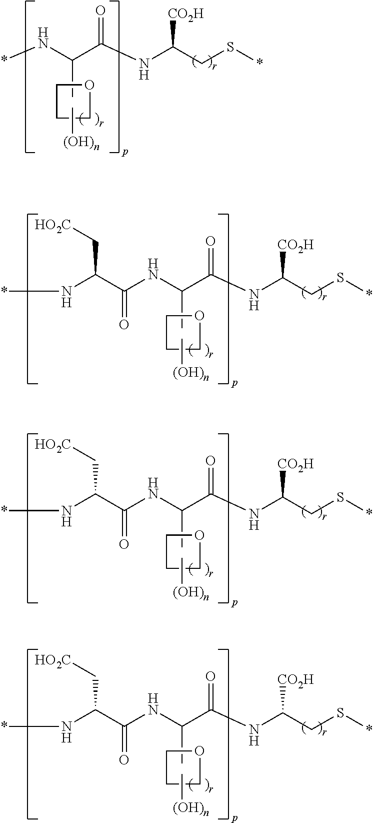

wherein n is an integer from 2 to about 5, p is an integer from 1 to about 5, and r is an integer from 1 to about 4, as described above.

It is to be further understood that in the foregoing embodiments, open positions, such as (*) atoms are locations for attachment of the binding ligand (B) or the nucleotide (A) to be delivered. In addition, it is to be understood that such attachment of either or both of B and N may be direct or through an intervening linker. Intervening linkers include other spacer linkers and/or releasable linkers. Illustrative additional spacer linkers and releasable linkers that are included in the conjugates described herein are described in U.S. Patent Application Ser. No. 10/765,335, the disclosure of which is incorporated herein by reference.

In another embodiment, the additional spacer linker can be 1-alkylenesuccinimid-3-yl, optionally substituted with a substituent X¹, as defined below, and the releasable linkers can be methylene, 1-alkoxyalkylene, 1-alkoxycycloalkylene, 1-alkoxyalkylenecarbonyl, 1-alkoxycycloalkylenecarbonyl, wherein each of the releasable linkers is optionally substituted with a substituent X², as defined below, and wherein the spacer linker and the releasable linker are each bonded to the spacer linker to form a succinimid-1-ylalkyl acetal or ketal.

The additional spacer linkers can be carbonyl, thionocarbonyl, alkylene, cycloalkylene, alkylenecycloalkyl, alkylenecarbonyl, cycloalkylenecarbonyl, carbonylalkylcarbonyl, 1-alkylenesuccinimid-3-yl, 1-(carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl, and 1-(carbonyltetrahydrofuranyl)succinimid-3-yl, wherein each of the spacer linkers is optionally substituted with a substituent X¹, as defined below. In this embodiment, the spacer linker may include an additional nitrogen, and the spacer linkers can be alkylenecarbonyl, cycloalkylenecarbonyl, carbonylalkylcarbonyl, 1-(carbonylalkyl)succinimid-3-yl, wherein each of the spacer linkers is optionally substituted with a substituent X¹, as defined below, and the spacer linker is bonded to the nitrogen to form an amide. Alternatively, the spacer linker may include an additional sulfur, and the spacer linkers can be alkylene and cycloalkylene, wherein each of the spacer linkers is optionally substituted with carboxy, and the spacer linker is bonded to the sulfur to form a thiol. In another embodiment, the spacer linker can include sulfur, and the spacer linkers can be 1-alkylenesuccinimid-3-yl and 1-(carbonylalkyl)succinimid-3-yl, and the spacer linker is bonded to the sulfur to form a succinimid-3-ylthiol.

In an alternative to the above-described embodiments, the additional spacer linker can include nitrogen, and the releasable linker can be a divalent radical comprising alkyleneaziridin-1-yl, carbonylalkylaziridin-1-yl, sulfoxylalkylaziridin-1-yl, or sulfonylalkylaziridin-1-yl, wherein each of the releasable linkers is optionally substituted with a substituent X², as defined below. In this alternative embodiment, the spacer linkers can be carbonyl, thionocarbonyl, alkylenecarbonyl, cycloalkylenecarbonyl, carbonylalkylcarbonyl, 1-(carbonylalkyl)succinimid-3-yl, wherein each of the spacer linkers is optionally substituted with a substituent X¹, as defined below, and wherein the spacer linker is bonded to the releasable linker to form an aziridine amide.

The substituents X¹ can be alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides. In this embodiment the spacer linker can include nitrogen, and the substituent X¹ and the spacer linker to which they are bound to form an heterocycle.

In another embodiment, the releasable linker may be a divalent radical comprising alkyleneaziridin-1-yl, alkylenecarbonylaziridin-1-yl, carbonylalkylaziridin-1-yl, alkylenesulfoxylaziridin-1-yl, sulfoxylalkylaziridin-1-yl, sulfonylalkylaziridin-1-yl, or alkylenesulfonylaziridin-1-yl, wherein each of the releasable linkers is optionally substituted with a substituent X², as defined below.

Additional illustrative releasable linkers include methylene, 1-alkoxyalkylene, 1-alkoxycycloalkylene, 1-alkoxyalkylenecarbonyl, 1-alkoxycycloalkylenecarbonyl, carbonylarylcarbonyl, carbonyl(carboxyaryl)carbonyl, carbonyl(biscarboxyaryl)carbonyl, haloalkylenecarbonyl, alkylene(dialkylsilyl), alkylene(alkylarylsilyl), alkylene(diarylsilyl), (dialkylsilyl)aryl, (alkylarylsilyl)aryl, (diarylsilyl)aryl, oxycarbonyloxy, oxycarbonyloxyalkyl, sulfonyloxy, oxysulfonylalkyl, iminoalkylidenyl, carbonylalkylideniminyl, iminocycloalkylidenyl, carbonylcycloalkylideniminyl, alkylenethio, alkylenearylthio, and carbonylalkylthio, wherein each of the releasable linkers is optionally substituted with a substituent X², as defined below.

In the preceding embodiment, the releasable linker may include oxygen, and the releasable linkers can be methylene, 1-alkoxyalkylene, 1-alkoxycycloalkylene, 1-alkoxyalkylenecarbonyl, and 1-alkoxycycloalkylenecarbonyl, wherein each of the releasable linkers is optionally substituted with a substituent X², as defined below, and the releasable linker is bonded to the oxygen to form an acetal or ketal. Alternatively, the releasable linker may include oxygen, and the releasable linker can be methylene, wherein the methylene is substituted with an optionally-substituted aryl, and the releasable linker is bonded to the oxygen to form an acetal or ketal. Further, the releasable linker may include oxygen, and the releasable linker can be sulfonylalkyl, and the releasable linker is bonded to the oxygen to form an alkylsulfonate.

In another embodiment of the above releasable linker embodiment, the releasable linker may include nitrogen, and the releasable linkers can be iminoalkylidenyl, carbonylalkylideniminyl, iminocycloalkylidenyl, and carbonylcycloalkylideniminyl, wherein each of the releasable linkers is optionally substituted with a substituent X², as defined below, and the releasable linker is bonded to the nitrogen to form an hydrazone. In an alternate configuration, the hydrazone may be acylated with a carboxylic acid derivative, an orthoformate derivative, or a carbamoyl derivative to form various acylhydrazone releasable linkers.

Alternatively, the releasable linker may include alkylene(dialkylsilyl)oxy, alkylene(alkylarylsilyl)oxy, alkylene(diarylsilyl)oxy, oxy(dialkylsilyl)aryl, oxy(alkylarylsilyl)aryl, and oxy(diarylsilyl)aryl, wherein each of the releasable linkers is optionally substituted with a substituent X², as defined below.

Alternatively, the releasable linker may include diesters, ester-amides, and/or diamides of carbonylarylcarbonyl, carbonyl(carboxyaryl)carbonyl, and carbonyl(biscarboxyaryl)carbonyl.

Substituents X² can be alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides. In this embodiment the releasable linker can include nitrogen, and the substituent X² and the releasable linker can form an heterocycle.

Heterocycles can be pyrrolidines, piperidines, oxazolidines, isoxazolidines, thiazolidines, isothiazolidines, pyrrolidinones, piperidinones, oxazolidinones, isoxazolidinones, thiazolidinones, isothiazolidinones, and succinimides.

Nucleotide N can include a nitrogen atom, and the releasable linker can be haloalkylenecarbonyl, optionally substituted with a substituent X², and the releasable linker is bonded to the nucleotide nitrogen to form an amide. Nucleotide N can include a double-bonded nitrogen atom, and in this embodiment, the releasable linkers can be alkylenecarbonylamino and 1-(alkylenecarbonylamino)succinimid-3-yl, and the releasable linker can be bonded to the nucleotide nitrogen to form an hydrazone.

Nucleotide N can include an oxygen atom, and the releasable linker can be haloalkylenecarbonyl, optionally substituted with a substituent X², and the releasable linker is bonded to the nucleotide oxygen to form an ester. Nucleotide N can include a sulfur atom, and in this embodiment, the releasable linkers can be alkylenethio and carbonylalkylthio, and the releasable linker can be bonded to the nucleotide sulfur to form a disulfide.

Binding ligand B can be folate which includes a nitrogen, and in this embodiment, the spacer linkers can be alkylenecarbonyl, cycloalkylenecarbonyl, carbonylalkylcarbonyl, 1-alkylenesuccinimid-3-yl, 1-(carbonylalkyl)succinimid-3-yl, wherein each of the spacer linkers is optionally substituted with a substituent X¹, and the spacer linker is bonded to the folate nitrogen to form an imide or an alkylamide. In this embodiment, the substituents X¹ can be alkyl, hydroxyalkyl, amino, aminoalkyl, alkylaminoalkyl, dialkylaminoalkyl, sulfhydrylalkyl, alkylthioalkyl, aryl, substituted aryl, arylalkyl, substituted arylalkyl, carboxy, carboxyalkyl, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides.

The term cycloalkylene as used herein refers to a bivalent chain of carbon atoms, a portion of which forms a ring, such as cycloprop-1,1-diyl, cycloprop-1,2-diyl, cyclohex-1,4-diyl, 3-ethylcyclopent-1,2-diyl, 1-methylenecyclohex-4-yl, and the like.

The term heterocycle as used herein refers to a monovalent chain of carbon and heteroatoms, wherein the heteroatoms are selected from nitrogen, oxygen, and sulfur, a portion of which, including at least one heteroatom, form a ring, such as aziridine, pyrrolidine, oxazolidine, 3-methoxypyrrolidine, 3-methylpiperazine, and the like.

The term aryl as used herein refers to an aromatic mono or polycyclic ring of carbon atoms, such as phenyl, naphthyl, and the like. In addition, aryl may also include heteroaryl.

The term heteroaryl as used herein refers to an aromatic mono or polycyclic ring of carbon atoms and at least one heteroatom selected from nitrogen, oxygen, and sulfur, such as pyridinyl, pyrimidinyl, indolyl, benzoxazolyl, and the like.

The term optionally substituted as used herein refers to the replacement of one or more hydrogen atoms, generally on carbon, with a corresponding number of substituents, such as halo, hydroxy, amino, alkyl or dialkylamino, alkoxy, alkylsulfonyl, cyano, nitro, and the like. In addition, two hydrogens on the same carbon, on adjacent carbons, or nearby carbons may be replaced with a bivalent substituent to form the corresponding cyclic structure.

The term iminoalkylidenyl as used herein refers to a divalent radical containing alkylene as defined herein and a nitrogen atom, where the terminal carbon of the alkylene is double-bonded to the nitrogen atom, such as the formulae —(CH)═N—, —(CH₂)₂(CH)═N—, —CH₂C(Me)═N—, and the like.

The term amino acid as used herein refers generally to aminoalkylcarboxylate, where the alkyl radical is optionally substituted, such as with alkyl, hydroxy alkyl, sulfhydrylalkyl, aminoalkyl, carboxyalkyl, and the like, including groups corresponding to the naturally occurring amino acids, such as serine, cysteine, methionine, aspartic acid, glutamic acid, and the like. It is to be understood that such amino acids may be of a single stereochemistry or a particular mixture of stereochemisties, including racemic mixtures. In addition, amino acid refers to beta, gamma, and longer amino acids, such as amino acids of the formula:

—N(R)—(CR′R″)_{q}C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornithine, threonine, and the like. In another illustrative aspect of the vitamin receptor binding nucleotide delivery conjugate intermediate described herein, the nucleotide, or an analog or a derivative thereof, includes an alkylthiol nucleophile.

It is to be understood that the above-described terms can be combined to generate chemically-relevant groups, such as alkoxyalkyl referring to methyloxymethyl, ethyloxyethyl, and the like, haloalkoxyalkyl referring to trifluoromethyloxyethyl, 1,2-difluoro-2-chloroeth-1-yloxypropyl, and the like, arylalkyl referring to benzyl, phenethyl, a-methylbenzyl, and the like, and others.

The term amino acid derivative as used herein refers generally to an optionally substituted aminoalkylcarboxylate, where the amino group and/or the carboxylate group are each optionally substituted, such as with alkyl, carboxylalkyl, alkylamino, and the like, or optionally protected. In addition, the optionally substituted intervening divalent alkyl fragment may include additional groups, such as protecting groups, and the like.

The term peptide as used herein refers generally to a series of amino acids and/or amino acid analogs and derivatives covalently linked one to the other by amide bonds.

The term releasable linker as used herein refers to a linker that includes at least one bond that can be broken under physiological conditions (e.g., a pH-labile, acid-labile, oxidatively-labile, or enzyme-labile bond). It should be appreciated that such physiological conditions resulting in bond breaking include standard chemical hydrolysis reactions that occur, for example, at physiological pH, or as a result of compartmentalization into a cellular organelle such as an endosome having a lower pH than cytosolic pH.

The releasable linker includes at least one bond that can be broken or cleaved under physiological conditions, such as a pH-labile, acid-labile, oxidatively-labile, or enzyme-labile bond. The cleavable bond or bonds may be present in the interior of a cleavable linker and/or at one or both ends of a cleavable linker. It is appreciated that the lability of the cleavable bond may be adjusted by including functional groups or fragments within the bivalent linker L that are able to assist or facilitate such bond breakage, also termed anchimeric assistance. In addition, it is appreciated that additional functional groups or fragments may be included within the bivalent linker L that are able to assist or facilitate additional fragmentation of the receptor binding ligand agent conjugates after bond breaking of the releasable linker. The lability of the cleavable bond can be adjusted by, for example, substitutional changes at or near the cleavable bond, such as including alpha branching adjacent to a cleavable disulfide bond, increasing the hydrophobicity of substituents on silicon in a moiety having a silicon-oxygen bond that may be hydrolyzed, homologating alkoxy groups that form part of a ketal or acetal that may be hydrolyzed, and the like.

It is understood that a cleavable bond can connect two adjacent atoms within the releasable linker and/or connect other linkers or B and/or N, as described herein, at either or both ends of the releasable linker. In the case where a cleavable bond connects two adjacent atoms within the releasable linker, following breakage of the bond, the releasable linker is broken into two or more fragments. Alternatively, in the case where a cleavable bond is between the releasable linker and another moiety, such as an additional heteroatom, additional spacer linker, another releasable linker, the nucleotide N, or analog or derivative thereof, or the binding ligand B, or analog or derivative thereof, following breakage of the bond, the releasable linker is separated from the other moiety.

It is understood that each of the additional spacer and releasable linkers are bivalent. It should be further understood that the connectivity between each of the various additional spacer and releasable linkers themselves, and between the various additional spacer and releasable linkers and N and/or B, as defined herein, may occur at any atom found in the various additional spacer or releasable linkers.

In another embodiment, a folate receptor binding nucleotide delivery conjugate of the general formula B-L-N is described, wherein L comprises a hydrophilic spacer linker, and optional additional spacer linkers lₛ, and/or releasable linkers 1_{R}, and combinations thereof.

In one aspect of the various receptor binding nucleotide delivery conjugates described herein, the bivalent linker comprises an additional spacer linker and a releasable linker taken together to form 3-thiosuccinimid-1-ylalkyloxymethyloxy, where the methyl is optionally substituted with alkyl or substituted aryl.

In another aspect, the bivalent linker comprises an additional spacer linker and a releasable linker taken together to form 3-thiosuccinimid-1-ylalkylcarbonyl, where the carbonyl forms an acylaziridine with nucleotide N, or analog or derivative thereof.

In another aspect, the bivalent linker comprises an additional spacer linker and a releasable linker taken together to form 1-alkoxycycloalkylenoxy.

In another aspect, the bivalent linker comprises an additional spacer linker and a releasable linker taken together to form alkyleneaminocarbonyl(dicarboxylarylene) carboxylate.

In another aspect, the bivalent linker comprises a releasable linker, an additional spacer linker, and a releasable linker taken together to form dithioalkylcarbonylhydrazide, where the hydrazide forms an hydrazone with nucleotide N, or analog or derivative thereof.

In another aspect, the bivalent linker comprises an additional spacer linker and a releasable linker taken together to form 3-thiosuccinimid-1-ylalkylcarbonylhydrazide, where the hydrazide forms an hydrazone with nucleotide N, or analog or derivative thereof.

In another aspect, the bivalent linker comprises an additional spacer linker and a releasable linker taken together to form 3-thioalkylsulfonylalkyl(disubstituted silyl)oxy, where the disubstituted silyl is substituted with alkyl or optionally substituted aryl.

In another aspect, the bivalent linker comprises a plurality of additional spacer linkers selected from the group consisting of the naturally occurring amino acids and stereoisomers thereof.

In another aspect, the bivalent linker comprises a releasable linker, an additional spacer linker, and a releasable linker taken together to form 3-dithioalkyloxycarbonyl, where the carbonyl forms a carbonate with nucleotide N, or analog or derivative thereof.

In another aspect, the bivalent linker comprises a releasable linker, an additional spacer linker, and a releasable linker taken together to form 2-dithioalkyloxycarbonyl, where the carbonyl forms a carbonate with nucleotide N, or analog or derivative thereof.

In another aspect, the bivalent linker comprises a releasable linker, an additional spacer linker, and a releasable linker taken together to form 3-dithioarylalkyloxycarbonyl, where the carbonyl forms a carbonate with nucleotide N, or analog or derivative thereof, and the aryl is optionally substituted.

In another aspect, the bivalent linker comprises an additional spacer linker and a releasable linker taken together to form 3-thiosuccinimid-1-ylalkyloxyalkyloxyalkylidene, where the alkylidene forms an hydrazone with nucleotide N, or analog or derivative thereof, each alkyl is independently selected, and the oxyalkyloxy is optionally substituted with alkyl or optionally substituted aryl.

In another aspect, the bivalent linker comprises a releasable linker, an additional spacer linker, and a releasable linker taken together to form 3-dithioalkyloxycarbonylhydrazide.

In another aspect, the bivalent linker comprises a releasable linker, an additional spacer linker, and a releasable linker taken together to form 3-dithioalkyloxycarbonylhydrazide.

In another aspect, the bivalent linker comprises a releasable linker, an additional spacer linker, and a releasable linker taken together to form 2-dithioalkylamino, where the amino forms a vinylogous amide with nucleotide N, or analog or derivative thereof.

In another aspect, the bivalent linker comprises a releasable linker, an additional spacer linker, and a releasable linker taken together to form 3-dithioalkylamino, where the amino forms a vinylogous amide with nucleotide N, or analog or derivative thereof, and the alkyl is ethyl.

In another aspect, the bivalent linker comprises a releasable linker, an additional spacer linker, and a releasable linker taken together to form 3-dithioalkylaminocarbonyl, where the carbonyl forms a carbamate with nucleotide N, or analog or derivative thereof.

In another aspect, the bivalent linker comprises a releasable linker, an additional spacer linker, and a releasable linker taken together to form 3-dithioalkylaminocarbonyl, where the carbonyl forms a carbamate with nucleotide N, or analog or derivative thereof, and the alkyl is ethyl.

In another aspect, the bivalent linker comprises a releasable linker, an additional spacer linker, and a releasable linker taken together to form 3-dithioarylalkyloxycarbonyl, where the carbonyl forms a carbamate or a carbamoylaziridine with nucleotide N, or analog or derivative thereof.

In another embodiment, bivalent linker (L) includes a disulfide releasable linker. In another embodiment, bivalent linker (L) includes at least one releasable linker that is not a disulfide releasable linker.

In one aspect, the releasable and spacer linkers may be arranged in such a way that subsequent to the cleavage of a bond in the bivalent linker, released functional groups chemically assist the breakage or cleavage of additional bonds, also termed anchimeric assisted cleavage or breakage. An illustrative embodiment of such a bivalent linker or portion thereof includes compounds having the formulae:

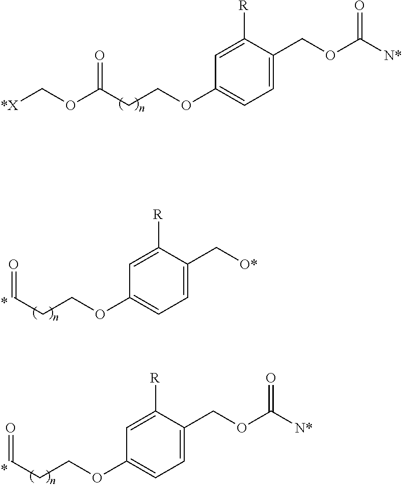

where X is an heteroatom, such as nitrogen, oxygen, or sulfur, or a carbonyl group; n is an integer selected from 0 to 4; illustratively 2; R is hydrogen, or a substituent, including a substituent capable of stabilizing a positive charge inductively or by resonance on the aryl ring, such as alkoxy and the like, including methoxy; and the symbol (*) indicates points of attachment for additional spacer, heteroatom, or releasable linkers forming the bivalent linker, or alternatively for attachment of the nucleotide, or analog or derivative thereof, or the vitamin, or analog or derivative thereof. In one embodiment, n is 2 and R is methoxy. It is appreciated that other substituents may be present on the aryl ring, the benzyl carbon, the alkanoic acid, or the methylene bridge, including but not limited to hydroxy, alkyl, alkoxy, alkylthio, halo, and the like. Assisted cleavage may include mechanisms involving benzylium intermediates, benzyne intermediates, lactone cyclization, oxonium intermediates, beta-elimination, and the like. It is further appreciated that, in addition to fragmentation subsequent to cleavage of the releasable linker, the initial cleavage of the releasable linker may be facilitated by an anchimeric ally assisted mechanism.

Illustrative examples of intermediates useful in forming such linkers include:

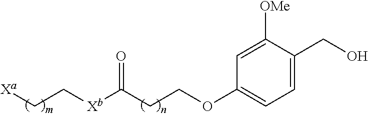

where X^{a} is an electrophilic group such as maleimide, vinyl sulfone, activated carboxylic acid derivatives, and the like, X^{b} is NH, O, or S; and m and n are each independently selected integers from 0-4. In one variation, m and n are each independently selected integers from 0-2. Such intermediates may be coupled to nucleotides, binding ligands, or other linkers via nucleophilic attack onto electrophilic group Xₐ, and/or by forming ethers or carboxylic acid derivatives of the benzylic hydroxyl group. In one embodiment, the benzylic hydroxyl group is converted into the corresponding activated benzyloxycarbonyl compound with phosgene or a phosgene equivalent. This embodiment may be coupled to nucleotides, binding ligands, or other linkers via nucleophilic attack onto the activated carbonyl group.

Illustrative mechanisms for cleavage of the bivalant linkers described herein include the following 1,4 and 1,6 fragmentation mechanisms

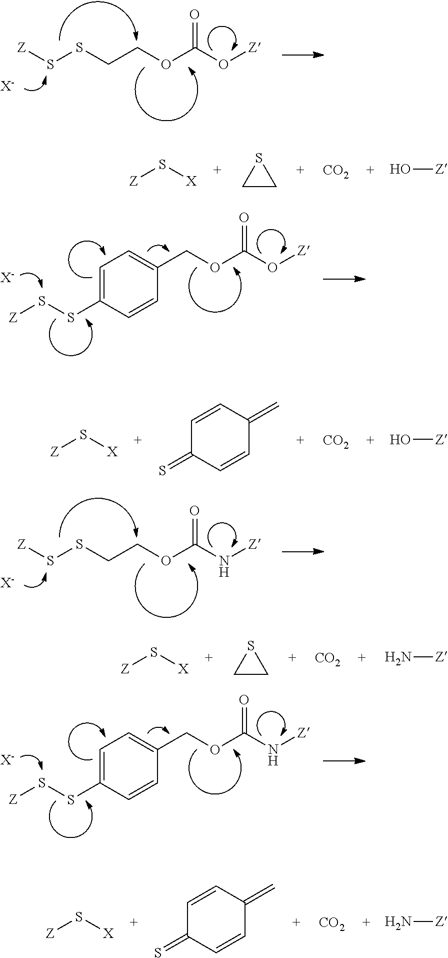

where X is an exogenous or endogenous nucleophile, glutathione, or bioreducing agent, and the like, and either of Z or Z′ is the vitamin, or analog or derivative thereof, or the nucleotide, or analog or derivative thereof, or a vitamin or nucleotide moiety in conjunction with other portions of the polyvalent linker. It is to be understood that although the above fragmentation mechanisms are depicted as concerted mechanisms, any number of discrete steps may take place to effect the ultimate fragmentation of the polyvalent linker to the final products shown. For example, it is appreciated that the bond cleavage may also occur by acid-catalyzed elimination of the carbamate moiety, which may be anchimerically assisted by the stabilization provided by either the aryl group of the beta sulfur or disulfide illustrated in the above examples. In those variations of this embodiment, the releasable linker is the carbamate moiety. Alternatively, the fragmentation may be initiated by a nucleophilic attack on the disulfide group, causing cleavage to form a thiolate. The thiolate may intermolecularly displace a carbonic acid or carbamic acid moiety and form the corresponding thiacyclopropane. In the case of the benzyl-containing polyvalent linkers, following an illustrative breaking of the disulfide bond, the resulting phenyl thiolate may further fragment to release a carbonic acid or carbamic acid moiety by forming a resonance stabilized intermediate. In any of these cases, the releasable nature of the illustrative polyvalent linkers described herein may be realized by whatever mechanism may be relevant to the chemical, metabolic, physiological, or biological conditions present.

Other illustrative mechanisms for bond cleavage of the releasable linker include oxonium-assisted cleavage as follows:

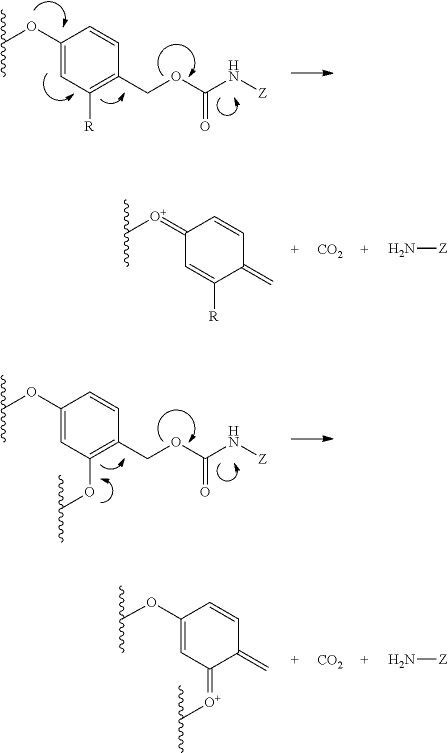

where Z is the vitamin, or analog or derivative thereof, or the nucleotide, or analog or derivative thereof, or each is a vitamin or nucleotide moiety in conjunction with other portions of the polyvalent linker, such as a nucleotide or vitamin moiety including one or more spacer linkers and/or other releasable linkers. Without being bound by theory, in this embodiment, acid catalysis, such as in an endosome, may initiate the cleavage via protonation of the urethane group. In addition, acid-catalyzed elimination of the carbamate leads to the release of CO₂ and the nitrogen-containing moiety attached to Z, and the formation of a benzyl cation, which may be trapped by water, or any other Lewis base.

Other illustrative linkers include compounds of the formulae:

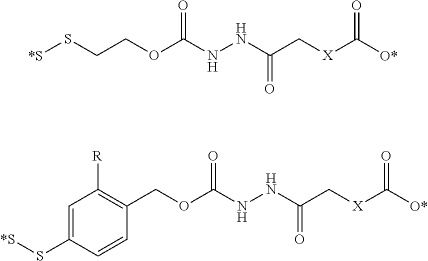

where X is NH, CH₂, or O; R is hydrogen, or a substituent, including a substituent capable of stabilizing a positive charge inductively or by resonance on the aryl ring, such as alkoxy and the like, including methoxy; and the symbol (*) indicates points of attachment for additional spacer, heteroatom, or releasable linkers forming the bivalent linker, or alternatively for attachment of the nucleotide, or analog or derivative thereof, or the vitamin, or analog or derivative thereof.

Illustrative mechanisms for cleavage of such bivalent linkers described herein include the following 1,4 and 1,6 fragmentation mechanisms followed by anchimerically assisted cleavage of the acylated Z′ via cyclization by the hydrazide group:

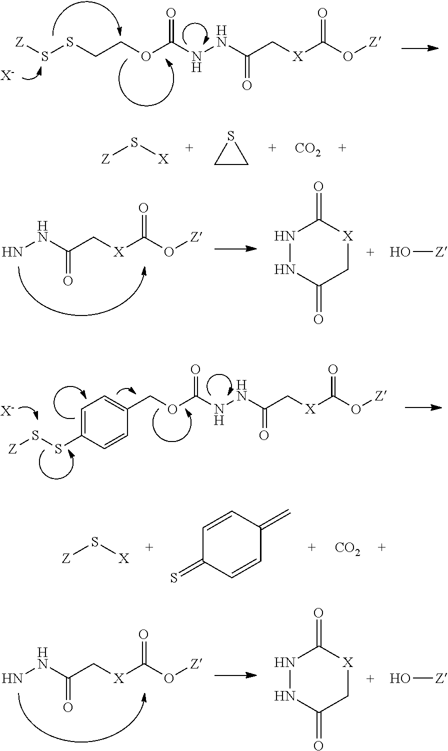

where X is an exogenous or endogenous nucleophile, glutathione, or bioreducing agent, and the like, and either of Z or Z′ is the vitamin, or analog or derivative thereof, or the nucleotide, or analog or derivative thereof, or a vitamin or nucleotide in conjunction with other portions of the polyvalent linker. It is to be understood that although the above fragmentation mechanisms are depicted as concerted mechanisms, any number of discrete steps may take place to effect the ultimate fragmentation of the polyvalent linker to the final products shown. For example, it is appreciated that the bond cleavage may also occur by acid-catalyzed elimination of the carbamate moiety, which may be anchimerically assisted by the stabilization provided by either the aryl group of the beta sulfur or disulfide illustrated in the above examples. In those variations of this embodiment, the releasable linker is the carbamate moiety. Alternatively, the fragmentation may be initiated by a nucleophilic attack on the disulfide group, causing cleavage to form a thiolate. The thiolate may intermolecularly displace a carbonic acid or carbamic acid moiety and form the corresponding thiacyclopropane. In the case of the benzyl-containing polyvalent linkers, following an illustrative breaking of the disulfide bond, the resulting phenyl thiolate may further fragment to release a carbonic acid or carbamic acid moiety by forming a resonance stabilized intermediate. In any of these cases, the releasable nature of the illustrative polyvalent linkers described herein may be realized by whatever mechanism may be relevant to the chemical, metabolic, physiological, or biological conditions present. Without being bound by theory, in this embodiment, acid catalysis, such as in an endosome, may also initiate the cleavage via protonation of the urethane group. In addition, acid-catalyzed elimination of the carbamate leads to the release of CO₂ and the nitrogen-containing moiety attached to Z, and the formation of a benzyl cation, which may be trapped by water, or any other Lewis base, as is similarly described herein.

In one embodiment, the polyvalent linkers described herein are compounds of the following formulae

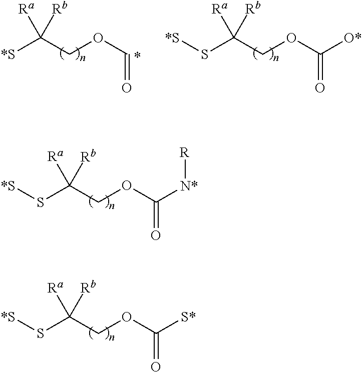

where n is an integer selected from 1 to about 4; IV and R^{b} are each independently selected from the group consisting of hydrogen and alkyl, including lower alkyl such as C₁-C₄ alkyl that are optionally branched; or R^{a} and R^{b} are taken together with the attached carbon atom to form a carbocyclic ring; R is an optionally substituted alkyl group, an optionally substituted acyl group, or a suitably selected nitrogen protecting group; and (*) indicates points of attachment for the nucleotide, vitamin, other polyvalent linkers, or other parts of the conjugate.

In another embodiment, the polyvalent linkers described herein include compounds of the following formulae

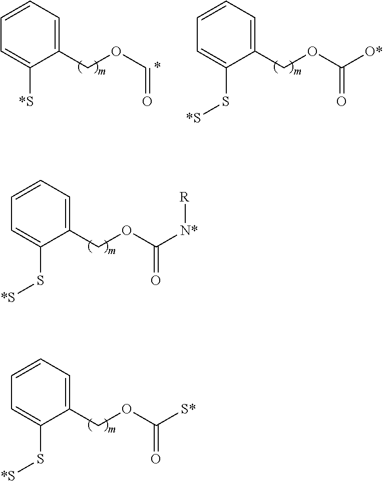

where m is an integer selected from 1 to about 4; R is an optionally substituted alkyl group, an optionally substituted acyl group, or a suitably selected nitrogen protecting group; and (*) indicates points of attachment for the nucleotide, vitamin, other polyvalent linkers, or other parts of the conjugate.

In another embodiment, the polyvalent linkers described herein include compounds of the following formulae

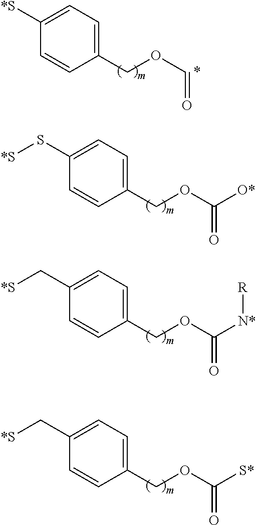

where m is an integer selected from 1 to about 4; R is an optionally substituted alkyl group, an optionally substituted acyl group, or a suitably selected nitrogen protecting group; and (*) indicates points of attachment for the nucleotide, vitamin, other polyvalent linkers, or other parts of the conjugate.

Another illustrative mechanism involves an arrangement of the releasable and spacer linkers in such a way that subsequent to the cleavage of a bond in the bivalent linker, released functional groups chemically assist the breakage or cleavage of additional bonds, also termed anchimeric assisted cleavage or breakage. An illustrative embodiment of such a bivalent linker or portion thereof includes compounds having the formula:

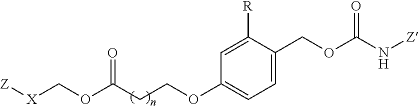

where X is an heteroatom, such as nitrogen, oxygen, or sulfur, n is an integer selected from 0, 1, 2, and 3, R is hydrogen, or a substituent, including a substituent capable of stabilizing a positive charge inductively or by resonance on the aryl ring, such as alkoxy, and the like, and either of Z or Z′ is the vitamin, or analog or derivative thereof, or the nucleotide, or analog or derivative thereof, or a vitamin or nucleotide moiety in conjunction with other portions of the bivalent linker. It is appreciated that other substituents may be present on the aryl ring, the benzyl carbon, the carbamate nitrogen, the alkanoic acid, or the methylene bridge, including but not limited to hydroxy, alkyl, alkoxy, alkylthio, halo, and the like. Assisted cleavage may include mechanisms involving benzylium intermediates, benzyne intermediates, lactone cyclization, oxonium intermediates, beta-elimination, and the like. It is further appreciated that, in addition to fragmentation subsequent to cleavage of the releasable linker, the initial cleavage of the releasable linker may be facilitated by an anchimerically assisted mechanism.

In this embodiment, the hydroxyalkanoic acid, which may cyclize, facilitates cleavage of the methylene bridge, by for example an oxonium ion, and facilitates bond cleavage or subsequent fragmentation after bond cleavage of the releasable linker. Alternatively, acid catalyzed oxonium ion-assisted cleavage of the methylene bridge may begin a cascade of fragmentation of this illustrative bivalent linker, or fragment thereof. Alternatively, acid-catalyzed hydrolysis of the carbamate may facilitate the beta elimination of the hydroxyalkanoic acid, which may cyclize, and facilitate cleavage of methylene bridge, by for example an oxonium ion. It is appreciated that other chemical mechanisms of bond breakage or cleavage under the metabolic, physiological, or cellular conditions described herein may initiate such a cascade of fragmentation. It is appreciated that other chemical mechanisms of bond breakage or cleavage under the metabolic, physiological, or cellular conditions described herein may initiate such a cascade of fragmentation.

In another embodiment, the polyvalent linker includes additional spacer linkers and releasable linkers connected to form a polyvalent 3-thiosuccinimid-1-ylalkyloxymethyloxy group, illustrated by the following formula

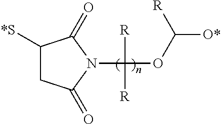

where n is an integer from 1 to 6, the alkyl group is optionally substituted, and the methyl is optionally substituted with an additional alkyl or optionally substituted aryl group, each of which is represented by an independently selected group R. The (*) symbols indicate points of attachment of the polyvalent linker fragment to other parts of the conjugates described herein.

In another embodiment, the polyvalent linker includes additional spacer linkers and releasable linkers connected to form a polyvalent 3-thiosuccinimid-1-ylalkylcarbonyl group, illustrated by the following formula

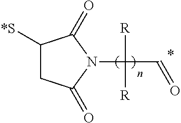

where n is an integer from 1 to 6, and the alkyl group is optionally substituted. The (*) symbols indicate points of attachment of the polyvalent linker fragment to other parts of the conjugates described herein. In another embodiment, the polyvalent linker includes spacer linkers and releasable linkers connected to form a polyvalent 3-thioalkylsulfonylalkyl(disubstituted silyl)oxy group, where the disubstituted silyl is substituted with alkyl and/or optionally substituted aryl groups.

In another embodiment, the polyvalent linker includes additional spacer linkers and releasable linkers connected to form a polyvalent dithioalkylcarbonylhydrazide group, or a polyvalent 3-thiosuccinimid-1-ylalkylcarbonylhydrazide, illustrated by the following formulae

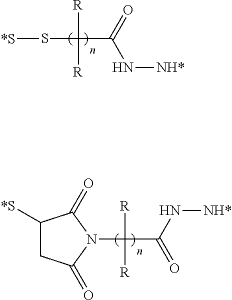

where n is an integer from 1 to 6, the alkyl group is optionally substituted, and the hydrazide forms an hydrazone with (B), (A) or another part of the polyvalent linker (L). The (*) symbols indicate points of attachment of the polyvalent linker fragment to other parts of the conjugates described herein.

In another embodiment, the polyvalent linker includes additional spacer linkers and releasable linkers connected to form a polyvalent 3-thiosuccinimid-1-ylalkyloxyalkyloxyalkylidene group, illustrated by the following formula

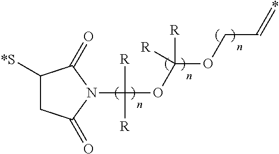

where each n is an independently selected integer from 1 to 6, each alkyl group independently selected and is optionally substituted, such as with alkyl or optionally substituted aryl, and where the alkylidene forms an hydrazone with (B), (A), or another part of the polyvalent linker (L). The (*) symbols indicate points of attachment of the polyvalent linker fragment to other parts of the conjugates described herein.

Additional illustrative additional spacer linkers include alkylene-amino alkylenecarbonyl, alkylene-thio-carbonylalkylsuccinimid-3-yl, and the like, as further illustrated by the following formulae:

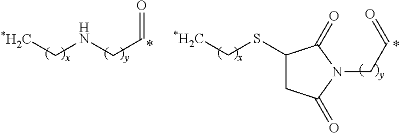

where the integers x and y are 1, 2, 3, 4, or 5:

In another illustrative embodiment, the linker includes one or more amino acids. Such amino acids are illustratively selected from the naturally occurring amino acids, or stereoisomers thereof. The amino acid may also be any other amino acid, such as any amino acid having the general formula:

—N(R)—(CR′R″)_{q}—C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornithine, threonine, and the like. In another illustrative aspect of the vitamin receptor binding nucleotide delivery conjugate intermediate described herein, the nucleotide, or an analog or a derivative thereof, includes an alkylthiol nucleophile.

Additional linkers are described in the following Tables, where the (*) atom is the point of attachment of additional spacer or releasable linkers, the nucleotide, and/or the binding ligand.

Illustrative additional spacer linkers include the following.

| 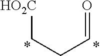 | 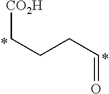 |
|---|---|
| 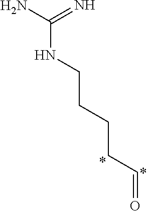 | 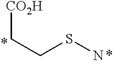 |
| 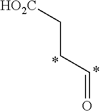 | 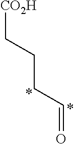 |
| 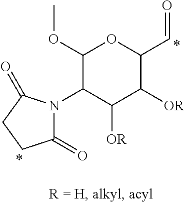 | 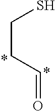 |
| 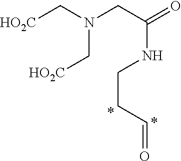 | |
| 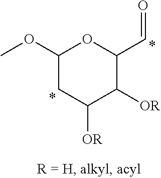 | |
| 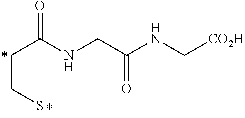 | |
| 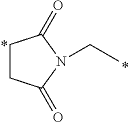 | 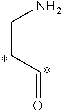 |
| 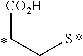 | 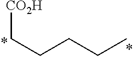 |
| 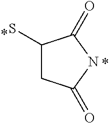 | |
| 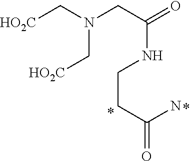 | |
| 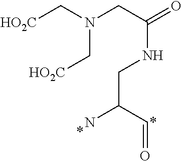 | |
| 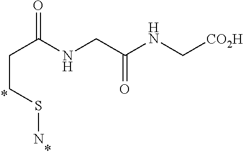 | |
| 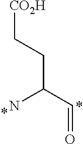 | 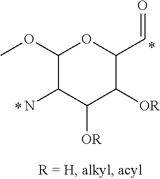 |
| 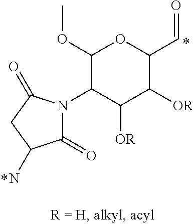 | |
| 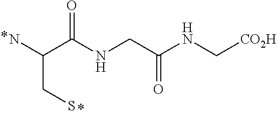 | |
| 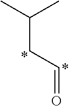 | 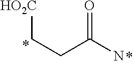 |
| 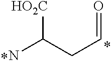 | 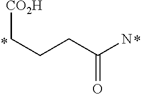 |
| 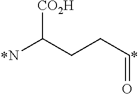 | 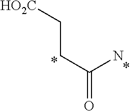 |
| 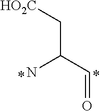 | |
| 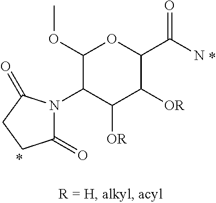 | |
| 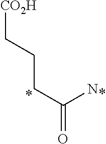 | 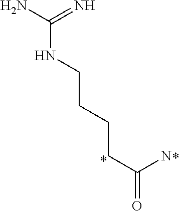 |
| 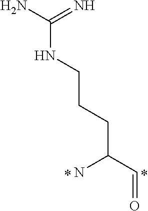 | |
| 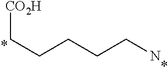 | 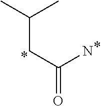 |
| 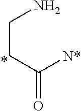 | 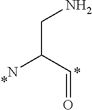 |
| 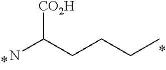 | 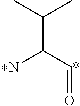 |
| 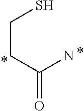 | 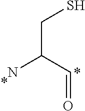 |
| 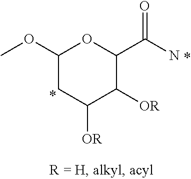 | |
| 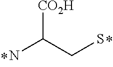 | 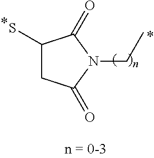 |
| 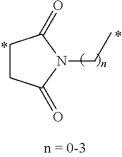 | 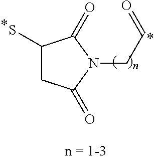 |

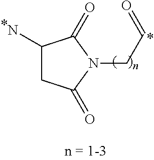

Illustrative additional releasable linkers include the following.

| 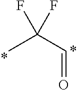 | 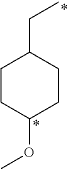 |
|---|---|
| 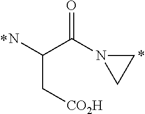 | 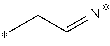 |
| 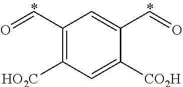 | |
| 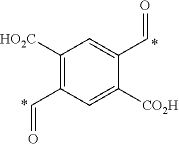 | |
| 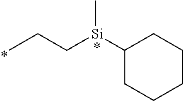 | 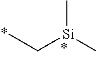 |
| 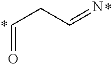 | 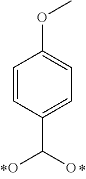 |
| 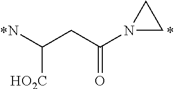 | 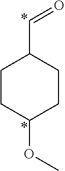 |
| 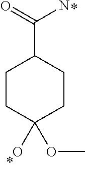 | 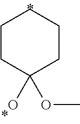 |
| 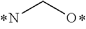 | 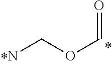 |
| 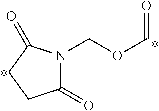 | |
| 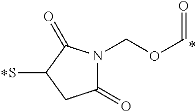 | |
| 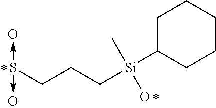 | |
| 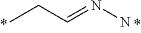 | 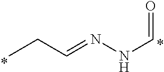 |
| 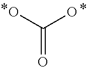 | 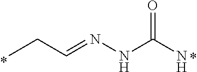 |
| 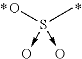 | 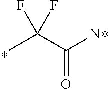 |
| 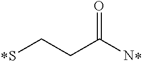 | 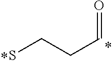 |
| 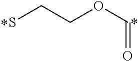 | 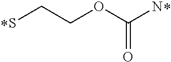 |
| 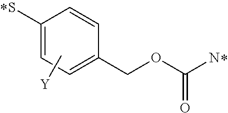 | |
| 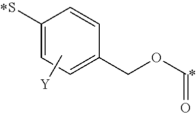 | |
| 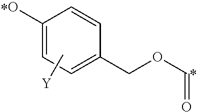 | |
| 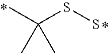 | 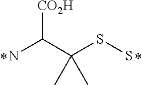 |

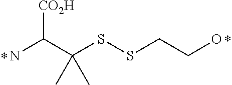

In another embodiment, multi-nucleotide conjugates are described herein. Several illustrative configurations of such multi-nucleotide conjugates are include herein, such as the compounds and compositions described in PCT international publication No. WO 2007/022494, the disclosure of which is incorporated herein by reference. Illustratively, the polyvalent linkers may connect the receptor binding ligand B to the two or more nucleotides N in a variety of structural configurations, including but not limited to the following illustrative general formulae:

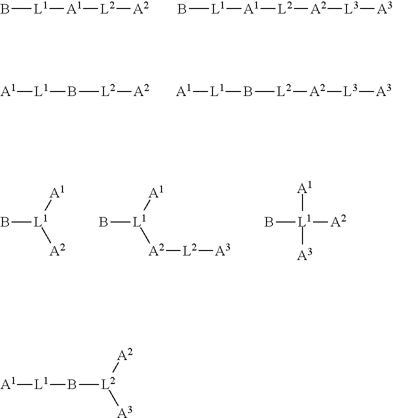

where B is the receptor binding ligand, each of (L¹), (L²), and (L³) is a polyvalent linker as described herein comprising a hydrophilic spacer linker, and optionally including one or more releasable linkers and/or additional spacer linkers, and each of (A¹), (A²), and (A³) is a nucleotide, or an analog or derivative thereof. Other variations, including additional nucleotides N, or analogs or derivatives thereof, additional linkers, and additional configurations of the arrangement of each of (B), (L), and (A).

In one variation, more than one receptor binding ligand B is included in the delivery conjugates described herein, including but not limited to the following illustrative general formulae:

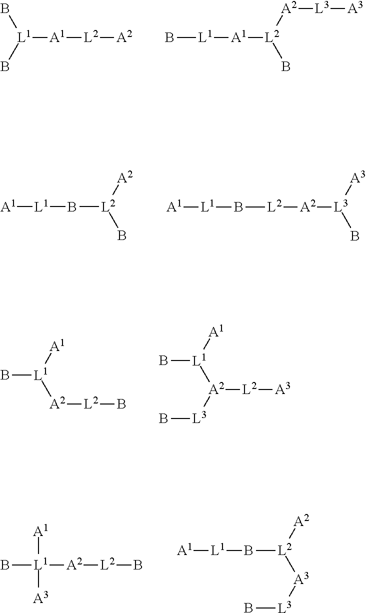

Where each B is a receptor binding ligand, each of (L¹), (L²), and (L³) is a polyvalent linker as described herein comprising a hydrophilic spacer linker, and optionally including one or more releasable linkers and/or additional spacer linkers, and each of (A¹), (A²), and (A³) is a nucleotide, or an analog or derivative thereof. Other variations, including additional nucleotides N, or analogs or derivatives thereof, additional linkers, and additional configurations of the arrangement of each of (B), (L), and (A), are also contemplated herein. It is to be understood that in variations of this embodiment, each of folate receptor binding ligands B may be the same or may be different.

Generally, any manner of forming a conjugate between bivalent linker (L) and binding ligand (B), between bivalent linker (L) and nucleotide N, including any intervening heteroatom linkers, can be utilized Also, any method of forming a conjugate between the hydrophilic spacer linkers, or any additional spacer linker, the one or more releasable linkers, and any additional heteroatoms to form bivalent linker (L) can be used. For example, covalent bonding can occur, for example, through the formation of amide, ester, disulfide, or imino bonds between acid, aldehyde, hydroxy, amino, sulfhydryl, or hydrazo groups.

The spacer and/or releasable linker, also referred to as a cleavable linker, can be any biocompatible linker. The cleavable linker can be, for example, a linker susceptible to cleavage under the reducing or oxidizing conditions present in or on cells, a pH-sensitive linker that may be an acid-labile or base-labile linker, or a linker that is cleavable by biochemical or metabolic processes, such as an enzyme-labile linker. Typically, the spacer and/or releasable linker comprises about 1 to about 30 carbon atoms, more typically about 2 to about 20 carbon atoms. Lower molecular weight linkers (i.e., those having an approximate molecular weight of about 30 to about 300) are typically employed. Precursors to such linkers are typically selected to have either nucleophilic or electrophilic functional groups, or both, optionally in a protected form with a readily cleavable protecting group to facilitate their use in synthesis of the intermediate species.

Additionally, more than one type of binding ligand nucleotide delivery conjugate can be used. Illustratively, for example, conjugates with different binding ligands B, but the same nucleotide N can be administered to the host animal. In other embodiments, conjugates comprising the same binding ligand B linked to different nucleotides N, or various binding ligands B linked to various nucleotides N can be administered to the host animal. In another illustrative embodiment, binding ligand nucleotide delivery conjugates with the same or different ligands B, and the same or different nucleotides N comprising multiple folates and multiple nucleotides as part of the same nucleotide delivery conjugate can be used.

It is to be understood that the terms linker, bivalent linker, divalent linker, and polyvalent linker can be used interchangeably herein.

The nucleotide delivery conjugates described herein can be prepared by a variety of synthetic methods. The synthetic methods are chosen depending upon the selection of the optionally addition heteroatoms or the heteroatoms that are already present on the spacer linkers, releasable linkers, nucleotides, and/or or binding ligands. In general, the relevant bond forming reactions are described in Richard C. Larock, “Comprehensive Organic Transformations, a guide to functional group preparations,” VCH Publishers, Inc. New York (1989), and in Theodora E. Greene & Peter G. M. Wuts, “Protective Groups ion Organic Synthesis,” 2d edition, John Wiley & Sons, Inc. New York (1991), the disclosures of which are incorporated herein by reference.

General amide and ester formation. For example, where the heteroatom linker is a nitrogen atom, and the terminal functional group present on the spacer linker or the releasable linker is a carbonyl group, the required amide group can be obtained by coupling reactions or acylation reactions of the corresponding carboxylic acid or derivative, where L is a suitably-selected leaving group such as halo, triflate, pentafluorophenoxy, trimethylsilyloxy, succinimide-N-oxy, and the like, and an amine, as illustrated in Scheme 1.

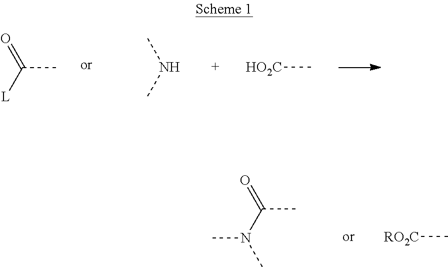

Coupling reagents include DCC, EDC, RRDQ, CGI, HBTU, TBTU, HOBT/DCC, HOBT/EDC, BOP—Cl, PyBOP, PyBroP, and the like. Alternatively, the parent acid can be converted into an activated carbonyl derivative, such as an acid chloride, a N-hydroxysuccinimidyl ester, a pentafluorophenyl ester, and the like. The amide-forming reaction can also be conducted in the presence of a base, such as triethylamine, diisopropylethylamine, N,N-dimethyl-4-aminopyridine, and the like. Suitable solvents for forming amides described herein include CH₂Cl₂, CHCl₃, THF, DMF, DMSO, acetonitrile, EtOAc, and the like. Illustratively, the amides can be prepared at temperatures in the range from about −15° C. to about 80° C., or from about 0° C. to about 45° C. Amides can be formed from, for example, nitrogen-containing aziridine rings, carbohydrates, and a-halogenated carboxylic acids. Illustrative carboxylic acid derivatives useful for forming amides include compounds having the formulae:

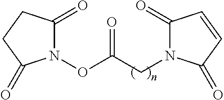

and the like, where n is an integer such as 1, 2, 3, or 4.

Similarly, where the heteroatom linker is an oxygen atom and the terminal functional group present on the spacer linker or the releasable linker is a carbonyl group, the required ester group can be obtained by coupling reactions of the corresponding carboxylic acid or derivative, and an alcohol.

Coupling reagents include DCC, EDC, CDI, BOP, PyBOP, isopropenyl chloroformate, EEDQ, DEAD, PPh₃, and the like. Solvents include CH₂Cl₂, CHCl₃, THF, DMF, DMSO, acetonitrile, EtOAc, and the like. Bases include triethylamine, diisopropyl-ethylamine, and N,N-dimethyl-4-aminopyridine. Alternatively, the parent acid can be converted into an activated carbonyl derivative, such as an acid chloride, a N-hydroxysuccinimidyl ester, a pentafluorophenyl ester, and the like.

General ketal and acetal formation. Furthermore, where the heteroatom linker is an oxygen atom, and the functional group present on the spacer linker or the releasable linker is 1-alkoxyalkyl, the required acetal or ketal group can be formed by ketal and acetal forming reactions of the corresponding alcohol and an enol ether, as illustrated in Scheme 2.

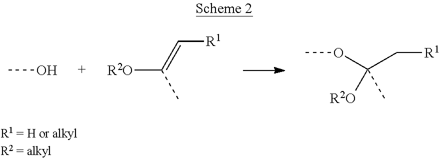

Solvents include alcohols, CH₂Cl₂, CHCl₃, THF, diethylether, DMF, DMSO, acetonitrile, EtOAc, and the like. The formation of such acetals and ketals can be accomplished with an acid catalyst. Where the heteroatom linker comprises two oxygen atoms, and the releasable linker is methylene, optionally substituted with a group X² as described herein, the required symmetrical acetal or ketal group can be illustratively formed by acetal and ketal forming reactions from the corresponding alcohols and an aldehyde or ketone, as illustrated in Scheme 3.

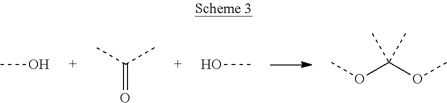

Alternative ketal and acetal formation. Where the methylene is substituted with an optionally-substituted aryl group, the required acetal or ketal may be prepared stepwise, where L is a suitably selected leaving group such as halo, trifluoroacetoxy, triflate, and the like, as illustrated in Scheme 4. The process illustrated in Scheme 4 is a conventional preparation, and generally follows the procedure reviewed by R. R. Schmidt et al., Chem. Rev., 2000, 100, 4423-42, the disclosure of which is incorporated herein by reference.

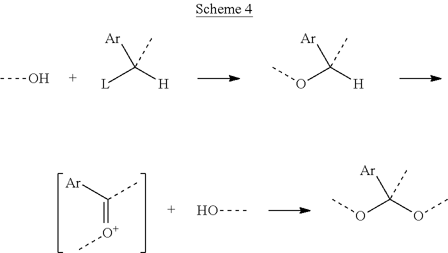

The resulting arylalkyl ether is treated with an oxidizing agent, such as DDQ, and the like, to generate an intermediate oxonium ion that is subsequently treated with another alcohol to generate the acetal or ketal.

General succinimide formation. Furthermore, where the heteroatom linker is, for example, a nitrogen, oxygen, or sulfur atom, and the functional group present on the spacer linker or the releasable linker is a succinimide derivative, the resulting carbon-heteroatom bond can be formed by a Michael addition of the corresponding amine, alcohol, or thiol, and a maleimide derivative, where X is the heteroatom linker, as illustrated in Scheme 5.

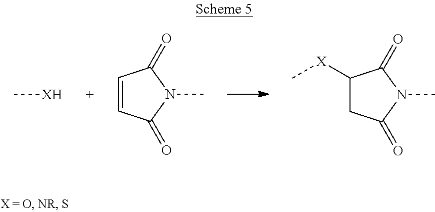

Solvents for performing the Michael addition include THF, EtOAc, CH₂Cl₂, DMF, DMSO, H₂O and the like. The formation of such Michael adducts can be accomplished with the addition of equimolar amounts of bases, such as triethylamine, Hünig's base or by adjusting the pH of water solutions to 6.0-7.4. It is appreciated that when the heteroatom linker is an oxygen or nitrogen atom, reaction conditions may be adjusted to facilitate the Michael addition, such as, for example, by using higher reaction temperatures, adding catalysts, using more polar solvents, such as DMF, DMSO, and the like, and activating the maleimide with silylating reagents.

General silyloxy formation. Furthermore, where the heteroatom linker is an oxygen atom, and the functional group present on the spacer linker or the releasable linker is a silyl derivative, the required silyloxy group may be formed by reacting the corresponding silyl derivative, and an alcohol, where L is a suitably selected leaving group such as halo, trifluoroacetoxy, triflate, and the like, as illustrated in Scheme 6.

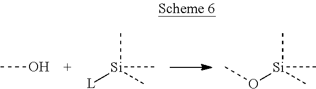

Silyl derivatives include properly functionalized silyl derivatives such as vinylsulfonoalkyl diaryl, or diaryl, or alkyl aryl silyl chloride. Instead of a vinylsulfonoalkyl group, a β-chloroethylsulfonoalkyl precursor may be used. Any aprotic and anhydrous solvent and any nitrogen-containing base may serve as a reaction medium. The temperature range employed in this transformation may vary between −78° C. and 80° C.

General hydrazone formation. Furthermore, where the heteroatom linker is a nitrogen atom, and the functional group present on the spacer linker or the releasable linker is an iminyl derivative, the required hydrazone group can be formed by reacting the corresponding aldehyde or ketone, and a hydrazine or acylhydrazine derivative, as illustrated in Scheme 7, equations (1) and (2) respectively.

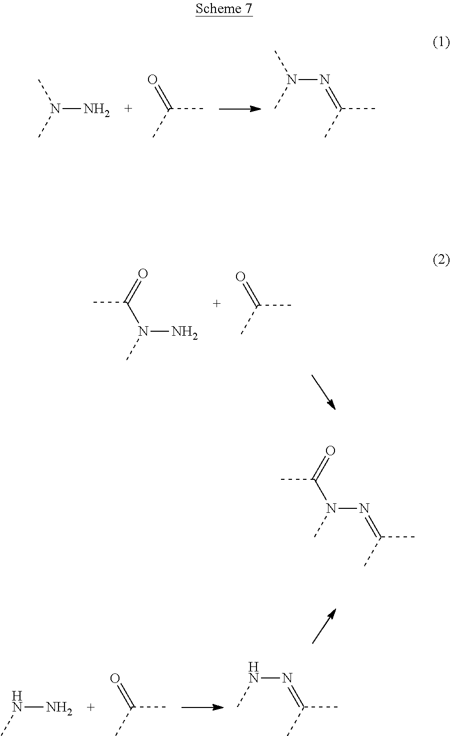

Solvents that can be used include THF, EtOAc, CH₂Cl₂, CHCl₃, CCl₄, DMF, DMSO, MeOH and the like. The temperature range employed in this transformation may vary between 0° C. and 80° C. Any acidic catalyst such as a mineral acid, H₃CCOOH, F₃CCOOH, p-TsOH.H₂O, pyridinium p-toluene sulfonate, and the like can be used. In the case of the acylhydrazone in equation (2), the acylhydrazone may be prepared by initially acylating hydrazine with a suitable carboxylic acid or derivative, as generally described above in Scheme 1, and subsequently reacting the acylhydrazide with the corresponding aldehyde or ketone to form the acylhydrazone. Alternatively, the hydrazone functionality may be initially formed by reacting hydrazine with the corresponding aldehyde or ketone. The resulting hydrazone may subsequently be acylated with a suitable carboxylic acid or derivative, as generally described above in Scheme 1.

General disulfide formation. Furthermore, where the heteroatom linker is a sulfur atom, and the functional group present on the releasable linker is an alkylenethiol derivative, the required disulfide group can be formed by reacting the corresponding alkyl or aryl sulfonylthioalkyl derivative, or the corresponding heteroaryldithioalkyl derivative such as a pyridin-2-yldithioalkyl derivative, and the like, with an alkylenethiol derivative, as illustrated in Scheme 8.

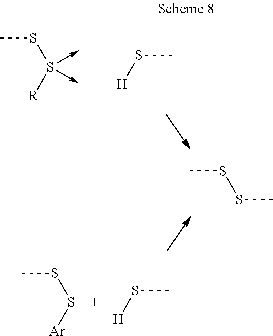

Solvents that can be used are THF, EtOAc, CH₂Cl₂, CHCl₃, CCl₄, DMF, DMSO, and the like. The temperature range employed in this transformation may vary between 0° C. and 80° C. The required alkyl or aryl sulfonylthioalkyl derivative may be prepared using art-recognized protocols, and also according to the method of Ranasinghe and Fuchs, Synth. Commun. 18(3), 227-32 (1988), the disclosure of which is incorporated herein by reference. Other methods of preparing unsymmetrical dialkyl disulfides are based on a transthiolation of unsymmetrical heteroaryl-alkyl disulfides, such as 2-thiopyridinyl, 3-nitro-2-thiopyridinyl, and like disulfides, with alkyl thiol, as described in WO 88/01622, European Patent Application No. 0116208A1, and U.S. Pat. No. 4,691,024, the disclosures of which are incorporated herein by reference.

General carbonate formation. Furthermore, where the heteroatom linker is an oxygen atom, and the functional group present on the spacer linker or the releasable linker is an alkoxycarbonyl derivative, the required carbonate group can be formed by reacting the corresponding hydroxy-substituted compound with an activated alkoxycarbonyl derivative where L is a suitable leaving group, as illustrated in Scheme 9.

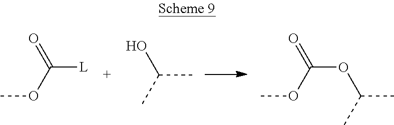

Solvents that can be used are THF, EtOAc, CH₂Cl₂, CHCl₃, CCl₄, DMF, DMSO, and the like. The temperature range employed in this transformation may vary between 0° C. and 80° C. Any basic catalyst such as an inorganic base, an amine base, a polymer bound base, and the like can be used to facilitate the reaction.

General semicarbazone formation. Furthermore, where the heteroatom linker is a nitrogen atom, and the functional group present on one spacer linker or the releasable linker is an iminyl derivative, and the functional group present on the other spacer linker or the other releasable linker is an alkylamino or arylaminocarbonyl derivative, the required semicarbazone group can be formed by reacting the corresponding aldehyde or ketone, and a semicarbazide derivative, as illustrated in Scheme 10.

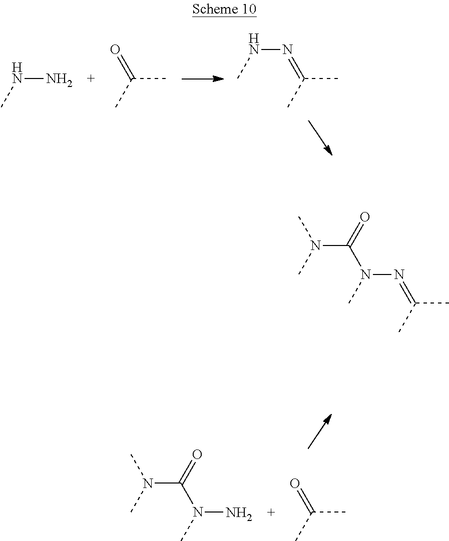

Solvents that can be used are THF, EtOAc, CH₂Cl₂, CHCl₃, CCl₄, DMF, DMSO, MeOH and the like. The temperature range employed in this transformation may vary between 0° C. and 80° C. Any acidic catalyst such as a mineral acid, H₃CCOOH, F₃CCOOH, p-TsOH.H₂O, pyridinium p-toluene sulfonate, and the like can be used. In addition, in forming the semicarbazone, the hydrazone functionality may be initially formed by reacting hydrazine with the corresponding aldehyde or ketone. The resulting hydrazone may subsequently by acylated with an isocyanate or a carbamoyl derivative, such as a carbamoyl halide, to form the semicarbazone. Alternatively, the corresponding semicarbazide may be formed by reacting hydrazine with an isocyanate or carbamoyl derivative, such as a carbamoyl halide to form a semicarbazide. Subsequently, the semicarbazide may be reacted with the corresponding aldehyde or ketone to form the semicarbazone.

General sulfonate formation. Furthermore, where the heteroatom linker is an oxygen atom, and the functional group present on the spacer linker or the releasable linker is sulfonyl derivative, the required sulfonate group can be formed by reacting the corresponding hydroxy-substituted compound with an activated sulfonyl derivative where L is a suitable leaving group such as halo, and the like, as illustrated in Scheme 11.

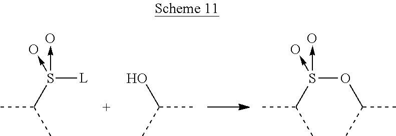

Solvents that can be used are THF, EtOAc, CH₂Cl₂, CHCl₃, CCl₄, and the like. The temperature range employed in this transformation may vary between 0° C. and 80° C. Any basic catalyst such as an inorganic base, an amine base, a polymer bound base, and the like can be used to facilitate the reaction.

General formation of folate-peptides. The folate-containing peptidyl fragment Pte-Glu-(AA)ₙ-NH(CHR₂)CO₂H (3) is prepared by a polymer-supported sequential approach using standard methods, such as the Fmoc-strategy on an acid-sensitive Fmoc-AA-Wang resin (1), as shown in Scheme 12.

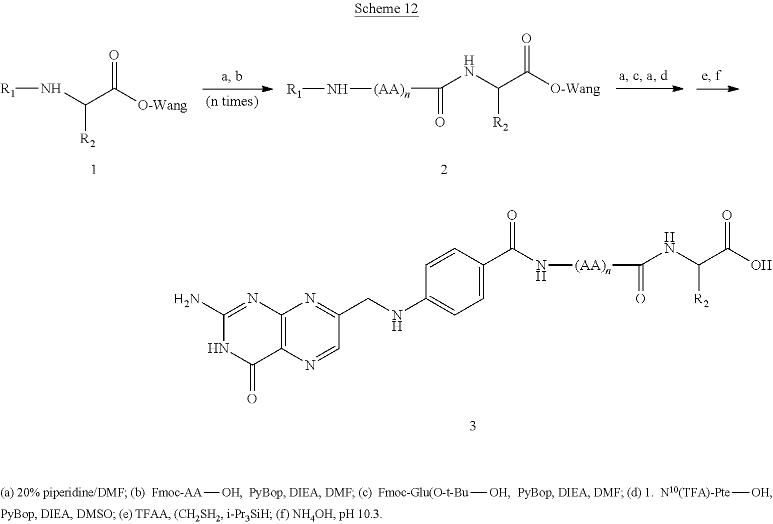

In this illustrative embodiment of the processes described herein, R₁ is Fmoc, R₂ is the desired appropriately-protected amino acid side chain, and DIPEA is diisopropylethylamine. Standard coupling procedures, such as PyBOP and others described herein or known in the art are used, where the coupling agent is illustratively applied as the activating reagent to ensure efficient coupling. Fmoc protecting groups are removed after each coupling step under standard conditions, such as upon treatment with piperidine, tetrabutylammonium fluoride (TBAF), and the like. Appropriately protected amino acid building blocks, such as Fmoc-Glu-OtBu, N¹⁰-TFA-Pte-OH, and the like, are used, as described in Scheme 12, and represented in step (b) by Fmoc-AA-OH. Thus, AA refers to any amino acid starting material, that is appropriatedly protected. It is to be understood that the term amino acid as used herein is intended to refer to any reagent having both an amine and a carboxylic acid functional group separated by one or more carbons, and includes the naturally occurring alpha and beta amino acids, as well as amino acid derivatives and analogs of these amino acids. In particular, amino acids having side chains that are protected, such as protected serine, threonine, cysteine, aspartate, and the like may also be used in the folate-peptide synthesis described herein. Further, gamma, delta, or longer homologous amino acids may also be included as starting materials in the folate-peptide synthesis described herein. Further, amino acid analogs having homologous side chains, or alternate branching structures, such as norleucine, isovaline, β-methyl threonine, β-methyl cysteine, β,β-dimethyl cysteine, and the like, may also be included as starting materials in the folate-peptide synthesis described herein.

The coupling sequence (steps (a) & (b)) involving Fmoc-AA-OH is performed “n” times to prepare solid-support peptide 2, where n is an integer and may equal 0 to about 100. Following the last coupling step, the remaining Fmoc group is removed (step (a)), and the peptide is sequentially coupled to a glutamate derivative (step (c)), deprotected, and coupled to TFA-protected pteroic acid (step (d)). Subsequently, the peptide is cleaved from the polymeric support upon treatment with trifluoroacetic acid, ethanedithiol, and triisopropylsilane (step (e)). These reaction conditions result in the simultaneous removal of the t-Bu, t-Boc, and Trt protecting groups that may form part of the appropriately-protected amino acid side chain. The TFA protecting group is removed upon treatment with base (step (f)) to provide the folate-containing peptidyl fragment 3.

In addition, the following illustrative process may be used to prepare compounds described herein, where is an integer such as 1 to about 10.

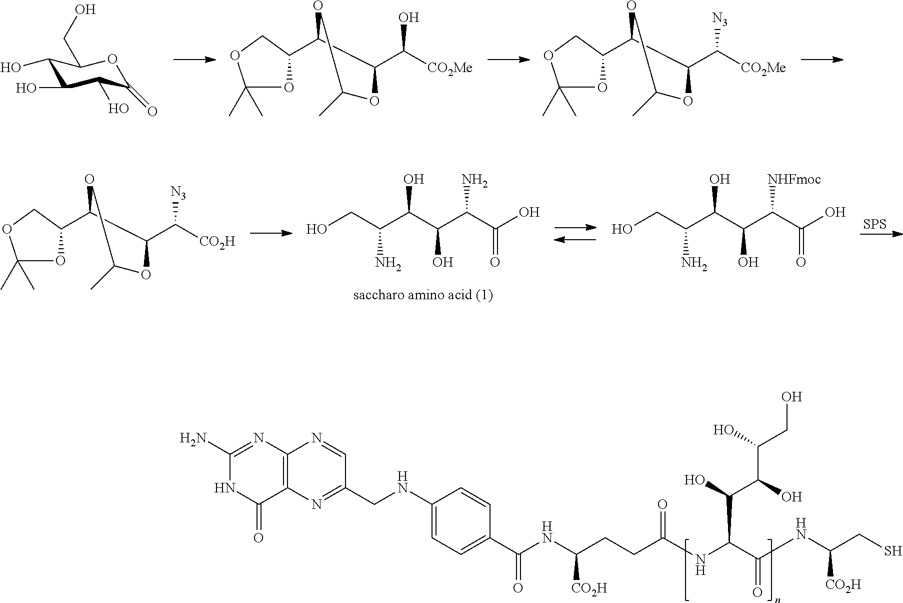

It is to be understood that although the foregoing synthetic process is illustrated for selected compounds, such as the particular saccharopeptides shown, additional analogous compounds may be prepared using the same or similar process by the routine selection of starting materials and the routine optimization of reaction conditions.

The compounds described herein may be prepared using conventional synthetic organic chemistry. In addition, the following illustrative process may be used to prepare compounds described herein, where is an integer such as 1 to about 10.

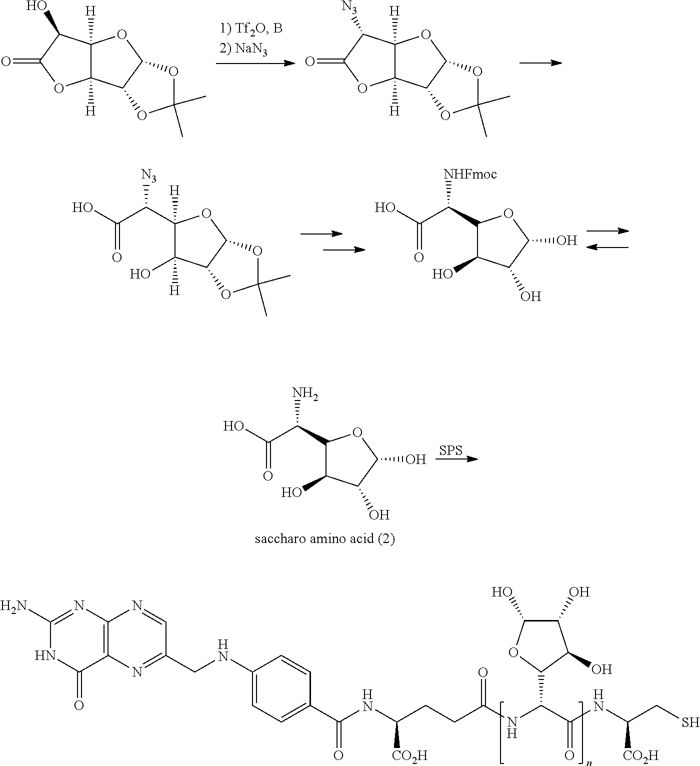

It is to be understood that although the foregoing synthetic process is illustrated for selected compounds, such as the particular saccharopeptides shown, additional analogous compounds may be prepared using the same or similar process by the routine selection of starting materials and the routine optimization of reaction conditions.

In addition, the following illustrative process may be used to prepare compounds described herein.

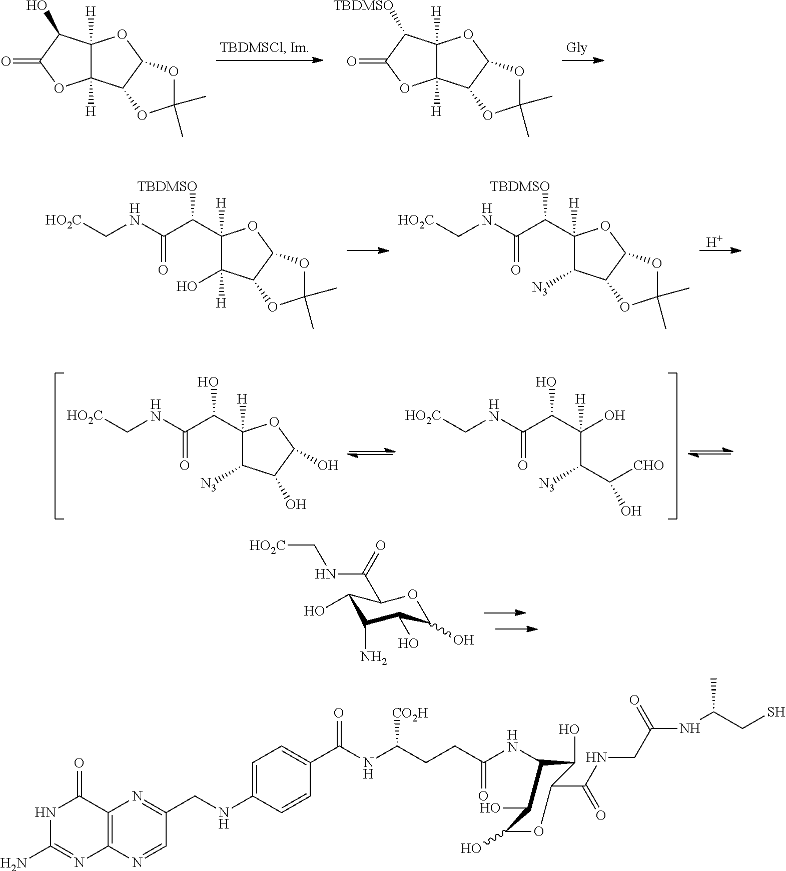

It is to be understood that although the foregoing synthetic process is illustrated for selected compounds, additional analogous compounds (i.e., nucleotide conjugates) may be prepared using the same or similar process by the routine selection of starting materials and the routine optimization of reaction conditions.

In each of the foregoing synthetic processes, the intermediates may be coupled with any additional hydrophilic spacer linkers, other spacer linkers, releasable linkers, or the nucleotide N. In variations of each of the foregoing processes, additional hydrophilic spacer linkers, other spacer linkers, or releasable linkers may be inserted between the binding ligand B and the indicated hydrophilic spacer linkers. In addition, it is to be understood that the left-to-right arrangement of the bivalent hydrophilic spacer linkers is not limiting, and accordingly, the nucleotide N, the binding ligand B, additional hydrophilic spacer linkers, other spacer linkers, and/or releasable linkers may be attached to either end of the hydrophilic spacer linkers described herein.

In various embodiments of the methods, compounds, and compositions described herein, pharmaceutically acceptable salts of the conjugates described herein are described. Pharmaceutically acceptable salts of the conjugates described herein include the acid addition and base salts thereof (e.g., pharmaceutically acceptable salts of a ligand, such as folate).

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the conjugates described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In various embodiments of the methods, compounds, and compositions described herein, the binding ligand nucleotide delivery conjugates may be administered in combination with one or more other drugs (or as any combination thereof).

In one embodiment, the conjugates described herein may be administered as a formulation in association with one or more pharmaceutically acceptable carriers. The carriers can be excipients. The term “carrier” is used herein to describe any ingredient other than a conjugate described herein. The choice of carrier will to a large extent depend on factors such as the particular mode of administration, the effect of the carrier on solubility and stability, and the nature of the dosage form. Pharmaceutical compositions suitable for the delivery of conjugates described herein and methods for their preparation will be readily apparent to those skilled in the art. Such compositions and methods for their preparation may be found, for example, in Remington: The Science & Practice of Pharmacy, 21th Edition (Lippincott Williams & Wilkins, 2005), incorporated herein by reference.

In one illustrative aspect, a pharmaceutically acceptable carrier includes any and all solvents, dispersion media, coatings, antibacterial and antifungal agents, isotonic and absorption delaying agents, and the like, and combinations thereof, that are physiologically compatible. In some embodiments, the carrier is suitable for parenteral administration. Pharmaceutically acceptable carriers include sterile aqueous solutions or dispersions and sterile powders for the extemporaneous preparation of sterile injectable solutions or dispersions. Supplementary active compounds can also be incorporated into compositions of the invention.

In various embodiments, liquid formulations may include suspensions and solutions. Such formulations may comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

In one embodiment, an aqueous suspension may contain the active materials in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents.

In one illustrative embodiment, dispersible powders and granules suitable for preparation of an aqueous suspension by the addition of water provide the active ingredient in admixture with a dispersing or wetting agent, suspending agent and one or more preservatives. Additional excipients, for example, coloring agents, may also be present.

Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate.

In other embodiments, isotonic agents, for example, sugars, polyalcohols such as mannitol, sorbitol, or sodium chloride can be included in the composition. Prolonged absorption of the injectable compositions can be brought about by including in the composition an agent which delays absorption, for example, monostearate salts and gelatin.

In one aspect, a conjugate as described herein may be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. In other embodiments, any of the liquid formulations described herein may be adapted for parenteral administration of the conjugates described herein. The preparation of parenteral formulations under sterile conditions, for example, by lyophilization under sterile conditions, may readily be accomplished using standard pharmaceutical techniques well known to those skilled in the art. In one embodiment, the solubility of a conjugate used in the preparation of a parenteral formulation may be increased by the use of appropriate formulation techniques, such as the incorporation of solubility-enhancing agents.

In various embodiments, formulations for parenteral administration may be formulated to be for immediate and/or modified release. In one illustrative aspect, the conjugates of the invention may be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art. In another embodiment, the conjugates described herein or compositions comprising the conjugates may be continuously administered, where appropriate.

In one embodiment, a kit is provided. If a combination of active compounds is to be administered, two or more pharmaceutical compositions may be combined in the form of a kit suitable for sequential administration or co-administration of the compositions. Such a kit comprises two or more separate pharmaceutical compositions, at least one of which contains a conjugate described herein, and means for separately retaining the compositions, such as a container, divided bottle, or divided foil packet. In another embodiment, compositions comprising one or more conjugates described herein, in containers having labels that provide instructions for use of the conjugates are provided.

In one embodiment, sterile injectable solutions can be prepared by incorporating the conjugates in the required amount in an appropriate solvent with one or a combination of ingredients described above, as required, followed by filtered sterilization. Typically, dispersions are prepared by incorporating the conjugates into a sterile vehicle which contains a dispersion medium and any additional ingredients from those described above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The composition can be formulated as a solution, microemulsion, liposome, or other ordered structure. The carrier can be a solvent or dispersion medium containing, for example, water, ethanol, polyol (for example, glycerol, propylene glycol, and liquid polyethylene glycol, and the like), and suitable mixtures thereof. In one embodiment, the proper fluidity can be maintained, for example, by the use of a coating such as lecithin, by the maintenance of the required particle size in the case of dispersion and by the use of surfactants.

Any effective regimen for administering the conjugates can be used. For example, the conjugates can be administered as single doses, or can be divided and administered as a multiple-dose daily regimen. Further, a staggered regimen, for example, one to five days per week can be used as an alternative to daily treatment, and for the purpose of the methods described herein, such intermittent or staggered daily regimen is considered to be equivalent to every day treatment and is contemplated. In one illustrative embodiment the patient is treated with multiple injections of the conjugate to treat tumors or inflammation. In one embodiment, the patient is injected multiple times (preferably about 2 up to about 50 times) with the conjugate, for example, at 12-72 hour intervals or at 48-72 hour intervals. Additional injections of the conjugate can be administered to the patient at an interval of days or months after the initial injections(s) and the additional injections can prevent recurrence of the cancer or inflammation.

Any suitable course of therapy with the conjugate can be used. In one embodiment, individual doses and dosage regimens are selected to provide a total dose administered during a month of about 15 mg. In one illustrative example, the conjugate is administered in a single daily dose administered on M, Tu, W, Th, and F, in weeks 1, 2, and 3 of each 4 week cycle, with no dose administered in week 4. In an alternative example, the conjugate is administered in a single daily dose administered on M, W, and F, of weeks 1, and 3 of each 4 week cycle, with no dose administered in weeks 2 and 4.

The unitary daily dosage of the conjugate can vary significantly depending on the patient condition, the disease state being treated, the molecular weight of the conjugate, its route of administration and tissue distribution, and the possibility of co-usage of other therapeutic treatments such as radiation therapy or additional drugs in combination therapies. The effective amount to be administered to a patient is based on body surface area, mass, and physician assessment of patient condition. Effective doses can range, for example, from about 1 ng/kg to about 1 mg/kg, from about 1 μg/kg to about 500 μg/kg, and from about 1 μg/kg to about 100 μg/kg. These doses are based on an average patient weight of about 70 kg.

The conjugates described herein can be administered in a dose of from about 1.0 ng/kg to about 1000 μg/kg, from about 10 ng/kg to about 1000 μg/kg, from about 50 ng/kg to about 1000 μg/kg, from about 100 ng/kg to about 1000 μg/kg, from about 500 ng/kg to about 1000 μg/kg, from about 1 ng/kg to about 500 μg/kg, from about 1 ng/kg to about 100 μg/kg, from about 1 μg/kg to about 50 μg/kg, from about 1 μg/kg to about 10 μg/kg, from about 5 μg/kg to about 500 μg/kg, from about 10 μg/kg to about 100 μg/kg, from about 20 μg/kg to about 200 μg/kg, from about 10 μg/kg to about 500 μg/kg, or from about 50 μg/kg to about 500 μg/kg. The total dose may be administered in single or divided doses and may, at the physician's discretion, fall outside of the typical range given herein. These dosages are based on an average patient weight of about 70 kg. The physician will readily be able to determine doses for subjects whose weight falls outside this range, such as infants and the elderly.

The conjugates described herein may contain one or more chiral centers, or may otherwise be capable of existing as multiple stereoisomers. Accordingly, it is to be understood that the present invention includes pure stereoisomers as well as mixtures of stereoisomers, such as enantiomers, diastereomers, and enantiomerically or diastereomerically enriched mixtures. The conjugates described herein may be capable of existing as geometric isomers. Accordingly, it is to be understood that the present invention includes pure geometric isomers or mixtures of geometric isomers.

It is appreciated that the conjugates described herein may exist in unsolvated forms as well as solvated forms, including hydrated forms. In general, the solvated forms are equivalent to unsolvated forms and are encompassed within the scope of the present invention. The conjugates described herein may exist in multiple crystalline or amorphous forms. In general, all physical forms are equivalent for the uses contemplated by the present invention and are intended to be within the scope of the present invention.

### METHODS AND EXAMPLES

### Compound Examples

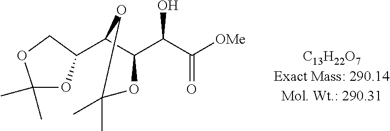

### Example

(3,4), (5,6)-Bisacetonide-D-Gluconic Acid Methyl Ester. In a dry 250 mL round bottom flask, under argon δ-gluconolactone (4.14 g, 23.24 mmol) was suspended in acetone-methanol (50 mL). To this suspension dimethoxypropane (17.15 mL, 139.44 mmol) followed by catalytic amount of p-toulenesulfonic acid (200 mg) were added. This solution was stirred at room temperature for 16 h. TLC (50% EtOAc in petroleum ether) showed that all of the starting material had been consumed and product had been formed. Acetone-methanol was removed under reduced pressure. The residue of the reaction was dissolved in EtOAc and washed with water. The organic layer was washed with brine, dried over Na₂SO₄, and concentrated to dryness. This material was then loaded onto a SiO₂ column and chromatographed (30% EtOAc in petroleum ether) to yield pure (3,4), (5,6)-bisacetonide-D-gluconic acid methyl ester (3.8 g, 56%) and regio-isomer (2,3), (5,6)-bisacetonide-D-gluconic acid methyl ester (0.71 g, 10%). ¹H NMR data was in accordance with the required products.

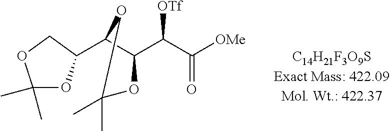

### Example

(3,4), (5,6)-Bisacetonide-2-OTf-D-Gluconic Acid Methyl Ester. In a dry 100 mL round bottom flask, under argon (3,4), (5,6)-bisacetonide-D-gluconic acid methyl ester (3.9 g, 13.43 mmol) was dissolved in methylene chloride (40 mL) and cooled to −20° C. to −25° C. To this solution pyridine (3.26 mL, 40.29 mmol) followed by triflic anhydride (3.39 mL, 20.15 mmol) were added. This white turbid solution was stirred at −20° C. for 1 h. TLC (25% EtOAc in petroleum ether) showed that all of the starting material had been consumed and product had been formed. The reaction mixture was poured into crushed-ice and extracted with diethyl ether. The organic layer was washed with water, brine, dried over Na₂SO₄, and concentrated to yield (3,4), (5,6)-bisacetonide-2-OTf-D-gluconic acid methyl ester (5.5 g, 97%). This material was used in the next reaction without further purification.

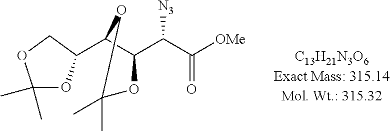

### Example

(3,4), (5,6)-Bisacetonide-2-Deoxy-2-Azido-D-Mannonic Acid Methyl Ester. In a dry 100 mL round bottom flask, under argon (3,4), (5,6)-bisacetonide-2-OTf-D-gluconic acid methyl ester (5.5 g g, 13.02 mmol) was dissolved in DMF (20 mL). To this solution NaN₃ (0.93 g, 14.32 mmol) was added. This solution was stirred at room temperature for 1 h. TLC (8% EtOAc in petroleum ether, triple run) showed that all of the starting material had been consumed and product had been formed. DMF was removed under reduced pressure. The reaction mixture was diluted with brine and extracted with EtOAc. The organic layer was washed with water, brine, dried over Na₂SO₄, and concentrated to dryness. This crude material was then loaded onto a SiO₂ column and chromatographed (20% EtOAc in petroleum ether) to yield pure (3,4), (5,6)-bisacetonide-2-deoxy-2-azido-D-mannonic acid methyl ester (3.8 g, 93%). ¹H NMR data was in accordance with the product.

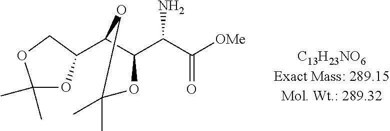

### Example

(3,4), (5,6)-Bisacetonide-2-Deoxy-2-Amino-D-Mannonic Acid Methyl Ester. In a Parr hydrogenation flask, (3,4), (5,6)-bisacetonide-2-deoxy-2-azido-D-mannonic acid methyl ester (3.5 g g, 11.10 mmol) was dissolved in methanol (170 mL). To this solution 10% Pd on carbon (800 mg, 5 mol %) was added. Hydrogenation was carried out using Parr-hydrogenator at 25 PSI for 1 h. TLC (10% methanol in methylene chloride) showed that all of the starting material had been consumed and product had been formed. The reaction mixture was filtered through a celite pad and concentrated to dryness. This crude material was then loaded onto a SiO₂ column and chromatographed (2% methanol in methylene chloride) to yield pure (3,4), (5,6)-bisacetonide-2-deoxy-2-amino-D-mannonic acid methyl ester (2.61 g, 81%). ¹H NMR data was in accordance with the product.

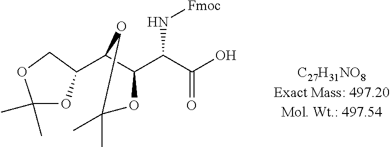

### Example

(3,4), (5,6)-Bisacetonide-2-Deoxy-2-Fmoc-Amino-D-Mannonic Acid. In a dry 100 mL round bottom flask, (3,4), (5,6)-bisacetonide-2-deoxy-2-amino-D-mannonic acid methyl ester (1.24 g, 4.29 mmol) was dissolved in THF/MeOH (20 mL/5 mL). To this solution LiOH.H₂O (215.8 mg, 5.14 mmol) in water (5 mL) was added. This light yellow solution was stirred at room temperature for 2 h. TLC (10% methanol in methylene chloride) showed that all of the starting material had been consumed and product had been formed. THF/MeOH was removed under reduced pressure. The aqueous phase was re-suspended in sat. NaHCO₃ (10 mL). To this suspension Fmoc-OSu (1.74 g, 5.14 mmol) in 1,4-dioxane (10 mL) was added. This heterogeneous solution was stirred at room temperature for 18 h. TLC (10% methanol in methylene chloride) showed that most of the starting material had been consumed and product had been formed. Dioxane was removed under reduced pressure. The aqueous layer was extracted with diethyl ether to remove less polar impurities. Then the aqueous layer was acidified to pH 6 using 0.2N HCl, and re-extracted with EtOAc. The EtOAc layer was washed with brine, dried over Na₂SO₄, and concentrated to yield (3,4), (5,6)-bisacetonide-2-deoxy-2-Fmoc-amino-D-mannonic acid (1.6 g, 76%). This material was used in the next reaction without further purification. ¹H NMR data was in accordance with the product.

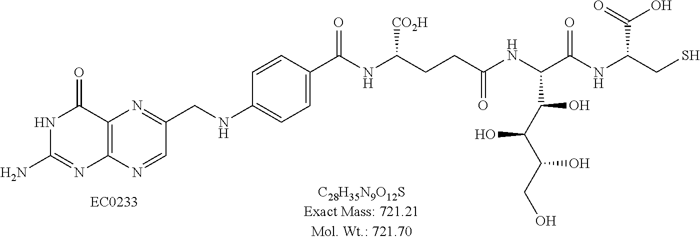

### Example

EC0233 was synthesized by SPPS in three steps according to the general peptide synthesis procedure described herein starting from H-Cys(4-methoxytrityl)-2-chlorotrityl-Resin, and the following SPPS reagents:

| Reagents | mmol | Equivalent | MW | amount | |
|---|---|---|---|---|---|
| H-Cys(4-methoxytrityl)-2- | 0.56 | | | 1.0 | g |
| chlorotrityl-Resin | | | | | |
| (loading 0.56 mmol/g) | | | | | |
| (3,4), (5,6)-bisacetonide-2- | 0.7 | 1.25 | 497.54 | 0.348 | g |
| deoxy-2-Fmoc-amino- | | | | | |
| D-mannonic acid | | | | | |
| Fmoc-Glu-OtBu | 1.12 | 2 | 425.5 | 0.477 | g |
| N¹⁰TFA-Pteroic Acid | 0.70 | 1.25 | 408 | 0.286 | g |
| (dissolve in 10 ml DMSO) | | | | | |
| DIPEA | 2.24 | 4 | 129.25 | 0.390 | mL |
| | | | (d = 0.742) | | |
| PyBOP | 1.12 | 2 | 520 | 0.583 | g |

Coupling steps. In a peptide synthesis vessel add the resin, add the amino acid solution, DIPEA, and PyBOP. Bubble argon for 1 hr. and wash 3× with DMF and IPA. Use 20% piperidine in DMF for Fmoc deprotection, 3× (10 min), before each amino acid coupling. Continue to complete all 3 coupling steps. At the end wash the resin with 2% hydrazine in DMF 3× (5 min) to cleave TFA protecting group on Pteroic acid.

Cleavage step. Cleavage Reagent: 92.5% (50 ml) TFA, 2.5% (1.34 ml) H₂O, 2.5% (1.34 ml) triisopropylsilane, 2.5% (1.34 ml) ethanedithiol. Add 25 ml cleavage reagent and bubble argon for 20 min, drain, and wash 3× with remaining reagent. Rotavap until 5 ml remains and precipitate in ethyl ether. Centrifuge and dry.

HPLC Purification step. Column: Waters NovaPak C₁₈ 300×19 mm; Buffer A=10 mM ammonium acetate, pH 5; B=ACN; Method: 1% B to 20% B in 40 minutes at 15 ml/min; yield ˜202 mg, 50%

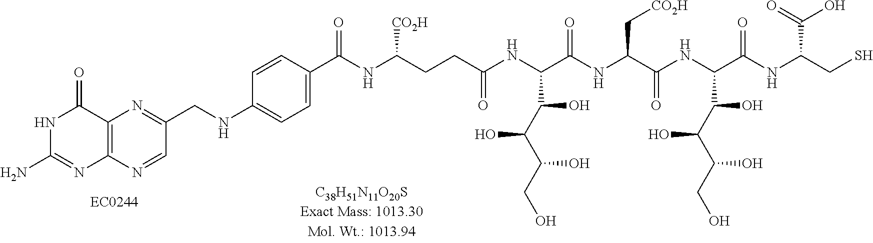

### Example

Bis-Saccharo-Folate Linker EC0244. EC0244 was synthesized by SPPS in five steps according to the general peptide synthesis procedure described herein starting from H-Cys(4-methoxytrityl)-2-chlorotrityl-Resin, and the following SPPS reagents:

| Reagents | mmol | Equivalent | MW | amount |
|---|---|---|---|---|
| H-Cys(4-methoxytrityl)-2-chlorotrityl-Resin | 0.56 | | | 1.0 g |
| (loading 0.56 mmol/g) | | | | |
| (3,4), (5,6)-bisacetonide-2-deoxy-2-Fmoc-amino- | 0.7 | 1.25 | 497.54 | 0.348 g |
| D-mannonic acid | | | | |
| Fmoc-Asp(OtBu)—OH | 1.12 | 2 | 411.5 | 0.461 g |
| (3,4), (5,6)-bisacetonide-2-deoxy-2-Fmoc-amino- | 0.7 | 1.25 | 497.54 | 0.348 g |
| D-mannonic acid | | | | |
| Fmoc-Glu-OtBu | 1.12 | 2 | 425.5 | 0.477 g |
| N¹⁰TFA-Pteroic Acid | 0.70 | 1.25 | 408 | 0.286 g |
| (dissolve in 10 ml DMSO) | | | | |
| DIPEA | 2.24 | 4 | 129.25 | 0.390 mL |
| | | | (d = 0.742) | |
| PyBOP | 1.12 | 2 | 520 | 0.583 g |

The Coupling steps, Cleavage step, Cleavage Reagent, and HPLC Purification step were identical to those described above; yield ˜284 mg, 50%.

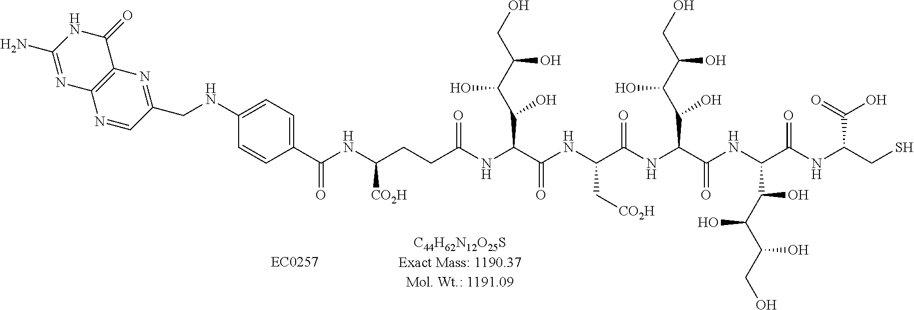

### Example

EC0257 was synthesized by SPPS in six steps according to the general peptide synthesis procedure described herein starting from H-Cys(4-methoxytrityl)-2-chlorotrityl-Resin, and the following SPPS reagents:

| Reagents | mmol | Equivalent | MW | amount |
|---|---|---|---|---|
| H-Cys(4-methoxytrityl)-2-chlorotrityl-Resin | 0.2 | | | 0.333 g |
| (loading 0.56 mmol/g) | | | | |
| (3,4), (5,6)-bisacetonide-2-deoxy-2-Fmoc-amino- | 0.25 | 1.25 | 497.54 | 0.124 g |
| D-mannonic acid | | | | |
| (3,4), (5,6)-bisacetonide-2-deoxy-2-Fmoc-amino- | 0.25 | 1.25 | 497.54 | 0.124 g |
| D-mannonic acid | | | | |
| Fmoc-Asp(OtBu)—OH | 0.4 | 2 | 411.5 | 0.165 g |
| (3,4), (5,6)-bisacetonide-2-deoxy-2-Fmoc-amino- | 0.25 | 1.25 | 497.54 | 0.124 g |
| D-mannonic acid | | | | |
| Fmoc-Glu-OtBu | 0.4 | 2 | 425.5 | 0.170 g |
| N¹⁰TFA-Pteroic Acid | 0.25 | 1.25 | 408 | 0.119 g |
| (dissolve in 10 ml DMSO) | | | | |
| DIPEA | 0.8 | 4 | 129.25 | 0.139 mL |
| | | | (d = 0.742) | |
| PyBOP | 0.4 | 2 | 520 | 0.208 g |

The Coupling steps, Cleavage step, Cleavage Reagent, and HPLC Purification step were identical to those described above; yield ˜170 mg, 71%.

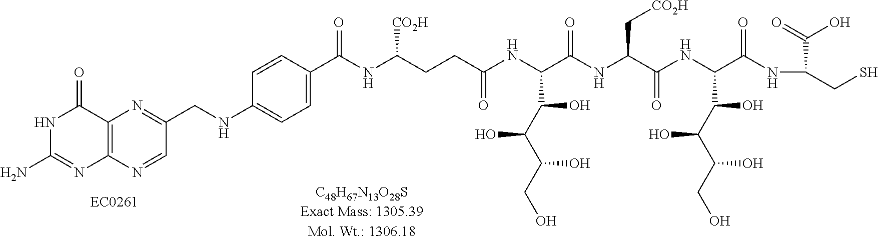

### Example

EC0261 was synthesized by SPPS in seven steps according to the general peptide synthesis procedure described herein starting from H-Cys(4-methoxytrityl)-2-chlorotrityl-Resin, and the following SPPS reagents:

| Reagents | mmol | equivalent | MW | Amount |
|---|---|---|---|---|
| H-Cys(4-methoxytrityl)-2-chlorotrityl-Resin | 0.2 | | | 0.333 g |
| (loading 0.56 mmol/g) | | | | |
| (3,4), (5,6)-bisacetonide-2-deoxy-2-Fmoc-amino- | 0.25 | 1.25 | 497.54 | 0.124 g |
| D-mannonic acid | | | | |
| Fmoc-Asp(OtBu)—OH | 0.4 | 2 | 411.5 | 0.165 g |
| (3,4), (5,6)-bisacetonide-2-deoxy-2-Fmoc-amino- | 0.25 | 1.25 | 497.54 | 0.124 g |
| D-mannonic acid | | | | |
| Fmoc-Asp(OtBu)—OH | 0.4 | 2 | 411.5 | 0.165 g |
| (3,4), (5,6)-bisacetonide-2-deoxy-2-Fmoc-amino- | 0.25 | 1.25 | 497.54 | 0.124 g |
| D-mannonic acid | | | | |
| Fmoc-Glu-OtBu | 0.4 | 2 | 425.5 | 0.170 g |
| N¹⁰TFA-Pteroic Acid | 0.25 | 1.25 | 408 | 0.119 g |
| (dissolve in 10 ml DMSO) | | | | |
| DIPEA | 0.8 | 4 | 129.25 | 0.139 mL |
| | | | (d = 0.742) | |
| PyBOP | 0.4 | 2 | 520 | 0.208 g |

The Coupling steps, Cleavage step, Cleavage Reagent, and HPLC Purification step were identical to those described above; yield ˜170 mg, 65%.

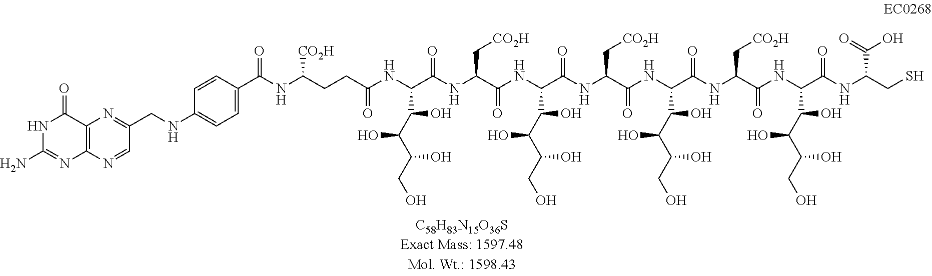

### Example

Tetra-Saccharo-Tris-Asp-Folate Linker EC0268. EC0268 was synthesized by SPPS in nine steps according to the general peptide synthesis procedure described herein starting from H-Cys(4-methoxytrityl)-2-chlorotrityl-Resin, and the following SPPS reagents:

| Reagents | mmol | equivalent | MW | Amount |
|---|---|---|---|---|
| H-Cys(4-methoxytrityl)-2-chlorotrityl-Resin | 0.1 | | | 0.167 g |
| (loading 0.56 mmol/g) | | | | |
| (3,4), (5,6)-bisacetonide-2-deoxy-2-Fmoc-amino- | 0.125 | 1.25 | 497.54 | 0.062 g |
| D-mannonic acid | | | | |
| Fmoc-Asp(OtBu)—OH | 0.2 | 2 | 411.5 | 0.082 g |
| (3,4), (5,6)-bisacetonide-2-deoxy-2-Fmoc-amino- | 0.125 | 1.25 | 497.54 | 0.062 g |
| D-mannonic acid | | | | |
| Fmoc-Asp(OtBu)—OH | 0.2 | 2 | 411.5 | 0.082 g |
| (3,4), (5,6)-bisacetonide-2-deoxy-2-Fmoc-amino- | 0.125 | 1.25 | 497.54 | 0.062 g |
| D-mannonic acid | | | | |
| Fmoc-Asp(OtBu)—OH | 0.2 | 2 | 411.5 | 0.082 g |
| (3,4), (5,6)-bisacetonide-2-deoxy-2-Fmoc-amino- | 0.125 | 1.25 | 497.54 | 0.062 g |
| D-mannonic acid | | | | |
| Fmoc-Glu-OtBu | 0.2 | 2 | 425.5 | 0.085 g |
| N¹⁰TFA-Pteroic Acid | 0.125 | 1.25 | 408 | 0.059 g |
| (dissolve in 10 ml DMSO) | | | | |
| DIPEA | 0.4 | 4 | 129.25 | 0.070 mL |
| | | | (d = 0.742) | |
| PyBOP | 0.2 | 2 | 520 | 0.104 g |

The Coupling steps, Cleavage step, Cleavage Reagent, and HPLC Purification step were identical to those described above; yield ˜100 mg, 63%.

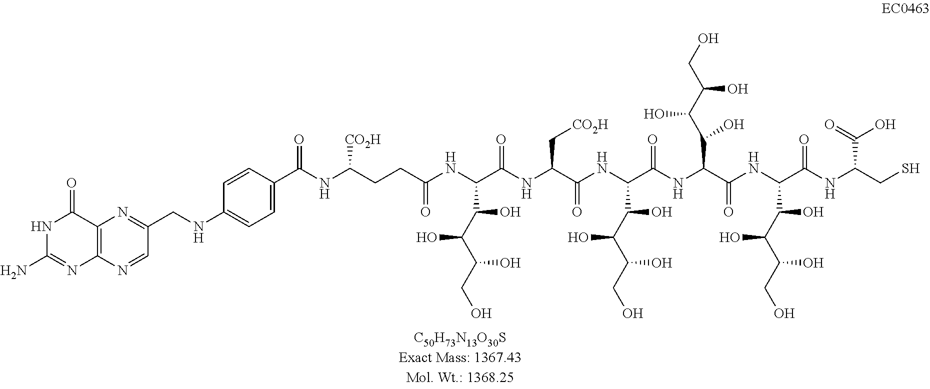

### Example

Tetra-Saccharo-Asp-Folate Linker EC0463. EC0463 was synthesized by SPPS in seven steps according to the general peptide synthesis procedure described herein starting from H-Cys(4-methoxytrityl)-2-chlorotrityl-Resin, and the following SPPS reagents:

| Reagents | mmol | equivalent | MW | Amount |
|---|---|---|---|---|
| H-Cys(4-methoxytrityl)-2-chlorotrityl-Resin | 0.1 | | | 0.167 g |
| (loading 0.56 mmol/g) | | | | |
| (3,4), (5,6)-bisacetonide-2-deoxy-2-Fmoc-amino- | 0.125 | 1.25 | 497.54 | 0.062 g |
| D-mannonic acid | | | | |
| (3,4), (5,6)-bisacetonide-2-deoxy-2-Fmoc-amino- | 0.125 | 1.25 | 497.54 | 0.062 g |
| D-mannonic acid | | | | |
| (3,4), (5,6)-bisacetonide-2-deoxy-2-Fmoc-amino- | 0.125 | 1.25 | 497.54 | 0.062 g |
| D-mannonic acid | | | | |
| Fmoc-Asp(OtBu)—OH | 0.2 | 2 | 411.5 | 0.082 g |
| (3,4), (5,6)-bisacetonide-2-deoxy-2-Fmoc-amino- | 0.125 | 1.25 | 497.54 | 0.062 g |
| D-mannonic acid | | | | |
| Fmoc-Glu—OtBu | 0.2 | 2 | 425.5 | 0.085 g |
| N¹⁰TFA-Pteroic Acid | 0.125 | 1.25 | 408 | 0.059 g |
| (dissolve in 10 ml DMSO) | | | | |
| DIPEA | 0.4 | 4 | 129.25 | 0.070 mL |
| | | | (d = 0.742) | |
| PyBOP | 0.2 | 2 | 520 | 0.104 g |

The Coupling steps, Cleavage step, Cleavage Reagent, and HPLC Purification step were identical to those described above; yield ˜63 mg, 46%

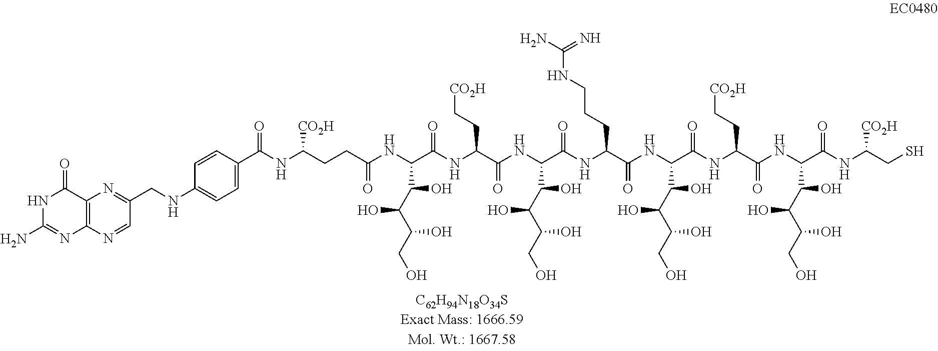

### Example

Tetra-Saccharo-Bis-α-Glu-Arg-Folate Linker EC0480. EC0480 was synthesized by SPPS in nine steps according to the general peptide synthesis procedure described herein starting from H-Cys(4-methoxytrityl)-2-chlorotrityl-Resin, and the following SPPS reagents:

| Reagents | mmol | equivalent | MW | Amount |
|---|---|---|---|---|
| H-Cys(4-methoxytrityl)-2-chlorotrityl-Resin | 0.2 | | | 0.333 g |
| (loading 0.56 mmol/g) | | | | |
| (3,4), (5,6)-bisacetonide-2-deoxy-2-Fmoc-amino- | 0.250 | 1.25 | 497.54 | 0.124 g |
| D-mannonic acid | | | | |
| Fmoc-Glu(OtBu)—OH | 0.4 | 2 | 425.5 | 0.170 g |
| (3,4), (5,6)-bisacetonide-2-deoxy-2-Fmoc-amino- | 0.250 | 1.25 | 497.54 | 0.124 g |
| D-mannonic acid | | | | |
| Fmoc-Arg(Pbf)-OH | 0.4 | 2 | 648.78 | 0.260 g |
| (3,4), (5,6)-bisacetonide-2-deoxy-2-Fmoc-amino- | 0.250 | 1.25 | 497.54 | 0.124 g |
| D-mannonic acid | | | | |
| Fmoc-Glu(OtBu)—OH | 0.4 | 2 | 425.5 | 0.170 g |
| (3,4), (5,6)-bisacetonide-2-deoxy-2-Fmoc-amino- | 0.250 | 1.25 | 497.54 | 0.124 g |
| D-mannonic acid | | | | |
| Fmoc-Glu-OtBu | 0.4 | 2 | 425.5 | 0.170 g |
| N¹⁰TFA-Pteroic Acid | 0.250 | 1.25 | 408 | 0.119 g |
| (dissolve in 10 ml DMSO) | | | | |
| DIPEA | 0.8 | 4 | 129.25 | 0.139 mL |
| | | | (d = 0.742) | |
| PyBOP | 0.4 | 2 | 520 | 0.208 g |

The Coupling steps, Cleavage step, Cleavage Reagent, and HPLC Purification step were identical to those described above; yield ˜100 mg, 33%

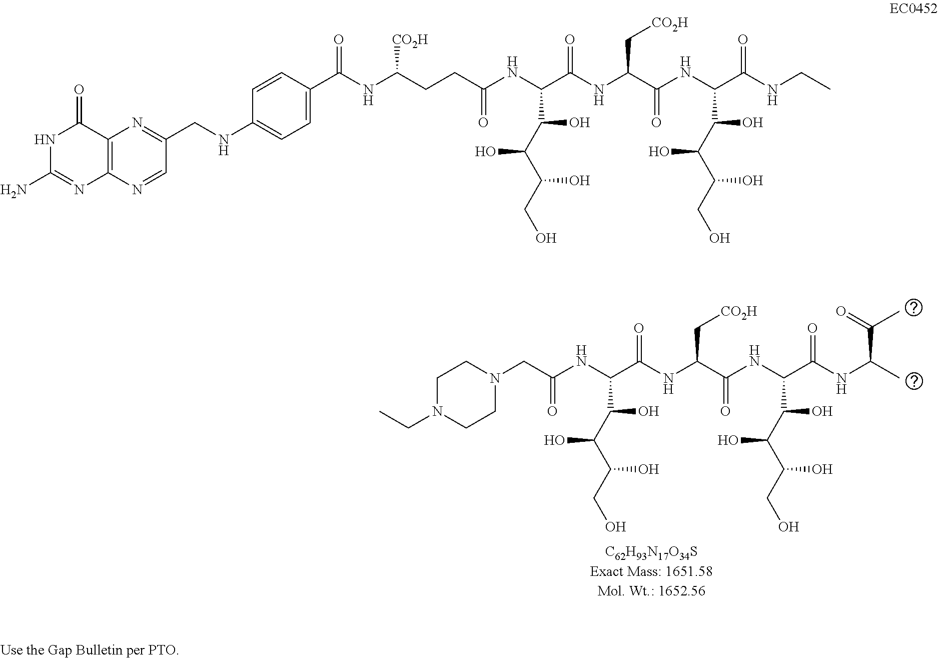

### Example

Tetra-Saccharo-Bis-Asp-Folate Linker EC0452. EC0452 was synthesized by SPPS in nine steps according to the general peptide synthesis procedure described herein starting from H-Cys(4-methoxytrityl)-2-chlorotrityl-Resin, and the following SPPS reagents:

| Reagents | mmol | equivalent | MW | Amount |
|---|---|---|---|---|
| H-Cys(4-methoxytrityl)-2-chlorotrityl-Resin | 0.15 | | | 0.250 g |
| (loading 0.6 mmol/g) | | | | |
| (3,4), (5,6)-bisacetonide-2-deoxy-2-Fmoc-amino- | 0.188 | 1.25 | 497.54 | 0.094 g |
| D-mannonic acid | | | | |
| Fmoc-Asp(OtBu)—OH | 0.3 | 2 | 411.5 | 0.123 g |
| (3,4), (5,6)-bisacetonide-2-deoxy-2-Fmoc-amino- | 0.188 | 1.25 | 497.54 | 0.094 g |
| D-mannonic acid | | | | |
| Fmoc-4-(2-aminoethyl)-1-carboxymethyl- | 0.3 | 2 | 482.42 | 0.145 g |
| piperazine dihydrochloride | | | | |
| (3,4), (5,6)-bisacetonide-2-deoxy-2-Fmoc-amino- | 0.188 | 1.25 | 497.54 | 0.094 g |
| D-mannonic acid | | | | |
| Fmoc-Asp(OtBu)—OH | 0.3 | 2 | 411.5 | 0.123 g |
| (3,4), (5,6)-bisacetonide-2-deoxy-2-Fmoc-amino- | 0.188 | 1.25 | 497.54 | 0.094 g |
| D-mannonic acid | | | | |
| Fmoc-Glu-OtBu | 0.3 | 2 | 425.5 | 0.128 g |
| N¹⁰TFA-Pteroic Acid | 0.188 | 1.25 | 408 | 0.077 g |
| (dissolve in 10 ml DMSO) | | | | |
| DIPEA | 0.6 | 4 | 129.25 | 0.105 mL |
| | | | (d = 0.742) | |
| PyBOP | 0.3 | 2 | 520 | 0.156 g |

The Coupling steps, Cleavage step, and Cleavage Reagent were identical to those described above. HPLC Purification step. Column: Waters NovaPak C₁₈ 300×19 mm; Buffer A=10 mM ammonium acetate, pH 5; B=ACN; Method: 1% B to 20% B in 40 minutes at 25 ml/min; yield ˜98 mg, 40%

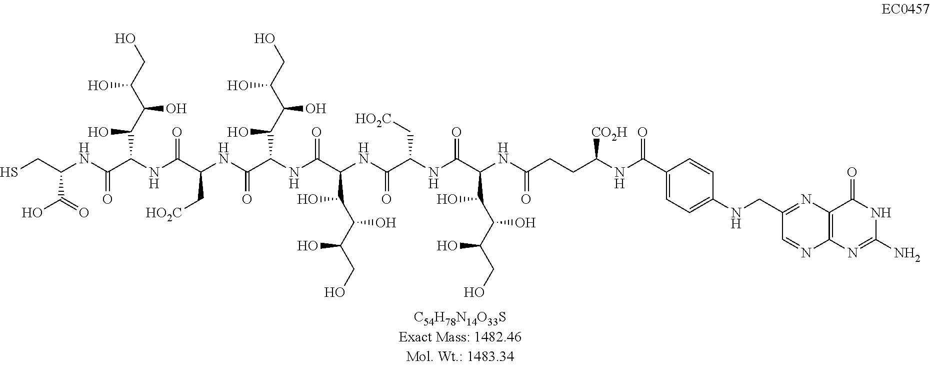

### Example

Tetra-Saccharo-bis-Asp-Folate Linker EC0457. EC0457 was synthesized by SPPS in eight steps according to the general peptide synthesis procedure described herein starting from H-Cys(4-methoxytrityl)-2-chlorotrityl-Resin, and the following SPPS reagents:

| Reagents | mmol | equivalent | MW | Amount |
|---|---|---|---|---|
| H-Cys(4-methoxytrityl)-2-chlorotrityl- | 0.20 | | | 0.333 g |
| Resin (loading 0.6 mmol/g) | | | | |
| (3,4), (5,6)-bisacetonide-2-deoxy-2-Fmoc- | 0.25 | 1.25 | 497.54 | 0.124 g |
| amino-D-mannonic acid | | | | |
| Fmoc-Asp(OtBu)—OH | 0.30 | 1.5 | 411.5 | 0.123 g |
| (3,4), (5,6)-bisacetonide-2-deoxy-2-Fmoc- | 0.25 | 1.25 | 497.54 | 0.124 g |
| amino-D-mannonic acid | | | | |
| (3,4), (5,6)-bisacetonide-2-deoxy-2-Fmoc- | 0.25 | 1.25 | 497.54 | 0.124 g |
| amino-D-mannonic acid | | | | |
| Fmoc-Asp(OtBu)—OH | 0.30 | 1.5 | 411.5 | 0.123 g |
| (3,4), (5,6)-bisacetonide-2-deoxy-2-Fmoc- | 0.25 | 1.25 | 497.54 | 0.124 g |
| amino-D-mannonic acid | | | | |
| Fmoc-Glu-OtBu | 0.30 | 1.5 | 425.5 | 0.128 g |
| N¹⁰TFA-Pteroic Acid | 0.25 | 1.25 | 408 | 0.102 g |
| (dissolve in 10 ml DMSO) | | | | |
| DIPEA | | 2 eq. of amino | 129.25 | 87 μL or 105 |
| | | acid | (d = 0.742) | μL |
| PyBOP | | 2 eq. of amino | 520 | 260 mg or |
| | | acid | | 312 mg |

The Coupling steps, Cleavage step, and Cleavage Reagent were identical to those described above. HPLC Purification step. Column: Waters NovaPak C₁₈ 300×19 mm; Buffer A=10 mM ammonium acetate, pH 5; B=ACN; Method: 0% B to 20% B in 40 minutes at 25 ml/min; yield ˜210 mg, 71%

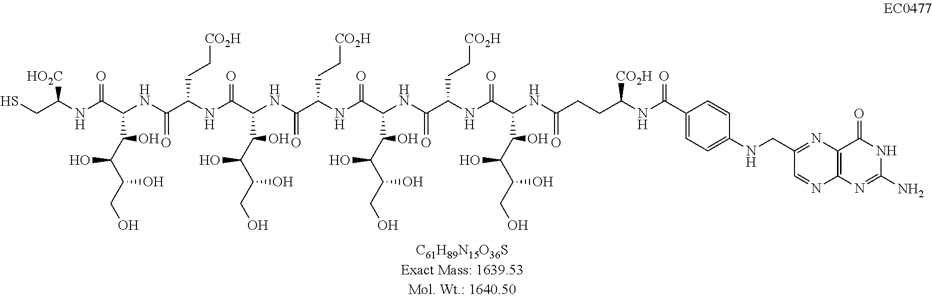

### Example

Tetra-Saccharo-tris-Glu-Folate Linker EC0477. EC0477 was synthesized by SPPS in nine steps according to the general peptide synthesis procedure described herein starting from H-Cys(4-methoxytrityl)-2-chlorotrityl-Resin, and the following SPPS reagents:

| Reagents | mmol | equivalent | MW | Amount |
|---|---|---|---|---|
| H-Cys(4-methoxytrityl)-2-chlorotrityl- | 0.20 | | | 0.333 g |
| Resin (loading 0.6 mmol/g) | | | | |
| (3,4), (5,6)-bisacetonide-2-deoxy-2-Fmoc- | 0.25 | 1.25 | 497.54 | 0.124 g |
| amino-D-mannonic acid | | | | |
| Fmoc-Glu(OtBu)—OH | 0.30 | 1.5 | 425.5 | 0.128 g |
| (3,4), (5,6)-bisacetonide-2-deoxy-2-Fmoc- | 0.25 | 1.25 | 497.54 | 0.124 g |
| amino-D-mannonic acid | | | | |
| Fmoc-Glu(OtBu)—OH | 0.30 | 1.5 | 425.5 | 0.128 g |
| (3,4), (5,6)-bisacetonide-2-deoxy-2-Fmoc- | 0.25 | 1.25 | 497.54 | 0.124 g |
| amino-D-mannonic acid | | | | |
| Fmoc-Glu(OtBu)—OH | 0.30 | 1.5 | 425.5 | 0.128 g |
| (3,4), (5,6)-bisacetonide-2-deoxy-2-Fmoc- | 0.25 | 1.25 | 497.54 | 0.124 g |
| amino-D-mannonic acid | | | | |
| Fmoc-Glu-OtBu | 0.30 | 1.5 | 425.5 | 0.128 g |
| N¹⁰TFA-Pteroic Acid | 0.25 | 1.25 | 408 | 0.102 g |
| (dissolve in 10 ml DMSO) | | | | |
| DIPEA | | 2 eq. of amino | 129.25 | 87 μL or 105 |
| | | acid | (d = 0.742) | μL |
| PyBOP | | 1 eq. of amino | 520 | 130 mg or |
| | | acid | | 156 mg |

The Coupling steps, Cleavage step, and Cleavage Reagent were identical to those described above. HPLC Purification step. Column: Waters NovaPak C₁₈ 300×19 mm; Buffer A=10 mM ammonium acetate, pH 5; B=ACN; Method: 0% B to 20% B in 40 minutes at 25 ml/min; yield ˜220 mg, 67%

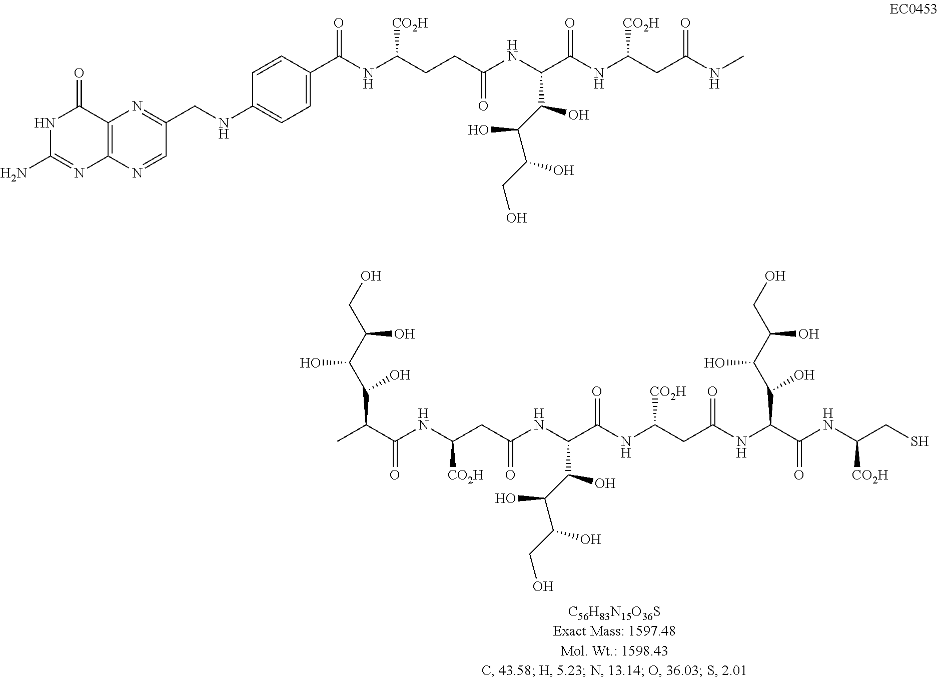

### Example

EC0453 was synthesized by SPPS according to the general peptide synthesis procedure described herein starting from H-Cys(4-methoxytrityl)-2-chlorotrityl-Resin, and the following SPPS reagents:

| Reagents | mmol | equivalent | MW | Amount |
|---|---|---|---|---|
| H-Cys(4-methoxytrityl)-2-chlorotrityl-Resin | 0.162 | | | 0.290 g |
| (loading 0.56 mmol/g) | | | | |
| (3,4), (5,6)-bisacetonide-2-deoxy-2-Fmoc- | 0.203 | 1.25 | 497.54 | 0.101 g |
| amino-D-mannonic acid | | | | |
| Fmoc-Asp(OtBu)—OH | 0.324 | 2 | 411.5 | 0.133 g |
| (3,4), (5,6)-bisacetonide-2-deoxy-2-Fmoc- | 0.203 | 1.25 | 497.54 | 0.101 g |
| amino-D-mannonic acid | | | | |
| Fmoc-Asp(OtBu)—OH | 0.324 | 2 | 411.5 | 0.133 g |
| (3,4), (5,6)-bisacetonide-2-deoxy-2-Fmoc- | 0.203 | 1.25 | 497.54 | 0.101 g |
| amino-D-mannonic acid | | | | |
| Fmoc-Asp(OtBu)—OH | 0.324 | 2 | 411.5 | 0.133 g |
| (3,4), (5,6)-bisacetonide-2-deoxy-2-Fmoc- | 0.203 | 1.25 | 497.54 | 0.101 g |
| amino-D-mannonic acid | | | | |
| Fmoc-Glu-OtBu | 0.324 | 2 | 425.5 | 0.138 g |
| N¹⁰TFA-Pteroic Acid | 0.203 | 1.25 | 408 | 0.083 g |
| (dissolve in 10 ml DMSO) | | | | |
| DIPEA | | 2 eq of | | 71 μL or 85 |
| | | AA | | μL |
| PyBOP | | 1 eq of | | 211 mg or 253 |
| | | AA | | mg |

Coupling steps. In a peptide synthesis vessel add the resin, add the amino acid solution, DIPEA, and PyBOP. Bubble argon for 1 hr. and wash 3× with DMF and IPA. Use 20% piperidine in DMF for Fmoc deprotection, 3× (10 min), before each amino acid coupling. Continue to complete all 9 coupling steps. At the end treat the resin with 2% hydrazine in DMF 3× (5 min) to cleave TFA protecting group on Pteroic acid, wash the resin with DMF (3×), IPA (3×), MeOH (3×), and bubble the resin with argon for 30 min.

Cleavage step. Cleavage Reagent: 92.5% TFA, 2.5% H₂O, 2.5% triisopropylsilane, 2.5% ethanedithiol. Treat the resin with cleavage reagent 3 times (15 min, 5 min, 5 min) with argon bubbling, drain, collect, and combine the solution. Rotavap until 5 ml remains and precipitate in diethyl ether (35 mL). Centrifuge, wash with diethyl ether, and dry. The crude solid was purified by HPLC.

HPLC Purification step. Column: Waters Xterra Prep MS C₁₈ 10 μm 19×250 mm; Solvent A: 10 mM ammonium acetate, pH 5; Solvent B: ACN; Method: 5 min 0% B to 40 min 20% B 25 mL/min; Fractions containing the product was collected and freeze-dried to give 60 mg EC0453 (23% yield). ¹H NMR and LC/MS were consistent with the product.

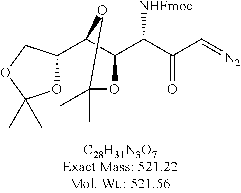

### Example

(3,4), (5,6)-Bisacetonide-2-deoxy-2-Fmoc-amino-D-Mannonic acid-diazo-ketone. In a dry 100 mL round bottom flask, (3,4), (5,6)-bisacetonide-2-deoxy-2-Fmoc-amino-D-mannonic acid (1.0 g, 2.01 mmol) was dissolved in THF (10 mL, not fully dissolved) under Argon atmosphere. The reaction mixture was cooled to −25° C. To this solution NMM (0.23 mL, 2.11 mmol) and ethylchloroformate (228.98 mg, 2.11 mmol) were added. This solution was stirred at −20° C. for 30 min. The resulting white suspension was allowed to warm to 0° C., and a solution of diazomethane in ether was added until yellow color persisted. Stirring was continued as the mixture was allowed to warm to room temperature. Stirred for 2 h, excess diazomethane was destroyed by the addition of few drops of acetic acid with vigorous stirring. The mixture was diluted with ether, washed with sat. aq. NaHCO₃ solution, sat. aq. NH₄Cl, brine, dried over Na₂SO₄, and concentrated to dryness. This crude material was then loaded onto a SiO₂ column and chromatographed (30% EtOAc in petroleum ether) to yield pure (3,4), (5,6)-bisacetonide-2-deoxy-2-Fmoc-amino-D-mannonic acid-diazo-ketone (0.6 g, 57%). ¹H NMR data was in accordance with the product.

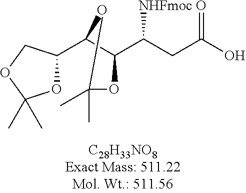

### Example

(3R, 4R, 5S, 6R)-(4,5),(6,7)-Bisacetonide-3-Fmoc-Amino-Heptanoic acid. In a dry 25 mL round bottom flask, (3,4), (5,6)-bisacetonide-2-deoxy-2-Fmoc-amino-D-mannonic acid-diazo-ketone (0.15 g, 0.29 mmol) was dissolved in THF (1.6 mL) under Argon atmosphere. To this solution silver trifluoroacetate (6.6 mg, 0.03 mmol) in water (0.4 mL) was added in the dark. The resulting mixture was stirred at room temperature for 16 h. TLC (10% MeOH in methylene chloride) showed that all of the starting material had been consumed and product had been formed. Solvent (THF) was removed under reduced pressure, the residue was diluted with water (pH was 3.5-4.0) and extracted with EtOAc. The organic layer was washed with brine, dried over Na₂SO₄, and concentrated to dryness. This crude material was then loaded onto a SiO₂ column and chromatographed (gradient elution from 1% MeOH in methylene chloride to 5% MeOH in methylene chloride) to yield pure (3R, 4R, 5S, 6R)-(4,5),(6,7)-bisacetonide-3-Fmoc-amino-heptanoic acid (0.10 g, 68%). ¹H NMR data was in accordance with the product.

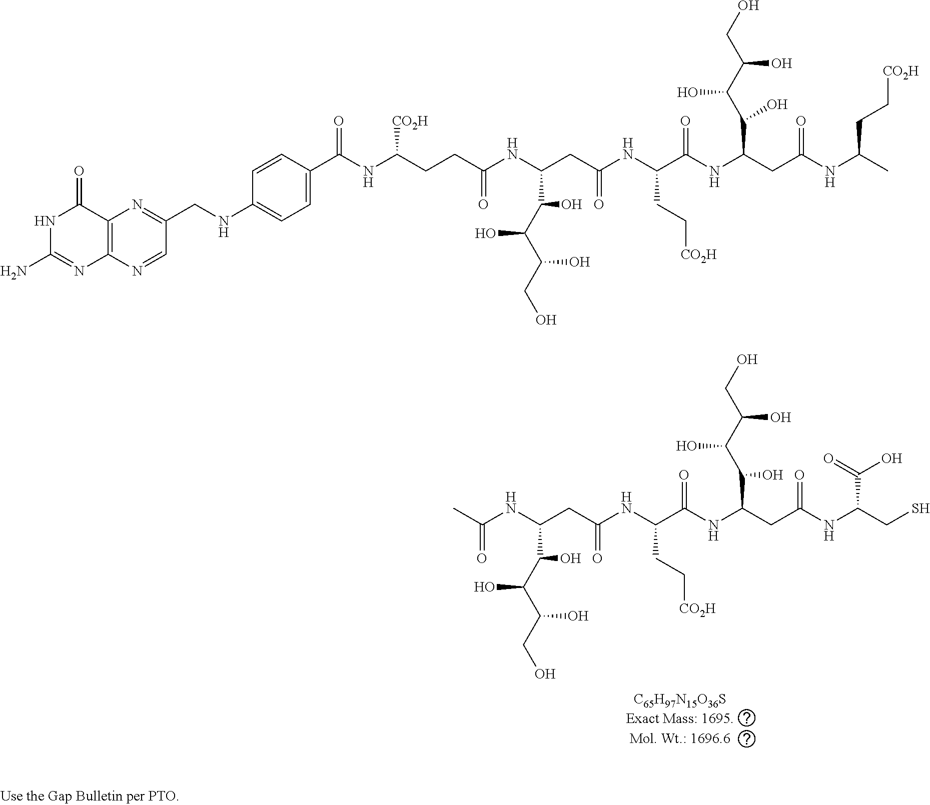

### Example

Tetra-Homosaccharo-Tris-αGlu-Folate Spacer EC0478. EC0478 was synthesized by SPPS in nine steps according to the general peptide synthesis procedure described herein starting from H-Cys(4-methoxytrityl)-2-chlorotrityl-Resin, and the following SPPS reagents:

| Reagents | mmol | equivalent | MW | Amount |
|---|---|---|---|---|
| H-Cys(4-methoxytrityl)- | 0.1 | | | 0.167 g |
| 2-chlorotrityl-Resin | | | | |
| (loading 0.56 mmol/g) | | | | |
| Homo sugar | 0.12 | 1.2 | 511.56 | 0.061 g |
| Fmoc-Glu(OtBu)—OH | 0.2 | 2 | 425.5 | 0.085 g |
| Homo sugar | 0.12 | 1.2 | 511.56 | 0.061 g |
| Fmoc-Glu(OtBu)—OH | 0.2 | 2 | 425.5 | 0.085 g |
| Homo sugar | 0.12 | 1.2 | 511.56 | 0.061 g |
| Fmoc-Glu(OtBu)—OH | 0.2 | 2 | 425.5 | 0.085 g |
| Homo sugar | 0.12 | 1.2 | 511.56 | 0.061 g |
| Fmoc-Glu-OtBu | 0.2 | 2 | 425.5 | 0.085 g |
| N¹⁰TFA-Pteroic | 0.12 | 1.2 | 408 | 0.049 g |
| Acid•TFA (dissolve | | | | |
| in 10 ml DMSO) | | | | |
| DIPEA | 0.4 | 4 | 129.25 | 0.070 mL |
| | | | (d = 0.742) | |
| PyBOP | 0.2 | 2 | 520 | 0.104 g |

The Coupling steps, Cleavage step, and Cleavage Reagent were identical to those described above. HPLC Purification step: Column: Waters NovaPak C₁₈ 300×19 mm; Buffer A=10 mM ammonium acetate, pH 5; B=ACN; Method: 100% A for 5 min then 0% B to 20% B in 20 minutes at 26 ml/min; yield ˜88 mg, 52%

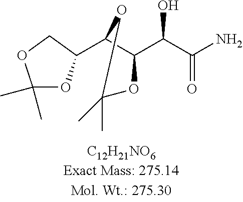

### Example

(3,4), (5,6)-Bisacetonide-D-Gluconic Amide. 20 g of the methyl ester was dissolved in 100 mL methanol, cooled the high-pressure reaction vessel with dry ice/acetone, charged with 100 mL liquid ammonia, warmed up to room temperature and heated to 160° C./850 PSI for 2 hours. The reaction vessel was cooled to room temperature and released the pressure. Evaporation of the solvent gave brownish syrup, and minimum amount of isopropyl alcohol was added to make the homogeneous solution with reflux. The solution was cooled to −20° C. and the resulting solid was filtered to give 8.3 g of solid. The mother liquid was evaporated, and to the resulting residue, ether was added and refluxed until homogeneous solution was achieved. The solution was then cooled to −20° C. and the resulting solid was filtered to give 4.0 g product. The solid was combined and recrystallized in isopropyl alcohol to give 11.2 g (59%) of the white amide product.

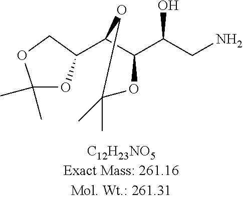

### Example

(3,4), (5,6)-Bisacetonide-1-Deoxy-1-Amino-D-Glucitol. In a dry 100 mL round bottom flask, under argon, LiAlH₄ (450 mg, 11.86 momol)) was dissolved in THF (10 mL) and cooled to 0° C. To this suspension (3,4), (5,6)-bisacetonide-D-gluconic amide (1.09 g, 3.96 mmol) in THF (30 mL) was added very slowly over 15 min. This mixture was refluxed for 5 h. TLC (10% MeOH in methylene chloride) showed that all of the starting material had been consumed and product had been formed. The reaction mixture was cooled to room temperature, and then cooled to ice-bath temperature, diluted with diethyl ether (40 mL), slowly added 0.5 mL of water, 0.5 mL of 15% aq. NaOH, and then added 1.5 mL of water. The reaction mixture was warmed to room temperature and stirred for 30 min. MgSO₄ was added and stirred for additional 15 min and filtered. The organic layer was concentrated to dryness to yield (3,4), (5,6)-bisacetonide-1-deoxy-1-amino-D-glucitol. ¹H NMR data was in accordance with the product.

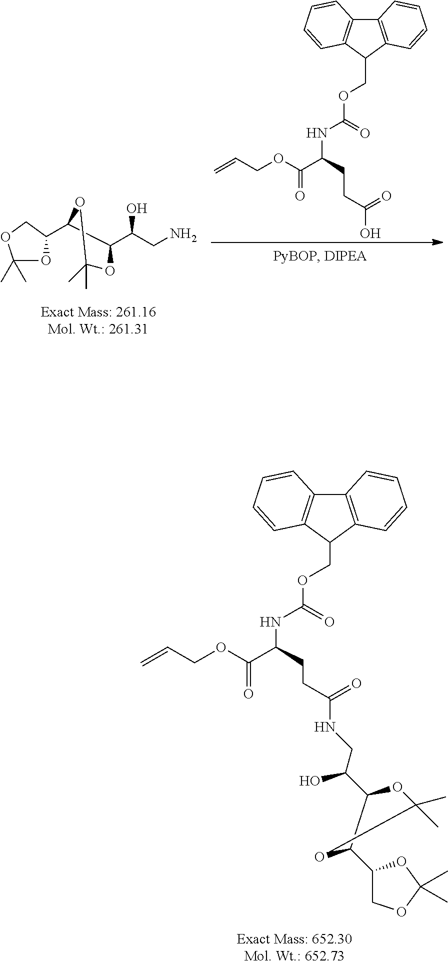

### Example

EC0475. Fmoc-Glu-OAll (2.17 g, 1 eq), PyBOP (2.88 g, 1 eq), and DIPEA (1.83 mL, 2 eq) were added to a solution of the aminosugar (1.4 g, 5.3 mmol) in dry DMF (6 mL) and the RM was stirred at RT under Ar for 2 h. The solution was diluted with EtOAc (50 mL), washed with brine (10 mL×3), organic layer separated, dried (MgSO₄), filtered and concentrated to give a residue, which was purified by a flash column (silica gel, 60% EtOAc/petro-ether) to afford 1.72 g (50%) allyl-protected EC0475 as a solid.

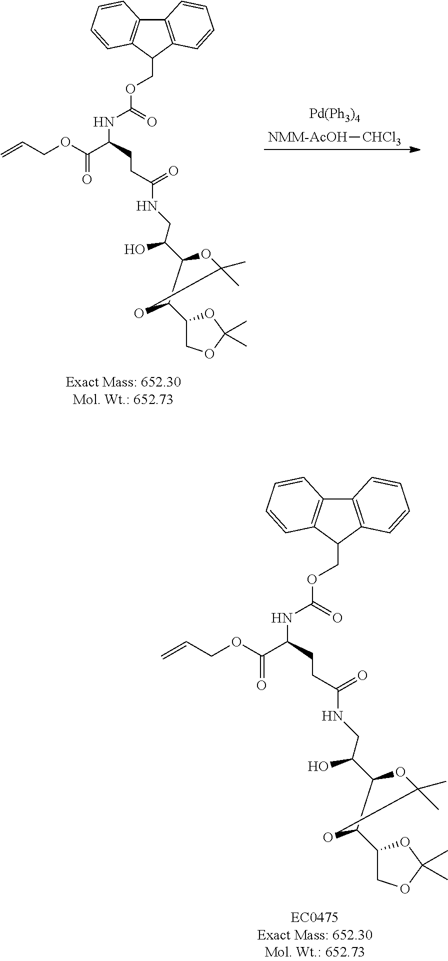

Pd(Ph₃)₄ (300 mg, 0.1 eq) was added to a solution of allyl-protected EC0475 (1.72 g, 2.81 mmol) in NMM/AcOH/CHCl₃ (2 mL/4 mL/74 mL). The resulting yellow solution was stirred at RT under Ar for 1 h, to which was added a second portion of Pd(Ph₃)₄ (300 mg, 0.1 eq). After stirring for an additional 1 h, the RM was washed with 1N HCl (50 mL×3) and brine (50 mL), organic layer separated, dried (MgSO4), filtered, and concentrated to give a yellow foamy solid, which was subject to chromatography (silica gel, 1% MeOH/CHCl₃ followed by 3.5% MeOH/CHCl₃) to give 1.3 g (81%) EC0475 as a solid material.

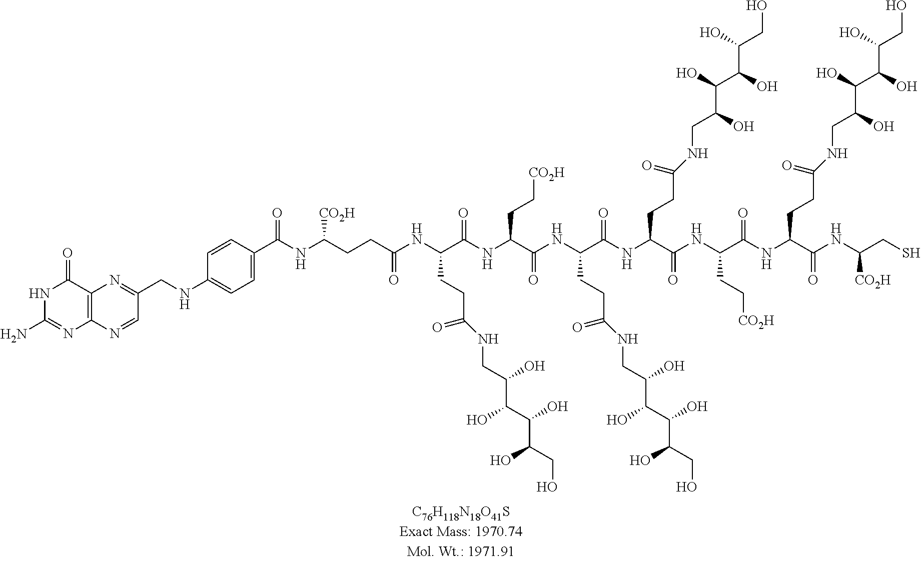

### Example

Tetra-Saccharoglutamate-Bis-αGlu-Folate Spacer EC0491. EC0491 was synthesized by SPPS in eight steps according to the general peptide synthesis procedure described herein starting from H-Cys(4-methoxytrityl)-2-chlorotrityl-Resin, and the following SPPS reagents:

| Reagents | Mmol | equivalent | MW | Amount |
|---|---|---|---|---|
| H-Cys(4-methoxy- | 0.1 | | | 0.167 g |
| trityl)-2-chloro- | | | | |
| trityl-Resin | | | | |
| (loading 0.56 mmol/g) | | | | |
| EC0475 | 0.13 | 1.3 | 612.67 | 0.080 g |
| Fmoc-Glu(OtBu)—OH | 0.2 | 2 | 425.5 | 0.085 g |
| EC0475 | 0.13 | 1.3 | 612.67 | 0.080 g |
| EC0475 | 0.13 | 1.3 | 612.67 | 0.080 g |
| Fmoc-Glu(OtBu)—OH | 0.2 | 2 | 425.5 | 0.085 g |
| EC0475 | 0.13 | 1.3 | 612.67 | 0.080 g |
| Fmoc-Glu-OtBu | 0.2 | 2 | 425.5 | 0.085 g |
| N¹⁰TFA-Pteroic | 0.2 | 2 | 408 | 0.105 g |
| Acid•TFA (dis- | | | | |
| solve in 10 ml DMSO) | | | | |
| DIPEA | 0.4 | 4 | 129.25 | 0.070 mL |
| | | | (d = 0.742) | |
| PyBOP | 0.2 | 2 | 520 | 0.104 g |

The Coupling steps, Cleavage step, and Cleavage Reagent were identical to those described above. HPLC Purification step: Column: Waters NovaPak C₁₈ 300×19 mm; Buffer A=10 mM ammonium acetate, pH 5; B=ACN; Method: 100% A for 5 min then 0% B to 20% B in 20 minutes at 26 ml/min; yield ˜100 mg, 51%.

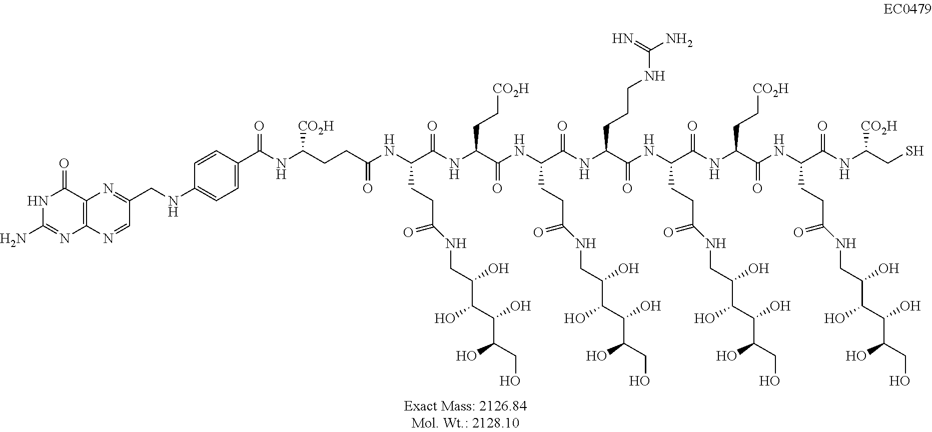

### Example

EC0479 was synthesized by SPPS according to the general peptide synthesis procedure described herein starting from H-Cys(4-methoxytrityl)-2-chlorotrityl-Resin, and the following SPPS reagents:

| Reagents | mmol | equivalent | MW | Amount |
|---|---|---|---|---|
| H-Cys(4-methoxytrityl)- | 0.094 | | | 0.16 g |
| 2-chlorotrityl-Resin | | | | |
| (loading 0.6 mmol/g) | | | | |
| EC0475 | 0.13 | 1.4 | 612.67 | 0.082 g |
| Fmoc-Glu(OtBu)—OH | 0.19 | 2.0 | 425.47 | 0.080 g |
| EC0475 | 0.13 | 1.4 | 612.67 | 0.082 g |
| Fmoc-Arg(Pbf)-OH | 0.19 | 2.0 | 648.77 | 0.12 g |
| EC0475 | 0.13 | 1.4 | 612.67 | 0.082 g |
| Fmoc-Glu(OtBu)—OH | 0.19 | 2.0 | 425.47 | 0.080 g |
| EC0475 | 0.13 | 1.4 | 612.67 | 0.082 g |
| Fmoc-Glu-OtBu | 0.19 | 2.0 | 425.47 | 0.080 g |
| N¹⁰TFA-Pteroic Acid | 0.16 | 1.7 | 408.29 | 0.066 g |
| (dissolve in 10 ml DMSO) | | | | |
| DIPEA | | 2.0 eq of | | 41 μL or 49 μL |
| | | AA | | |
| PyBOP | | 1.0 eq of | | 122 mg or |
| | | AA | | 147 mg |

Coupling steps. In a peptide synthesis vessel add the resin, add the amino acid solution, DIPEA, and PyBOP. Bubble argon for 1 hr. and wash 3× with DMF and IPA. Use 20% piperidine in DMF for Fmoc deprotection, 3× (10 min), before each amino acid coupling. Continue to complete all 9 coupling steps. At the end treat the resin with 2% hydrazine in DMF 3× (5 min) to cleave TFA protecting group on Pteroic acid, wash the resin with DMF (3×), IPA (3×), MeOH (3×), and bubble the resin with argon for 30 min.

Cleavage step. Reagent: 92.5% TFA, 2.5% H₂O, 2.5% triisopropylsilane, 2.5% ethanedithiol. Treat the resin with cleavage reagent for 15 min with argon bubbling, drain, wash the resin once with cleavage reagent, and combine the solution. Rotavap until 5 ml remains and precipitate in diethyl ether (35 mL). Centrifuge, wash with diethyl ether, and dry. The crude solid was purified by HPLC.

HPLC Purification step. Column: Waters Atlantis Prep T3 10 μm OBD 19×250 mm; Solvent A: 10 mM ammonium acetate, pH 5; Solvent B: ACN; Method: 5 min 0% B to 20 min 20% B 26 mL/min. Fractions containing the product was collected and freeze-dried to give ˜70 mg EC0479 (35% yield). ¹H NMR and LC/MS were consistent with the product.

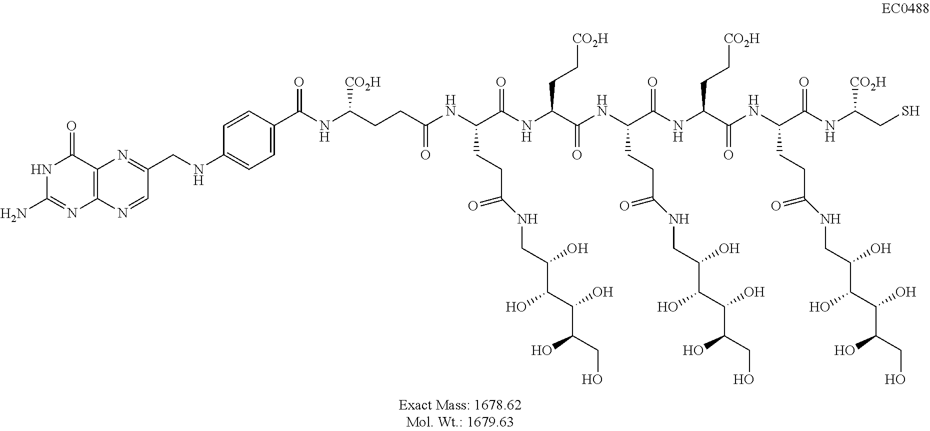

### Example

EC0488 was prepared by SPPS according to the general peptide synthesis procedure described herein starting from H-Cys(4-methoxytrityl)-2-chlorotrityl-Resin, and the following SPPS reagents:

| Reagents | mmol | equivalent | MW | amount |
|---|---|---|---|---|
| H-Cys(4-methoxytrityl)- | 0.10 | | | 0.17 g |
| 2-chlorotrityl-Resin | | | | |
| (loading 0.6 mmol/g) | | | | |
| EC0475 | 0.13 | 1.3 | 612.67 | 0.082 g |
| Fmoc-Glu(OtBu)—OH | 0.19 | 1.9 | 425.47 | 0.080 g |
| EC0475 | 0.13 | 1.3 | 612.67 | 0.082 g |
| Fmoc-Glu(OtBu)—OH | 0.19 | 1.9 | 425.47 | 0.080 g |
| EC0475 | 0.13 | 1.3 | 612.67 | 0.082 g |
| Fmoc-Glu-OtBu | 0.19 | 1.9 | 425.47 | 0.080 g |
| N¹⁰TFA-Pteroic Acid | 0.16 | 1.6 | 408.29 | 0.066 g |
| (dissolve in 10 ml DMSO) | | | | |
| DIPEA | | 2.0 eq of AA | | |
| PyBOP | | 1.0 eq of AA | | |

Coupling steps. In a peptide synthesis vessel add the resin, add the amino acid solution, DIPEA, and PyBOP. Bubble argon for 1 hr. and wash 3× with DMF and IPA. Use 20% piperidine in DMF for Fmoc deprotection, 3× (10 min), before each amino acid coupling. Continue to complete all 9 coupling steps. At the end treat the resin with 2% hydrazine in DMF 3× (5 min) to cleave TFA protecting group on Pteroic acid, wash the resin with DMF (3×), IPA (3×), MeOH (3×), and bubble the resin with argon for 30 min.

Cleavage step. Reagent: 92.5% TFA, 2.5% H₂O, 2.5% triisopropylsilane, 2.5% ethanedithiol. Treat the resin with cleavage reagent 3×(10 min, 5 min, 5 min) with argon bubbling, drain, wash the resin once with cleavage reagent, and combine the solution. Rotavap until 5 ml remains and precipitate in diethyl ether (35 mL). Centrifuge, wash with diethyl ether, and dry. About half of the crude solid (−100 mg) was purified by HPLC.

HPLC Purification step. Column: Waters Xterra Prep MS C18 10 μm 19×250 mm; Solvent A: 10 mM ammonium acetate, pH 5; Solvent B: ACN; Method: 5 min 0% B to 25 min 20% B 26 mL/min. Fractions containing the product was collected and freeze-dried to give 43 mg EC0488 (51% yield). ¹H NMR and LC/MS (exact mass 1678.62) were consistent with the product.

The following Examples of targeting ligand-linker intermediates, EC0233, EC0244, EC0257, and EC0261, were also prepared as described herein.

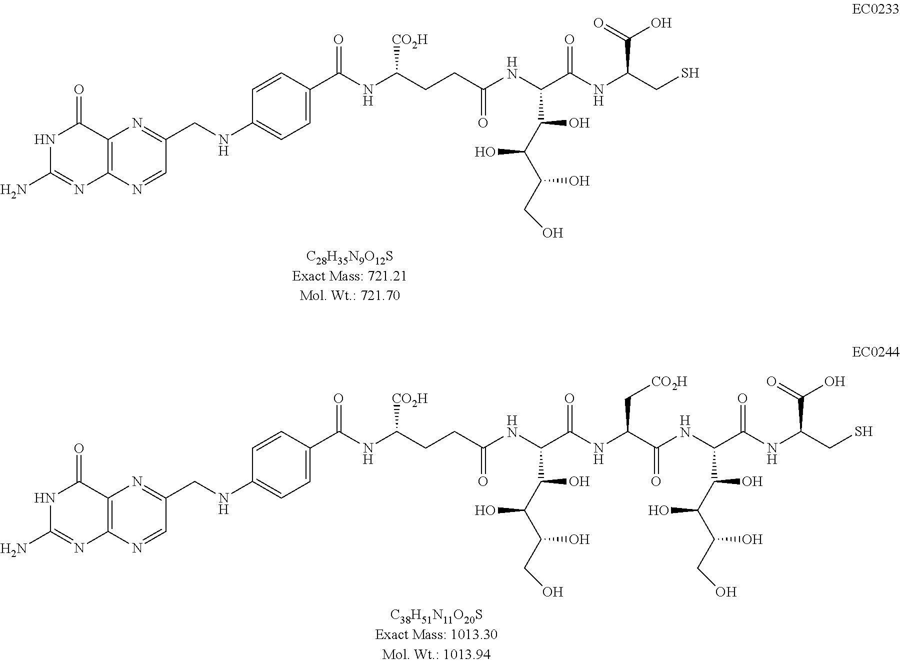

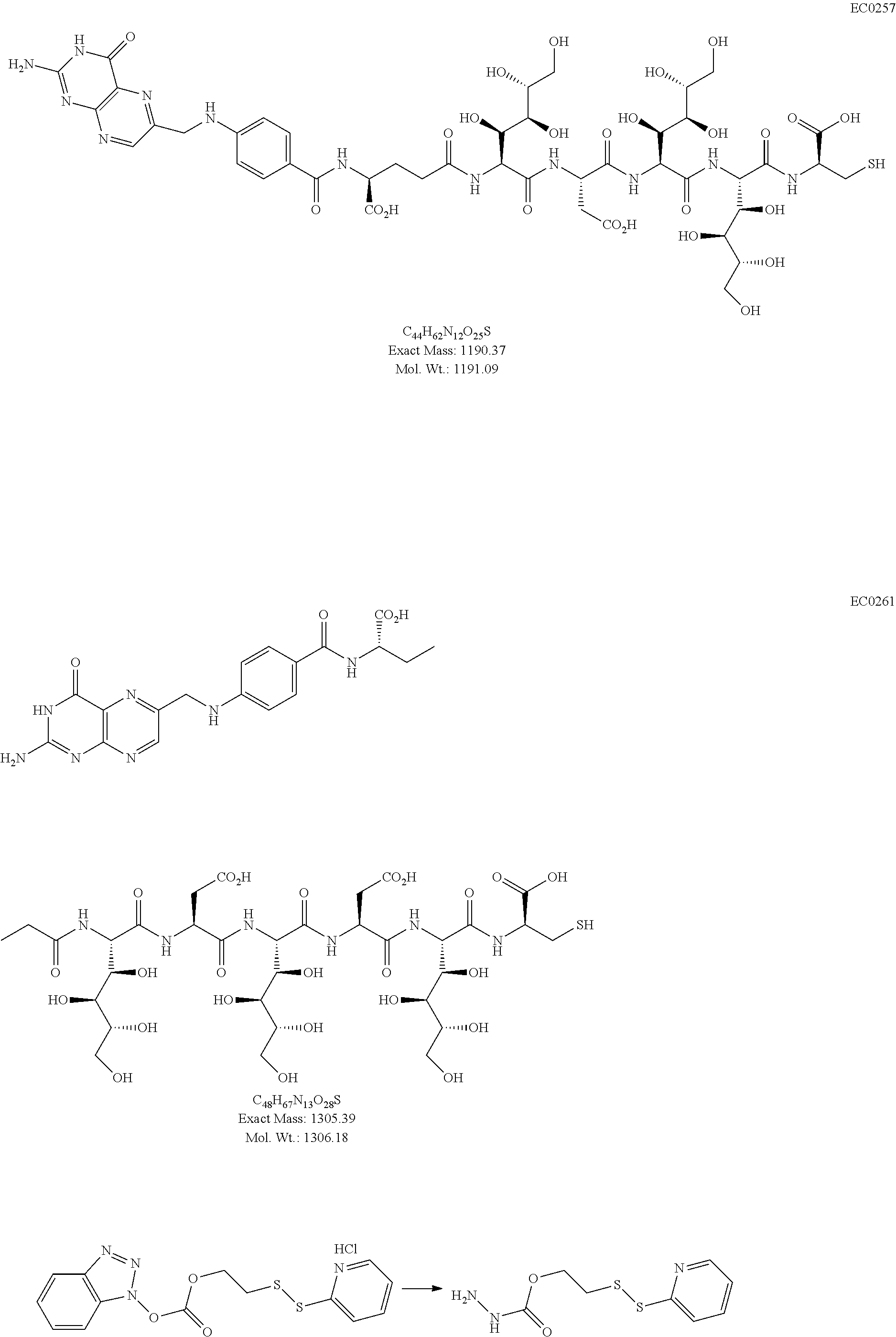

### Example

Synthesis of coupling reagent EC0311. DIPEA (0.60 mL) was added to a suspension of HOBt-OCO₂—(CH₂)₂—SS-2-pyridine HCl (685 mg, 91%) in anhydrous DCM (5.0 mL) at 0° C., stirred under argon for 2 minutes, and to which was added anhydrous hydrazine (0.10 mL). The reaction mixture was stirred under argon at 0° C. for 10 minutes and room temperature for an additional 30 minutes, filtered, and the filtrate was purified by flash chromatography (silica gel, 2% MeOH in DCM) to afford EC0311 as a clear thick oil (371 mg), solidified upon standing.

### Example

General Synthesis of Disulfide Containing Nucleotides. A binding ligand-linker intermediate containing a thiol group is taken in deionized water (ca. 20 mg/mL, bubbled with argon for 10 minutes prior to use) and the pH of the suspension is adjusted by saturated NaHCO₃ (bubbled with argon for 10 minutes prior to use) to about 6.9 (the suspension may become a solution when the pH increased). Additional deionized water is added (ca. 20-25%) to the solution as needed, and to the aqueous solution is added immediately a solution of the nucleic acid having an activated disulfide in THF (ca. 20 mg/mL). The reaction mixture becomes homogenous quickly. After stirring under argon, e.g. for 45 minutes, the reaction mixture is diluted with 2.0 mM sodium phosphate buffer (pH 7.0, ca 150 volume percent) and the THF is removed by evacuation. The resulting suspension is filtered and the filtrate may be purified by preparative HPLC (as described herein). Fractions are lyophilized to isolate the conjugates. The foregoing method is equally applicable for preparing a wide variety of conjugates of nucleic acids, oligonucleotides, and nucleotides by the appropriate selection of the nucleotide starting compound.

### Example

General Method 2 for Preparing Conjugates (one-pot). DIPEA and isobutyl chloroformate (3.1 μL) are added with the help of a syringe in tandem into a solution of a nucleotide, or analog or derivative thereof, having a free carboxylic acid group in anhydrous EtOAc (0.50 mL) at −15° C. After stirring for 35 minutes at −15° C. under argon, to the reaction mixture is added a solution of the linker intermediate having an activated disulfide, such as coupling reagent EC0311, in anhydrous EtOAc (0.50 mL). The cooling is removed and the reaction mixture is stirred under argon for an additional 45 minutes, concentrated under reduced pressure, and the residue is dissolved in THF (2.0 mL). Meanwhile, EC0488, or an equivalent binding ligand linker intermediate is dissolved in deionized water (bubbled with argon for 10 minutes prior to use) and the pH of the aqueous solution is adjusted to 6.9 by saturated NaHCO₃. Additional deionized water is added to the EC0488 solution to make a total volume of 2.0 mL and to which is added immediately the THF solution containing the activated nucleotide. The reaction mixture, which became homogeneous quickly, is stirred under argon for 50 minutes and quenched with 2.0 mM sodium phosphate buffer (pH 7.0, 15 mL). The resulting cloudy solution is filtered and the filtrate is injected into a preparative HPLC for purification. Fractions are collected and lyophilized to afford the conjugate. The foregoing method is equally applicable for preparing a wide variety of conjugates of nucleic acids, oligonucleotides, and nucleotides by the appropriate selection of the nucleotide starting compound.

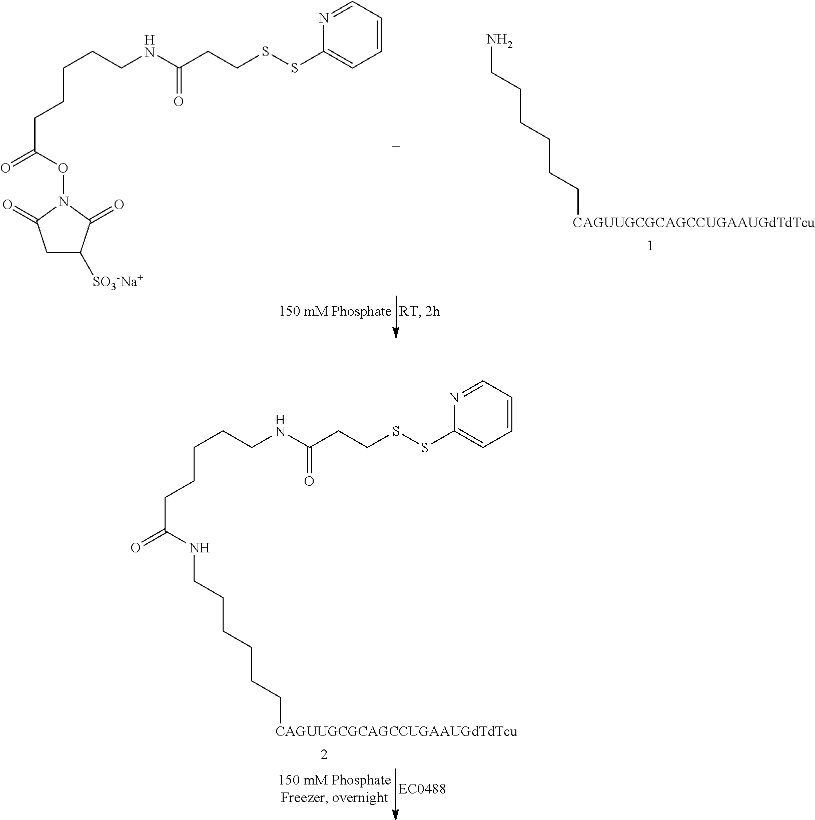

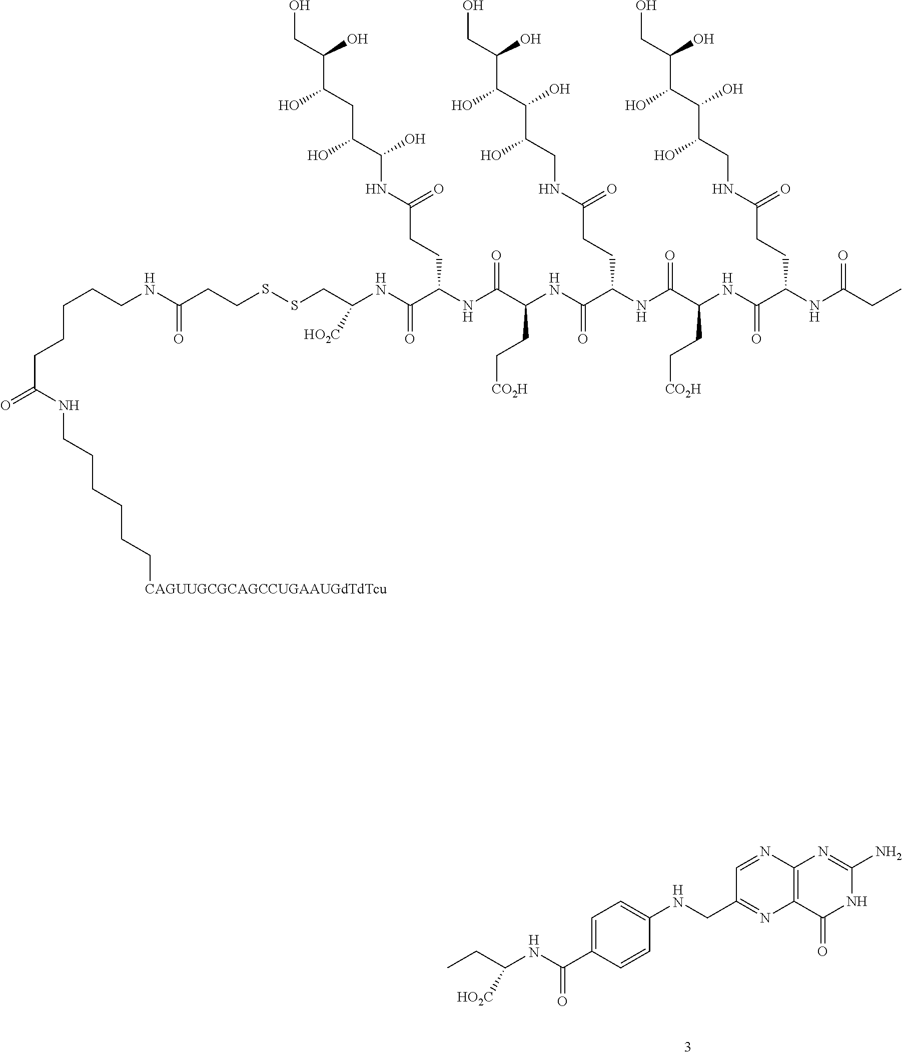

### Example

Preparation of Compounds 2 and 3: Preparation of these compounds was carried out as shown in the above scheme (lower case nucleotide abbreviations indicate 2′-F). To a solution of 400 nmol of the 5′-amino modified single strand siRNA 1 in 100 4 of 150 mM phosphate buffer (pH=7.4, sterilized) was added 21 mg (100 molar equivalents) of 3-sulfo-succinimidyl 6-(3-[2-pyridyldithio]-propionamido)hexanoate (sulfo-LC-SPDP). The reaction mixture was shaken for one hour at room temperature. HPLC analysis (Waters ATLANTIS T3 column, 3.0 μm, 3.0×50 mm; solvent A, 20 mM NH₄HCO₃ buffer, pH=7; solvent B, acetonitrile; gradient, 5% B to 50% B in 5 min; 260 nm) confirmed the formation of pyridyl-disulfide-activated adduct 2 (>90% conversion). The by-products were removed by gel permeation chromatography (D-SALT Dextran Desalting Columns, Pierce, Rockford, Ill.) using 150 mM phosphate as the eluting solvent. The fractions containing adduct 2 were then pooled, and Ar was bubbled through the solution for 10 min. In a separate vial, 6.7 mg of folate spacer EC0488 was dissolved in 300 mL of 150 mM phosphate buffer which had been previously purged with argon. The mixture was quickly shaken to dissolve the EC0488, and 30 4 of this solution (1 molar equivalent) was added to the vial containing 2. The resulting solution was placed in the freezer overnight. HPLC analysis indicated that the reaction was 50-60% complete. The mixture was purified by preparative HPLC (Waters XBRIDGE C18 column, 5 μm, 4.6×250 mm; solvent A, 100 mM TEAA buffer, pH=7; solvent B, acetonitrile; gradient, 10% B to 35% B in 10 min), resulting in the isolation of the folate conjugate of siRNA 3 (peak 1, retention time=1.74 min, area=96%; peak 2, retention time=2.16 min, area=4%). MALDI-MS of compound 3: Expected mass, 9358.25 m/z; found, 9356.73 m/z (M-H)⁻.

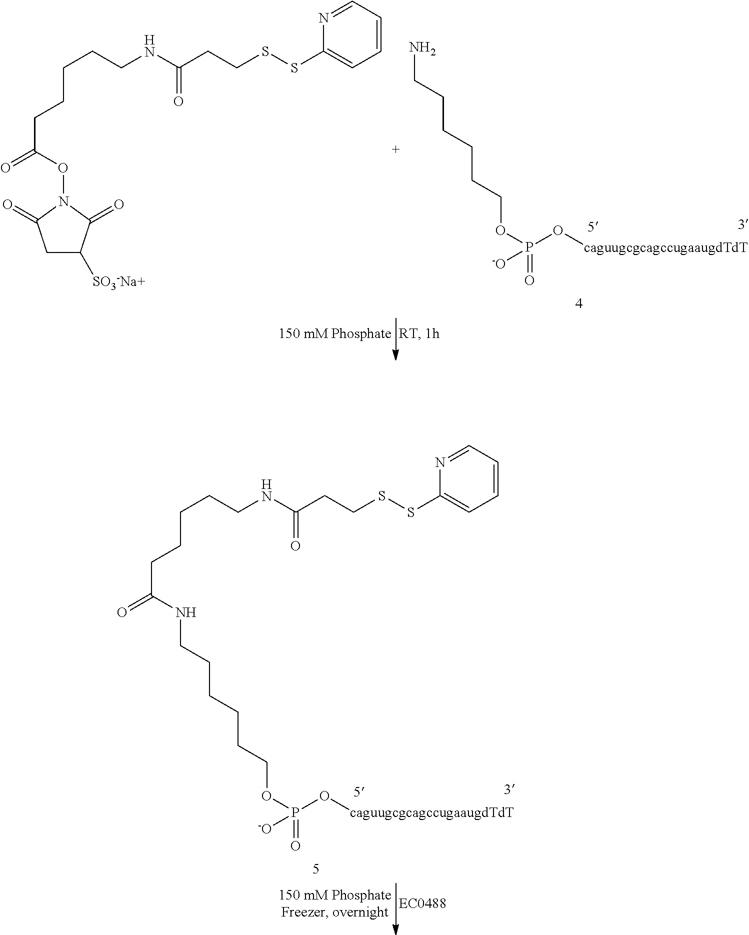

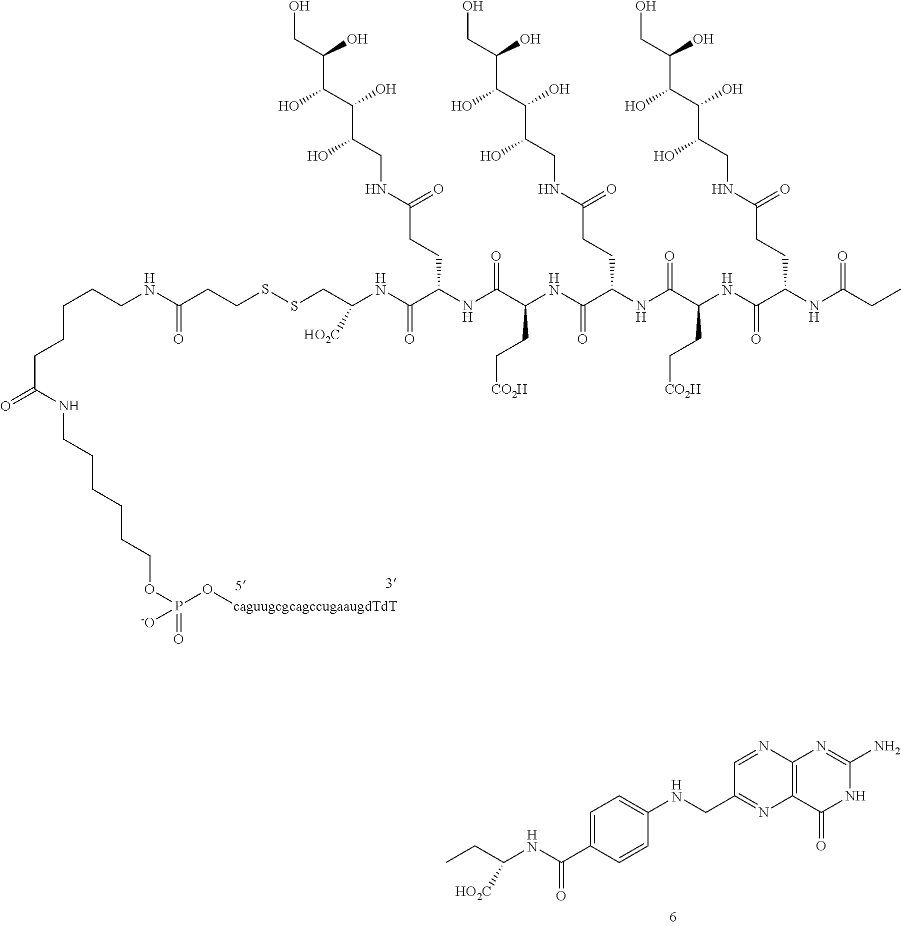

### Example

Preparation of Compounds 5 and 6: Preparation of these compounds was carried out as shown in the above scheme (lower case nucleotide abbreviations indicate 2′-OMe). To a solution of 400 nmol of the 5′-amino modified single strand siRNA 4 in 100 4 of 150 mM phosphate buffer (pH=7.4, sterilized) was added 21 mg (100 molar equivalents) of sulfo-LC-SPDP. The reaction mixture was shaken for 1 hour at room temperature. HPLC analysis (Waters ATLANTIS T3 column, 3.0 μm, 3.0×50 mm; solvent A, 20 mM NH₄HCO₃ buffer, pH=7; solvent B, acetonitrile; gradient, 5% B to 80% B in 5 min; 260 nm) confirmed the formation of pyridyl-disulfide-activated adduct 5 (>90% conversion). The by-products were removed by gel permeation chromatography (D-SALT Dextran Desalting Columns, Pierce, Rockford, Ill.) using 150 mM phosphate as the eluting solvent. The fractions containing adduct 5 were then pooled. Half of the combined fractions were frozen for later use. The remaining solution was deoxygenated with Ar for 10 min. In a separate vial, 6.7 mg of folate spacer EC0488 was dissolved in 300 mL of 150 mM phosphate buffer which had been previously purged with Ar. The mixture was quickly shaken to dissolve the EC0488, and 45 μL of this solution (3 molar equivalents) was added to the vial containing 5. The resulting solution was placed in the freezer overnight. HPLC analysis indicated that the reaction was complete. The mixture was purified by preparative HPLC (Waters XBRIDGE C18 column, 5 μm, 4.6×250 mm; solvent A, 100 mM TEAA buffer, pH=7; solvent B, acetonitrile; gradient, 10% B to 35% B in 10 min), resulting in the isolation of the folate conjugate of siRNA 6 (27% yield over two steps) (peak 1, retention time=1.79 min, area=100%). MALDI-MS of compound 6: Expected exact mass, 9009.44 m/z; found, 9005.14 m/z (M-H)⁻.

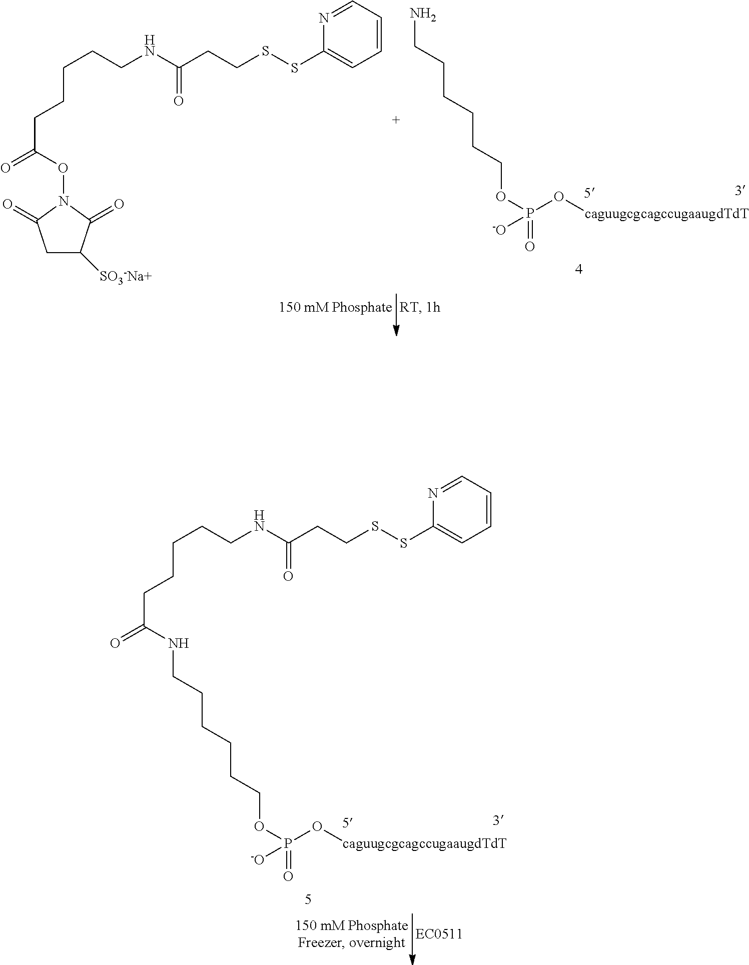

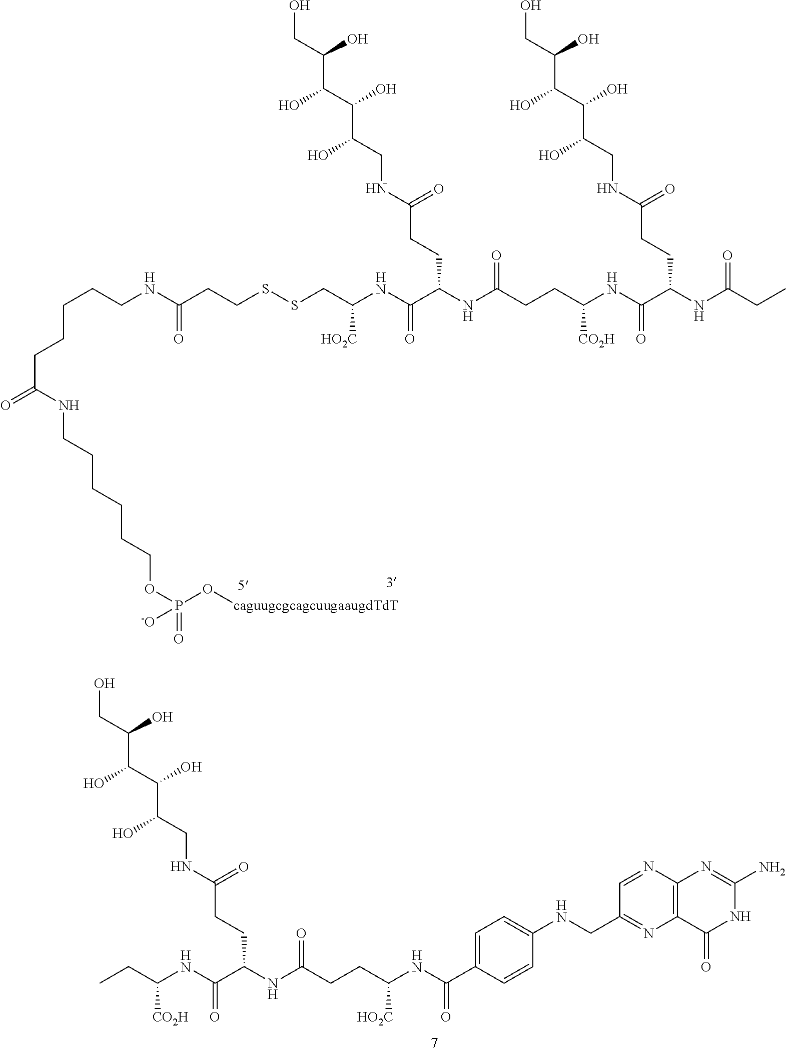

### Example

Preparation of Compound 7: Preparation of this compound was carried out as shown in the above scheme (lower case nucleotide abbreviations indicate 2′-OMe). To a solution of 400 nmol of the 5′-amino modified single strand siRNA 4 in 100 pt of 150 mM phosphate buffer (pH=7.4, sterilized) was added 21 mg (100 molar equivalents) of sulfo-LC-SPDP. The reaction mixture was shaken for one hour at room temperature. HPLC analysis (Waters Atlantis™ T3 column, 3.0 μm, 3.0×50 mm; solvent A, 20 mM NH₄HCO₃ buffer, pH=7; solvent B, acetonitrile; gradient, 5% B to 80% B in 5 min; 260 nm) confirmed the formation of pyridyl-disulfide-activated adduct 5 (>90% conversion). The by-products were removed by gel permeation chromatography (D-Salt™ Dextran Desalting Columns, Pierce, Rockford, Ill.) using 150 mM phosphate as the eluting solvent. The fractions containing adduct 5 were then pooled. Half of the combined fractions were frozen for later use. The remaining solution was deoxygenated with Ar for 10 min. In a separate vial, 6.7 mg of folate spacer EC0511 was dissolved in 300 mL of 150 mM phosphate buffer which had been previously purged with Ar. The mixture was quickly shaken to dissolve the EC0511, and 30 μL of this solution (2 molar equivalents) was added to the vial containing 5. The resulting solution was placed in the freezer overnight. HPLC analysis indicated that the reaction was complete. The mixture was purified by preparative HPLC (Waters XBridge™ C18 column, 5 μm, 4.6×250 mm; solvent A, 100 mM TEAA buffer, pH=7; solvent B, acetonitrile; gradient, 10% B to 35% B in 10 min), resulting in the isolation of the clean folate conjugate of siRNA 7 (39% yield over two steps) (peak 1, retention time=1.79 min, area=100%). MALDI-MS of compound 7: Expected exact mass, 9009.44 m/z; found, 9007.54 m/z (M-H)⁻.

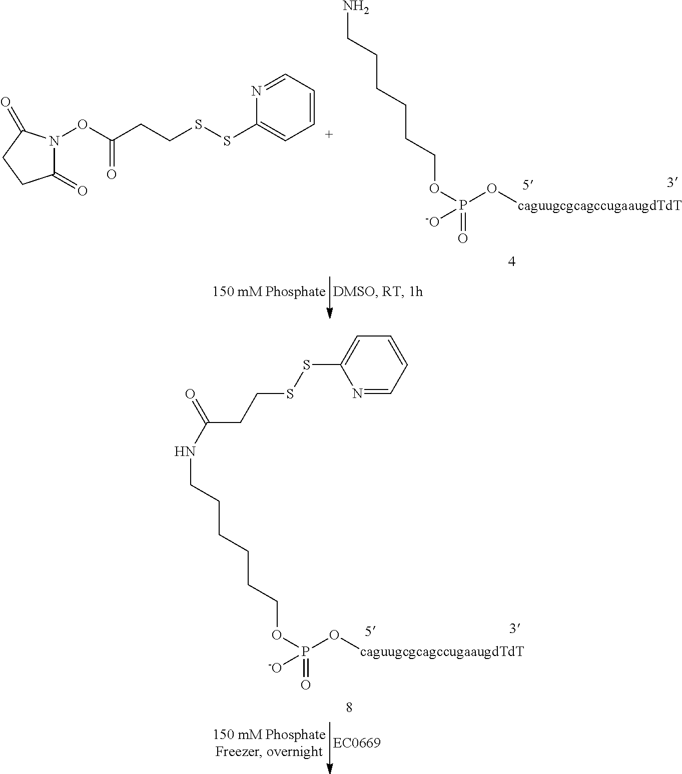

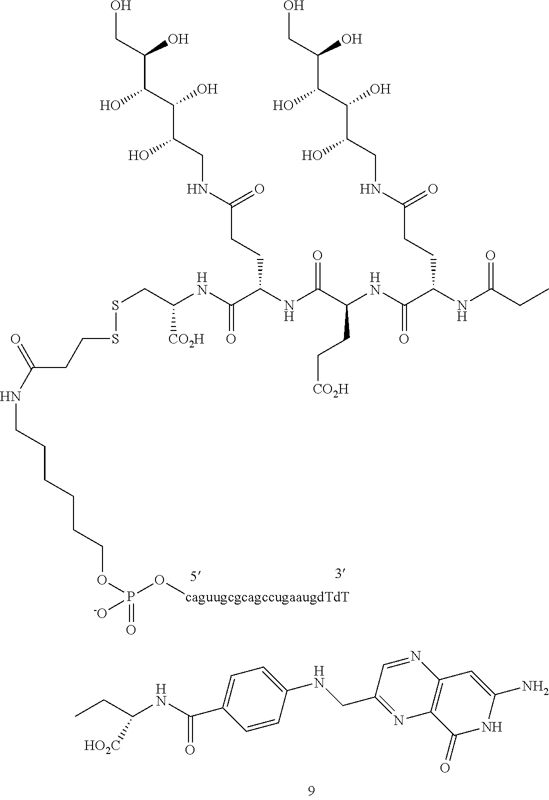

### Example

Preparation of Compounds 8 and 9: Preparation of these compounds was carried out as shown in the above scheme (lower case nucleotide abbreviations indicate 2′-OMe). To a solution of 204 nmol of the 5′-amino modified single strand siRNA 4 in 400 μL of 150 mM phosphate buffer (pH=7.4, sterilized) was added 6.37 mg (100 molar equivalents) of succinimidyl 6-(3-[2-pyridyldithio]-propionate (SPDP) in 50 μL of DMSO. The reaction mixture was shaken for one hour at room temperature. HPLC analysis (Waters Atlantis™ T3 column, 3.0 μm, 3.0×50 mm; solvent A, 20 mM NH₄HCO₃ buffer, pH=7; solvent B, acetonitrile; gradient, 5% B to 80% B in 5 min; 280 nm) confirmed the formation of pyridyl-disulfide-activated adduct 8 (>90% conversion). The by-products were removed by gel permeation chromatography (D-Salt™ Dextran Desalting Columns, Pierce, Rockford, Ill.) using 150 mM phosphate as the eluting solvent. The fractions containing adduct 8 were then pooled and deoxygenated with Ar for 10 min. 0.64 mg of folate spacer EC0669 (2.5 molar equivalents) in 150 mM phosphate buffer was then added. The resulting solution was placed in the freezer overnight. HPLC analysis indicated that the reaction was complete. The mixture was purified by preparative HPLC (Waters XBridge™ C18 column, 5 μm, 19×50 mm; solvent A, 100 mM TEAA buffer, pH=7; solvent B, acetonitrile; gradient, 1% B to 50% B in 25 min), resulting in the isolation of the clean folate conjugate of siRNA 9 (48% yield over two steps) (peak 1, retention time=2.51 min, area=100%). MALDI-MS of compound 9: Expected exact mass, 8474.89 m/z; found, 8474.67 m/z (M-H)⁻.

The compounds described herein may be prepared using the process and syntheses described herein, as well as using general organic synthetic methods. In particular, methods for preparing the compounds are described in U.S. patent application publication 2005/0002942, the disclosure of which is incorporated herein by reference.

General formation of folate-peptides. The folate-containing peptidyl fragment Pte-Glu-(AA)ₙ-NH(CHR₂)CO₂H (3) is prepared by a polymer-supported sequential approach using standard methods, such as the Fmoc-strategy on an acid-sensitive Fmoc-AA-Wang resin (1), as shown in the following Scheme:

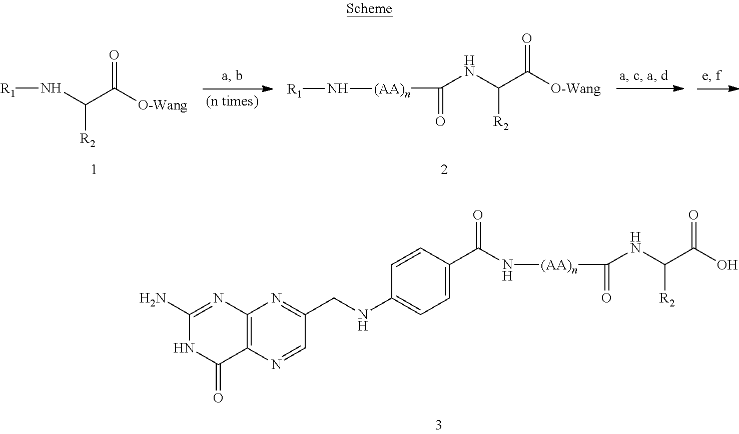

(a) 20% piperidine/DMF; (b) Fmoc-AA-OH, PyBop, DIPEA, DMF; (c) Fmoc-Glu(O-t-Bu)—OH, PyBop, DIPEA, DMF; (d) 1. N¹⁰(TFA)-Pte-OH; PyBop, DIPEA, DMSO; (e) TFAA, (CH₂SH)₂, i-Pr₃SiH; (f) NH₄OH, pH 10.3.

In this illustrative embodiment of the processes described herein, R₁ is Fmoc, R₂ is the desired appropriately-protected amino acid side chain, and DIPEA is diisopropylethylamine. Standard coupling procedures, such as PyBOP and others described herein or known in the art are used, where the coupling agent is illustratively applied as the activating reagent to ensure efficient coupling. Fmoc protecting groups are removed after each coupling step under standard conditions, such as upon treatment with piperidine, tetrabutylammonium fluoride (TBAF), and the like. Appropriately protected amino acid building blocks, such as Fmoc-Glu-OtBu, N¹⁰-TFA-Pte-OH, and the like, are used, as described in the Scheme, and represented in step (b) by Fmoc-AA-OH. Thus, AA refers to any amino acid starting material, that is appropriately protected. It is to be understood that the term amino acid as used herein is intended to refer to any reagent having both an amine and a carboxylic acid functional group separated by one or more carbons, and includes the naturally occurring alpha and beta amino acids, as well as amino acid derivatives and analogs of these amino acids. In particular, amino acids having side chains that are protected, such as protected serine, threonine, cysteine, aspartate, and the like may also be used in the folate-peptide synthesis described herein. Further, gamma, delta, or longer homologous amino acids may also be included as starting materials in the folate-peptide synthesis described herein. Further, amino acid analogs having homologous side chains, or alternate branching structures, such as norleucine, isovaline, β-methyl threonine, β-methyl cysteine, β,β-dimethyl cysteine, and the like, may also be included as starting materials in the folate-peptide synthesis described herein.

The coupling sequence (steps (a) & (b)) involving Fmoc-AA-OH is performed “n” times to prepare solid-support peptide (2), where n is an integer and may equal 0 to about 100. Following the last coupling step, the remaining Fmoc group is removed (step (a)), and the peptide is sequentially coupled to a glutamate derivative (step (c)), deprotected, and coupled to TFA-protected pteroic acid (step (d)). Subsequently, the peptide is cleaved from the polymeric support upon treatment with trifluoroacetic acid, ethanedithiol, and triisopropylsilane (step (e)). These reaction conditions result in the simultaneous removal of the t-Bu, t-Boc, and Trt protecting groups that may form part of the appropriately-protected amino acid side chain. The TFA protecting group is removed upon treatment with base (step (f)) to provide the folate-containing peptidyl fragment (3).

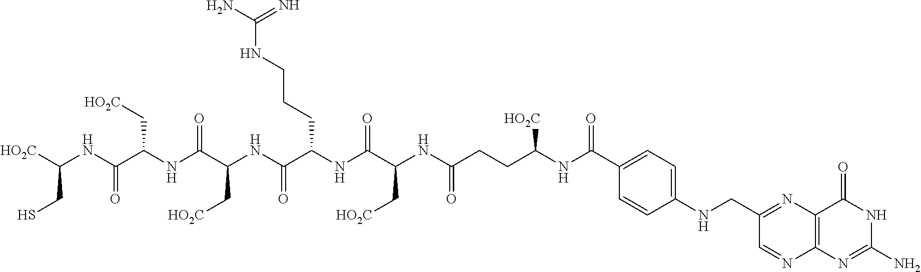

According to the general procedure described herein, Wang resin bound 4-methoxytrityl (MTT)-protected Cys-NH₂ was reacted according to the following sequence: 1) a. Fmoc-Asp(OtBu)—OH, PyBOP, DIPEA; b. 20% Piperidine/DMF; 2) a. Fmoc-Asp(OtBu)—OH, PyBOP, DIPEA; b. 20% Piperidine/DMF; 3) a. Fmoc-Arg(Pbf)-OH, PyBOP, DIPEA; b. 20% Piperidine/DMF; 4) a. Fmoc-Asp(OtBu)—OH, PyBOP, DIPEA; b. 20% Piperidine/DMF; 5) a. Fmoc-Glu-OtBu, PyBOP, DIPEA; b. 20% Piperidine/DMF; 6) N¹⁰-TFA-pteroic acid, PyBOP, DIPEA. The MTT, t-Bu, and Pbf protecting groups were removed with TFA/H₂O/TIPS/EDT (92.5:2.5:2.5:2.5), and the TFA protecting group was removed with aqueous NH₄OH at pH=9.3. Selected ¹H NMR (D₂O) δ (ppm) 8.68 (s, 1H, FA H-7), 7.57 (d, 2H, J=8.4 Hz, FA H-12 &16), 6.67 (d, 2H, J=9 Hz, FA H-13 &15), 4.40-4.75 (m, 5H), 4.35 (m, 2H), 4.16 (m, 1H), 3.02 (m, 2H), 2.55-2.95 (m, 8H), 2.42 (m, 2H), 2.00-2.30 (m, 2H), 1.55-1.90 (m, 2H), 1.48 (m, 2H); MS (ESI, m+H⁺) 1046.

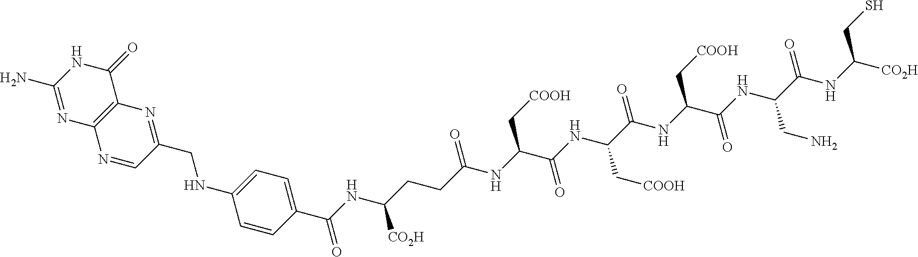

According to the general procedure described herein, Wang resin bound 4-methoxytrityl (MTT)-protected Cys-NH₂ was reacted according to the following sequence: 1) a. Fmoc-β-aminoalanine(NH-MTT)-OH, PyBOP, DIPEA; b. 20% Piperidine/DMF; 2) a. Fmoc-Asp(OtBu)—OH, PyBOP, DIPEA; b. 20% Piperidine/DMF; 3) a. Fmoc-Asp(OtBu)—OH, PyBOP, DIPEA; b. 20% Piperidine/DMF; 4) a. Fmoc-Asp(OtBu)—OH, PyBOP, DIPEA; b. 20% Piperidine/DMF; 5) a. Fmoc-Glu-OtBu, PyBOP, DIPEA; b. 20% Piperidine/DMF; 6) N¹⁰-TFA-pteroic acid, PyBOP, DIPEA. The MTT, tBu, and TFA protecting groups were removed with a. 2% hydrazine/DMF; b. TFA/H₂O/TIPS/EDT (92.5:2.5:2.5:2.5).

The reagents shown in the following table were used in the preparation:

| Reagent | (mmol) | equivalents | Amount |
|---|---|---|---|
| H-Cys(4-methoxytrityl)- | 0.56 | 1 | 1.0 g |
| 2-chlorotrityl-Resin | | | |
| (loading 0.56 mmol/g) | | | |
| Fmoc-β-aminoalanine(NH- | 1.12 | 2 | 0.653 g |
| MTT)-OH | | | |
| Fmoc-Asp(OtBu)—OH | 1.12 | 2 | 0.461 g |
| Fmoc-Asp(OtBu)—OH | 1.12 | 2 | 0.461 g |
| Fmoc-Asp(OtBu)—OH | 1.12 | 2 | 0.461 g |
| Fmoc-Glu-OtBu | 1.12 | 2 | 0.477 g |
| N¹⁰TFA-Pteroic Acid | 0.70 | 1.25 | 0.286 g |
| (dissolve in 10 ml DMSO) | | | |
| DIPEA | 2.24 | 4 | 0.390 mL |
| PyBOP | 1.12 | 2 | 0.583 g |

The coupling step was performed as follows: In a peptide synthesis vessel add the resin, add the amino acid solution, DIPEA, and PyBOP. Bubble argon for 1 hr. and wash 3× with DMF and IPA. Use 20% piperidine in DMF for Fmoc deprotection, 3× (10 min), before each amino acid coupling. Continue to complete all 6 coupling steps. At the end wash the resin with 2% hydrazine in DMF 3× (5 min) to cleave TFA protecting group on Pteroic acid.

Cleave the peptide analog from the resin using the following reagent, 92.5% (50 ml) TFA, 2.5% (1.34 ml) H₂O, 2.5% (1.34 ml) Triisopropylsilane, 2.5% (1.34 ml) ethanedithiol, the cleavage step was performed as follows: Add 25 ml cleavage reagent and bubble for 1.5 hr, drain, and wash 3× with remaining reagent. Evaporate to about 5 mL and precipitate in ethyl ether. Centrifuge and dry. Purification was performed as follows: Column-Waters NovaPak C₁₈ 300×19 mm; Buffer A=10 mM Ammonium Acetate, pH 5; B=CAN; 1% B to 20% B in 40 minutes at 15 ml/min, to 350 mg (64%); HPLC-RT 10.307 min., 100% pure, ¹H HMR spectrum consistent with the assigned structure, and MS (ES−): 1624.8, 1463.2, 1462.3, 977.1, 976.2, 975.1, 974.1, 486.8, 477.8.

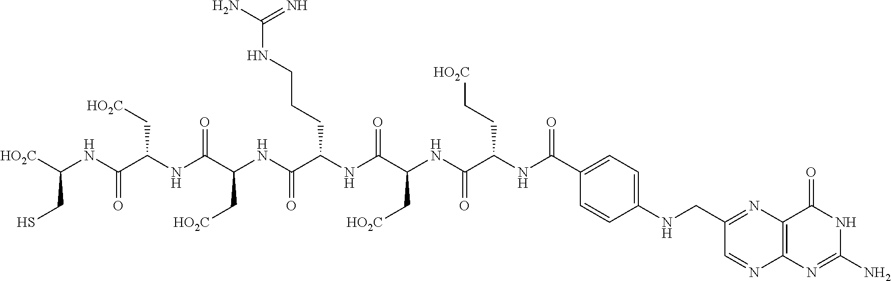

According to the general procedure described herein, Wang resin bound MTT-protected Cys-NH₂ was reacted according to the following sequence: 1) a. Fmoc-Asp(OtBu)—OH, PyBOP, DIPEA; b. 20% Piperidine/DMF; 2) a. Fmoc-Asp(OtBu)—OH, PyBOP, DIPEA; b. 20% Piperidine/DMF; 3) a. Fmoc-Arg(Pbf)-OH, PyBOP, DIPEA; b. 20% Piperidine/DMF; 4) a. Fmoc-Asp(OtBu)—OH, PyBOP, DIPEA; b. 20% Piperidine/DMF; 5) a. Fmoc-Glu(γ-OtBu)—OH, PyBOP, DIPEA; b. 20% Piperidine/DMF; 6) N¹⁰-TFA-pteroic acid, PyBOP, DIPEA. The MTT, tBu, and Pbf protecting groups were removed with TFA/H₂O/TIPS/EDT (92.5:2.5:2.5:2.5), and the TFA protecting group was removed with aqueous NH₄OH at pH=9.3. The ¹H NMR spectrum was consistent with the assigned structure.

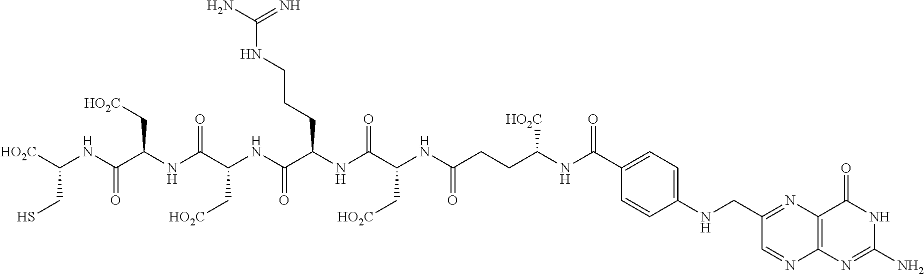

According to the general procedure described herein, Wang resin bound MTT-protected D-Cys-NH₂ was reacted according to the following sequence: 1) a. Fmoc-D-Asp(OtBu)—OH, PyBOP, DIPEA; b. 20% Piperidine/DMF; 2) a. Fmoc-D-Asp(OtBu)—OH, PyBOP, DIPEA; b. 20% Piperidine/DMF; 3) a. Fmoc-D-Arg(Pbf)-OH, PyBOP, DIPEA; b. 20% Piperidine/DMF; 4) a. Fmoc-D-Asp(OtBu)—OH, PyBOP, DIPEA; b. 20% Piperidine/DMF; 5) a. Fmoc-D-Glu-OtBu, PyBOP, DIPEA; b. 20% Piperidine/DMF; 6) N¹⁰-TFA-pteroic acid, PyBOP, DIPEA. The MTT, tBu, and Pbf protecting groups were removed with TFA/H₂O/TIPS/EDT (92.5:2.5:2.5:2.5), and the TFA protecting group was removed with aqueous NH₄OH at pH=9.3. The ¹H NMR spectrum was consistent with the assigned structure. Similarly, EC089

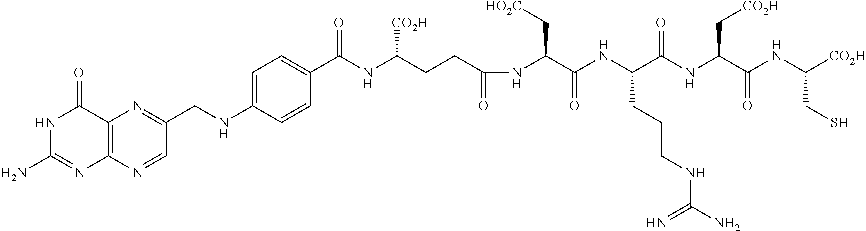

was prepared as described herein.

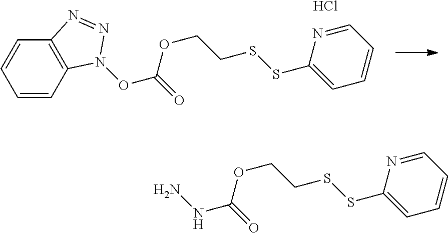

Synthesis of coupling reagent EC0311. DIPEA (0.60 mL) was added to a suspension of HOBt-OCO₂—(CH₂)₂—SS-2-pyridine HCl (685 mg, 91%) in anhydrous DCM (5.0 mL) at 0° C., stirred under argon for 2 minutes, and to which was added anhydrous hydrazine (0.10 mL). The reaction mixture was stirred under argon at 0° C. for 10 minutes and room temperature for an additional 30 minutes, filtered, and the filtrate was purified by flash chromatography (silica gel, 2% MeOH in DCM) to afford EC0311 as a clear thick oil (371 mg), solidified upon standing. Similarly EC0351

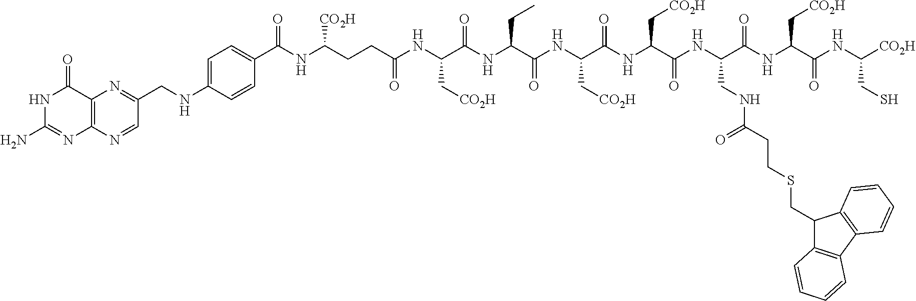

was prepared as described herein.

General Synthesis of Disulfide Containing Conjugates. A binding ligand-linker intermediate containing a thiol group is taken in deionized water (ca. 20 mg/mL, bubbled with argon for 10 minutes prior to use) and the pH of the suspension was adjusted by saturated NaHCO₃ (bubbled with argon for 10 minutes prior to use) to about 6.9 (the suspension may become a solution when the pH increased). Additional deionized water is added (ca. 20-25%) to the solution as needed, and to the aqueous solution is added immediately a solution of the activated thiol intermediate of a nucleotide in an appropriate solvent (ca. 20 mg/mL). The reaction mixture becomes homogenous quickly. After stirring under argon, e.g. for 45 minutes, the reaction mixture is diluted with 2.0 mM sodium phosphate buffer (pH 7.0, ca 150 volume percent) and the THF is removed by evacuation. The resulting suspension is filtered and the filtrate may be purified by preparative HPLC (as described herein). Fraction are lyophilized to isolate the conjugates.

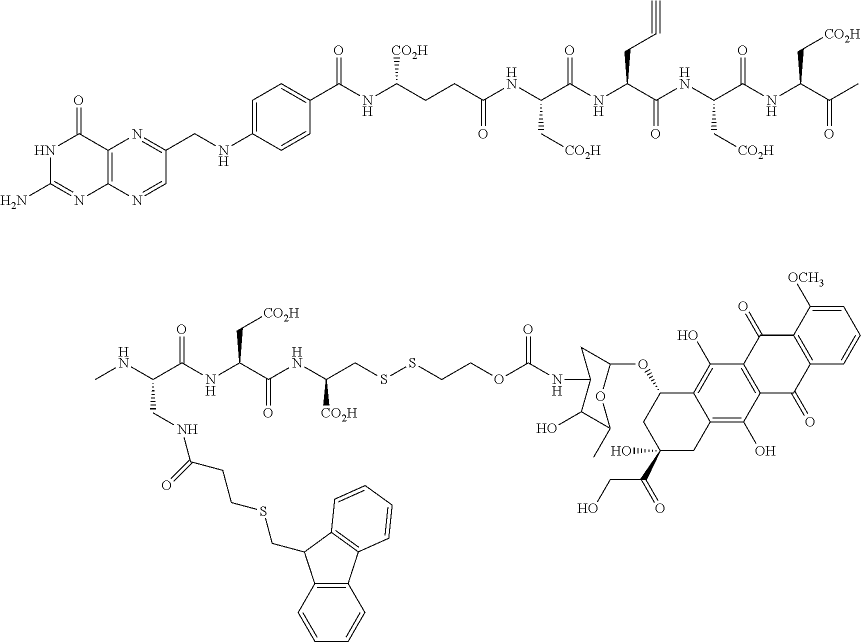

EC0352. Similarly, this compound was prepared as described herein. EC0352 was prepared by forming a disulfide bond between hydroxydaunorubucin pyridyldisulfide and EC0351 in 55% yield.

The following illustrative example

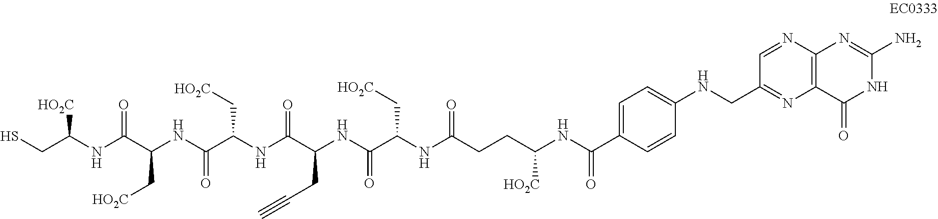

was also prepared using the processes and syntheses described herein.

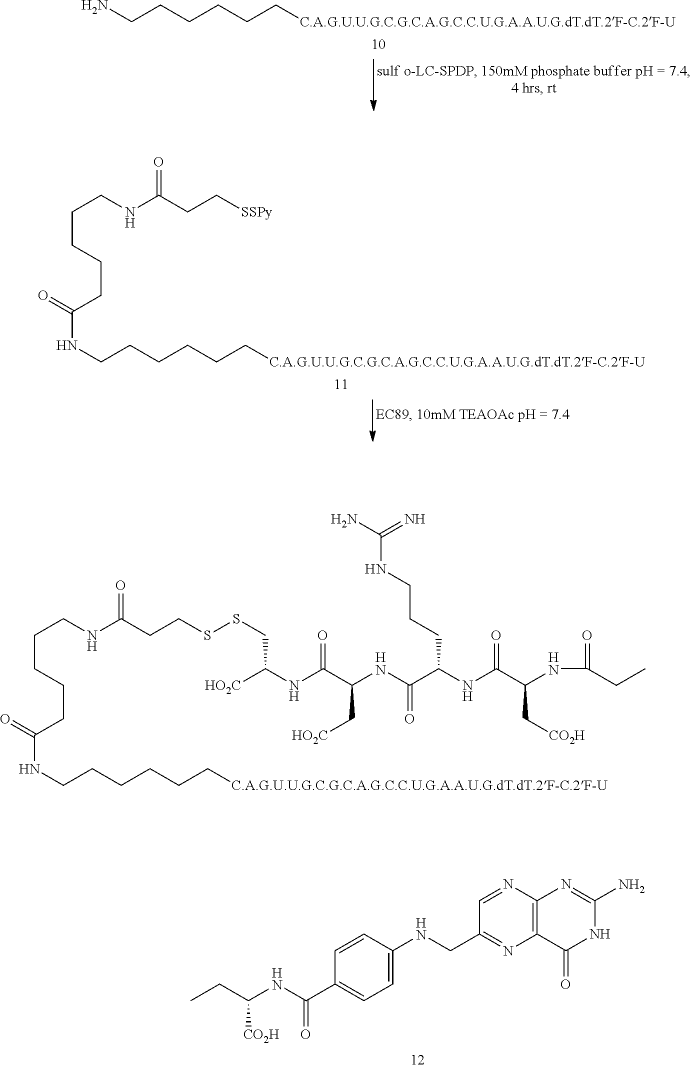

### Example

Preparation of Compounds 11 and 12. Preparation of these compounds was carried out as shown in the above scheme. To a solution of 400 nmol of the 5′-amino modified single strand siRNA 10 in 150 4, of 150 mM phosphate buffer (pH=7.4, sterilized) was added 3.1 mg (15 molar equivalents) of 3-sulfo-succinimidyl 64342-pyridyldithio]-propionamido)hexanoate (sulfo-LC-SPDP). The reaction mixture was shaken for several hours, then was diluted to 700 μL by addition of 150 mM phosphate buffer. The contents of the mixture were then loaded into a Slide-a-LYSER (3 mL capacity, 3500 MWCO) and dialysis overnight with 10 mM triethylammonium acetate (TEAA) was undertaken at 4° C. HPLC analysis (Waters XBRIDGE C18 column, 3.5 μm, 3.0×50 mm; solvent A, 10 min TEAA buffer, pH=7; solvent B, acetonitrile; gradient, 5% B to 80% B in 10 min; 280 nm) indicated that all of the small molecule reagents and bi-products had been removed by dialysis with only unreacted single strand siRNA 1 and its pyridyl-disulfide-activated adduct 11 remaining in solution. The contents of the SLIDE-A-LYSER were then transferred to a 4 mL sterile vial and a stir bar added. Argon was bubbled through the solution for 10 minutes. In a separate tube, 1.08 mg of folate spacer EC89 was dissolved in 2 mL of 150 mM phosphate buffer which had been previously purged with Argon. The mixture was quickly shaken to dissolve the EC89, and 334 μL of this solution (0.18 mg of EC89) was added to the vial containing the mixture of siRNAs. This solution was allowed to stir at room temperature for 4 hours and then placed into the freezer overnight. HPLC indicated that the reaction was complete. The reaction mixture was purified by preparative HPLC (Waters XTERRA C18 column, 5 μm, 19×50 mm; solvent A, 10 mM TEAA buffer, pH=7; solvent B, acetonitrile; gradient, 1% B to 50% B in 25 min), resulting in the isolation of the folate conjugate of siRNA 12 (peak 1, retention time=6.00 min, area=84%; peak 2, retention time=7.35 min, area=16%). MALDI-MS for compound 12: Expected exact mass, 8605.26 m/z; found, 8605.32 m/z (M-H)⁻.

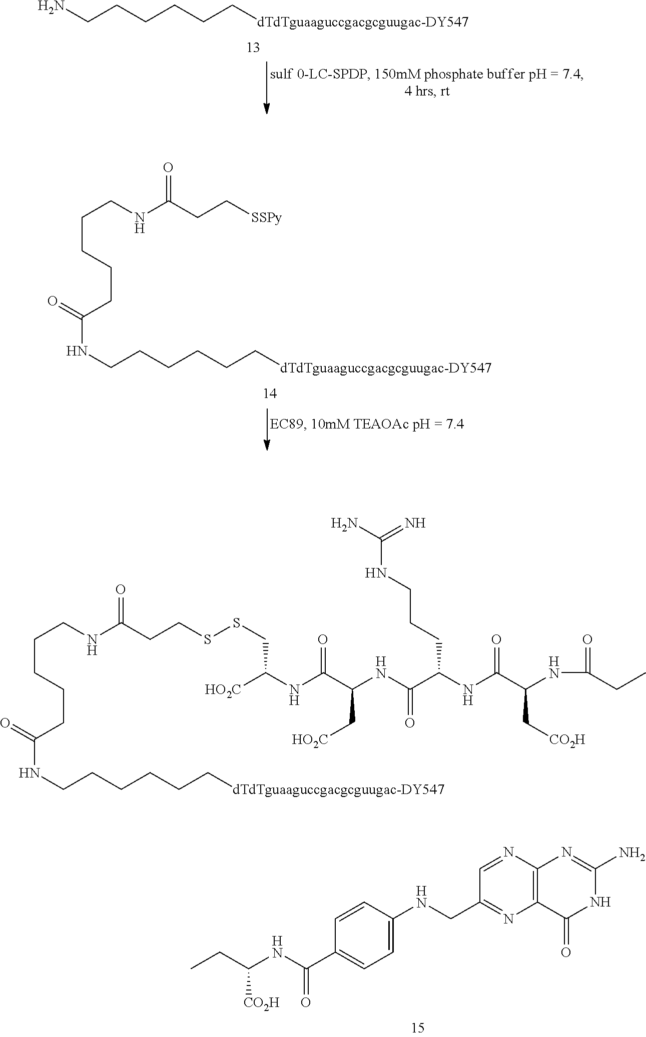

### Example

Preparation of Compounds 14 and 15. Preparation of these compounds was carried out as shown in the above scheme. To a solution of 150 nmol of the 5′-DY547-3′-amino modified single strand siRNA 13 in 200 pt of 150 mM phosphate buffer (pH=7.4, sterilized) was added 1.1 mg (15 molar equivalents) of sulfo-LC-SPDP. The reaction mixture was shaken for one hour. Analytical HPLC (Waters XBRIDGE C18 column, 3.5 μm, 3.0×50 mm; solvent A, 10 mM TEAA buffer, pH=7; solvent B, acetonitrile; gradient, 5% B to 80% B in 10 min; 546 nm) indicated that only 25% of 13 had converted to the desired pyridyl-disulfide-activated adduct 14. An additional 45 molar equivalents of sulfo-LC-SPDP were added and the mixture was shaken for an additional 3 hours. The crude reaction mixture was then purified by preparative HPLC to recover adduct 14 in TEAA/acetonitrile buffer. The buffer solution containing 14 was concentrated to 2 mL and transferred into a sterile vial. A stir bar was added and the solution was bubbled with argon for 10 minutes. In a separate tube, 2.0 mg of folate spacer EC89 was dissolved in 3 mL of 150 mM phosphate buffer which was previously purged with argon. The mixture was quickly shaken to dissolve the EC89 and 67 μL of this solution (45 μg of EC89) was added to the vial containing 14. This solution was allowed to stir at room temperature for 4 hours and then placed in a freezer overnight. HPLC analysis indicated that the reaction was complete. The mixture was purified by preparative HPLC (Waters XTERRA C18 column, 5 μm, 19×50 mm; solvent A, 10 mM TEAA buffer, pH=7; solvent B, acetonitrile; gradient, 1% B to 50% B in 25 min), resulting in the isolation of the clean folate conjugate of siRNA 6, at 100% purity by analytical HPLC (peak 1, retention time=7.04 min, area=100%). MALDI-MS for compound 15: Expected exact mass, 8752.29 m/z; found, 8751.18 m/z (M-H)⁻.

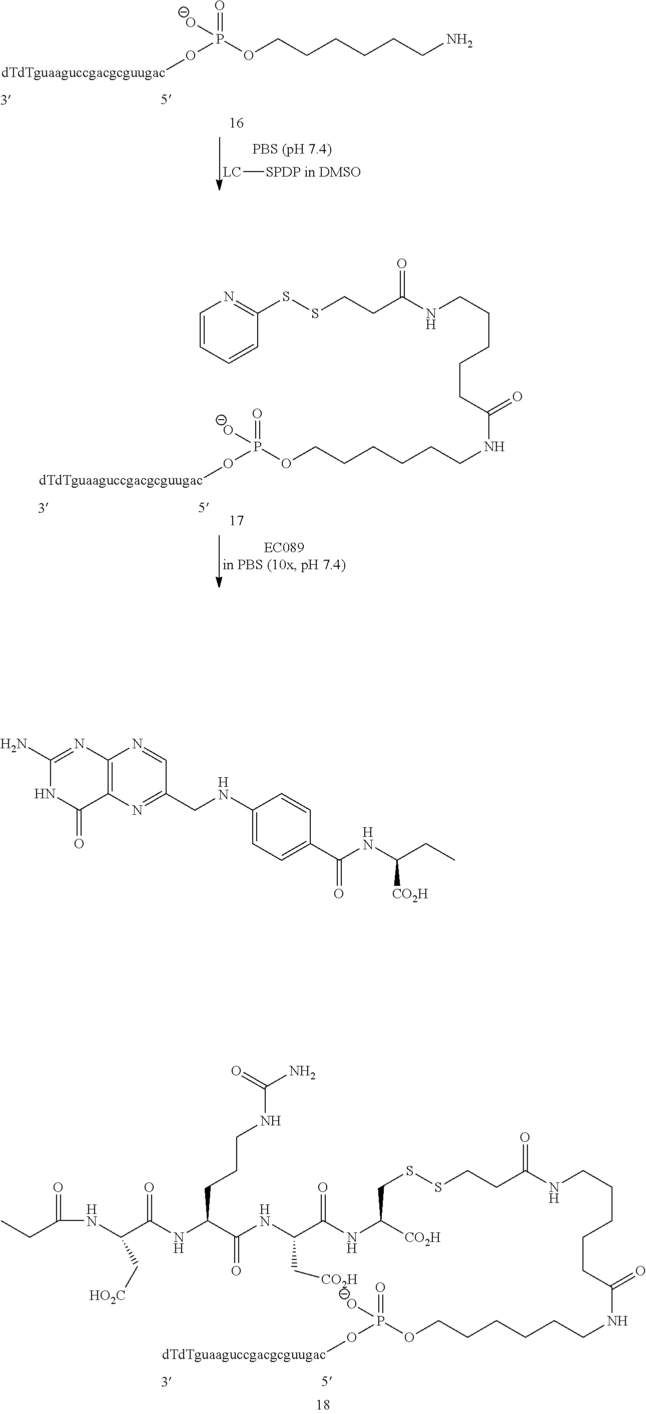

### Example

Synthesis of siRNA-Folate Conjugate 18. Preparation of this compound was carried out as shown in the above scheme. 17.4 mg of succinimidyl 6-(3-[2-pyridyldithio]-propionamido)hexanoate (LC-SPDP) in DMSO (50 μL) was added into the solution of 5′-amino modified single strand siRNA 7 (409 nmol) in PBS (pH 7.4, 500 μL). The reaction mixture was incubated at room temperature for 2 h. Analytical HPLC (Waters XBRIDGE C18 column, 3.5 μm, 3.0×50 mm; solvent A, 100 mM TEAA buffer, pH=7; solvent B, acetonitrile; gradient, 5% B to 80% B in 10 min; 280 nm) indicated ca. 50% conversion. The reaction mixture was diluted to 800 μL with PBS and an additional 8.7 mg of LC-SPDP in DMSO (30 μL) was added. The reaction proceeded for another 3 h with more than 75% conversion by HPLC. The NHS by-product and unreacted LC-SPDP were removed by gel permeation chromatography (D-SALT Dextran Desalting Columns Pierce, Rockford, Ill.) using water as the eluting solvent. The product was subjected to the next step reaction without further purification. The aqueous solution (4 mL) of the 2-pyridyl disulfide activated siRNA 17 was degassed and 48 μL of a solution of the folate spacer EC089 in PBS was added under argon (2.6 mg of EC089 was dissolved in 300 μL of degassed PBS (pH 7.4)). The reaction mixture was left in a freezer for 18 h. 90% conversion was observed by HPLC. The product was purified on preparative HPLC (Waters XTERRA C18 column, 5 μm, 19×50 mm; solvent A, 100 mM TEAA buffer, pH=7.0; solvent B, acetonitrile; gradient, 1% B to 50% B in 25 min) to give the clean folate conjugate of siRNA 18, at 100% purity by analytical HPLC (peak 1, retention time=6.26 min; area=100%). MALDI-MS for compound 18: Expected exact mass, 8260.72 m/z; found, 8261.64 m/z (M-H)⁻.

### Example

Similarly, the following conjugate

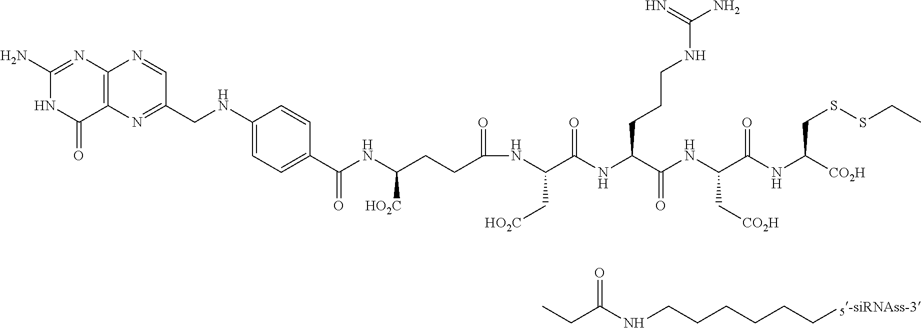

was prepared, where 5′-siRNAss-3′ is 5′-mCmAmGmUmUmGmCmGmCmAmGmCmCmUmGmAmAmUmGdTdT-3′.

### Example

Similarly, the following conjugate

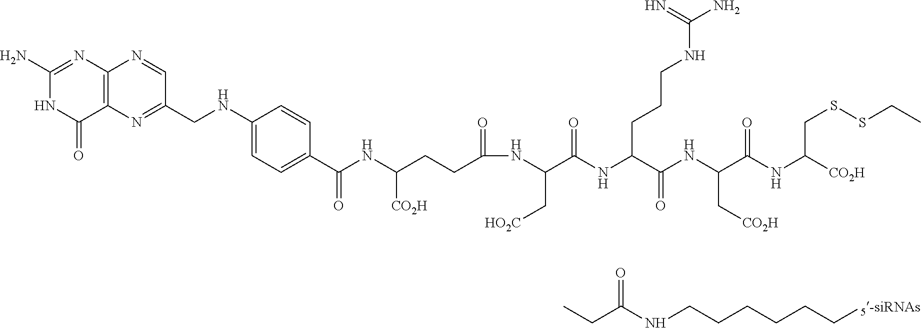

was prepared, where 5′-siRNAss-3′ is 5′-mCmAmGmUmUmGmCmGmCmAmGmCmCmUmGmAmAmUmGdTdT-3′.

### Example

Similarly, the following conjugate

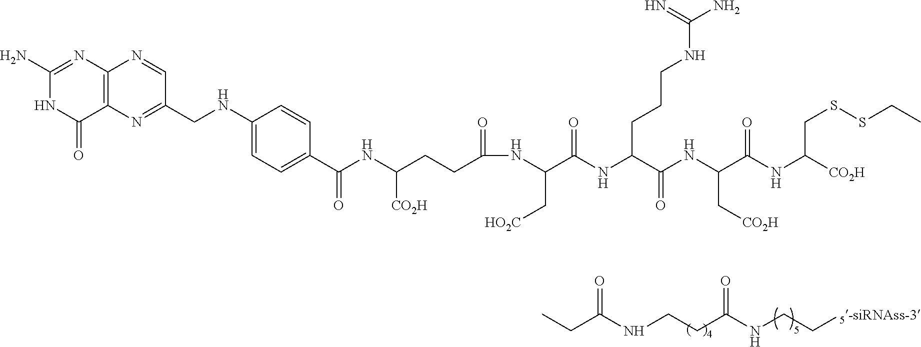

was prepared, where 5′-siRNAss-3′ is 5′-mCmAmGmUmUmGmCmGmCmAmGmCmCmUmGmAmAmUmGdTdT-3′.

### Example

Similarly, the following conjugate

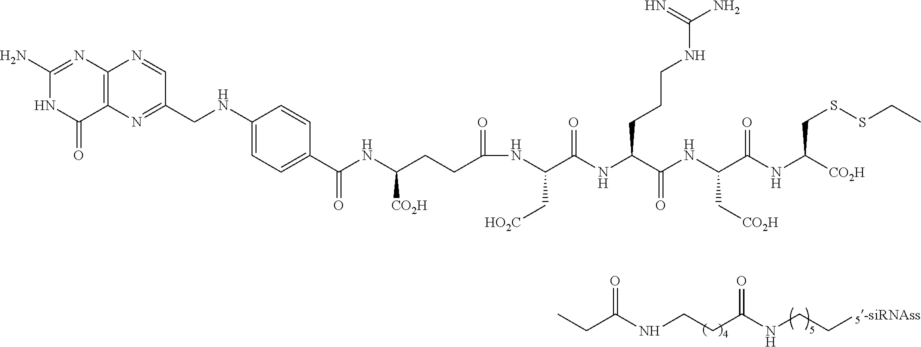

was prepared, where 5′-siRNAss-3′ is 5′-mCmAmGmUmUmGmCmGmCmAmGmCmCmUmGmAmAmUmGdTdT-3′.

### Example

Similarly, the following conjugate

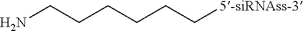

was prepared, where 5′-siRNAss-3′ is 5′-mCmAmGmUmUmGmCmGmCmAmGmCmCmUmGmAmAmUmGdTdT-3′.

### Example

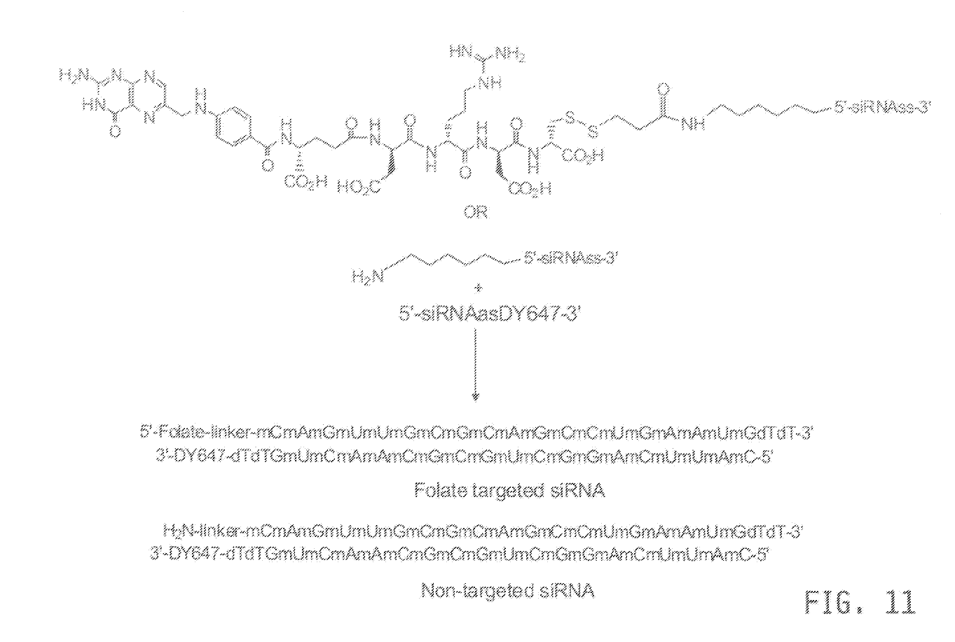

The conjugate depicted in FIG. 11 was prepared using a procedure similar to those above.

### Example

Hybridization of targeted siRNA. The folate linked conjugate of the sense strand was hybridized with the anti-sense strand (5′-siRNAas-DY647-3′) under standard conditions

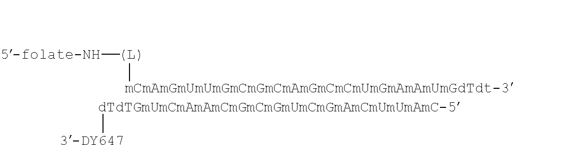

where L is Asp-Arg-Asp-Cys-SS—(CH₂)₂—C(O)NH—(CH₂)₆, and 5′-siRNAas-DY647-3′ is 3′-dTdTGmUmCmAmAmCmGmCmGmUmCmGmGmAmCmUmUmAmC-5′. The complementary DyLight 647 strand (5′-siRNAas-DY647-3′) was purchased from Dharmacon (Lafayette, Colo., USA). The duplex was administered without further purification.

### Example

Hybridization of targeted siRNA. The folate linked conjugate of the sense strand was hybridized with the anti-sense strand (5′-siRNAas-DY647-3′) under standard conditions

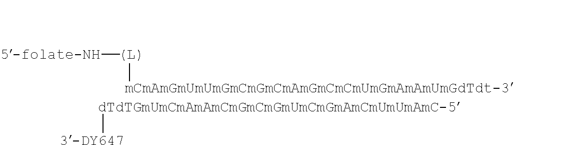

where L is Asp-Arg-Asp-Cys-SS—(CH₂)₂—C(O)NH—(CH₂)₆, or L is Asp-Arg-Asp-Cys-SS—(CH₂)₂—C(O)NH—(CH₂)₅C(O)NH—(CH₂)₆ and 5′-siRNAas-DY647-3′ is 3′-DY647-dTdTGmUmCmAmAmCmGmCmGmUmCmGmGmAmCmUmUmAmC-5′. The complementary DyLight 647 strand (5′-siRNAas-DY647-3′) was purchased from Dharmacon (Lafayette, Colo., USA). The duplex was administered without further purification.

### Example

Hybridization of untargeted siRNA. The amino terminated linker conjugate of the sense strand was hybridized with the anti-sense strand (5′-siRNAas-DY647-3′) under standard conditions

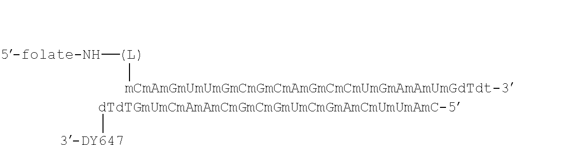

where L is Asp-Arg-Asp-Cys-SS—(CH₂)₂—C(O)NH—(CH₂)₆, and 5′-siRNAas-DY647-3′ is 3′-dTdTGmUmCmAmAmCmGmCmGmUmCmGmGmAmCmUmUmAmC-5′. The duplex was administered without further purification.

### Example

Hybridization of untargeted siRNA. The amino terminated linker conjugate of the sense strand was hybridized with the anti-sense strand (5′-siRNAas-DY647-3′) under standard conditions

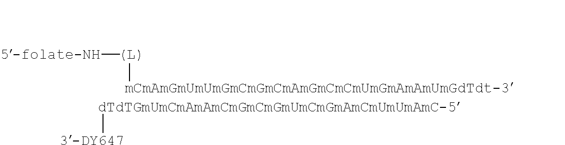

where L is Asp-Arg-Asp-Cys-SS—(CH₂)₂—C(O)NH—(CH₂)₆ or L is Asp-Arg-Asp-Cys-SS—(CH₂)₂—C(O)NH—(CH₂)₅C(O)NH—(CH₂)₆, and 5′-siRNAas-DY647-3′ is 3′-DY647-dTdTGmUmCmAmAmCmGmCmGmUmCmGmGmAmCmUmUmAmC-5′. The duplex was administered without further purification.

### Method Examples

Relative Affinity Assay. The affinity for folate receptors (FRs) relative to folate was determined according to a previously described method (Westerhof, G. R., J. H. Schornagel, et al. (1995) Mol. Pharm. 48: 459-471) with slight modification. Briefly, FR-positive KB cells were heavily seeded into 24-well cell culture plates and allowed to adhere to the plastic for 18 h. Spent incubation media was replaced in designated wells with folate-free RPMI (FFRPMI) supplemented with 100 nM ³H-folic acid in the absence and presence of increasing concentrations of test article or folic acid. Cells were incubated for 60 min at 37° C. and then rinsed 3 times with PBS, pH 7.4. Five hundred microliters of 1% SDS in PBS, pH 7.4, were added per well. Cell lysates were then collected and added to individual vials containing 5 mL of scintillation cocktail, and then counted for radioactivity. Negative control tubes contained only the ³H-folic acid in FFRPMI (no competitor). Positive control tubes contained a final concentration of 1 mM folic acid, and CPMs measured in these samples (representing non-specific binding of label) were subtracted from all samples. Notably, relative affinities were defined as the inverse molar ratio of compound required to displace 50% of ³H-folic acid bound to the FR on KB cells, and the relative affinity of folic acid for the FR was set to 1.

METHOD. In vivo dosing of the animals. KB tumors were induced in female athymic nu/nu mice by subcutaneous injection of 1.0×106 KB cells suspended in cell culture media (folate free RPMI). When the tumors reached appropriate size (about 2 weeks), the mice were divided into different experimental groups. Under anesthesia, the tumor bearing mice were injected intraperitonially with the either of the following: (a) 7.5 or 15 n moles of DY647-folate β-Gal siRNA duplex in 200 μl of PBS; (b) 15 n moles of DY647 13-Gal siRNA duplex in 200 ul of PBS. For competition experiments, 100× molar equivalents of EC89 in PBS was injected i.p 10 minutes prior to the intraperitonial injection of 15 n moles of DY647 β-Gal-folate siRNA. Twenty four hours after the injection of the siRNAs, the animals were euthanized by CO2 inhalation and imaged soon thereafter. After whole body imaging, tumors and major organs were excised and imaged.

METHOD. Imaging protocol. Fluorescence imaging was performed with a Kodak Image Station In-Vivo FX equipped with a CCD camera. DY647 band-pass excitation (625 nm) and emission (700 nm) filters (both Kodak) were used for all the experiments. Identical illumination settings (lamp voltage, exposure time, f-stop, binning) were used for all the imaging experiments.

METHOD: Cell lines. Cell lines were obtained from ATCC. RAW264.7, KB, and GFP HeLa cells were grown as monolayers using folate free 1640 RPMI medium containing 10% heat inactivated fetal bovine serum plus 100 units/ml penicillin and 100 μg/ml streptomycin in a 5% CO_{2:95}% air-humidified atmosphere at 37° C.

### Example

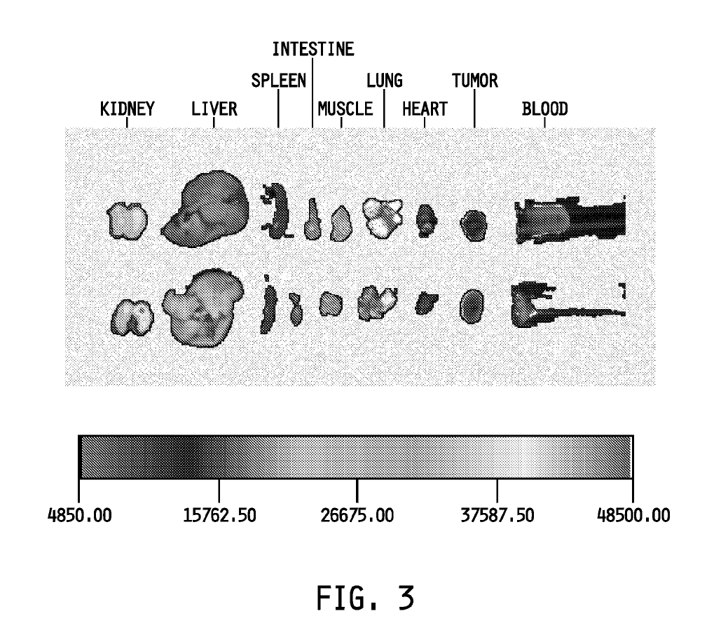

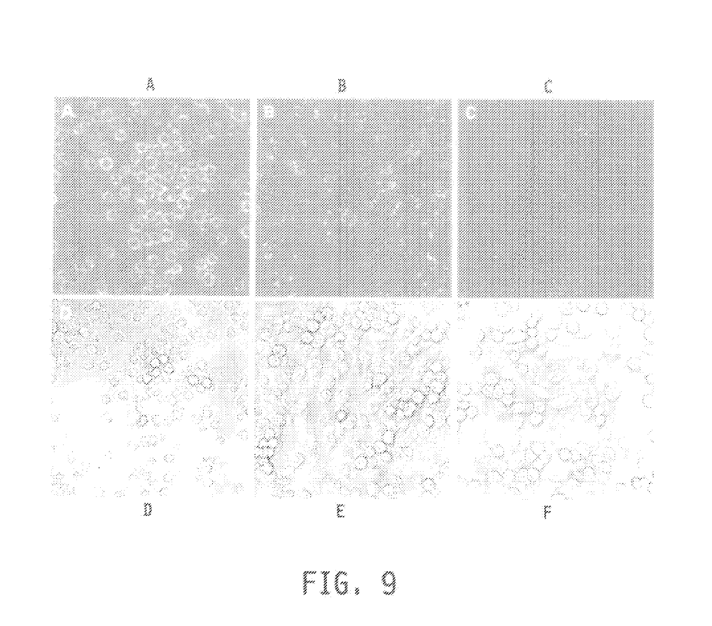

Cellular uptake studies. FR over-expressing RAW264.7 cells were incubated for 1 h with DY647-labeled, folate-conjugated siRNA duplex, a fluorescent form of the folate-siRNA conjugate.Internalization of targeted SiRNA. Uptake of the conjugate is shown in panel A of FIG. 9. the folate targeted siRNA was internalized efficiently by RAW264.7 cells and rapidly trafficked to endosomes. The endosomes are believed to move along microtubules to a recycling center, see FIG. 3. Folate-siRNA uptake and endocytosis was inhibited by addition of 100× free folic acid.

### Example

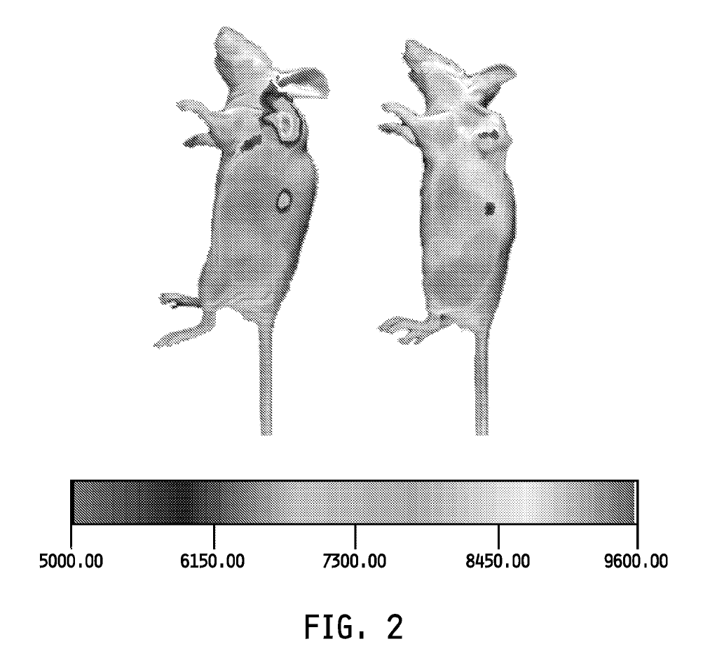

Uptake of double-stranded DNA. RAW264.7 cells were incubated for 2 hours with a Cy5-labeled folate-conjugated 21-mer deoxyriboucleotide duplex with 3′-overhangs. Good uptake of the folate-conjugated oligonucleotide is observed, see panel B of FIG. 2. No significant uptake was seen in the case of the unconjugated, control oligonucleotide, see panel C FIG. 2.

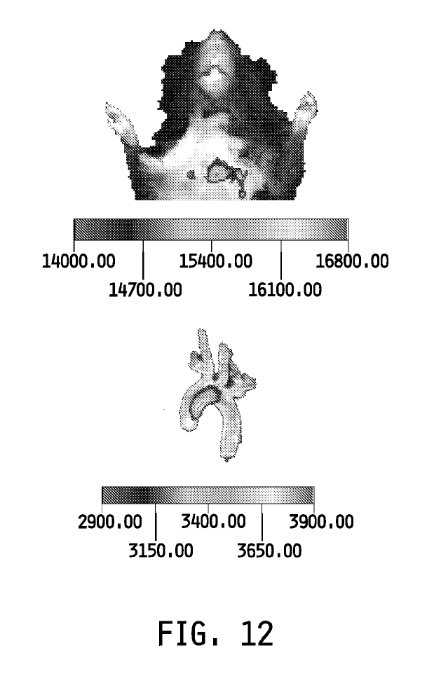

METHOD. An example of an siRNA conjugate is shown in FIG. 11. siRNA targeting to murine atherosclerotic plaque model of heart disease. To generate the mouse atherosclerotic plaque model of heart disease, (ApoE−/−) mice were maintained on western diet. For siRNA targeting 15 nmols of DY647-Folate β-Gal siRNA in 200 μl PBS was injected retroorbitally in to mice under anesthesia. Four hours after the injection of siRNA, the mice were euthanized and imaged using a Kodak Image Station In-Vivo FX equipped with a CCD camera. DY647 band-pass excitation (625 nm) and emission (700 nm) filters (both Kodak) were used for the experiments. After whole body imaging, the aorta were excised and imaged with same excitation and emission parameters for the DY647 fluorophore. The results are displayed in FIG. 12, showing preferential siRNA targeting to murine atherosclerotic plaque.

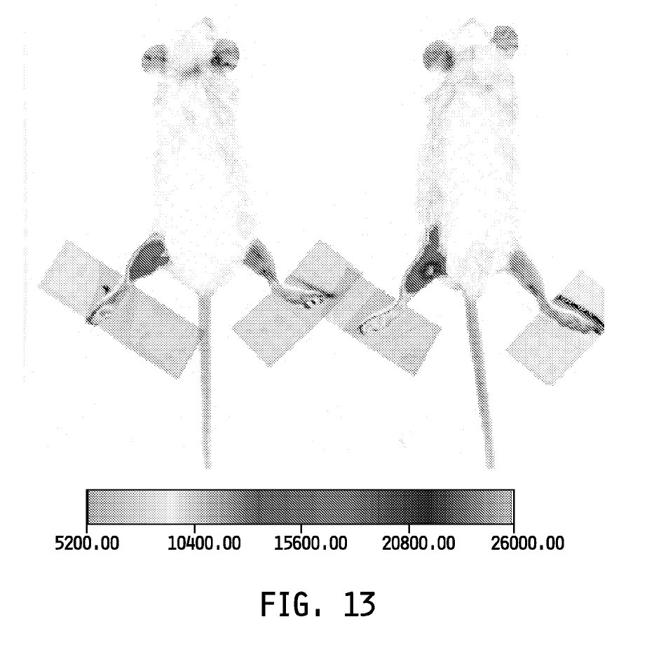

METHOD. SiRNA targeting to murine Muscle Trauma Model. To generate murine skeletal muscle injury model, 100 μl of 10 μM cardiotoxin in PBS from Naja naja mossambica was injected in to the tibialis anterior muscle of male C57BL/6 mice under anesthesia. Fourty eight hours post injection of cardiotoxin, 15 nmols of DY647-Folate β-Gal siRNA in 200 μl PBS was injected retroorbitally in to mice under anesthesia. Four hours after the injection of siRNA, the mice were euthanized and imaged using a Kodak Image Station In-Vivo FX equipped with a CCD camera. DY647 band-pass excitation (625 nm) and emission (700 nm) filters (both Kodak) were used for the experiments. Preferential uptake of folate-conjugate siRNA by the injured muscles (left, in both cases) is evident from the higher mean fluorescence intensity: mean ROI left/right=4.5 (see FIG. 13).

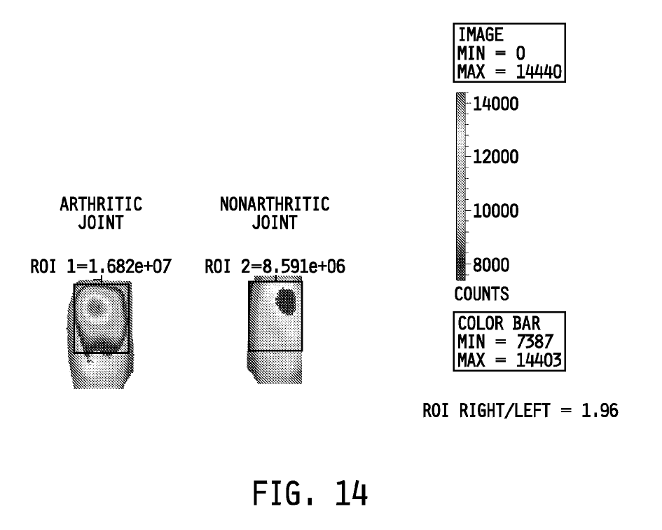

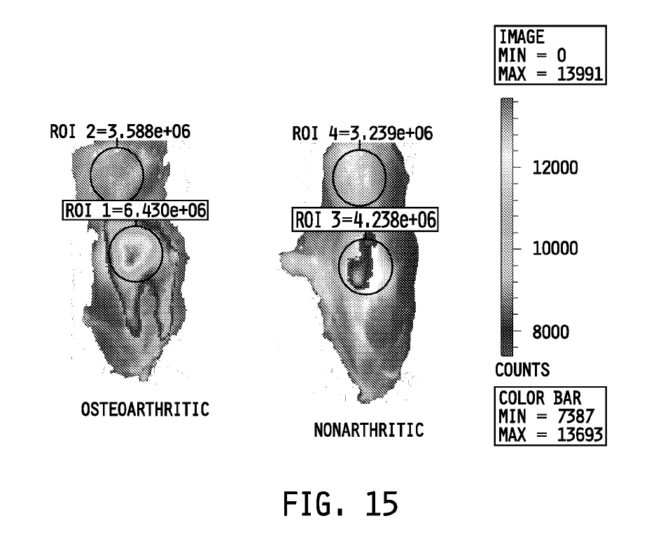

METHOD. siRNA targeting to osteoarthritic joints in the guinea pig model. Two year old male guinea pigs that had developed spontaneous osteoarthritis as evidenced by X-ray imaging were injected intraperitonially with 15 n moles of DY647-Folate β-Gal siRNA in 200 μl PBS under anesthesia. Four hours after injection, the guinea pigs were euthanized and the joints were excised for imaging. Fluorescence images were obtained using a Xenogen Vivo Vision IVIS imager. The excitation and emission filters used were 640 nm and 720 nm respectively. Preferential uptake of folate-conjugate siRNA by the inflamed joint (left) is evident from higher mean fluorescence intensity: ROI left/right=2 (see FIG. 14). Another example of siRNA targeting to osteoarthritic joints is shown in FIG. 15, exhibiting preferential uptake of the folate-siRNA conjugate by the inflamed joint.

1. A compound of the formula
B-L-N
wherein B is a vitamin receptor binding ligand that binds to a vitamin receptor, where the vitamin receptor is overexpressed or selectively expressed on a pathogenic cell, L is a linker that comprises one or more hydrophilic spacer linkers, and N is an oligonucleotide, an iRNA, an siRNA, a microRNA, a ribozyme, an antisense molecule, or an analog or derivative thereof; and wherein L is a chain of atoms selected from the group consisting of C, N, O, S, Si, and P that covalently connects the binding ligand B to N.

2. The compound of claim 1 wherein N is selected from the group consisting of
N comprising about 15 to about 49 bases,
N comprising about 19 to about 25 bases,
N comprising about 15 to about 23 bases,
N comprising about 21 to about 23 bases, and
N comprising a ribonucleotide.

3-6. (canceled)

7. The compound of claim 1 wherein N is double stranded.

8. The compound of claim 7 wherein N is a blunt-ended or wherein N includes an overhang of about 2 to about 3 bases.

9. The compound of claim 1 wherein N is an siRNA.

10. The compound of claim 1 wherein the hydrophilic spacer linker is formed primarily from carbon, hydrogen, and oxygen, and has a carbon/oxygen ratio of about 3:1 or less, or of about 2:1 or less.

11. The compound of claim 1 wherein the hydrophilic spacer linker is formed primarily from carbon, hydrogen, and nitrogen, and has a carbon/nitrogen ratio of about 3:1 or less, or of about 2:1 or less.

12. (canceled)

13. The compound of claim 1 wherein the hydrophilic spacer linker comprises a formula selected from the group consisting of 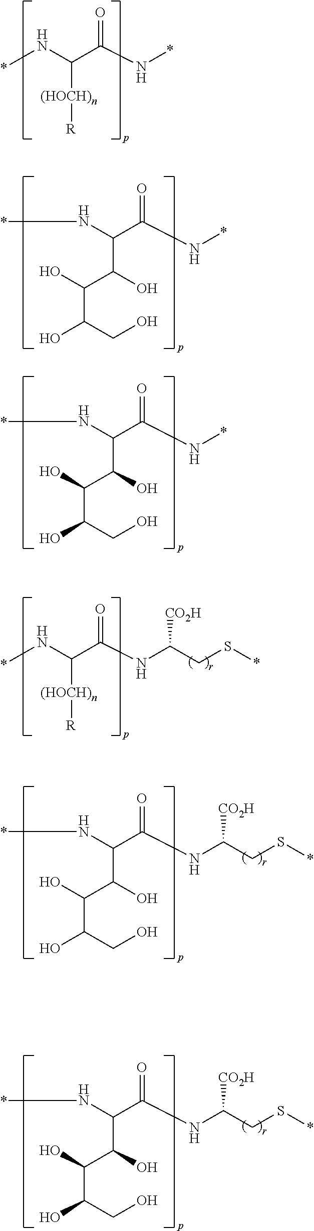 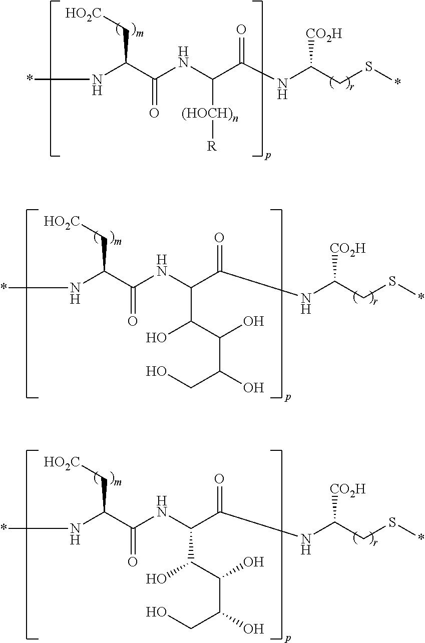
wherein R is H, alkyl, cycloalkyl, or arylalkyl; m is an integer from 1 to about 3; n is an integer from 1 to about 5, p is an integer from 1 to about 5, and r is an integer selected from 1 to about 3.

14-25. (canceled)

26. The compound of claim 1 wherein the linker L further comprises a releasable linker.

27. The compound of claim 1 wherein the linker L further comprises a releasable linker selected from the group consisting of
a disulfide releasable linker
a carbonate releasable linker;
a silyloxy releasable linker;
an acetal or ketal releasable linker;
a succinimid-1-ylalkyl acetal or ketal releasable linker;
a 3-thiosuccinimid-1-ylalkyloxymethyloxy releasable linker, where the methyl is optionally substituted with alkyl or substituted aryl;
a releasable linker comprising an ester-amide of one or more bivalent radicals selected from the group consisting of carbonylarylcarbonyl, carbonyl(carboxyaryl)carbonyl, and carbonyl(biscarboxyaryl)carbonyl; and
an acyl hydrazide or acyl hydrazone releasable linker.

28-39. (canceled)

40. The compound of claim 1 wherein the linker L further comprises a disulfide releasable linker.

41-49. (canceled)

50. The compound of claim 1 wherein the linker L further comprises one or more bivalent radicals selected from the group consisting of carbonyl, thionocarbonyl, alkylene, cycloalkylene, alkylenecycloalkyl, alkylenecarbonyl, cycloalkylenecarbonyl, carbonylalkylcarbonyl, 1-alkylenesuccinimid-3-yl, 1-(carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl, and 1-(carbonyltetrahydrofuranyl)succinimid-3-yl, each of which is optionally substituted with one or more substituents X¹;
wherein each substituent X¹ is independently selected from the group consisting of alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from the group consisting of an amino acid, an amino acid derivative, and a peptide, and wherein R⁶ and R⁷ are each independently selected from the group consisting of an amino acid, an amino acid derivative, and a peptide.

51-55. (canceled)

56. The compound of claim 1 wherein the linker L further comprises at least 2 amino acids selected from the group consisting of asparagine, aspartic acid, cysteine, glutamic acid, lysine, glutamine, arginine, serine, ornitine, and threonine.

57. (canceled)

58. The compound of claim 1 wherein the linker L further comprises a tripeptide, tetrapeptide, pentapeptide, or hexapeptide consisting of amino acids selected from the group consisting of aspartic acid, cysteine, glutamic acid, lysine, arginine, and ornithine, and combinations thereof.

59. A compound comprising a vitamin receptor binding ligand; a linker; and a moiety N; wherein the vitamin receptor binding ligand is covalently attached to the linker; the moiety N is attached to the linker; the linker comprises at least one releasable linker; and wherein the vitamin receptor is overexpressed or selectively expressed on pathogenic cells.

60-71. (canceled)

72. The compound of claim 59 wherein the releasable linker includes a disulfide.

73-90. (canceled)

91. The compound of claim 59 wherein N is an siRNA.

92. The compound of claim wherein the vitamin receptor binding ligand is a folate.

93-108. (canceled)

109. The compound of claim 40 wherein the disulfide is formed with the thiol group of a compound selected from the group consisting of the following compounds:

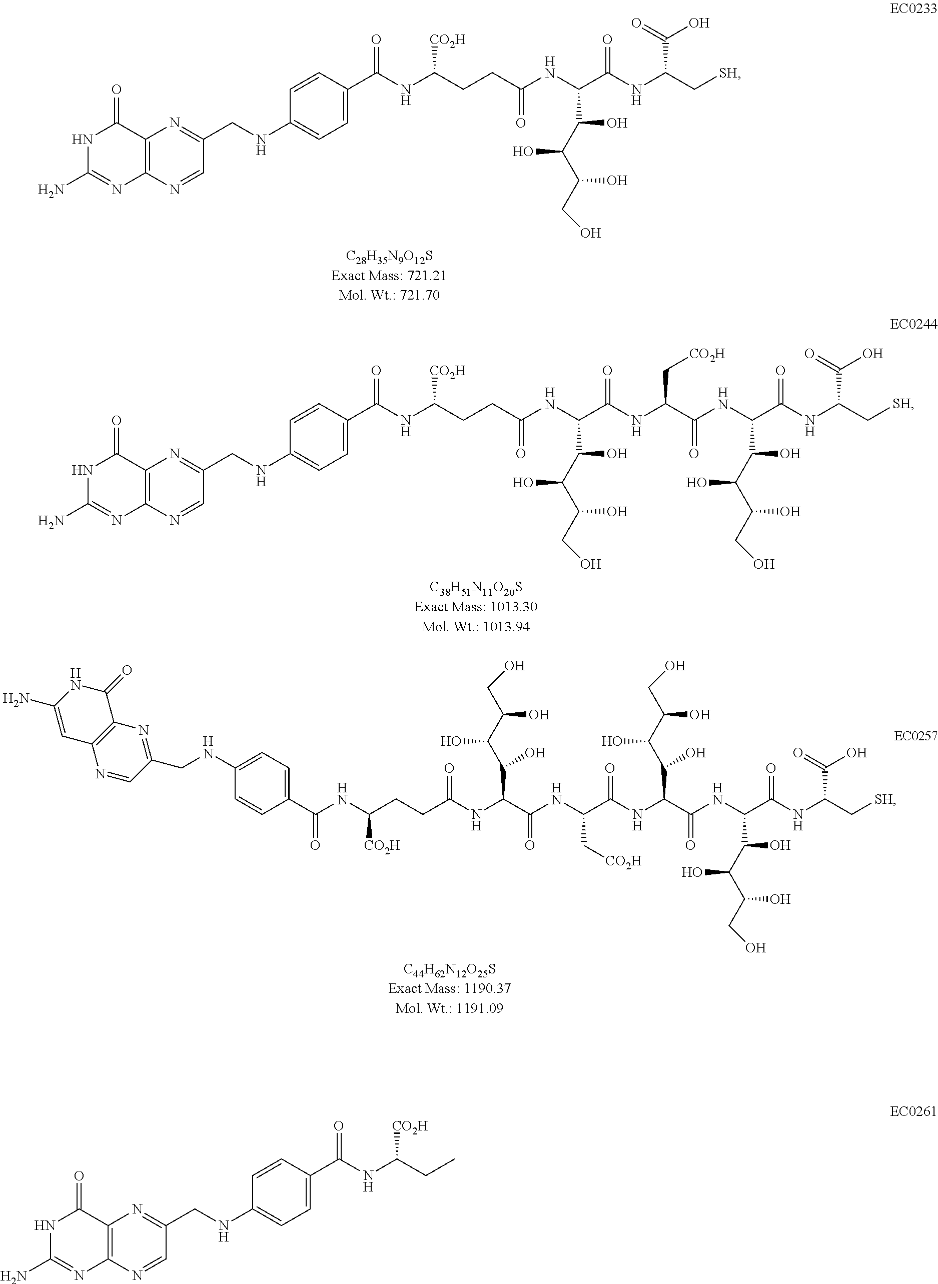

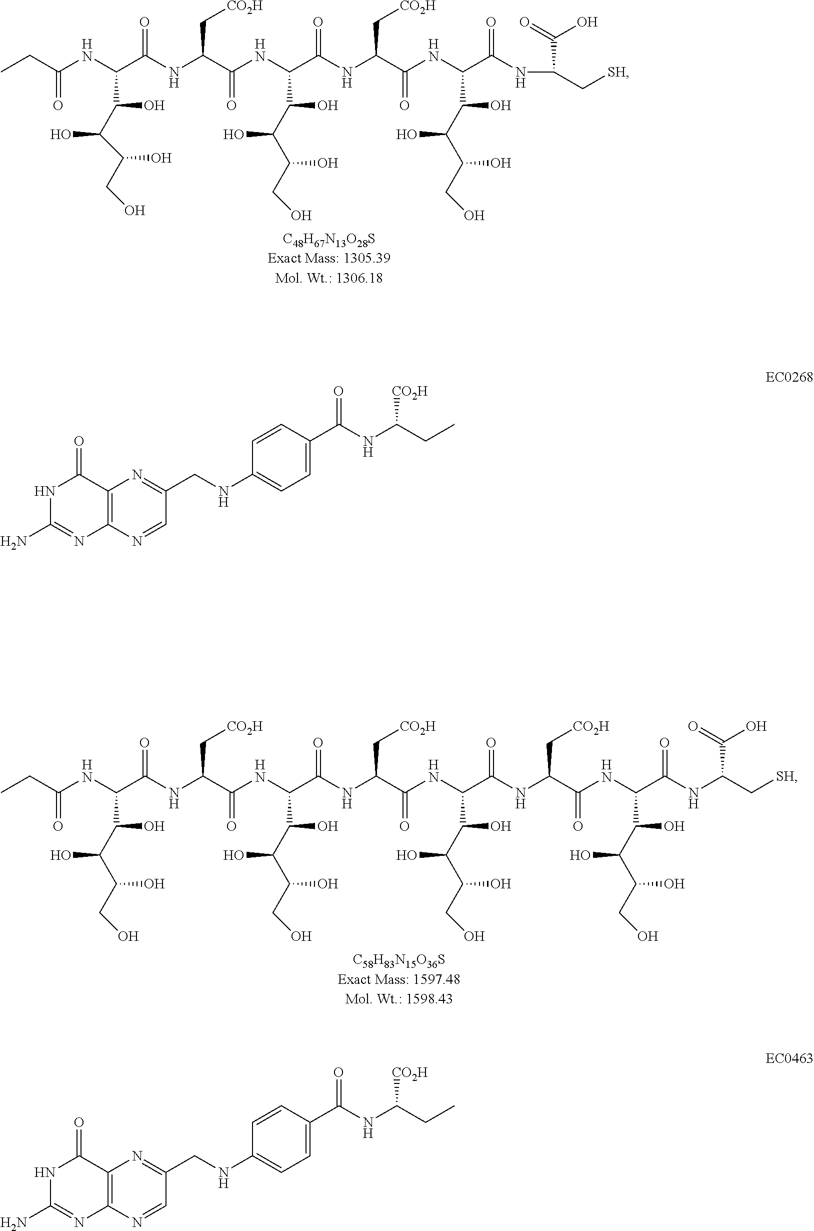

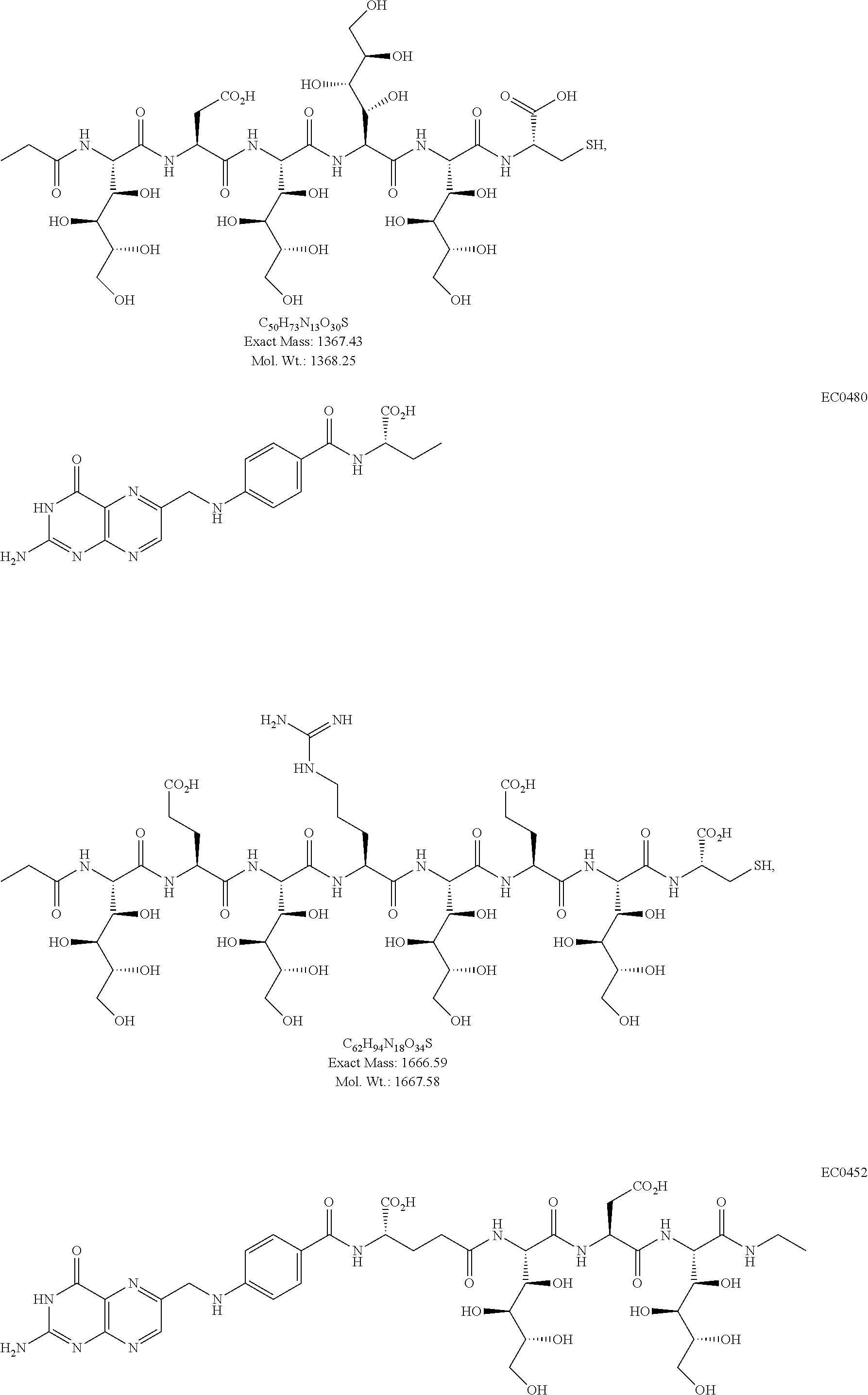

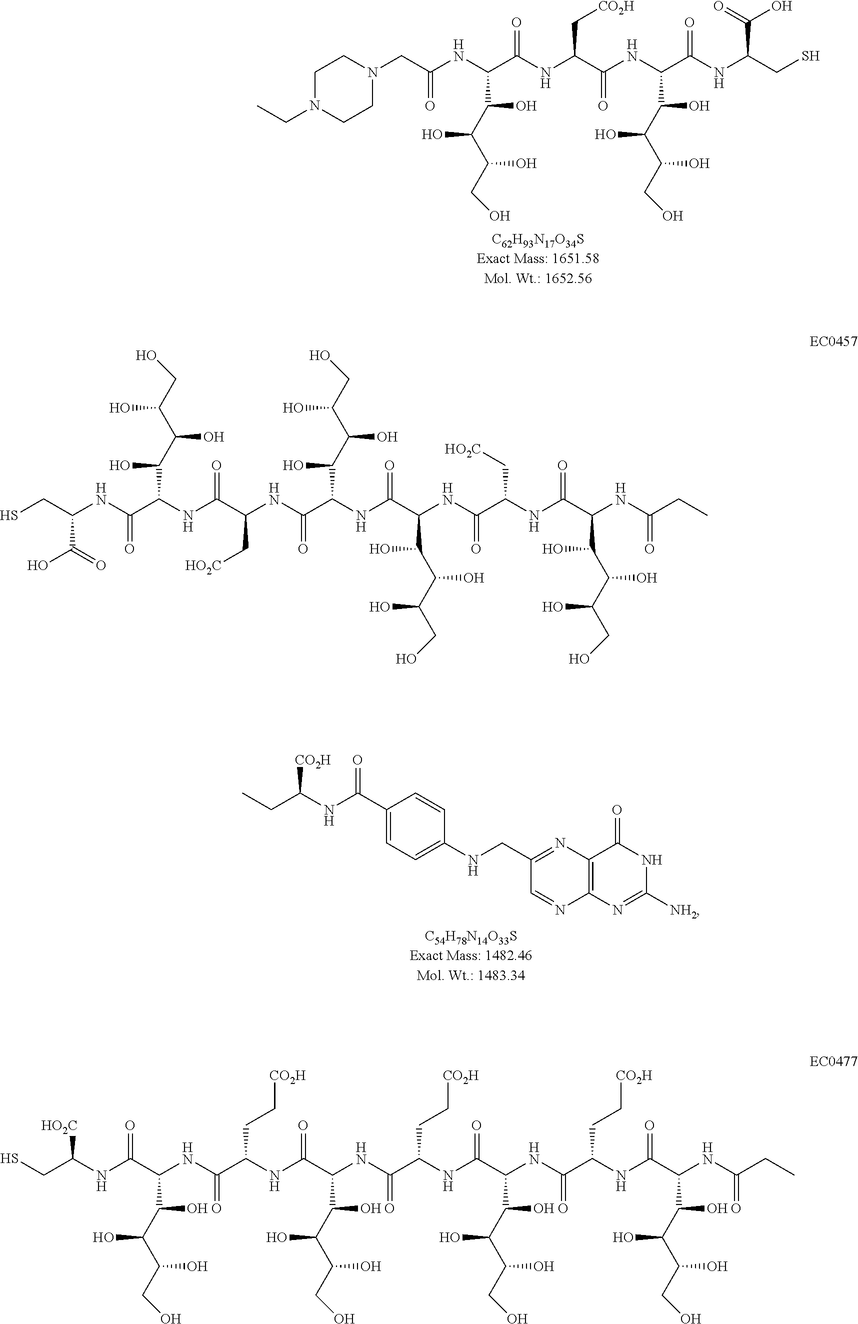

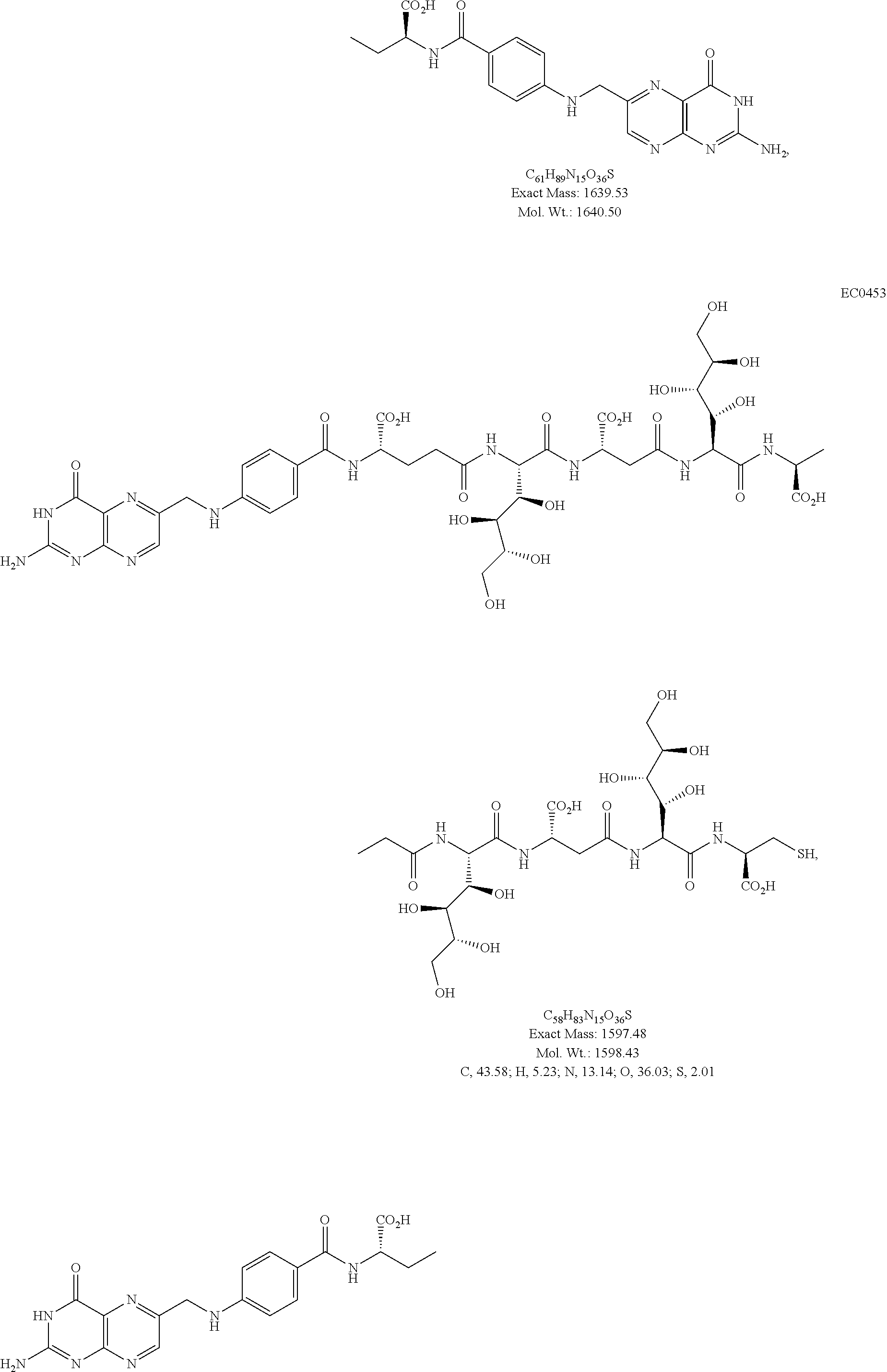

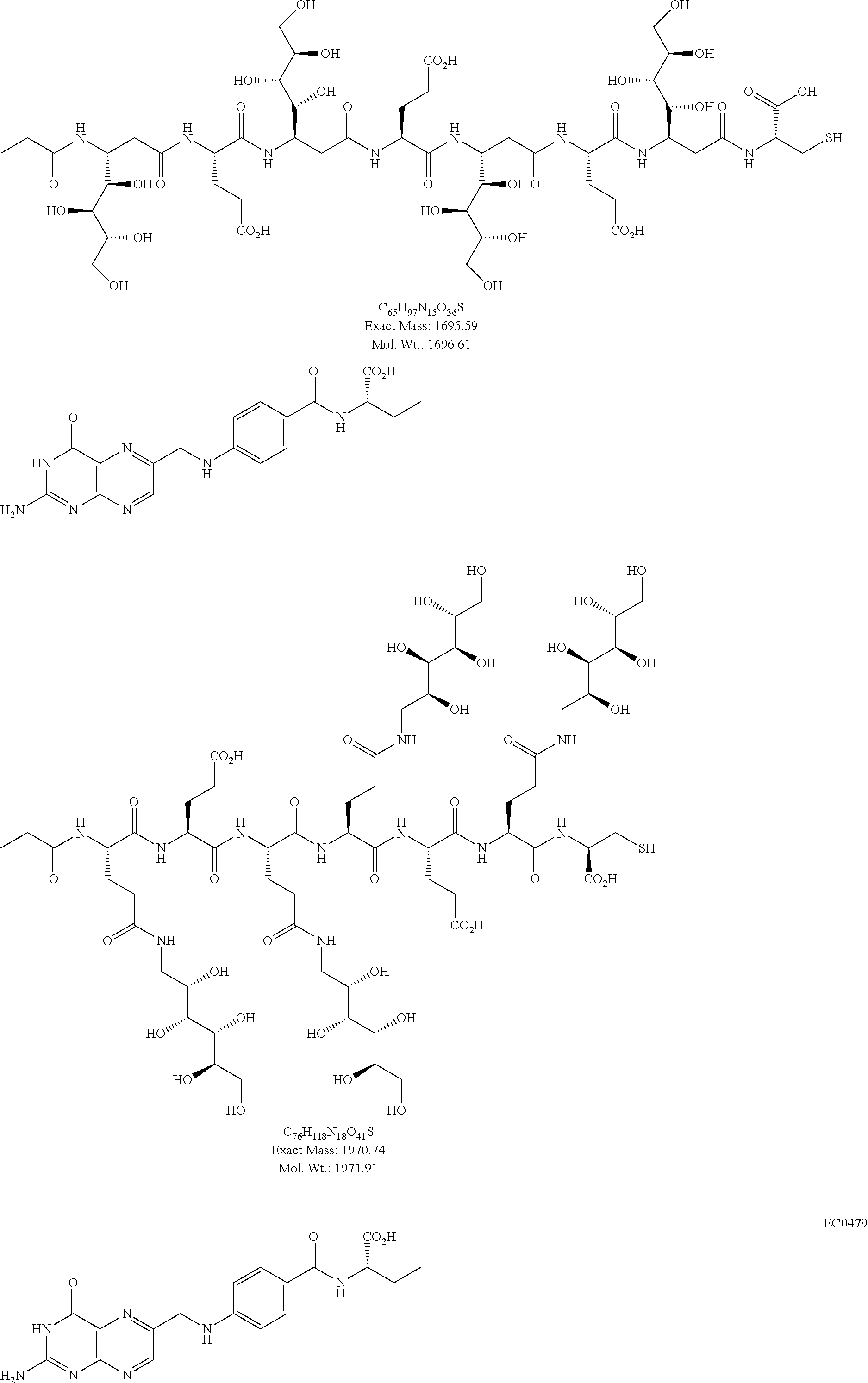

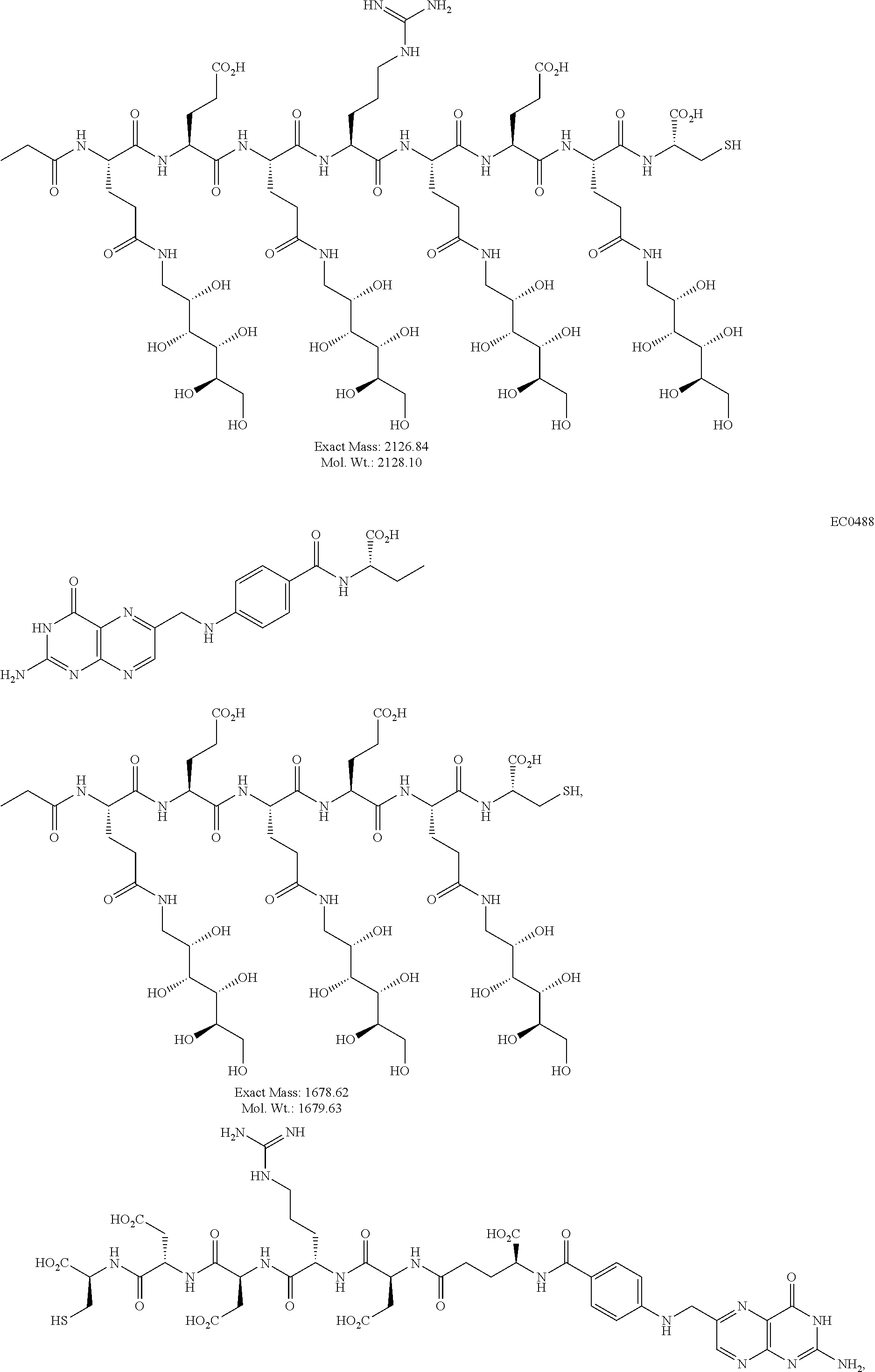

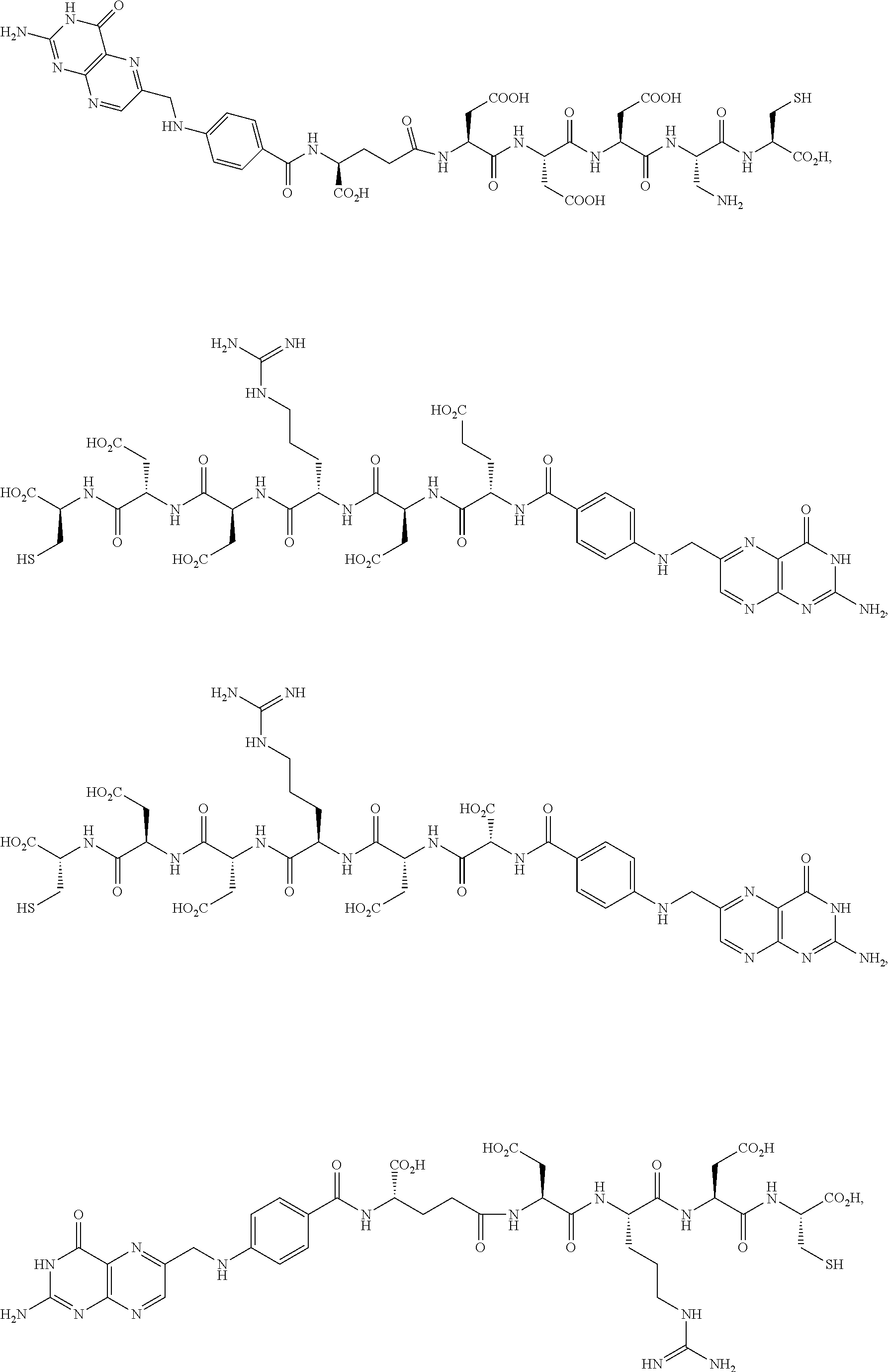

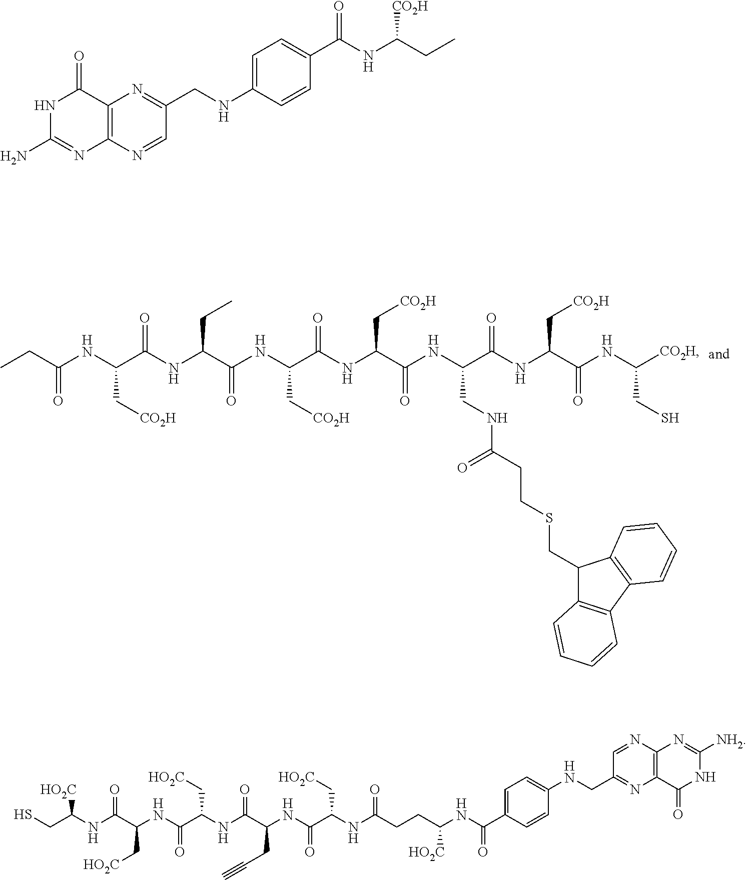

110. The compound of claim 59 wherein the vitamin receptor binding ligand is a folate.
